SEQUENCE LISTING (236 sequences)

SEQ ID NO: 1 - DNA, 510 bp, Zea mays
ggtttcccccaggtccagttagttgttcatggtggagtgataaatatatcagagcatcttttgcctcttcccccagtttttgtcgcacatccctgacagttctgtttgtgcagcccctgatgacgcaacctctaatgataaggaagacaacaagcccgagccttgagatgtcagcaagattgatggttgctaacaatgaccttgtgctgtttcttaccgggttttgacgtgttggatttgtgattaccactgattgctattgtacttcaaacaggaaggctggaaatgcaactcggcttctcttgagaccttgtcatttgctgtagttcgttcgcaactgtatattgtagcttggaagactctgtgccgtggtgcgtgtatttgagaaatttctatgcaaagtgagctggcgataacattggatggcgcagcaaagcatcgcgcgcagtgtttcctaggcatcatccagtgcggctcgtggatcctttatggtcatagctggtccctccc

SEQ ID NO: 2 - DNA, 694 bp, Zea mays
ttgggttaaatctggggtttaaatttgtaagcttaatcaaggataaggggttcaacatccagtcatccagtattgattatggtgatagtatttgcttttgatgagtagaagatgcacgttgatgcatgtatattcaattagtttctgttaaaacattgctacaataggagagtctggaggtagttactgcatctttgcttggtgactccagcttctcatgaccttgctaaactgatatatcttgtttaggtacccgaacttgaagagtgtcagggagttgatctacaagaggggctacggaaaactgaacaagcagaggatccctctgtctaacaaccaagtcatcgaggaggtttgcaatcttgaactctgcacctggatcctttgtgatctgtttgtatttgacaatttacatgatgatctccaccatttggtgttctatcagggcttgggcaagcacaacatcatctgtattgaggatcttgttcacgagatcatgactgttggcccacacttcaaggaggcgaacaacttcctttggccatttaagctgaaggcaccgctgggaggtctgaagaagaagaggaaccactatgtggagggtggtgatgccggtaaccgtgagaattacatcaacgagctcatcaaaaggatgaattagttcacgatcaagctctatgactttccgtaaata

SEQ ID NO: 3 - DNA, 532 bp, Zea mays
tatatttytttttttctaaggttgattggataaaagggggatgcaggtcttgacagcaatgggttgcactcactaaggagaacagtggcagggcatcaccaactaaacagcaccagcagtataagaagaagcctttgctgaagagattcggtggtctgctaaaaaagaaaagcgaaaattagcataaaaccgtctgatgatattctttgttctatcatttgacatttctttgattagatatctagttcccgagtcttcccccatattatggtaaactaagtgatggatgcttcaaagaatacaaaatgtcgactttatttacataattgcctctcttgagttagggagtgttcgcagttcagttcagctgtctggtgtgagctgtcggaaaacagttgtgagctgcctgctgtgaaaaactgttgtgagtaaactaaaagaaagtctttggttggagcttcggtaaaacattagattttttatgatttatctgtattgcttctgagattgttatggtcaacctgtcccttcc

SEQ ID NO: 4 - DNA, 522 bp, Zea mays
aaagacccatagaacgttgtgctgtgggagctcataactggcatgctcccttttgctaatatgacagcagtgcaggctgctttcgctgtggtgaacaagggtgtccgcccagctataccccaggactgcctgcccacccttgctgagatcatgaccaggtgctgggatccaaatcctgatgtccgtccgccattcactgaagttgtgaggatgctggagcatgctgagatggagatcctgagcactgtccgcaaggcccgatttcggtgttgcatgtcccaaccgatgactaccgactgaatcaaacaagagagttgaaatgaactccatggaagcgtaattgagtgtatttatcatgtgtccaaacttcgctcagctgaagtagaaagcacacctgagtttatggctgtatgtgtgtatactcaggtgtaagccttgttgtctttgaaatattcctgcacttagaatatacctagttcgcgttttcagactcttgagatgttttaggctatctattcctga

SEQ ID NO: 5 - DNA, 652 bp, Zea mays
tttttttacccctgccctttataccccgaatcgagcagatagctgatctgattggggatgctggcaacacagtatgctccggaacttggtccaccatagctgagcttttctttaatcgttgattattatgaccgtaacttccgttatctcaattttctatctgaagtttgagctcctgaaaaattttaggaacgacaagatgtctataagccggcaataatttttcgtatcctgtagggatcatcaaggctcacattctgtatcggtggaccatacggtctcgggttacaagtgcgagagcgtgcagatgcaacgattaggctgtcctcactagttttgaaccatcaagttgccttgatagtcctcatggagcagctctacaggtaagcaattagcctatctgatgctgtttctgcacttaccacagtttctgtggagcatacccctttatggctgtatggcctatggtatgagaaaggcacaatgtaacactaccatttaaataccttctttcaactatgaccggtcactagtgacaattgcattaattttgcagggcatggactataataaagggacagaagtatcaccattaggcctaagtgctatcatcattcgctgctgtttaggacttggaaaatggaaaactgcc

SEQ ID NO: 6 - DNA, 159 bp, Zea mays
  misc_feature (62)..(62): n is unknown
gccggcaagatcgagaaygtccccgccccggccatcgccatcgactastggcgcctccccgntaacgccacgctcaaggacgtncgcgccgacgaggctcaccaccgcgacgtcaaccactttgcatcggtacggrtacttccraattccaataccagc

SEQ ID NO: 7 - DNA, 280 bp, Zea mays
ccgatgcacaacagaaagaggaaagctgatgacaagaaacagcaacatcaaagaatgtctgagaagtcaagaacrggaatttcgagcatccatgaactgctgcaggatttcctggtgcagcaacagcacattgatgtccggtggcgggagatgatcgagagacgygcccaggagcgggtggttttygaacaacaatggyggctgacaatgcagaggctggagcaggagcggttgttgctggaacactcstggatggaacgggaggagcgaagaaggatga

SEQ ID NO: 8 - DNA, 480 bp, Zea mays
ggaaaatcccagtcagaacgctgaatctggagtttctgtcaaaacgaaactgaagcgacctggtggtgactggtcatctcgggagtctgacgacaaggacgatgatggtgaagaaagtgatgatgagaagccgatgcacaacagaaagaggaaagctgatgacaagaaacagcaacatcaaagaatgtctgagaagtcaagaacaggaatttcgagcatccatgaactgctgcaggatttcctggtgcagcaacagcacattgatgtccggtggcgggagatgatcgagagacgtgcccaggagcgggtggttttcgaacaacaatggcggctgacaatgcagaggctggagcaggagcggttgttgctggaacactcgtggatggaacgggaggagcgaagaaggatgagagaagaagcacgagctgaaaaaaggatgcactcctgaccactctgtgaacaaactcctgcagaatatta

SEQ ID NO: 9 - DNA, 643 bp, Zea mays
cacatactcacatttcaggcacgtcttcgctacatctaaccctgtaccaacaaaccaaaggtattgccacctaagaccttgtttgtttacaccaatccagctctggattagaatggattggaattaaatccatgtcccaaataaaccaagcctactcaattttttttatttggctaaacccatcatgaattataacccaagggtttatgatttttttaaactatggaaggtatggattctatccataactcattaggtatggaacaaatccatgaagatattgcacaagtttatattagaactgaaactgaaaggcaatataggcatatagcactatagcagaactgaaactgaaatattgaatacaaggctacaatcagtaatgcagtacctactacctagagcatatcatcatccaagcaaaaagcagcagcagcttctccaacatattcagattcatcagaattcagacaataggaaagataggaaagggggagaaggggggaaccttgagatgaggagctcatctcgtcgctagtgttctggagccgccgccggtgttctggagctactgctggtgttctaaagccgcagcctgtgttctggactcggcaaaggggaaaaatttcaagggttaaaaggg

SEQ ID NO: 10 - DNA, 694 bp, Zea mays
gacccctaattttctcgtgactttgaatttgtgggacctatgaatttgtgcaggaactgggggaggagggtgacttcgtttctatttggggaacactgctcgcgtcctgcaaagcacaggacaagcaggaattggtgaatttggtgacagagagattgatctgcattgagaagaagtatggccatgctggttacagcgttttgttgtcacatatttttgctgctgagggcaactggagtagtgctgatagcctgaggaaggagatgaggttgagaggattgagcaagatggcaggttctagttggattaaagtccagcatgcagcattgcaaagctaccctaaaaatggccatgaacactcattactgcatgtagttgattacggtagagatgaaatcatctgacatgaatcagtactgcagcagtggaaagcttgctgatctggtgtttgttcatgtcccatgacgtgatcagctcaggctatgacagattggccttttggttatctgcagcattgacaccttgtcaccttgacgaaattggggcatttcggaacatttacatatatatgaacaacaaactgaactcccgcactactcgtaagcggtgaaataaccctgcaggttaaaaccctgatggcctggacctggatgcagtcatgcaggaaggatatcgcattagttgaatacttaaa

SEQ ID NO: 11 - DNA, 710 bp, Zea mays
ttaagggccggaaggcaagccaaggggttgttgttcgaaacggtggcggccgtgccgggcatggtgggcggcatgttcttcaacctgggttcgttccgccgtttcgagcacagcggcggctggatccgcgcgctgctcgaggaggccgagaacgagcgcatgcacctcatgacgttcctcgaggtcacgcagccgcgctggtgggagcgcgcgctcgtgctcaccgcgcagggcgtcttcttcaacgcctacttcgtcggctacctcctctcccccaagttcgcgcaccgcgtcgtcggctacctcgaggaggaggcagtgcactcgtacaccgagtacctcaaggacctcgaggccggcatcatcgacaacaccccggcgccggccatcgccatcgactactggcgcctccccgccgacgccaagctcaaggacgtcgtcaccgtcgtgcgcgccgacgaggcgcaccaccgcgacgtcaaccacttcgcgtcggtacgcactctgcaccttgcaacaggattcattgctgtgagcaatctccagcagttctagctaattcattggtttatgtttgcttaatggagtacattattttgcaggacatccattaccaggggatgaagctcaaggacacgcccgcaccgctcagttatcactgacaagtaggcgttgcctgcctgctgctcaattcggaagttggttaaaaa

SEQ ID NO: 12 - DNA, 423 bp, Zea mays
tagggtacatggaccctgrgtacttccagacaagccaactgactgagaagagtgatgtstacagctttggcgtcgtactcatcgagctactgacaagraagaagcctatcatggatgatatcrcggaagacattagaagcctagcgctgcaatttagtatgctattccatggaartaagctgttggaaatcgttgatcctgtagtagctgaagaagctggagtcagacatgttgaaacggtttcgaagttggcgttacgatgcttaaggttgaaaggggaagaacgcccaaggatgatagatgttgcgattgaacttgaagcactgagaaggctgatgaaacaacacttcatcttgaagaacgagtctttgcttcaggagtyatgttgcaatgaagaaatgagcatcgacgcaccatcaagtt

SEQ ID NO: 13 - DNA, 423 bp, Zea mays; identical to SEQ ID NO: 12

SEQ ID NO: 14 - DNA, 503 bp, Zea mays
aaaggggaaggtcccagtcacaacgtacattagtgcaagggacactagggtacatggaccctgagtacttccagacaagccaactgactgagaagagtgatgtgtacagctttggcgtcgtactcatcgagctactgacaaggaagaagcctatcatggatgatatcacggaagacattagaagcctagcgctgcaatttagtatgctattccatggaaataagctgttggaaatcgttgatcctgtagtagctgaagaagctggagtcagacatgttgaaacggtttcgaagttggcgttacgatgcttaaggttgaaaggggaagaacgcccaaggatgatagatgttgcgattgaacttgaagcactgagaaggctgatgaaacaacacttcatcttgaagaacgagtctttgcttcaggagtcatgttgcaatgaagaaatgagcatcgacgcaccatcaagtttgttccttgcgttaatgcatttacttttcggtata

SEQ ID NO: 15 - DNA, 613 bp, Zea mays
  misc_feature (2)..(4): n is unknown
annnggcttnacgacttacccatacctcgttacaccgccgccgccgtcaccgtaccaacctactcgtacccgccgccgccgcagccgcagccgcggccacaccagcaagcagaactagcagccatgccgcccaaattggacccctctcaggtggtggaggtcttcgtccgcgtgacgggaggcgaggtcggcgcggcgtcgtcgctggcccccaagatcggcccgctcggtctctccccgaagaagatcggcgaggacatcgccaaggagaccgccaaggactggaagggcctccgcgtcaccgtcaagctcaccgtgcagaaccggcaggccaaggtctccgtcgtcccctccgccgcggcgctcgtcatcaaggcgctcaaggaacccgagagggacaggaagaaggtcaagaacatcaagcacagcggcaacatcagcctcgacgacgtcatcgagatcgccaagaccatgcggaacaggtccatggccaaggagttggccgggactgtcaaggagatcctggggacctgcgtcagcgtcgggtgcactgtcgatgggaaggaccccaaggacttgcagcaggagatcgatatggtcatagcttgctctt

SEQ ID NO: 16 - DNA, 469 bp, Zea mays
  misc_feature (306)..(306): n is unknown
tgacaagctgcagcgaagaaggtgaacctagctgatattggcatcgtcggtggccttggcgatgggtccgatgagaagrcmctgccctcttggaccatgggcgccgkatccggcctaggaatgtctggtattccaccgtcaacacaacaagctggtggcatcgagagcttggccaactacaacaagcatcatttcggcttcaaataggcctcgatctttcatactggaaaatacccgtcatctgcggtttcctcctcwgtcggcctgcttcttacaygtgctgccctattgatttaatcacttttntttgnttttntggttnnttnnggtgatyacattacatggtrtcgaccaatcttggccccgtcttgtcacrcgtgtatgttatttgtcgggtttgtggntaagcatgcaactacannnncatcacacccccnnktgttccagytcgatrataggtggyatgttg

SEQ ID NO: 17 - DNA, 469 bp, Zea mays; identical to SEQ ID NO: 16
  misc_feature (306)..(306): n is unknown

SEQ ID NO: 18 - DNA, 704 bp, Zea mays
ccaatcagagagcctagggcaaagaaagacaattttcaggtcaagtccggcatatgggcaaactcgctgagccggggattgattgatctgaacataactgcacgtatgttcctcgttcctgtttcccctgttgccatggcatctatcagcttggtgtaaatttgtttatggttcagacatgttcatggttctccttgtttctgacaagctgcagcgaagaaggtgaacctagctgatattggcatcgtcggtggccttggcgatgggtccgatgagaaggccctgccctcttggaccatgggcgccggatccggcctaggaatgtctggtattccaccgtcaacacaacaagctggtggcatcgagagcttggccaactacaacaagcatcatttcggcttcaaataggcctcgatctttcatactggaaaatacccgtcatctgcggtttcctcctctgtcggcctgcttcttacatgtgctgccctattgatttaatcactttttttgtttttggttttggtgattacattacatggtgtcgaccaatcttggccccgtcttgtcacgcgtgtatgttatttgtcgggtttgtgggtaagcatgcaactacatacacatcacaccccctgtgttccagctcgatgataggtggtatgttggccatgcagtttgtgaaattccggccgaacttggttatttaa

SEQ ID NO: 19 - DNA, 406 bp, Zea mays
  misc_feature (123)..(126): n is unknown
cagcaacgttagcgacctggaagccacggagtggatcgaggargacgagccgggcgtgtgcctcaccatccgcgagctcggcgayggcacycgcgarctccgccgcatccggttcaggtatgnnnngcactgaycagtcatggacatgcggaagcatacatcactggytcagtnnnaaccaaaatccttcttgatcactcggttcattcatgtgatcatgtctgttccatgtttctgtgrtgctgcagccgggagatattcggcgaggatagggccaaggtgtggtgggagcagaacagggagagaatacaggcggaatatctgtagcaagcgatcagacactgagctgatgcaattttcaggcctgatgggataatcaaatatgtttgtgagaggatagattagg

SEQ ID NO: 20 - DNA, 372 bp, Zea mays
  misc_feature (153)..(155): n is unknown
gccacggagtggatcgaggargacgagccgggcgtgtgcctcaccatccgcgagctcggcgayggcacycgcgarctccgccgcatccggttcaggtatgcatggcactgatcagtcatggacatgcggaagcatacatcactggctcagtannnaccaaaatccttcttgatcactcggttcattcatgtgatcatgtctgttccatgtttctgtgrtgctgcagccgggagatattcggcgaggatagggccaaggtgtggtgggagcagaacagggagagaatacaggcggaatatctgtagcaagcgatcagacactgagctgatgcaattttcaggcctgatgggataatcaaatatgtttgtgaga

SEQ ID NO: 21 - DNA, 582 bp, Zea mays
ccaagtcaaaaagctcaactccgctgctgcggcggccggcacgtcagctgcgatgggcggcggggccccgtcgtcctacgacccgtcgcgcgccaccacgtcgtccagggacgaggcctccgtgtccctcagcaacgttagcgacctggaagccacggagtggatcgaggaggacgagccgggcgtgtgcctcaccatccgcgagctcggcgacggcactcgcgagctccgccgcatccggttcaggtatgcatggcactgatcagtcatggacatgcggaagcatacatcactggctcagtagtaaccaaaatccttcttgatcactcggttcattcatgtgatcatgtctgttccatgtttctgtgatgctgcagccgggagatattcggcgaggatagggccaaggtgtggtgggagcagaacagggagagaatacaggcggaatatctgtagcaagcgatcagacactgagctgatgcaattttcaggcctgatgggataatcaaatatgtttgtgagaggatagattagggagatgataccgctatatacatgtaaaaccactaatttggtaaaaaa

SEQ ID NO: 22 - DNA, 171 bp, Zea mays
  misc_feature (62)..(62): n is unknown
gccggcaagatcgagaaygtccccgccccggccatcgccatcgactastggcgcctccccgntaacgccacgctcaaggacgtmgtcaccgtcgtscgcgccgacgaggctcaccaccgcgacgtcaaccactttgcatcggtacggrtacttccraattccaataccagc

SEQ ID NO: 23 - DNA, 171 bp, Zea mays; identical to SEQ ID NO: 22
  misc_feature (62)..(62): n is unknown

SEQ ID NO: 24 - DNA, 171 bp, Zea mays; identical to SEQ ID NO: 22
  misc_feature (62)..(62): n is unknown

SEQ ID NO: 25 - DNA, 557 bp, Zea mays
ggatwaggggagggtcccagtcacgacgatccactcgtacaccgagtacctcaaggatctggaggccggcaagatcgagaacgtccccgccccggccatcgccatcgactactggcgcctccccgctaacgccacgctcaaggacgtagtcaccgtcgtccgcgccgacgaggctcaccaccgcgacgtcaaccactttgcatcggtacggatacttccgaattccaataccagcagcaactctgcttgatctcgctcgccgggcacgcgtatctcgttatgggattggttctgaaatctgaattggtatgagcttgtgccgtgcaggacatccattgccagggaatgcagctgaagcagtcccctgcgccgatcggataccactgaggaagtgatgctgtttgtgctcttcttaattttgcatcgctaataagcaaatgagtgtcttgtctttaagggaaggaaaggatgcttattgagttacgagtactgctacggcgattaggaggatattttccaaacccagtttttggggaaatttgtaagtaataaggtta

SEQ ID NO: 26 - DNA, 794 bp, Zea mays
  misc_feature (307)..(699): n is unknown
attgaaaygcatttgagtgatgagagtattaccttttgggagaagtttgagtcgtttcaagtctttatgcatgaccaggtgagttagtrgttttactttttwcctcaatgccgtgtgaatgtgaggttcatatawttttttmtgctaatctttgtagaaggactcaagggttattattctattccttgaaagtcttctttcttggcttgagcgtcgagaccytccagaaaatatggatgttcaattattcgtagagatcaggcacatatgcagtcaatttcaagagaagtatcttaggtatgtttcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagcatttatgawacnaattagtagttgggtattagagtgtgatgtttaactatcagcatccttctgttgatgcatgaagtattcttgtaaaagtt

SEQ ID NO: 27 - DNA, 810 bp, Zea mays
  misc_feature (89)..(89): n is either ggttttactttttt or agttttacttttta
attgaaaygcatttgagtgatgagagtattaccttttgggagaagtttgagtcgtttcaagtctttatgcatgaccaggtgagttagtncctcaatgccgtgtgaatgtgaggttcatatawttttttmtgctaatctttgtagaaggactcaagggttattattctattccttgaaagtcttctttcttggcttgagcgtcgagaccytccagaaaatatggatgttcaattattcgtagagatcaggcacatatgcagtcaatttcaagagaagtatcttaggtatgtttcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagcatttatgawacygtwctggtattatartgcacstactctnnaattagtagttgggtattagagtgtgatgtttaactatcagcatccttctgttgatgcatgaagtattcttgtaaaagtt

SEQ ID NO: 28 - DNA, 823 bp, Zea mays
  misc_feature (307)..(699): n is unknown
attgaaaygcatttgagtgatgagagtattaccttttgggagaagtttgagtcgtttcaagtctttatgcatgaccaggtgagttagtrgttttactttttwcctcaatgccgtgtgaatgtgaggttcatatawttttttmtgctaatctttgtagaaggactcaagggttattattctattccttgaaagtcttctttcttggcttgagcgtcgagaccytccagaaaatatggatgttcaattattcgtagagatcaggcacatatgcagtcaatttcaagagaagtatcttaggtatgtttcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagcatttatgawacygtwctggtattatartgcacstactctnnaattagtagttgggtattagagtgtgatgtttaactatcagcatccttctgttgatgcatgaagtattcttgtaaaagtt

SEQ ID NO: 29 - DNA, 583 bp, Zea mays
gagatcaggcacatatgcagtcaatttcaagagaagtatcttaggtatgtttctgtcaagggcgttaaacccttggctggctggaccgcggctacggagctcgtagccgtcggccgtcttctccggtgaggttattcccctctaccgtaggctttagaaaataagagagagagatgaggctaataatctgcttgctttaatggtcctggttacaagaatatatagggccatagcccaactaactggagtaacaaactcctgaaattatggaactgataactcctaaatccatctgccttatctactcgatcagctcgagccagctgtgcgcgcgtcccccctggccgcacgttcctctgctcggcgtacgtccctggccctgcgcctcctagtgtagacttgtccccacatgacagtttcagcatttatgaaaccgttctggtattatagtgcacgtactctctaattagtagttgggtattagagtgtgatgtttaactatcagcatccttctgttgatgcatgaagtattcttgtaaaagttttcctgcaaaatcagtaattagtatcaatttagggttaa

SEQ ID NO: 30 - DNA, 669 bp, Zea mays
gtggaacgtctcaaaggtaacagctactatgttcaaaatggaactgaggactagtggatgaatccagctgtgtatcttcacatgaagagactaacgccccgtttggatccttggaattgaatttcattctaaaaatcataatttagtcacaaatcaatttaagttaatatggttttatacggaatctatttgtgtaccctattagccatatggggtacatatttatatgctagaattctattatagagtagcgagtcaaagagtgtgttataaattgtagagtagaaacatagcctggagatacataaaatcaatttccatccctccactctatgaatttgagatagacttatatttgaactttggaaagtggtaggatgttaaattccaagctaaatagactactctattaagtaaatttcgattcctccaaaatgaagggatccaaactgcccctaatagaattttgtttctggctatttacatttttaaagttgtggttccgttcaggacttcgcccatatacgttttggtttgtgctttgaccttttagttgtgaacttgtgattatttcatttatgggattagaattattattattaaataacgagattgaataacaagagccatgacttgattgaatttcataagcgctagcaaattatttgg

SEQ ID NO: 31 - DNA, 359 bp, Zea mays
  misc_feature (30)..(30): n is unknown
tagttttyccctcttcctaacgtgtttaanctcttataagagtgtgtntncatggtccgacaaacttttgcttcycctcatgagatsatttgtgcycttatctctgcagataaagggcatcagtgagctcctygagggccttattccgtattcgcagaggcatttcagcagagtggacagactagtccgaagcacgtttctgttggactatacgctgaygcgaatgtccgtggtagayccagatgtggatgcggggtcaatcaaagacgaaatgaatggttcgtctgtggagaacggtgaacttgcagagcctcggcctgcttcacctgtgccagagaagtcaagcaagaagagaaaat

SEQ ID NO: 32 - DNA, 591 bp, Zea mays
tcatgtttcagttgtgcttttcgggtattgaggagtttctctcccactgatatcctggaggtagtagtttttccctcttcctaacgtgtttaaactcttataagagtgtgttcatggtccgacaaacttttgcttctcctcatgagatgatttgtgctcttatctctgcagataaagggcatcagtgagctccttgagggccttattccgtattcgcagaggcatttcagcagagtggacagactagtccgaagcacgtttctgttggactatacgctgacgcgaatgtccgtggtagacccagatgtggatgcggggtcaatcaaagacgaaatgaatggttcgtctgtggagaacggtgaacttgcagagcctcggcctgcttcacctgtgccagagaagtcaagcaagaagagaaaatctggcaaatcaagtaaaaagggaaaggagaaggtgatgaagcttgcctcgagtggacttggcaagggtgtttctgttgaagcctgaaaatctagctgagaatctggttttgcttatgcatgcatgcatccaattttgtagcagctgttgaaactgactttctaacatggt

SEQ ID NO: 33 - DNA, 298 bp, Zea mays
  misc_feature (188)..(188): n is either gtacgaagtcgatcg or atacgaagtcgatca
tgttgccaggaactcctggatcagaaactttcagagagaagttcatcaaggtcgatgacgaaaactacatcaaggagacggtggtcactgaaggaggccttctggatcacggctttcggaagtacatggttcgaatcgagatcgtgggwagagaagagaagrcatccatcgtaaggtcracaattcantgagcatgcaggttcacacgcaccccctgtgttcagtaccgatgggytrgctaccattgccgaggccatcaccaagyatatcaaggagmagagaggctctgagtccgtaa

SEQ ID NO: 34 - DNA, 669 bp, Zea mays
tcaggaaaatttgccatgagttggaaaccgggctcccagctgccgccgtgtgggaggtcataatggaagcctcctcttcgggaaactgatgccccagctgctccctgaagtggtctcgaaggttgagcttgtggagggagacggtggcgctggaacggtcctgcttgttaccttccctccaggttagaagaacaacctgaaaacacatacgagcttcttttgaggtgtggtacgatgatgcggtctcggtctcggctccaaacagtttttgctttcttgtggttcatggcttcatgcatgttgccaggaactcctggatcagaaactttcagagagaagttcatcaaggtcgatgacgaaaactacatcaaggagacggtggtcactgaaggaggccttctggatcacggctttcggaagtacatggttcgaatcgagatcgtgggaagagaagagaagacatccatcgtaaggtcaacaattcagtacgaagtcgatcgtgagcatgcaggttcacacgcaccccctgtgttcagtaccgatgggttggctaccattgccgaggccatcaccaagtatatcaaggagaagagaggctctgagtccgtaagctctcccaagtaattaactcaagtaattgaactctggaattaaaatttggggtaaaaa

SEQ ID NO: 35 - DNA, 517 bp, Zea mays
ccccaaaaaccccagtcacaacgtgtgccctctcgtaatcctcctcgcctgctccacgtccaacgcttccgtcctacaagacgcgtgcaagtccttcgccgctaagatcccggacaccggctacgcctactgcatcaagttcttccaggccgacaggggaagcgccggcgcggacaagcgtggcctcgccgccatcgccgtgaggatcatgggggcagccgccaagagcaccgccagtcacatcgccgccctgcgggcctccgagaaggacaaggagcggctggcgtgcctcagcgattgctccgaggtgtacgcgcaggccgtggaccagaccggcgtggcggcgaagggcatcgcctcgggcacgccccggggccgcgcggacgcggtgatggcgctcagcacggtggaggatgcccccggcacctgtgagcaggggttccaggacctgggcgtgcgttcgccgctggcctcggagacgccggttccgaagatctcagaattttttgaaaaagaa

SEQ ID NO: 36 - DNA, 647 bp, Zea mays
ccagtcagaacggaggttttacttgagaacaagagtaatctctttctctctcgcacagtgtgtgtatatatgaaccacctaattaccgctgtagtctcttaatcccatcaatcaatcaatggcgcactgcatgttgacacacgctaatacatacaggtatctacgaggccaagacagatctcggcaagaacacgtgcaccatcgagggtgtcattgaggaggacaagctcgtcaagtacatctacgagaggatgcgcaagaagggcgtcgtcgacaaggtcgagaagaaggtgatcatcaaggaggagaaggtcttagtgaagaaggcggataaggagaaggagaagaaggagaaggagaaggagaaagaaaaggagaaggccaaggagaaggtgaaggaggctgtcgacaaggtcaaggaggtcatcgccccctacttcatcccctgcacgcacccgaacttcgtcgactactcgcacccctggcaccgcggcggcggcggctactgctcgtcgtacggtgacggttacggctacggctacggcgggggctgcggagggtacccaccgtacggtttcagctacacacactctgagctcaaaggctaccatgacacgtcgttctgcactcacacaacttgggggtaa

SEQ ID NO: 37 - DNA, 589 bp, Zea mays
aggatcccagtcaggacgatggcaggcggattctgttaaccactaaagctggacgtgtccatgtgctagattcattccatggcaatagcgtaagcatacaacctactgtaccttgtatcttgcatataccaacactagtaagacatggttcttatgattccttttttctttcagattgcgtcgtgcaatgtgaagccagtggtaaccaactcaacactggaggcgtcgttcagccctgatggaaaccatatcatatctggttagagaccatccctatcgtgctttacaaagagttgccatcttctctcctgcttagcaatgcgttatggtcttctcttgcaggctccggtgacggtagtgtttatgcttggaatgttaggagtggaaaggtgcagaaagatatatcatgtcgatattgaaatcacttcgtatgttatatctatttgacttgtatatggtaaaacactttgacatgcaggtcgcgcgctggggaagcacagacgacgaaccgccgctggtaaggtgggctccaggatccttgatgttcgtgacagatcatcagaactgtcatgctgtactgggcttatta

SEQ ID NO: 38 - DNA, 1180 bp, Zea mays
  misc_feature (1164)..(1164): n is unknown
tttgattcttgtcaatagcaagttcactgcagcaacttttgcaaggaccttttctggtttacatgctagaggaatcgagcctggtgttctttatccagctgtctctgttgagcagtttcacgaaccccatgcttataagtaacttcatgcttgcatatctccttacatttaggtctaacttattcagtttatagactaaacagatctttttaccattatcatattaatatttagctaaagtagacaacaatcttttgtacaggttgaatttcctatcaatcaaccggtttgagaggaaaaagaatcttgatcttgccatttcagcatttgctttgctccgttctgctgcttggactctacctggtgatgctctacaagaagcaacattaacagtggcaggtgtttatattttatttttccttctagttgcatgttcaatgttacaacaccaccggttttaaaccatatgaaatattgactgctgatttcctaccatgcctattatttaggtggctatgataagcgtctcaaggaaaatgttgaataccttgaggaactcaaaagactcgcattgacggaaggggtttctggacaggttaawtttgttacatcttgctcaacatctgaaagaaacgagcttctctccaactgcctctgcgttttatacactccaaaggtaagtgcctagggcttacatccagctaagcagtttgtttacctttaatttaacaagcctgcgtctcttatccaggatgaacatttcggtattgtacctcttgaagccatggccgcccataagccggtaattgcctgcaatagtggtggcccagtggaaacagttgtgaatgaagtaacagggtttctgtgtgatccctctcccgcagaattctccaaagccatgctgaaacttgtgaatgatcatgatcttgctgtcagattgggtgaacaagcacgtgaccatgtggtgcaaaaattctcgaccaagacatttggtgatctcctcaacagctacgtcttgaacatctaccatgagaggatggaatgatctataatattgggtcagccatgccatatgaaaacaatttgttcaatacaaggttttttttgcacctttacgtctaatctgattttgatggacacacataatgacaatgacattccatngaatccctttggcata

SEQ ID NO: 39 - DNA, 601 bp, Zea mays
  misc_feature (10)..(10): n is unknown
cccccaaaanccccaaaattctwraaaaattccagaacaccttcttttaagaagcaacattaacagtggcaggtgtttatattttatttttccttctagttgcatgttcaatgttacaacaccaccggttttaaaccatatgaaatattgactgctgatttcctaccatgcccattatttaggtggctatgataagcgtctcaaggaaaatgttgaataccttgaggaactcaaaagactcgcattgacggaaggggtttctggacaggttaattttgttacatcttgctcaacatctgaaagaaacgagcttctctccaactgcctctgcgttttatacactccaaaggtaagtgcctagtggcttacatccagctaagcagtttgtttaccttaatttaacaagcctgcgtctcttacttatccaggatgaacatttcggtattgtacctcttgaagccatggccgcccataagccggtaattgcctgcaatagtggtggcccagtggaaacagttgtgaatgaagtaacagggtttctgtgtgatccctctcccgcagaattctccaaagccatgctgaaatggtcatagctgccctt

SEQ ID NO: 40 - DNA, 369 bp, Zea mays
  misc_feature (54)..(56): n is unknown
gcggcggacatggaactggaygatgcttcagccgagacaccaaccagtggaacnnntgggaagctgtcttcccatgccgcagaagaagcrgagacatggtgcccgtggctggtcggtannncacagcagtttctgtactatctccctcrgggagaggtgttctctatgcatcctggttgccagttcctaaactacggtaacggaagcatatcctacwcagcgttggacgcacrgacagttacctcaaacaagcagcggagccrrccatggacggagtcgatcgaracctccagcagcgtgcctgnaacagctcagaattcaratcctgyagaatctacgaaagtaaacagaggtgaagacaaagtggct

SEQ ID NO: 41 - DNA, 462 bp, Zea mays
  misc_feature (54)..(56): n is unknown
gcggcggacatggaactggaygatgcttcagccgagacaccaaccagtggaacnnntgggaagctgtcttcccatgccgcagaagaagcrgagacatggtgcccgtggctggtcggtannncacagcagtttctgtactatctccctcrgggagaggtgttctctatgcatcctggttgccagttcctaaactacggtaacggaagcatatcctacwcagcgttggacgcacrgacagttacctcaaacaagcagcggagccrrccatggacggagtcgatcgaracctccagcagcgtgcctgnaacagctcagaattcaratcctgyagaatctacgaaagtaaacagaggtgaagacaaagtggctgtacccgttccaggttcaaggaaatgcgcgrgcgcaattccagcctgccgtcgaggttttgtaccgtacaagaagtgcacagctcggagcaag

SEQ ID NO: 42 - DNA, 669 bp, Zea mays
gagaaaaacccctggtttgaatttgacagcagcaagctcaatactgggagcctacaacacgcggcggacatggaactggatgatgcttcagccgagacaccaaccagtggaactgggaagctgtcttcccatgccgcagaagaagcagagacatggtgcccgtggctggtcggtagtacacagcagtttctgtactatctccctcggggagaggtgttctctatgcatcctggttgccagttcctaaactacggtaacggaagcatatcctacacagcgttggacgcacggacagttacctcaaacaagcagcggagccaaccatggacggagtcgatcgaaacctccagcagcgtgcctgaaacagctcagaattcagatcctgcagaatctacgaaagtaaacagaggtgaagacaaagtggctgtacccgttccaggttcaaggaaatgcgcgagcgcaattccagcctgccgtcgaggttttgtaccgtacaagaagtgcacagctcggagcaaggtgctggcgctgcagcctgtggcacctggcgaggaggcagatagagagctgacaaggctgtgcctgtagaattctgggcccttgccacccacctctactctgtggataatttttgctgctactcgaacacttaatggtcatagctgtttt

SEQ ID NO: 43 - DNA, 498 bp, Zea mays
aagtcccagtcacaacgaaagaacattacgaatgccattgtgatcgaggtccgagtattagcgtaggattgcgtacccggtggacgtaggagtccggcatgagcgaacggagcgcggcgaggtactcgttcacctggcggcggcggttgcgctcgacggcgatgtgggtcatccgctggctctcggcgttcttggtgctcctctgccggcgccgccgccgccgcttcatccgcccctgctgcacgctgttcgcctgggacgccccccgcggcggtgccacctccgccgatggtgcattgccggaaaagctcccccgtccccccgcagaagcggcagacgcgtcggcggcgttgaaggtgtcgtagatgagatgagtacgtcgcggcacaaagcttccagcgtcatgtcgatcggcgatttggcggcctgtggacttgtggtatggcacggaggccagcaagaacctacgcgcggaccgccgcccgattttttgaattt

SEQ ID NO: 44 - DNA, 608 bp, Zea mays
agtcagaacgcaacagagaaaaattcatgtacaggtaaaaaaaaccaccaccgatttagagactagagtatccgggggggtcccttcccctagaagaagagatctccgtccgtggcatggaggaacggacgaatggttcgctgacggcggtgtggctgtttcttttttttgctatcgcagaggtgacctgccgttctgcagcgaggagtgccggcaggagcagatcgagatcgacgaggcgcgagagcagcggctgaagcagacggggcgggccgagcagcagcggcagcggcagcagaagcagagcccccagaggatccccatctgggcgtggtaggatgagaaaaattttgggcgccgggaaaacgaacgaacgtagtaggatttagctgcacctcaagaagaaccccccaagcaagggcgacttgctgcgtgtggaaacaaacggccgtggatcaccgccggctcgacgcaggaagaaggccacgcgccacggcacaggccgggcagggcagggcatccaaccggctgcgtctttttaccttcgttggttgacgaaaccgaatgacctctctcctcctatctccgtagtaactttggtaaaataa

SEQ ID NO: 45 - DNA, 637 bp, Zea mays
aaggtctataggggaggcaaacttgaaaaagcgagaattcgctgttgatccatccacagctcgtttccacctttcggaacctttccgccggcctgttctgacgtcagaaccatctatatcttcaagaattcttggttcgcaagaccgttttttgatctttgcatcggatggactatgggagcacctctcaaaccagcaagctgttgaaattgtccacaatagtccacgagaagtatggttttcttctttctcgctgcattttgtcttgaactggcaactcttctcatatcgctcttgctgatgatagcattcctgttgcggcaacgctgaaatgggttttccttgtttttgtagggtgttgcaaggagattggtacaaacagctctaaaagaagctgcgaggaagagggaaatgaggtatggcgatattaagaagctcgaaaaaggagtccggcgctacttccacgacgacataacagttgtcgtcgtcttcatagaccatgaactgcgggcggagcattcttcctcgacctctgttcctgaactctcggtccgtgggttcgttgatgcgggggcacgctccagcttttcagggatgaacgacattacttatacagtaaccttgttaatttttaaaa

SEQ ID NO: 46 - DNA, 631 bp, Zea mays
tccgccccccccagtcaggtttttgaagctctttgaaaactcacttttattgtccgccccattacatcctggaaaagattattctcaatgttactatctaatttggagaaatctttaggtgtcaacattttaatgactagctcaatgataatgatacaagactgataaactggtcccatgttgtagaaaactgaatcattaagtgatattaggtcattctcagtgatcagttttaatgtactgtttccaaaactgccacatcagatttaaactagataaccgtgtatgcagagttctcccccatgaaactctaccatcttccatgaaatggtgaaactctccctatatctctcttattaattgcactgccatgtcactaagtgtgatgatgtgtcaccgcatttaatgagtatgaaaatcccattgagaatggtcttacaagaaattccatgtccactaagttgtgaaagcctcattggaggagaacgcaagtgacgtcaatagatggaccatggatgcgctaggacgtaatttagataacatcttctacttcacacccmctcaatctatctgatgctgaatcgtgaaactacaccatggtgcaaaccaaacgcatcaatgcaacyatggt

SEQ ID NO: 47 - DNA, 672 bp, Zea mays
gggcattcatggaaaaggcccaaccgggtttttcagggaaggggattccttacctccagatgttgatccagatacagttagatggattccagcaaaccatccttttgctgctgcgtcaagtgaagtagacgaggagaccgccaaacaaaatgtctatcaaaaggatggcgtcccatcccgtgttaaggctgagcatgaagctttgcaggcaaggctagaggcttcaaacgatgtgagtgccttgccgatgaacatgatctccttgacatttaaaataacaacaatatatctattattagttcctctcttctgcaaaattgattatattgattgataaaactccaagcttagtttgacaaaagagccaatcacattgtgtatttttgttaatcaccagccttccagaataacgattcctgaatcccaacactgctttgcaggttaccagactccctccggatccaaggagtatgcagcgtaatgagagacaaatggaattgtcaggcaagccatctgaaaatcttcagggctccaagtttgagaaccaagatagacaactggttatcgagtctggtaaacatagctccgatggaagtttacaatcaaatgagccggaagggcaataaaatttggtactcaagcgcttgatacattgtaattggttgtattatt

SEQ ID NO: 48 - DNA, 450 bp, Zea mays
taaaattaaaggggaagtcccagtcaaaacggcatgtgctagattatcttctttaagatggggtaagcaacttcatgctcaggtgattcggaacctaccgcacattgatccatatgttgcaagtgctcttgttgaactgtatgcaaaatgtggctgtttcaaggaagctaagggggtcttcaactctttacatgaccgtaacaatgtggcttggacagttctcatctcaggattcttgcagtatgggtgtttcactgaatctgttgaattgttcaaccagatgagagctgagttgatgacacttgatcagttcgctttggctactcttataagtggctgctgcagcaggatggatttgtgccttgggaggcaactacattcactttgtctgaaaagtgggcagattcaagctgtagtcgtctccaatttttatttcactttctggtaata

SEQ ID NO: 49 - DNA, 18 bp, Artificial Sequence
  Primer
tctctctcgcgtgtgtgc

SEQ ID NO: 50 - DNA, 20 bp, Artificial Sequence
  Primer
tgggtctccttctccgtcta

SEQ ID NO: 51 - DNA, 20 bp, Artificial Sequence
  Primer
gaaaggctcgctagtcgcta

SEQ ID NO: 52 - DNA, 20 bp, Artificial Sequence
  Primer
aattcctatcgatcctggcc

SEQ ID NO: 53 - DNA, 24 bp, Artificial Sequence
  Primer
agaagtgcgtatgctacagtggtg

SEQ ID NO: 54 - DNA, 24 bp, Artificial Sequence
  Primer
cctagtggtggagttctaggcaaa

SEQ ID NO: 55 - DNA, 20 bp, Artificial Sequence
  Primer
gtgaagctctgcaccacgct

SEQ ID NO: 56 - DNA, 24 bp, Artificial Sequence
  Primer
atcgtccaaagaagaagagggaga

SEQ ID NO: 57 - DNA, 20 bp, Artificial Sequence
  Primer
aaggggagcaaacaaggtag

SEQ ID NO: 58 - DNA, 22 bp, Artificial Sequence
  Primer
atttaagtagtgcatggtggag

SEQ ID NO: 59 - DNA, 19 bp, Artificial Sequence
  Primer
aaagcatccacgagccgca

SEQ ID NO: 60 - DNA, 19 bp, Artificial Sequence
  Primer
atgcatggcacgaacacag

SEQ ID NO: 61 - DNA, 20 bp, Artificial Sequence
  Primer
tctatgtcagtcctgaggca

SEQ ID NO: 62 - DNA, 21 bp, Artificial Sequence
  Primer
aggctaccattaacatgcttc

SEQ ID NO: 63 - DNA, 22 bp, Artificial Sequence
  Primer
acacaatagagcttgatcgtga

SEQ ID NO: 64 - DNA, 22 bp, Artificial Sequence
  Primer
aaattgggagagcacagaaagt

SEQ ID NO: 65 - DNA, 22 bp, Artificial Sequence
  Primer
aaatgtgggagaacagcaagtt

SEQ ID NO: 66 - DNA, 22 bp, Artificial Sequence
  Primer
acaggttgatttggaatcaaac

SEQ ID NO: 67 - DNA, 22 bp, Artificial Sequence
  Primer
aacaatctcagaagcaatacag

SEQ ID NO: 68 - DNA, 21 bp, Artificial Sequence
  Primer
cgttctattacggtcatttgc

SEQ ID NO: 69 - DNA, 18 bp, Artificial Sequence
  Primer
ctatcgttggatggcacc

SEQ ID NO: 70 - DNA, 20 bp, Artificial Sequence
  Primer
ttgtgctgtgggagctcata

SEQ ID NO: 71 - DNA, 21 bp, Artificial Sequence
  Primer
atgtttcgacaggacatagac

SEQ ID NO: 72 - DNA, 19 bp, Artificial Sequence
  Primer
aggcacattccagtagcag

SEQ ID NO: 73 - DNA, 21 bp, Artificial Sequence
  Primer
agcaactcaagtctgacgatt

SEQ ID NO: 74 - DNA, 20 bp, Artificial Sequence
  Primer
gggaaggatgtcatatccga

SEQ ID NO: 75 - DNA, 19 bp, Artificial Sequence
  Primer
acaccaatccagaagggga

SEQ ID NO: 76 - DNA, 22 bp, Artificial Sequence
  Primer
gaatggaaagtacaatgagtcc

SEQ ID NO: 77 - DNA, 22 bp, Artificial Sequence
  Primer
ttcacagagcgtgctagaaata

SEQ ID NO: 78 - DNA, 21 bp, Artificial Sequence
  Primer
ctgaatctggagtttctgtca

SEQ ID NO: 79 - DNA, 22 bp, Artificial Sequence
  Primer
tatagatcttcctgcaggagtt

SEQ ID NO: 80 - DNA, 20 bp, Artificial Sequence
  Primer
ctgcagctaaaccctgatga

SEQ ID NO: 81 - DNA, 21 bp, Artificial Sequence
  Primer
tcagggagtggtatttccttg

SEQ ID NO: 82 - DNA, 22 bp, Artificial Sequence
  Primer
tatcaggtcagcaaatttccaa

SEQ ID NO: 83 - DNA, 20 bp, Artificial Sequence
  Primer
cattcctcctgtccacaaca

SEQ ID NO: 84 - DNA, 22 bp, Artificial Sequence
  Primer
tttacgactgcaatgaatcaac

SEQ ID NO: 85 - DNA, 22 bp, Artificial Sequence
  Primer
tggcagcaatttcagcatgtaa

SEQ ID NO: 86 - DNA, 22 bp, Artificial Sequence
  Primer
gaggaagcttatgaatttgtgc

SEQ ID NO: 87 - DNA, 21 bp, Artificial Sequence
  Primer
ctggtcttgtaaacactcact

SEQ ID NO: 88 - DNA, 21 bp, Artificial Sequence
  Primer
tgcaccgagcaaatacctttg

SEQ ID NO: 89 - DNA, 20 bp, Artificial Sequence
  Primer
tacctgatcgtcaagtcgct

SEQ ID NO: 90 - DNA, 19 bp, Artificial Sequence
  Primer
tgcccatggacctcttctt

SEQ ID NO: 91 - DNA, 20 bp, Artificial Sequence
  Primer
actctgaattgagcagcagg

SEQ ID NO: 92 - DNA, 21 bp, Artificial Sequence
  Primer
actttggctagcacaacaaca

SEQ ID NO: 93 - DNA, 18 bp, Artificial Sequence
  Primer
acttagctgcaaagatgc

SEQ ID NO: 94 - DNA, 21 bp, Artificial Sequence
  Primer
aacattggtgcaagggacact

SEQ ID NO: 95 - DNA, 20 bp, Artificial Sequence
  Primer
cagaaatgccatcaacgcca

SEQ ID NO: 96 - DNA, 22 bp, Artificial Sequence
  Primer
ggaatgatctccatgctttcat

SEQ ID NO: 97 - DNA, 19 bp, Artificial Sequence
  Primer
ccagtccaaaccctacggc

SEQ ID NO: 98 - DNA, 19 bp, Artificial Sequence
  Primer
cgaccaaaccctactcgta

SEQ ID NO: 99 - DNA, 20 bp, Artificial Sequence
  Primer
atcgatctcctgctgcaagt

SEQ ID NO: 100 - DNA, 21 bp, Artificial Sequence
  Primer
aataataggcgattgaggtgc

SEQ ID NO: 101 - DNA, 21 bp, Artificial Sequence
  Primer
aataataggcgattgaggtgc

SEQ ID NO: 102 - DNA, 20 bp, Artificial Sequence
  Primer
agcctagcgcaaagaaagac

SEQ ID NO: 103 - DNA, 21 bp, Artificial Sequence
  Primer
caggccacggaatttacacaa

SEQ ID NO: 104 - DNA, 20 bp, Artificial Sequence
  Primer
tcgctgcgtagagtactgta

SEQ ID NO: 105 - DNA, 18 bp, Artificial Sequence
  Primer
acccgtcggaccacgtct

SEQ ID NO: 106 - DNA, 18 bp, Artificial Sequence
  Primer
gctcaactccgctgctgc

SEQ ID NO: 107 - DNA, 22 bp, Artificial Sequence
  Primer
tcacgttgttttcaccatgtat

SEQ ID NO: 108 - DNA, 22 bp, Artificial Sequence
  Primer
tattagtttaggcaactaagga

SEQ ID NO: 109 - DNA, 20 bp, Artificial Sequence
  Primer
aacgcatacttcctcggcta

SEQ ID NO: 110 - DNA, 20 bp, Artificial Sequence
  Primer
atccactcgtacaccgagta

SEQ ID NO: 111 - DNA, 21 bp, Artificial Sequence
  Primer
tcactctcaaacaactggttg

SEQ ID NO: 112 - DNA, 21 bp, Artificial Sequence
  Primer
gaagaagtgataaacgccaga

SEQ ID NO: 113 - DNA, 22 bp, Artificial Sequence
  Primer
tactttgtccagaagagcagaa

SEQ ID NO: 114 - DNA, 22 bp, Artificial Sequence
  Primer
tgtggaaatttgtctattgcta

SEQ ID NO: 115 - DNA, 21 bp, Artificial Sequence
  Primer
aggatgctttcattactgatt

SEQ ID NO: 116 - DNA, 22 bp, Artificial Sequence
  Primer
aataggcagataccaaggcaat

SEQ ID NO: 117 - DNA, 20 bp, Artificial Sequence
  Primer
aaatgaatcaaagggtcgga

SEQ ID NO: 118 - DNA, 19 bp, Artificial Sequence
  Primer
ccctacatgtcggcaaccg

SEQ ID NO: 119 - DNA, 21 bp, Artificial Sequence
  Primer
gttcctgttcatgttcccgaa

SEQ ID NO: 120 - DNA, 22 bp, Artificial Sequence
  Primer
aatttacaaggttctatcggtt

SEQ ID NO: 121 - DNA, 19 bp, Artificial Sequence
  Primer
gcttctagagtacgcccgt

SEQ ID NO: 122 - DNA, 21 bp, Artificial Sequence
  Primer
catgttgcgcagtttgtgctt

SEQ ID NO: 123 - DNA, 20 bp, Artificial Sequence
  Primer
gttgggcaacgtcagtttca

SEQ ID NO: 124 - DNA, 21 bp, Artificial Sequence
  Primer
gccttctaataaacgcagcaa

SEQ ID NO: 125 - DNA, 20 bp, Artificial Sequence
  Primer
ctacggagctcaaagctatg

SEQ ID NO: 126 - DNA, 20 bp, Artificial Sequence
  Primer
atctgccatgagttcgagac

SEQ ID NO: 127 - DNA, 22 bp, Artificial Sequence
  Primer
tcttcaattcccagagttcaat

SEQ ID NO: 128 - DNA, 20 bp, Artificial Sequence
  Primer
ttcacgctatggccctttct

SEQ ID NO: 129 - DNA, 22 bp, Artificial Sequence
  Primer
atttggatttgcattgtcagtc

SEQ ID NO: 130 - DNA, 19 bp, Artificial Sequence
  Primer
tgtgccctctcgtcatcct

SEQ ID NO: 131 - DNA, 18 bp, Artificial Sequence
  Primer
cgatggacgcatccttcc

SEQ ID NO: 132 - DNA, 19 bp, Artificial Sequence
  Primer
ttacaacgcggccgttaca

SEQ ID NO: 133 - DNA, 21 bp, Artificial Sequence
  Primer
cataactgtaaagctgccgtt

SEQ ID NO: 134 - DNA, 22 bp, Artificial Sequence
  Primer
gaggttttacttgagaacaaga

SEQ ID NO: 135 - DNA, 20 bp, Artificial Sequence
  Primer
tgtgtgcagtgcaagaacga

SEQ ID NO: 136 - DNA, 20 bp, Artificial Sequence
  Primer
gttctcctcgctgatgaact

SEQ ID NO: 137 - DNA, 22 bp, Artificial Sequence
  Primer
tctgttggtaataacgattcag

SEQ ID NO: 138 - DNA, 20 bp, Artificial Sequence
  Primer
atggcaggcggattctgtta

SEQ ID NO: 139 - DNA, 20 bp, Artificial Sequence
  Primer
gaacccagcatgacagttct

SEQ ID NO: 140 - DNA, 22 bp, Artificial Sequence
  Primer
ttcacctacttgctaatggtaa

SEQ ID NO: 141 - DNA, 21 bp, Artificial Sequence
  Primer
tttgctttgctccgttctgct

SEQ ID NO: 142 - DNA, 22 bp, Artificial Sequence
  Primer
gctctacaagaagcaacattaa

SEQ ID NO: 143 - DNA, 21 bp, Artificial Sequence
  Primer
ttcagcatggctttggagaat

SEQ ID NO: 144 - DNA, 21 bp, Artificial Sequence
  Primer
cacccaatctgacagcaagat

SEQ ID NO: 145 - DNA, 21 bp, Artificial Sequence
  Primer
gctatttggcaagagggttgt

SEQ ID NO: 146 - DNA, 22 bp, Artificial Sequence
  Primer
gttagtaatttagaccagcagc

SEQ ID NO: 147 - DNA, 21 bp, Artificial Sequence
  Primer
tatgtgttcgagtagcagcaa

SEQ ID NO: 148 - DNA, 21 bp, Artificial Sequence
  Primer
aaatggaaacactagtcccga

SEQ ID NO: 149 - DNA, 20 bp, Artificial Sequence
  Primer
ccagtgtgccactatctgaa

SEQ ID NO: 150 - DNA, 22 bp, Artificial Sequence
  Primer
aaagaacactacgaatgccatt

SEQ ID NO: 151 - DNA, 18 bp, Artificial Sequence
  Primer
accgggcggcggtcgcgc

SEQ ID NO: 152 - DNA, 19 bp, Artificial Sequence
  Primer
tagcacccgtagtggcata

SEQ ID NO: 153 - DNA, 20 bp, Artificial Sequence
  Primer
cactacctcgacatctgctt

SEQ ID NO: 154 - DNA, 22 bp, Artificial Sequence
  Primer
caacagagacatcttcatgtac

SEQ ID NO: 155 - DNA, 21 bp, Artificial Sequence
  Primer
tccatctttcggagatcagga

SEQ ID NO: 156 - DNA, 21 bp, Artificial Sequence
  Primer
aaactcacgcacaaaccaacc

SEQ ID NO: 157 - DNA, 21 bp, Artificial Sequence
  Primer
cgcatcaaaggcatcatacag

SEQ ID NO: 158 - DNA, 21 bp, Artificial Sequence
  Primer
ttcaaggtctataggtgatgc

SEQ ID NO: 159 - DNA, 21 bp, Artificial Sequence
  Primer
gcaacacaaggttactgtatc

SEQ ID NO: 160 - DNA, 21 bp, Artificial Sequence
  Primer
actacgcgctagtaacaccaa

SEQ ID NO: 161 - DNA, 21 bp, Artificial Sequence
  Primer
gtgcctctctataatagtgcc

SEQ ID NO: 162 - DNA, 21 bp, Artificial Sequence
  Primer
agctggtcttctgaagctctt

SEQ ID NO: 163 - DNA, 21 bp, Artificial Sequence
  Primer
ggttgcattgatgcgtttggt

SEQ ID NO: 164 - DNA, 19 bp, Artificial Sequence
  Primer
tcccatgtgagcgtgtcga

SEQ ID NO: 165 - DNA, 22 bp, Artificial Sequence
  Primer
ggaacaagaaatgcatcagaag

SEQ ID NO: 166 - DNA, 20 bp, Artificial Sequence
  Primer
ggagatcacgtgcatatcag

SEQ ID NO: 167 - DNA, 22 bp, Artificial Sequence
  Primer
caaagcaaatgtcatcaaagcg

SEQ ID NO: 168 - DNA, 21 bp, Artificial Sequence
  Primer
ctatgtaaaccggtaagccca

SEQ ID NO: 169 - DNA, 21 bp, Artificial Sequence
  Primer
gggtgcgtcttgattcaacaa

SEQ ID NO: 170 - DNA, 22 bp, Artificial Sequence
  Primer
gcatgtgctagattatcttctt

SEQ ID NO: 171 - DNA, 21 bp, Artificial Sequence
  Primer
ggagatgagagaattggagac

SEQ ID NO: 172 - DNA, 22 bp, Artificial Sequence
  Primer
gattgtcgcactttgcatacat

SEQ ID NO: 173 - DNA, 21 bp, Artificial Sequence
  Primer
gctgcaggatttcctggtgca

SEQ ID NO: 174 - DNA, 18 bp, Artificial Sequence
  Primer
aaccgctcctgctccagc

SEQ ID NO: 175 - DNA, 21 bp, Artificial Sequence
  Primer
gctgcaggatttcctggtgca

SEQ ID NO: 176 - DNA, 18 bp, Artificial Sequence
  Primer
aaccgctcctgctccagc

SEQ ID NO: 177 - DNA, 24 bp, Artificial Sequence
  Primer
cttccagacaagccaactgactga

SEQ ID NO: 178 - DNA, 30 bp, Artificial Sequence
  Primer
caacgatttccaacagcttayttccatgga

SEQ ID NO: 179 - DNA, 24 bp, Artificial Sequence
  Primer
cttccagacaagccaactgactga

SEQ ID NO: 180 - DNA, 30 bp, Artificial Sequence; identical to SEQ ID NO: 178
  Primer

SEQ ID NO: 181 - DNA, 30 bp, Artificial Sequence
  Primer
aaataggcctcgatctttcatactggaaaa

SEQ ID NO: 182 - DNA, 26 bp, Artificial Sequence
  Primer
ggccaagattggtcgayaccatgtaa

SEQ ID NO: 183 - DNA, 30 bp, Artificial Sequence; identical to SEQ ID NO: 181
  Primer

SEQ ID NO: 184 - DNA, 26 bp, Artificial Sequence
  Primer
ggccaagattggtcgayaccatgtaa

SEQ ID NO: 185 - DNA, 22 bp, Artificial Sequence
  Primer
agtggatcgaggargacgagcc

SEQ ID NO: 186 - DNA, 19 bp, Artificial Sequence
  Primer
ccttggccctatcctcgcc

SEQ ID NO: 187 - DNA, 23 bp, Artificial Sequence
  Primer
cggaagcatacatcactggctca

SEQ ID NO: 188 - DNA, 19 bp, Artificial Sequence
  Primer
ccttggccctatcctcgcc

SEQ ID NO: 189 - DNA, 17 bp, Artificial Sequence
  Primer
cggccatcgccatcgac

SEQ ID NO: 190 - DNA, 23 bp, Artificial Sequence
  Primer
cgtaccgatgcaaagtggttgac

SEQ ID NO: 191 - DNA, 17 bp, Artificial Sequence
  Primer
cggccatcgccatcgac

SEQ ID NO: 192 - DNA, 23 bp, Artificial Sequence
  Primer
cgtaccgatgcaaagtggttgac

SEQ ID NO: 193 - DNA, 17 bp, Artificial Sequence
  Primer
cggccatcgccatcgac

SEQ ID NO: 194 - DNA, 23 bp, Artificial Sequence
  Primer
cgtaccgatgcaaagtggttgac

SEQ ID NO: 195 - DNA, 33 bp, Artificial Sequence
  Primer
gcagtcaatttcaagagaagtatcttaggtatg

SEQ ID NO: 196 - DNA, 33 bp, Artificial Sequence
  Primer
aacttttacaagaatacttcatgcatcaacaga

SEQ ID NO: 197 - DNA, 31 bp, Artificial Sequence
  Primer
gcatttgagtgatgagagtattaccttttgg

SEQ ID NO: 198 - DNA, 28 bp, Artificial Sequence
  Primer
cccttgagtccttctacaaagattagca

SEQ ID NO: 199 - DNA, 27 bp, Artificial Sequence
  Primer
ttcaagtctttatgcatgaccaggtga

SEQ ID NO: 200 - DNA, 29 bp, Artificial Sequence
  Primer
aattgactgcatatgtgcctgatctctac

SEQ ID NO: 201 - DNA, 27 bp, Artificial Sequence
  Primer
ccgacaaacttttgcttcycctcatga

SEQ ID NO: 202 - DNA, 22 bp, Artificial Sequence
  Primer
tgcttcggactagtctgtccac

SEQ ID NO: 203 - DNA, 26 bp, Artificial Sequence
  Primer
tttcggaagtacatggttcgaatcga

SEQ ID NO: 204 - DNA, 26 bp, Artificial Sequence
  Primer
ggtagcyarcccatcggtactgaaca

SEQ ID NO: 205 - DNA, 30 bp, Artificial Sequence
  Primer
aaaaagaatcttgatcttgccatttcagca

SEQ ID NO: 206 - DNA, 22 bp, Artificial Sequence
  Primer
gccctaggcacttacctttgga

SEQ ID NO: 207 - DNA, 23 bp, Artificial Sequence
  Primer
gaactggaygatgcttcagccga

SEQ ID NO: 208 - DNA, 23 bp, Artificial Sequence
  Primer
ggtacagccactttgtcttcacc

SEQ ID NO: 209 - DNA, 23 bp, Artificial Sequence
  Primer
gaactggaygatgcttcagccga

SEQ ID NO: 210 - DNA, 23 bp, Artificial Sequence
  Primer
ggtacagccactttgtcttcacc

SEQ ID NO: 211 - DNA, 880 bp, Zea mays
catcgccgttccttcctggcgatcgccgctccctagctatccggtggccaaagacacggctagtggtaggctcgagcgagacgagctcttggtgaagagagaatgaatgtaacgttaccgcctcctggtcgtaggggtgtgtgtatgtgaggacaagaggaggagcgagaggaggagcgcagagcgtggcggggaaggagggcgtcatgtgtgcgaggaatctaggacgacttgttggcagctgggccggggtgcgtgcgagatgcaatgcaagaacaaagcggacgggcatctcgctcggccacgcttccaagtccatccggggggcgccactcggccgccgctcattgaggcccaggcgccaagacggcggctccacccacgtcacaattggcaacaagaagcacacggctggggctgggacgcgtcgaatttttcaccagaaaataccgtctgatcctggcgtttcgtgaacggcaaaacctagcagcagcagcagcattccacgggtcggatgacatatcatatcctcgtgcggagcggactctacggcgagtccagctgtggctgcggaatattccggcggaagcgcggggagagcgacggcggcctccggtgggacccggggcgagcgggagatgcggcgaagatgttcggcgctgatgtcgctggaatattcgcgccagctgtggctgccggtgcgacctgctgaccagacgaccaatggcagtggccaccgcctctccctcttgctgttggagttggatccacggaccactctccatccaacatccatcacagattggcggacgattagccgagactaatcgctattctcaacacttttaaaaccgtacgtgcaaaatgctaaggggccgtt

SEQ ID NO: 212 - DNA, 936 bp, Zea mays
catcgtcgttccttcctggcgatcgccgcttcctagctatccggtggccaaagacacggctagtggtaggctcgagtgagacgagctcttgctgaagagagaatgaatgtaacgttaccgcctcctggtcgtaggtgtaataagttgtaacgcgagcgtcgttagcaagcacaggggtttgtgtatgtgaggacaagaggaggagcgagaggaggagcgcagagcgtggcggggaaggagggcgtcatgtgtgcgaggaatctaggacgacttgttggcacttggcagctgggccggggtgcgtgcgagatgcaatgcaagaacaaagcggacgggcatcacgcctccaggtccaacccgggggcgccactcggccgccgctcattgaggcccaggcgccaagacggcggctccacccacatcacaattggcaacaagaagcacacggctggggttgggacgcgtcgaatttttcaccagaaaataccgtctgatcctggcgtttcgtcagatgctatgctacgtgaacggcaaaacctagcagcagcagcagcactcagactggacaagaggagggaaatctttgcgtgggaaccaaactgaacgcgaatcgcacgagtcggatgacatatcctcgtccggagcggactcgaccgcgagtccagctgtggctgcggaatattccggcggaagcgcggggagaacgacggcggcctccggtgggacccggggcgagcgggagatgcggcgaagatgttcggcgctgatgtcgctggaatattcgcgccagctgtggctgccggtgtgacctgctgaccagacgaccagtggcagtggccaccgcctctccatcacagattcgcggacgattagccgagactaatcgctattctcaacacttttaaaaccgtgcgtgcagaatgctaagggcgcgtt

SEQ ID NO: 213 - DNA, 3250 bp, Zea mays
  misc_feature (1458)..(1737): n is unknown
tcgcatctgcagcttcttttgcacctgattacagacataagcacttgtagcgtttatggaagaaagttttggagtgcagatctcatgacaatgatgtaaatctatcttgcctcagtttgttcttgtagtttcctttggacttgaatttgataccttagtgcatcgctaagtgctatttctctgattcacataagaaatgtgatacaaatggttagttcaatcaatgcagaaaagttcaatcaaataaaatgggcccactgcagtcaattaacaggcattcaataggattcacattcctgggcttctatatatggaagtttgcatacaatgttttggaaataaaatgaaatataaattgcttgcatttagtgtaagttaatactcgctcccttctcgaatatttgtcgtccgctagttcatttttgaactaaaacatgataaataaaaaaacggaaggagtacatgtttgtaacaggagagcccatgaatacttgcttgtaacaggtggagcgctaagtatgcttaggagaactttaggcaacttgtattctttagcacttcgacgcagtttgtatggtaatatctactgatagacagaatcctggttttggatttttaatttttcctgcttttggttacacctctacagtcccatactcgcagtccaatagtacatggtctgataataaaccaattaagaaggactcatgtctcagtcattatgacttgagcataggagttgagatcaagaaatatttgagttgcagcttaaggtccagagaggaaatccccatacacttgcttgtaacggtatgaatgtatgatcatttttttttcaaggtaacattttctagcatcttcacctgtctacttgactgaatgcagtatatattagttgtaataactactggccttctgctgtgcacaaaaggcgggtattaccacttgcagaaatttgtcgggtaaaggtaattgccagttaccttgtgttcttcccttgatcaggaacacctggaggaggatgcgctgtggttgaaccgaagccctgtgagcgaagtactgatgacagaaagagcggaagataagataagaaaggaacccttgcgcggcaaggcctggtgacatagaggtagtgcgaggctcataccgccgccgctggcaggttccaggcctgtgcttttcttgccctgtatccccagtctatacttctgcgcacatcagacgagcctcagtgtttcggcacagtggtgcaacagaaaaggagagtgctggtaggtaacgctgaggcggtgaagaaagagaggtcagacggacctggaggtggctttttaactggtaaagagtgaggtctttcatgcccatcaatctgagcaccgacttgggtgttgctcctgttcgcaggaagcacaagaaatggtcagtactccacaccataagcatgtcggtggtgtgttggannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaaaaaaaaaaaaaaaaagaatgcacaccgacatgctctgtagcacaagcaccatactggcgaactggagaggtctcggctcatcaagcaatcgccttggtgtcggacggggtcatcaagacaagacgactagacgagcactacatatagacgggaacgtacgggaggaaggaaggaaaacgagagcgaggactcactgtccggtccgcccagcttggtgacggcgtcgacgaagcgctggtggaggtccggcgtccagcgcagccgcggcttggggtcccgtgacgccgccccgtcgtagccgtagctcccctgcatcgtcgttccttcctggcgatcgccgcttcctagctatccggtggccaaagacacggctagtggtaggctcgagtgagacgagctcttgctgaagagagaatgaatgtaacgttaccgcctcctggtcgtaggtgtaataagttgtaacgcgagcgtcgttagcaagcacaggggtttgtgtatgtgaggacaagaggaggagcgagaggaggagcgcagagcgtggcggggaaggagggcgtcatgtgtgcgaggaatctaggacgacttgttggcacttggcagctgggccggggtgcgtgcgagatgcaatgcaagaacaaagcggacgggcatcacgcctccaggtccaacccgggggcgccactcggccgccgctcattgaggcccaggcgccaagacggcggctccacccacatcacaattggcaacaagaagcacacggctggggttgggacgcgtcgaatttttcaccagaaaataccgtctgatcctggcgtttcgtcagatgctatgctacgtgaacggcaaaacctagcagcagcagcagcactcagactggacaagaggagggaaatctttgcgtgggaaccaaactgaacgcgaatcgcacgagtcggatgacatatcctcgtccggagcggactcgaccgcgagtccagctgtggctgcggaatattccggcggaagcgcggggagaacgacggcggcctccggtgggacccggggcgagcgggagatgcggcgaagatgttcggcgctgatgtcgctggaatattcgcgccagctgtggctgccggtgtgacctgctgaccagacgaccagtggcagtggccaccgcctctccatcacagattcgcggacgattagccgagactaatcgctattctcaacacttttaaaaccgtgcgtgcagaatgctaagggcgcgttcgtttgcacagcaatagacatggatttatttcagctcatcaaaatctatataaattaaagaagtaatccggctagaaattaatccggagcttcaatccctaacaaccgaacagggtctaagcctgctagattcgagcatctgcgtgactctactttggctcttctcgtacgatgcgacttgacgatgcatttgggnnnnccnttagcgacactctcctgattagtcccacggaaacgcaactctaccactatcagccgccg

SEQ ID NO: 214 - DNA, 3653 bp, Zea mays
  misc_feature (2001)..(2142): n is unknown
tcgcatctgcagcttcttttgcacctgattacagacataagcacttgtagcgtttatggaagaaagttttggagtgcaaatctcatgacaatgatgtaaatctatcttgcctcagtttgttcttgtagtttcctttggacttgaatttgataccttagtgcatcgctaagtgctatttctctgattcacataagaaatgtgatacaaatggttagttcaatcaatgcagaaaagttcaaccaaataaaatgggcccactgcagtcaattaacaggcattcaataggattcacattcctgggcttctatatatggaagtttgcatacaaagtttttgaaataaaatggaatagaaattgcttgcatttagtgtaagttaatactagctccgttctcgaatatttgtcgtccgctagttcatttttgaactaaaacacgacaaataaaaaaacggaaggagtacatgtttgtaacaggtgagcccctgaatacttgcttgtaacaggtggagcactaagtatgcttagaactttaggcaacttgtattctttagcacttcgacgccgtttgtatggtaatatctactgatagacagaatcctggttttggattttttttttatttttcctgtttttggttacacctctacagtcccatactcgcagtccaataatacatggtctgataataaaccaattaagaaggactcatgtctcagtcattaggctgtctccaacaacgtcctctatattcatcctctatatctgtcctttacagtctcctctaaaaaatttcatcctatatatctcatttctctccaacaacgtcctctaaatcacgtcctctatactcaaatactcatattaaagacattttttaattttattttttatacatacgtaattatcatactctcaaatgtattgtgcatattttagttttgctaaaccggttatttaaagtagtcaaatggatagaggaccgtttagagaaactctatatatagagaattcagcagcgtcctctaaatttaaaggaccgtttagaggacgttgctggagagcgtagaggaccgtttggtcctctatatttagggtagagaaccctttagggggccttgttggagccagccttatgacttgagcataggagttgagatcaagaaatatgtgagttgcagcttaaggttcagagaggaaatccccatacacttgcttgtaacggtatgatcatatcttttcaaggtaacattttctagcatcttcagctgtctacttgactgaatgcagtatatattagttgtaataaatactgcccttctgctgtgcacaaaaggcgggtattaccacttgcagaaatttgtcgggtaaaggtaattgccagttaccttgtgttcttcccttgatcaggaacacctggaggaggatgcgctgtggttgaaccgaagccctgtgagcgaagtactgatgacagaaagagcggaagataagataagaaaggaacccttgcgcggcaaggcctggtgacatagaggtagtgcgaggctcataccgccgccgctggcaggttccaggcctgtgcttttcttgccctgtatccccagtctatacttctgcgcacatcagacgagcctcagtgtttcggcacagtggtgcaacagaaaaggagagtgctggtaggtaacgctgaggcggtgaagaaagagaggtcagacggacctggaggtggctttttaactggtaaagagtgaggtctttcatgcccatcaatctgagcaccgacttgggtgttgctcctgttcgcaggaagcacaagaaatggtcagtactccacagcgtaggcatgtcggtggtgtgttggaggaggcaagattcagatgattattatatgagctcgaaaagctagagaatggatgttcagacttgagagctctgatttgagaggaattgcacttgtcgttttcccaaggcgacgcggcctttttccagagtttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaaaaaaaaaaaaaaagaatgcacaccgacatgctctgtagcacaagcaccatactggcgaactggagaggtctcggctcatcaagcaatcgccttggtgtcggacggggtcatcaagacaagacgactagacgagcactacatatagacgggaacgtacgggaggaaggaaggaaaacgagagcgaggactcactgtccggtccgcccagcttggtgacggcgtcgacgaagcgctggtggaggtccggcgtccagcgcagccgcggcttggggtcccgtgacgccgccccgtcgtagccgtagctcccctgcatcgtcgttccttcctggcgatcgccgcttcctagctatccggtggccaaagacacggctagtggtaggctcgagtgagacgagctcttgctgaagagagaatgaatgtaacgttaccgcctcctggtcgtaggtgtaataagttgtaacgcgagcgtcgttagcaagcacaggggtttgtgtatgtgaggacaagaggaggagcgagaggaggagcgcagagcgtggcggggaaggagggcgtcatgtgtgcgaggaatctaggacgacttgttggcacttggcagctgggccggggtgcgtgcgagatgcaatgcaagaacaaagcggacgggcatcacgcctccaggtccaacccgggggcgccactcggccgccgctcattgaggcccaggcgccaagacggcggctccacccacatcacaattggcaacaagaagcacacggctggggttgggacgcgtcgaatttttcaccagaaaataccgtctgatcctggcgtttcgtcagatgctatgctacgtgaacggcaaaacctagcagcagcagcagcactcagactggacaagaggagggaaatctttgcgtgggaaccaaactgaacgcgaatcgcacgagtcggatgacatatcctcgtccggagcggactcgaccgcgagtccagctgtggctgcggaatattccggcggaagcgcggggagaacgacggcggcctccggtgggacccggggcgagcgggagatgcggcgaagatgttcggcgctgatgtcgctggaatattcgcgccagctgtggctgccggtgtgacctgctgaccagacgaccagtggcagtggccaccgcctctccatcacagattcgcggacgattagccgagactaatcgctattctcaacacttttaaaaccgtgcgtgcagaatgctaagggcgcgttcgtttgcacagcaatagacatggatttatttcagctcatcaaaatctatataaattaaagaagtaatccggctagaaattaatccggagcttcaatccctaacaaccgaacagggtctaagcctgctagattcgagcatctgcgtgactctactttggctcttctcgtacgatgcgacttgacgatgcatttgggnnnnnnnntagcgacactctcctgattagtcccacggaaacgcaactctaccactatcagccgccg

SEQ ID NO: 215 - DNA, 3586 bp, Zea mays
  misc_feature (2001)..(2139): n is unknown
tcgcatctgcagcttcttttgcacctgattacagacataagcacttgtagcgtttatggaagaaagttttggagtgcaaatctcatgacaatgatgtaaatctatcttgcctcagtttgttcttgtagtttcctttggacttgaatttgataccttagtgcatcgctaagtgctatttctctgattcacataagaaatgtgatacaaatggttagttcaatcaatgcagaaaagttcaaccaaataaaatgggcccactgcagtcaattaacaggcattcaataggattcacattcctgggcttctatatatggaagtttgcatacaaagtttttgaaataaaatggaatagaaattgcttgcatttagtgtaagttaatactagctccgttctcgaatatttgtcgtccgctagttcatttttgaactaaaacacgacaaataaaaaaacggaaggagtacatgtttgtaacaggtgagcccctgaatacttgcttgtaacaggtggagcactaagtatgcttagaactttaggcaacttgtattctttagcacttcgacgccgtttgtatggtaatatctactgatagacagaatcctggttttggattttttttttatttttcctgtttttggttacacctctacagtcccatactcgcagtccaataatacatggtctgataataaaccaattaagaaggactcatgtctcagtcattaggctgtctccaacaacgtcctctatattcatcctctatatctgtcctttacagtctcctctaaaaaatttcatcctatatatctcatttctctccaacaacgtcctctaaatcacgtcctctatactcaaatactcatattaaagacattttttaattttattttttatacatacgtaattatcatactctcaaatgtattgtgcatattttagttttgctaaaccggttatttaaagtagtcaaatggatagaggaccgtttagagaaactctatatatagagaattcagcagcgtcctctaaatttaaaggaccgtttagaggacgttgctggagagcgtagaggaccgtttggtcctctatatttagggtagagaaccctttagggggccttgttggagccagccttatgacttgagcataggagttgagatcaagaaatatgtgagttgcagcttaaggttcagagaggaaatccccatacacttgcttgtaacggtatgatcatatcttttcaaggtaacattttctagcatcttcagctgtctacttgactgaatgcagtatatattagttgtaataaatactgcccttctgctgtgcacaaaaggcgggtattaccacttgcagaaatttgtcgggtaaaggtaattgccagttaccttgtgttcttcccttgatcaggaacacctggaggaggatgcgctgtggttgaaccgaagccctgtgagcgaagtactgatgacagaaagagcggaagataagataagaaaggaacccttgcgcggcaaggcctggtgacatagaggtagtgcgaggctcataccgccgccgctggcaggttccaggcctgtgcttttcttgccctgtatccccagtctatacttctgcgcacatcagacgagcctcagtgtttcggcacagtggtgcaacagaaaaggagagtgctggtaggtaacgctgaggcggtgaagaaagagaggtcagacggacctggaggtggctttttaactggtaaagagtgaggtctttcatgcccatcaatctgagcaccgacttgggtgttgctcctgttcgcaggaagcacaagaaatggtcagtactccacagcgtaggcatgtcggtggtgtgttggaggaggcaagattcagatgattattatatgagctcgaaaagctagagaatggatgttcagacttgagagctctgatttgagaggaattgcacttgtcgttttcccaaggcgacgcggcctttttccagagtttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaaaaaaaaaaaaaaaaaagaatgcacaccgacatgctctgtagcacaagcaccatactggcgaactggagaggtctcggctcatcaagcaatcgccttggtgtcggacggggtcatcaagacaagacgactagacgagcactacatatagacgggaacgtacgggaggaaggaaggaaaacgagagcgaggactcactgtccggtccgcccagcttggtgacggcgtcgacgaagcgctggtggaggtccggcgtccagcgcagccgcggcttggggtcccgtgacgccgccccgtcgtagccgtagctcccctgcatcgtcgttccttcctggcgatcgccgcttcctagctatccggtggccaaagacacggctagtggtaggctcgagtgagacgagctcttgctgaagagagaatgaatgtaacgttaccgcctcctggtcgtaggtgtaataagttgtaacgcgagcgtcgttagcaagcacaggggtttgtgtatgtgaggacaagaggaggagcgagaggaggagcgcagagcgtggcggggaaggagggcgtcatgtgtgcgaggaatctaggacgacttgttggcacttggcagctgggccggggtgcgtgcgagatgcaatgcaagaacaaagcggacgggcatcacgcctccaggtccaacccgggggcgccactcggccgccgctcattgaggcccaggcgccaagacggcggctccacccacatcacaattggcaacaagaagcacacggctggggttgggacgcgtcgaatttttcaccagaaaataccgtctgatcctggcgtttcgtcagatgctatgctacgtgaacggcaaaacctagcagcagcagcagcactcagactggacaagaggagggaaatctttgcgtgggaaccaaactgaacgcgaatcgcacgagtcggatgacatatcctcgtccggagcggactcgaccgcgagtccagctgtggctgcggaatattccggcggaagcgcggggagaacgacggcggcctccggtgggacccggggcgagcgggagatgcggcgaagatgttcggcgctgatgtcgctggaatattcgcgccagctgtggctgccggtgtgacctgctgaccagacgaccagtggcagtggccaccgcctctccatcacagattcgcggacgattagccgagactaatcgctattctcaacacttttaaaaccgtgcgtgcagaatgctaagggcgcgttcgtttgcacagcaatagacatggatttatttcagctcatcaaaatctatataaattaaagaagtaatccggctagaaattaatccggagcttcaatccctaacaaccgaacagggtctaagcctgctagattcgagcatctgcgtgactctactttggctcttctcgtacgatgcgacttgacgatgcatttgg

SEQ ID NO: 216 - DNA, 3588 bp, Zea mays
  misc_feature (2000)..(2143): n is unknown
tcgcatctgcagcttcttttgcacctgattacagacataagcacttgtagcgtttatggaagaaagttttggagtgcaaatctcatgacaatgatgtaaatctatcttgcctcagtttgttcttgtagtttcctttggacttgaatttgataccttagtgcatcgctaagtgctatttctctgattcacataagaaatgtgatacaaatggttagttcaatcaatgcagaaaagttcaaccaaataaaatgggcccactgcagtcaattaacaggcattcaataggattcacattcctgggcttctatatatggaagtttgcatacaaagtttttgaaataaaatggaatagaaattgcttgcatttagtgtaagttaatactagctccgttctcgaatatttgtcgtccgctagttcatttttgaactaaaacacgacaaataaaaaaacggaaggagtacatgtttgtaacaggtgagcccctgaatacttgcttgtaacaggtggagcactaagtatgcttagaactttaggcaacttgtattctttagcacttcgacgccgtttgtatggtaatatctactgatagacagaatcctggttttggattttttttttatttttcctgtttttggttacacctctacagtcccatactcgcagtccaataatacatggtctgataataaaccaattaagaaggactcatgtctcagtcattaggctgtctccaacaacgtcctctatattcatcctctatatctgtcctttacagtctcctctaaaaaatttcatcctatatatctcatttctctccaacaacgtcctctaaatcacgtcctctatactcaaatactcatattaaagacattttttaattttattttttatacatacgtaattatcatactctcaaatgtattgtgcatattttagttttgctaaaccggttatttaaagtagtcaaatggatagaggaccgtttagagaaactctatatatagagaattcagcagcgtcctctaaatttaaaggaccgtttagaggacgttgctggagagcgtagaggaccgtttggtcctctatatttagggtagagaaccctttagggggccttgttggagccagccttatgacttgagcataggagttgagatcaagaaatatgtgagttgcagcttaaggttcagagaggaaatccccatacacttgcttgtaacggtatgatcatatcttttcaaggtaacattttctagcatcttcagctgtctacttgactgaatgcagtatatattagttgtaataaatactgcccttctgctgtgcacaaaaggcgggtattaccacttgcagaaatttgtcgggtaaaggtaattgccagttaccttgtgttcttcccttgatcaggaacacctggaggaggatgcgctgtggttgaaccgaagccctgtgagcgaagtactgatgacagaaagagcggaagataagataagaaaggaacccttgcgcggcaaggcctggtgacatagaggtagtgcgaggctcataccgccgccgctggcaggttccaggcctgtgcttttcttgccctgtatccccagtctatacttctgcgcacatcagacgagcctcagtgtttcggcacagtggtgcaacagaaaaggagagtgctggtaggtaacgctgaggcggtgaagaaagagaggtcagacggacctggaggtggctttttaactggtaaagagtgaggtctttcatgcccatcaatctgagcaccgacttgggtgttgctcctgttcgcaggaagcacaagaaatggtcagtactccacagcgtaggcatgtcggtggtgtgttggaggaggcaagattcagatgattattatatgagctcgaaaagctagagaatggatgttcagacttgagagctctgatttgagaggaattgcacttgtcgttttcccaaggcgacgcggcctttttccagagttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaaaaaaaaaaaaaagaatgcacaccgacatgctctgtagcacaagcaccatactggcgaactggagaggtctcggctcatcaagcaatcgccttggtgtcggacggggtcatcaagacaagacgactagacgagcactacatatagacgggaacgtacgggaggaaggaaggaaaacgagagcgaggactcactgtccggtccgcccagcttggtgacggcgtcgacgaagcgctggtggaggtccggcgtccagcgcagccgcggcttggggtcccgtgacgccgccccgtcgtagccgtagctcccctgcatcgtcgttccttcctggcgatcgccgcttcctagctatccggtggccaaagacacggctagtggtaggctcgagtgagacgagctcttgctgaagagagaatgaatgtaacgttaccgcctcctggtcgtaggtgtaataagttgtaacgcgagcgtcgttagcaagcacaggggtttgtgtatgtgaggacaagaggaggagcgagaggaggagcgcagagcgtggcggggaaggagggcgtcatgtgtgcgaggaatctaggacgacttgttggcacttggcagctgggccggggtgcgtgcgagatgcaatgcaagaacaaagcggacgggcatcacgcctccaggtccaacccgggggcgccactcggccgccgctcattgaggcccaggcgccaagacggcggctccacccacatcacaattggcaacaagaagcacacggctggggttgggacgcgtcgaatttttcaccagaaaataccgtctgatcctggcgtttcgtcagatgctatgctacgtgaacggcaaaacctagcagcagcagcagcactcagactggacaagaggagggaaatctttgcgtgggaaccaaactgaacgcgaatcgcacgagtcggatgacatatcctcgtccggagcggactcgaccgcgagtccagctgtggctgcggaatattccggcggaagcgcggggagaacgacggcggcctccggtgggacccggggcgagcgggagatgcggcgaagatgttcggcgctgatgtcgctggaatattcgcgccagctgtggctgccggtgtgacctgctgaccagacgaccagtggcagtggccaccgcctctccatcacagattcgcggacgattagccgagactaatcgctattctcaacacttttaaaaccgtgcgtgcagaatgctaagggcgcgttcgtttgcacagcaatagacatggatttatttcagctcatcaaaatctatataaattaaagaagtaatccggctagaaattaatccggagcttcaatccctaacaaccgaacagggtctaagcctgctagattcgagcatctgcgtgactctactttggctcttctcgtacgatgcgacttgacgatgcatttgggc

SEQ ID NO: 217 - DNA, 3548 bp, Zea mays
  misc_feature (1866)..(2143): n is unknown
tcgcatctgcagcttcttttgcacctgattacagacataagcacttgtagcgtttatggaagaaagttttggagtgcaaatctcatgacaatgatgtaaatctatcttgcctcagtttgttcttgtagtttcctttggacttgaatttgataccttagtgcatcgctaagtgctatttctctgattcacataagaaatgtgatacaaatggttagttcaatcaatgcagaaaagttcaaccaaataaaatgggcccactgcagtcaattaacaggcattcaataggattcacattcctgggcttctatatatggaagtttgcatacaaagtttttgaaataaaatggaatagaaattgcttgcatttagtgtaagttaatactagctccgttctcgaatatttgtcgtccgctagttcatttttgaactaaaacacgacaaataaaaaaacggaaggagtacatgtttgtaacaggtgagcccctgaatacttgcttgtaacaggtggagcactaagtatgcttagaactttaggcaacttgtattctttagcacttcgacgccgtttgtatggtaatatctactgatagacagaatcctggttttggattttttttttatttttcctgtttttggttacacctctacagtcccatactcgcagtccaataatacatggtctgataataaaccaattaagaaggactcatgtctcagtcattaggctgtctccaacaacgtcctctatattcatcctctatatctgtcctttacagtctcctctaaaaaatttcatcctatatatctcatttctctccaacaacgtcctctaaatcacgtcctctatactcaaatactcatattaaagacattttttaattttattttttatacatacgtaattatcatactctcaaatgtattgtgcatattttagttttgctaaaccggttatttaaagtagtcaaatggatagaggaccgtttagagaaactctatatatagagaattcagcagcgtcctctaaatttaaaggaccgtttagaggacgttgctggagagcgtagaggaccgtttggtcctctatatttagggtagagaaccctttagggggccttgttggagccagccttatgacttgagcataggagttgagatcaagaaatatgtgagttgcagcttaaggttcagagaggaaatccccatacacttgcttgtaacggtatgatcatatcttttcaaggtaacattttctagcatcttcagctgtctacttgactgaatgcagtatatattagttgtaataaatactgcccttctgctgtgcacaaaaggcgggtattaccacttgcagaaatttgtcgggtaaaggtaattgccagttaccttgtgttcttcccttgatcaggaacacctggaggaggatgcgctgtggttgaaccgaagccctgtgagcgaagtactgatgacagaaagagcggaagataagataagaaaggaacccttgcgcggcaaggcctggtgacatagaggtagtgcgaggctcataccgccgccgctggcaggttccaggcctgtgcttttcttgccctgtatccccagtctatacttctgcgcacatcagacgagcctcagtgtttcggcacagtggtgcaacagaaaaggagagtgctggtaggtaacgctgaggcggtgaagaaagagaggtcagacggacctggaggtggctttttaactggtaaagagtgaggtctttcatgcccatcaatctgagcaccgacttgggtgttgctcctgttcgcaggaagcacaagaaatggtcagtactccacagcgtaggcatgtcggtggtgtgttggaggaggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaaaaaaaaaaaaaagaatgcagaccgacatgctctgtagcacaagcaccatactggcgaactggagaggtctcggctcatcaagcaatcgccttggtgtcggacggggtcatcaagacaagacgactagacgagcactacatatagacgggaacgtacgggaggaaggaaggaaaacgagagcgaggactcactgtccggtccgcccagcttggtgacggcgtcgacgaagcgctggtggaggtccggcgtccagcgcagccgcggcttggggtcccgtgacgccgccccgtcgtagccgtagctcccctgcatcgccgttccttcctggcgatcgccgctccctagctatccggtggccaaagacacggctagtggtaggctcgagcgagacgagctcttggtgaagagagaatgaatgtaacgttaccgcctcctggtcgtaggggtgtgtgtatgtgaggacaagaggaggagcgagaggaggagcgcagagcgtggcggggaaggagggcgtcatgtgtgcgaggaatctaggacgacttgttggcagctgggccggggtgcgtgcgagatgcaatgcaagaacaaagcggacgggcatctcgctcggccacgcttccaagtccatccggggggcgccactcggccgccgctcattgaggcccaggcgccaagacggcggctccacccacgtcacaattggcaacaagaagcacacggctggggctgggacgcgtcgaatttttcaccagaaaataccgtctgatcctggcgtttcgtgaacggcaaaacctagcagcagcagcagcattccacgggtcggatgacatatcatatcctcgtgcggagcggactctacggcgagtccagctgtggctgcggaatattccggcggaagcgcggggagagcgacggcggcctccggtgggacccggggcgagcgggagatgcggcgaagatgttcggcgctgatgtcgctggaatattcgcgccagctgtggctgccggtgcgacctgctgaccagacgaccaatggcagtggccaccgcctctccctcttgctgttggagttggatccacggaccactctccatccaacatccatcacagattggcggacgattagccgagactaatcgctattctcaacacttttaaaaccgtacgtgcaaaatgctaaggggccgttcgtttcttagccggaatggcggtttgtttctctaatttatataagttttgattagctgtattgattcctgatccaattctgaacaaacgaacaaaacctgctagattcgagcatctgcgtgactctactttggcccttctcgtacgnnnnnnntggcgttcctctagcgtcactttcccccggaaacgcaactctaccactatcagccgccg

SEQ ID NO: 218 - DNA, 3548 bp, Zea mays
  misc_feature (2003)..(2144): n is unknown
tcgcatctgcagcttcttttgcacctgattacagacataagcacttgtagcgtttatggaagaaagttttggagtgcaaatctcatgacaatgatgtaaatctatcttgcctcagtttgttcttgtagtttcctttggacttgaatttgataccttagtgcatcgctaagtgctatttctctgattcacataagaaatgtgatacaaatggttagttcaatcaatgcagaaaagttcaaccaaataaaatgggcccactgcagtcaattaacaggcattcaataggattcacattcctgggcttctatatatggaagtttgcatacaaagtttttgaaataaaatggaatagaaattgcttgcatttagtgtaagttaatactagctccgttctcgaatatttgtcgtccgctagttcatttttgaactaaaacacgacaaataaaaaaacggaaggagtacatgtttgtaacaggtgagcccctgaatacttgcttgtaacaggtggagcactaagtatgcttagaactttaggcaacttgtattctttagcacttcgacgccgtttgtatggtaatatctactgatagacagaatcctggttttggattttttttttatttttcctgtttttggttacacctctacagtcccatactcgcagtccaataatacatggtctgataataaaccaattaagaaggactcatgtctcagtcattaggctgtctccaacaacgtcctctatattcatcctctatatctgtcctttacagtctcctctaaaaaatttcatcctatatatctcatttctctccaacaacgtcctctaaatcacgtcctctatactcaaatactcatattaaagacattttttaattttattttttatacatacgtaattatcatactctcaaatgtattgtgcatattttagttttgctaaaccggttatttaaagtagtcaaatggatagaggaccgtttagagaaactctatatatagagaattcagcagcgtcctctaaatttaaaggaccgtttagaggacgttgctggagagcgtagaggaccgtttggtcctctatatttagggtagagaaccctttagggggccttgttggagccagccttatgacttgagcataggagttgagatcaagaaatatgtgagttgcagcttaaggttcagagaggaaatccccatacacttgcttgtaacggtatgatcatatcttttcaaggtaacattttctagcatcttcagctgtctacttgactgaatgcagtatatattagttgtaataaatactgcccttctgctgtgcacaaaaggcgggtattaccacttgcagaaatttgtcgggtaaaggtaattgccagttaccttgtgttcttcccttgatcaggaacacctggaggaggatgcgctgtggttgaaccgaagccctgtgagcgaagtactgatgacagaaagagcggaagataagataagaaaggaacccttgcgcggcaaggcctggtgacatagaggtagtgcgaggctcataccgccgccgctggcaggttccaggcctgtgcttttcttgccctgtatccccagtctatacttctgcgcacatcagacgagcctcagtgtttcggcacagtggtgcaacagaaaaggagagtgctggtaggtaacgctgaggcggtgaagaaagagaggtcagacggacctggaggtggctttttaactggtaaagagtgaggtctttcatgcccatcaatctgagcaccgacttgggtgttgctcctgttcgcaggaagcacaagaaatggtcagtactccacagcgtaggcatgtcggtggtgtgttggaggaggcaagattcagatgattattatatgagctcgaaaagctagagaatggatgttcagacttgagagctctgatttgagaggaattgcacttgtcgttttcccaaggcgacgcggcctttttccagagtttttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaaaaaaaaaaaaagaatgcagaccgacatgctctgtagcacaagcaccatactggcgaactggagaggtctcggctcatcaagcaatcgccttggtgtcggacggggtcatcaagacaagacgactagacgagcactacatatagacgggaacgtacgggaggaaggaaggaaaacgagagcgaggactcactgtccggtccgcccagcttggtgacggcgtcgacgaagcgctggtggaggtccggcgtccagcgcagccgcggcttggggtcccgtgacgccgccccgtcgtagccgtagctcccctgcatcgccgttccttcctggcgatcgccgctccctagctatccggtggccaaagacacggctagtggtaggctcgagcgagacgagctcttggtgaagagagaatgaatgtaacgttaccgcctcctggtcgtaggggtgtgtgtatgtgaggacaagaggaggagcgagaggaggagcgcagagcgtggcggggaaggagggcgtcatgtgtgcgaggaatctaggacgacttgttggcagctgggccggggtgcgtgcgagatgcaatgcaagaacaaagcggacgggcatctcgctcggccacgcttccaagtccatccggggggcgccactcggccgccgctcattgaggcccaggcgccaagacggcggctccacccacgtcacaattggcaacaagaagcacacggctggggctgggacgcgtcgaatttttcaccagaaaataccgtctgatcctggcgtttcgtgaacggcaaaacctagcagcagcagcagcattccacgggtcggatgacatatcatatcctcgtgcggagcggactctacggcgagtccagctgtggctgcggaatattccggcggaagcgcggggagagcgacggcggcctccggtgggacccggggcgagcgggagatgcggcgaagatgttcggcgctgatgtcgctggaatattcgcgccagctgtggctgccggtgcgacctgctgaccagacgaccaatggcagtggccaccgcctctccctcttgctgttggagttggatccacggaccactctccatccaacatccatcacagattggcggacgattagccgagactaatcgctattctcaacacttttaaaaccgtacgtgcaaaatgctaaggggccgttcgtttcttagccggaatggcggtttgtttctctaatttatataagttttgattagctgtattgattcctgatccaattctgaacaaacgaacaaaacctgctagattcgngcatctgcgtgactctactttggcccttctcgtacgagcttttnggcgttcctctagcgtcactttcccccggaaacgcaactctaccactatcagccgccg

SEQ ID NO: 219 - DNA, 3482 bp, Zea mays
  misc_feature (2003)..(2143): n is unknown
tcgcatctgcagcttcttttgcacctgattacagacataagcacttgtagcgtttatggaagaaagttttggagtgcaaatctcatgacaatgatgtaaatctatcttgcctcagtttgttcttgtagtttcctttggacttgaatttgataccttagtgcatcgctaagtgctatttctctgattcacataagaaatgtgatacaaatggttagttcaatcaatgcagaaaagttcaaccaaataaaatgggcccactgcagtcaattaacaggcattcaataggattcacattcctgggcttctatatatggaagtttgcatacaaagtttttgaaataaaatggaatagaaattgcttgcatttagtgtaagttaatactagctccgttctcgaatatttgtcgtccgctagttcatttttgaactaaaacacgacaaataaaaaaacggaaggagtacatgtttgtaacaggtgagcccctgaatacttgcttgtaacaggtggagcactaagtatgcttagaactttaggcaacttgtattctttagcacttcgacgccgtttgtatggtaatatctactgatagacagaatcctggttttggattttttttttatttttcctgtttttggttacacctctacagtcccatactcgcagtccaataatacatggtctgataataaaccaattaagaaggactcatgtctcagtcattaggctgtctccaacaacgtcctctatattcatcctctatatctgtcctttacagtctcctctaaaaaatttcatcctatatatctcatttctctccaacaacgtcctctaaatcacgtcctctatactcaaatactcatattaaagacattttttaattttattttttatacatacgtaattatcatactctcaaatgtattgtgcatattttagttttgctaaaccggttatttaaagtagtcaaatggatagaggaccgtttagagaaactctatatatagagaattcagcagcgtcctctaaatttaaaggaccgtttagaggacgttgctggagagcgtagaggaccgtttggtcctctatatttagggtagagaaccctttagggggccttgttggagccagccttatgacttgagcataggagttgagatcaagaaatatgtgagttgcagcttaaggttcagagaggaaatccccatacacttgcttgtaacggtatgatcatatcttttcaaggtaacattttctagcatcttcagctgtctacttgactgaatgcagtatatattagttgtaataaatactgcccttctgctgtgcacaaaaggcgggtattaccacttgcagaaatttgtcgggtaaaggtaattgccagttaccttgtgttcttcccttgatcaggaacacctggaggaggatgcgctgtggttgaaccgaagccctgtgagcgaagtactgatgacagaaagagcggaagataagataagaaaggaacccttgcgcggcaaggcctggtgacatagaggtagtgcgaggctcataccgccgccgctggcaggttccaggcctgtgcttttcttgccctgtatccccagtctatacttctgcgcacatcagacgagcctcagtgtttcggcacagtggtgcaacagaaaaggagagtgctggtaggtaacgctgaggcggtgaagaaagagaggtcagacggacctggaggtggctttttaactggtaaagagtgaggtctttcatgcccatcaatctgagcaccgacttgggtgttgctcctgttcgcaggaagcacaagaaatggtcagtactccacagcgtaggcatgtcggtggtgtgttggaggaggcaagattcagatgattattatatgagctcgaaaagctagagaatggatgttcagacttgagagctctgatttgagaggaattgcacttgtcgttttcccaaggcgacgcggcctttttccagagtttttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaaaaaaaaaaaaaagaatgcagaccgacatgctctgtagcacaagcaccatactggcgaactggagaggtctcggctcatcaagcaatcgccttggtgtcggacggggtcatcaagacaagacgactagacgagcactacatatagacgggaacgtacgggaggaaggaaggaaaacgagagcgaggactcactgtccggtccgcccagcttggtgacggcgtcgacgaagcgctggtggaggtccggcgtccagcgcagccgcggcttggggtcccgtgacgccgccccgtcgtagccgtagctcccctgcatcgccgttccttcctggcgatcgccgctccctagctatccggtggccaaagacacggctagtggtaggctcgagcgagacgagctcttggtgaagagagaatgaatgtaacgttaccgcctcctggtcgtaggggtgtgtgtatgtgaggacaagaggaggagcgagaggaggagcgcagagcgtggcggggaaggagggcgtcatgtgtgcgaggaatctaggacgacttgttggcagctgggccggggtgcgtgcgagatgcaatgcaagaacaaagcggacgggcatctcgctcggccacgcttccaagtccatccggggggcgccactcggccgccgctcattgaggcccaggcgccaagacggcggctccacccacgtcacaattggcaacaagaagcacacggctggggctgggacgcgtcgaatttttcaccagaaaataccgtctgatcctggcgtttcgtgaacggcaaaacctagcagcagcagcagcattccacgggtcggatgacatatcatatcctcgtgcggagcggactctacggcgagtccagctgtggctgcggaatattccggcggaagcgcggggagagcgacggcggcctccggtgggacccggggcgagcgggagatgcggcgaagatgttcggcgctgatgtcgctggaatattcgcgccagctgtggctgccggtgcgacctgctgaccagacgaccaatggcagtggccaccgcctctccctcttgctgttggagttggatccacggaccactctccatccaacatccatcacagattggcggacgattagccgagactaatcgctattctcaacacttttaaaaccgtacgtgcaaaatgctaaggggccgttcgtttcttagccggaatggcggtttgtttctctaatttatataagttttgattagctgtattgattcctgatccaattctgaacaaacgaacaaaacctgctagattcgagcatctgcgtgactctactttggcccttctcgtacg

SEQ ID NO: 220 - DNA, 2977 bp, Zea mays
  misc_feature (1593)..(1739): n is unknown
tcgcatctgcagcttcttttgcacctgattacagacataagcacttgtagcgtttatggaagaaagttttggagtgcagatctcatgacaatgatgtaaatctatcttgcctcagtttgttcttgtagtttcctttggacttgaatttgataccttagtgcatcgctaagtgctatttctctgattcacataagaaatgcgatacaaatggttagttcagtcaatgcagaaaagttcaacaaaataaaatgggcccactgcagtcaattaacaggcattcaacagcattcacattcctgggcctctatatatggaagtttgcatacaaagttttggaaataaaatggaatagaaattgcttgcatttagtgtaagttaatacccgctccgttctcgaatatttgtcgcctgctagttcatttttgaactaaaacacgacaaataaaaaaaacggaaggagtacatgtttgtaacaggagagcccctgaatacttgcttgtaacaggtggagcgctaagtatgcttaggagaactttaggcaacttgtattctttagcacttcgacgccgtttgtatggtaatatctactgatagacagaatcctggttttggaatttttttatttttcctgcttttggttacacctctacagtcccatactcgcagtcgaataatacatggtctgataataaaccaattaaggactcatgtctcagtcattatgacttgagcataggagttcagatcgagaaatatttgagttgcagcttaaggttcagagaggaaacccccatacacttgcttgtaacggtatgatcattttttttgaaggtaacattttctagcatcttcagctgtctacttgactgaatgcagtatatattagttgtaataaatactgcccttctgctgtgcacaaaaggcgggtattaccacttgcagaaatttgtcgggtaaaggtaattgccagttaccttgtgttcttcccttcatcaggaacacctggaggaggatgcgctgtggttgaactgaagccctgcgagagaagtactgatgacagaaagagcggaagataagataagaaaggaaacccttgcgcggcagggcctggtgacatagaggtagtgcgaggctcataccgccgccgctggcaggttccaggcctgtgcttttcttgccctgtatccccagtctatacttctgcgcacatcagacgagcgtcagtgtttcggcacagtggtgcaacagaaaaggagagtgctggtaggtaacgctgaggcggtgaagaaagagaggtcagacggacctggaggtggctttttaactggtaaagagtgaggtctttcatgcccatcaatctgagcaccgacttgggtgttgctcctgttcgcaggaagcacaagaaatggtcagtactccacagcgtaggcatgtcggtggtgtgttggaggaggcaagattcagatgattattatatgagctcgaaaagctagagaatggatgttcagacttgagagctctgatttgagagaaattgcacttgtcgttttcccaaggcgacgcggccttttccagaggttttttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctgaaaaaaaaatgcacaccgacatgctctgtagcacaagcaccataccggcgaactggagaggtctcggctcatcaagcaatcgccttggtgtcggacggggtcatcaagacaaggcgactagaggagcactacatctacacggggtgaacggacgggagcagtggcggacccaggaactgatgacagccttggcgagaatacggtgtgatccccacgcctgtgctcgtgccacgtgctgcttgcttccgtgcactgtgctcgcgccttgcccattgcagccggcgagccagctcaggccaccgcctgcggtgcctggtgagtccgcccctggacgggaggaaggaaggaaaacgagagcgaggactcactgtccggtccgcccagcttggtgacggcgtcgacgaagcgctggtggaggtccggcgtccagcgcagccgcggcttggggtcccgtgacgccgccccgtcgtagccgtagctcccctgcatcgccgttccttcctggcgatcgccgctccctagctatccggtggccaaagacacggctagtggtaggctcgagcgagacgagctcttgctgaagagagaatgaatgtagcgttaccgcctcctggtcgtaggggtgtgggtatgtgaggacaagaggaggagcgagaggaggagcgcagagcgtggcggggaaggagggcgtcatgtgtgtgaggaatctaggacgacttgttggcagctgggccggggtgcgtgcgagatgcaatgcaagaacaaagcggacgggcatcacgcctccaagtccaacccgggggcgccactcggccgccgctcattgaggcccaggcgccaagacggcggctccacccacgtcacaattggcaacaagaagcacacggctggggctgggacgcgtcgaatttttcaccagaaaataccgtcggcgtttcgtcagatgctatgctacgtgaacggcaaaacctagcagcagcagcagcattcagactggacaagaggagggaaatctttgcgtgggaaccaaactgaacgcgaatcgcacgagtcggatgacatatcctcgtgcggagcggactcgaccgcgagtccagctgtggntgcggaatattccggcggaagcgcggggagaacgacggcggcctccggtgggacccggggcgagcgggagatgcggcgaagatgttcggcgctgatgtcgctggaatattcgcgccagctgtggctgccgg

SEQ ID NO: 221 - DNA, 3064 bp, Zea mays
  misc_feature (1464)..(1891): n is unknown
tcgcatctgcagcttcttttgcacctgattacagacataagcacttgtagcgtttatggaagaaaggtttggagtgcagatctcatgacaatgatgtaaatctatcttgcctcagtttgttcttgtagtttcctttggacttgaatttgataccttagtgcatcgctaagtgctatttctctgattcacataagaaatgtgatacaaatggttagctcaatcaatgcagaaaagttcaaccaaataaaatgggcccactgcagtcaattaacaggcattcaataggattcacattcctgggcttctatatatggaagtttgcatacaaagttttggaaataaaatggaatataaattgcttgcatttagtgtaagttaatacccgctctgttctcgaatatttgtcacccgctagttcatttttgaactaaaacacgacaaataaaaaaacggaaggagtacatgtttgtaacaggagagcccctgaatacttgcttgtaacaggtggagcgctaagtatgcttaggagaactttaggcaacttgtattctttagcacttcgacgccgtttgtatggtaatatctactgatagacagaatcctggttttggaattttttttatttttcctgcttttggttacacctctacagtcccatactcgcagtcgaataatacatggtctgataataaaccaattaaggactcatgtctcagtcattatgacttgagcataggagttgagatcaagaaatatttgagttgcagcttaaggttcagagaggaaatccccatacacgtgcttgtaacggtatggtcatttttttttcaaggtaacattttctagcatcttcagctgtctacttgactgaatgcagtatatattagttgtaataaatactggccttctgctgtgcacaaaaggcgggtattaccacttgcagaaatttgtcgggtcaaggtaattgccagttaccttgtgttcttcccttgatcaggaacacctggaggaggatgcgctgtggttgaactgaagccgccctgtgagcgaagtactgatgacagaaagagcggaagataagataagaaaggaacgcttgcgcggcaaggcctggtgacatagaggtagtgcgaggctcataccgccgccgctggcaggttcgaggcctgtgcttttcttgccctgtatccccagtctatacttctgcgcacatcagacgagcctcagtgtttcggcacagtggtgcaacagaaaaggagagtgctggtaggtaacgctgaggcggtgaagaaagagaggtcagacggacctggaggtggctttttaactggtaaagagtgaggtctttcatgcccatcaatctgagcaccgacttgggtgttgctcctgttcgcaggaagcacaagaaatggtcagtactccacagcgtaggcatgtcggtggtgtgttggaggaggcaagatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggacgggaggaaggaaggaaaacgagagcgaggactcactgtccggtccgcccagcttggtgacggcgtcgacgaagcgctggtggaggtccggcgtccagcgcagccgcggcttggggtcccgtgacgccgccccgtcgtagccgtagctcccctgcatcgccgttccttcctggcgatcgccgctccctagctatccggtggccaaagacacggctagtggtaggctcgagcgagacgagctcttgctgaagagagaatgaatgtagcgttaccgcctcctggtcgtaggggtgtgggtatgtgaggacaagaggaggagcgagaggaggagcgcagagcgtggcggggaaggagggcgtcatgtgtgtgaggaatctaggacgacttgttggcagctgggccggggtgcgtgcgagatgcaatgcaagaacaaagcggacgggcatcacgcctccaggtccaacccgggggcgccactcgatcggccgccgctcattgaggcccaggcgccaagacggcggctccacccacgtcacaattggcaataagaagcacacggctggggctgggacgcgtcgaatttttcaccagaaaataccgtctgatcctggcgtttcgtcagatgctatgctacgtgaacggcaaaacctagcagcagcagcactcagactggacaagaggagggaaatctttgcgtgggaaccaaactgaacgcgaatcgcacgggtcggatgacatatcatatcctcgtgcggagcggactcaacggcgagtccagctgtggctgcggaatattccggcggaagcgcggggagagcgacggcggcctccggtgggacccggggcgagcgggagatgcggcgaagatgttcggcgctgatgtcgctggaatattcgcgccagctgtggctgccggtgcgacctgctgaccagacgaccagtggcaatggccaccgcctctccatccaacctccatcacagattggcggacgattagccgagactaatcgctattctcaacactttaaaaaccgtgcgtgcagaatgctaagcctgctagattcgagcatctgcgtgactctactttggctcttctcgtacgatgcgacctgacgatgcatttgggcgncctntagcgtcacttcctgattagtcccccggaaacgcaac

SEQ ID NO: 222 - DNA, 3087 bp, Zea mays
  misc_feature (1281)..(1735): n is unknown
tcgcatctgcagcttcttttgcacctgattacagacataagcacttgtagcgtttatggaagaaaggtttggagtgcagatctcatgacaatgatgtaaatctatcttgcctcagtttgttcttgtagtttcctttggacttgaatttgataccttagtgcatcgctaagtgctatttctctgattcacataagaaatgtgatacaaatggttagctcaatcaatgcagaaaagttcaaccaaataaaatgggcccactgcagtcaattaacaggcattcaataggattcacattcctgggcttctatatatggaagtttgcatacaaagttttggaaataaaatggaatataaattgcttgcatttagtgtaagttaatacccgctctgttctcgaatatttgtcacccgctagttcatttttgaactaaaacacgacaaataaaaaaacggaaggagtacatgtttgtaacaggagagcccctgaatacttgcttgtaacaggtggagcgctaagtatgcttaggagaactttaggcaacttgtattctttagcacttcgacgccgtttgtatggtaatatctactgatagacagaatcctggttttggaatttttttttatttttcctgcttttggttacacctctacagtcccatactcgcagtcgaataatacatggtctgataataaaccaattaaggactcatgtctcagtcattatgacttgagcataggagttgagatcaagaaatatttgagttgcagcttaaggttcagagaggaaatccccatacacgtgcttgtaacggtatggtcattttttttttcaaggtaacattttctagcatcttcagctgtctacttgactgaatgcagtatatattagttgtaataaatactggccttctgctgtgcacaaaaggcgggtattaccacttgcagaaatttgtcgggtcaaggtaattgccagttaccttgtgttcttcccttcatcaggaacacctggaggaggatgcgctgtggttgaactgaagccgccctgtgagcgaagtactgatgacagaaagagcggaagataagataagaaaggaacgcttgcgcggcaaggcctggtgacatagaggtagtgcgaggctcataccgccgccgctggcaggttcgaggcctgtgcttttcttgccctgtttccccattctatacttctgcgcacatcagacgagcctcagtgtttcggcacagtggtgcaacaaaaaaagagagtgctggtaggtaaccctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaaaaaaaaaaaaaaaagaatgcagaccgacatgctctgtagcacaagcaccatactggcgaactggagaggtctcggctcatcaagcaatcgccttggtgtcggacggggtcatcaagacaagacgactagacgagcactacatatagacgggaacgtacgggaggaaggaaggaaaacgagagcgaggactcactgtccggtccgcccagcttggtgacggcgtcgacgaagcgctggtggaggtccggcgtccagcgcagccgcggcttggggtcccgtgacgccgccccgtcgtagccgtagctcccctgcatcgccgttccttcctggcgatcgccgctccctagctatccggtggccaaagacacggctagtggtaggctcgagcgagacgagctcttgctgaagagagaatgaatgtagcgttaccgcctcctggtcgtaggggtgtgggtatgtgaggacaagaggaggagcgagaggaggagcgcagagcgtggcggggaaggagggcgtcatgtgtgtgaggaatctaggacgacttgttggcagctgggccggggtgcgtgcgagatgcaatgcaagaacaaagcggacgggcatcacgcctccaggtccaacccgggggcgccactcgatcggccgccgctcattgaggcccaggcgccaagacggcggctccacccacgtcacaattggcaataagaagcacacggctggggctgggacgcgtcgaatttttcaccagaaaataccgtctgatcctggcgtttcgtcagatgctatgctacgtgaacggcaaaacctagcagcagcagcactcagactggacaagaggagggaaatctttgcgtgggaaccaaactgaacgcgaatcgcacgggtcggatgacatatcatatcctcgtgcggagcggactcaacggcgagtccagctgtggctgcggaatattccggcggaagcgcggggagagcgacggcggcctccggtgggacccggggcgagcgggagatgcggcgaagatgttcggcgctgatgtcgctggaatattcgcgccagctgtggctgccggtgcgacctgctgaccagacgaccagtggcaatggccaccgcctctccatccaacctccatcacagattggcggacgattagccgagactaatcgctattctcaacactttaaaaaccgtgcgtgcagaatgctaagcctgctagattcgagcatctgcgtgactctactttggctcttctcgtacgatgcgacctgacgatgcattgggcgnncctntagcgtcactttcctgattagtcccccggaaacgcaactctaccactatcagccgccg

SEQ ID NO: 223 - DNA, 2956 bp, Zea mays
  misc_feature (1584)..(1728): n is unknown
tcgcatctgcagcttcttttgcacctgattacagacataagcacttgtagcgtttatggaagaaagttttggagtgcaaatctcatgacaatgatgtaaatctgtcttgcctcagtttgttcttgtagtttcctttggacttgaatttgataccttagtgcatcgctaagtgctatttctctgattcgcataagaaatgcgatacaaatggttagttcaatcaatgcagaaaagttcaacaaaataaaatgggcccactgcagtcaattaacaggcattcaacagcattcacattcctgggcctctatatatggaagtttgcatacaaagttttggaaataaaatggaatagaaattgcttgcatttagtgtaagttaatactccatccgttcttaaatatttgtcggccgctagtttatttttgaactaaaacacgacaaataaaaaaaacggagggagtacatgtttataacaggtgagccgaatacttggttgtaacaggtggggcgctaagtatgcttaggagaactttaggcaacttgtattctgtagcacttcgacgccgtttgtatggtaatatctactgatagacagaatcctggtttttggaaaaaaaaaattcctgcttttggttacacctctacagtcccatactcgcagtcgaataatacatggtctgataataaaccaattaaggactcatgtctcagtcattatgacttgagcataggagttgagatcgagaaatatttgagttacagcttaaggttcagacttcagagaggaaatccccatacacttgcttgtaacggtatgatcattttttttcaaggtaacattttctagcatcttcacctgtctacttgactgaatgcagtatatattagttgtaataaatactgctcttctgctgtgcagaaaaggcgggtattaccacttgcagaaatttgtcgggtaaaggtaattgccagttaccttgtgttcttcccttcatcaggaacacctggaggaggatgcgctgtggttgaaccgaagccctgtgagcgaagtactgatgacagaaagagcggaagataagataagaaaggaacccttgcgcggcaaggcctggtgacatagaggtagtgcgaggctcataccgccgccgctggcaggttccaggcctgtgcttttcttgccctgtatccccagtctatacttctgcgcacatcagacgagcctcagtgtttcggcacagtggtgcaacagaaaaggagagtgctgctaacgctgaggcggtgaagaaagagaggtcggacggacctggaggtggctttttaactggtaaagagtgaggtctttcatgcccatcaatctgagcaccgacttgggtgttgctcctgttcgcaggaagcacaagaaatggtcagtactccacagcgtaggcatgtcggtgtgttcgaggaggcaagattcagatgattattatatgagctcgaaaagctagagaatggatgttcagacttgagaggtctgatttgagaggaattgcacttgtcgttttcccagggcgacgcggcctttttccagaggctttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaaaaaaaaaaaagaatgcagaccgacatgctctgtagcacaagcaccatactggcgaactggagaggtctcggctcatcaagcaatcgccttggtgtcggacggggtcatcaagacaagacgactagacgagcactacatatagacgggaacgtacgggaggaaggaaggaaaacgagagcgaggactcactgtccggtccgcccagcttggtgacggcgtcgacgaagcgctggtggaggtccggcgtccagcgcagccgcggcttggggtcccgtgacgcaaaccaacgtcgtagccgtagctcccctgcatcgccgttccttcctggcgatcgccgctccctagctatccggtggccaaagacacggctagtggtaggctcgagcgagacgagctcttggtgaagagagaatgaatgtaacgttaccgcctcctggtcgtaggggtgtgtgtatgtgaggacaagaggaggagcgagaggaggagcgcagagcgtggcggggaaggagggcgtcatgtgtgcgaggaatctcggacgacttgttggcagctgggccggggtgcgtgcgagatgcaatgcaagaacaaagcggacgggcatctcgctcggccacgcttccaagtccaaccggggggcgccactcggccgccgctcattgaggcccaggcgccaagacggcggctccacccacatcacaattggcaacaagaagcacacggctggggctgggacgcgtcgaatttttcaccagaaaataccgtcggcgtttcgtcagatgctatgctacgtgaacggcaaaacctagcagcagcagcactcagactggacaagaggagggaaatctttgcgtgggaaccaaactgaacgcgaatcgcacgagtcggatgacatatcctcgtccggagcggactcggccgcgagtccagctgtggctgcggaatattccggcggaatcgcggggagaacgacggcggcctccggtgggacccggggcgagcgggagatgcggcgaagatgttcggcgctgatgtcgctggaatattcgcgccagctgtggctgccggtgtgacctgctgaccagacgaccagtggcagtggccaccgcctctgcatcacagattggcggacgattagccgagactaattgccattctcaacacttttaaaaccgtgcgtgcagaatgctaagcctgctagattcgagcatctgcgtgactctactt

SEQ ID NO: 224 - DNA, 2965 bp, Zea mays
  misc_feature (1600)..(1902): n is unknown
tcgcatctgcagcttcttttgcacctgattacagacataagcacttgtagcgtttatggaagaaagttttggagtgcagatctcatgacaatgatgtaaatctatcttgcctcagtttgttcttgtagtttcctttggacttgaatttgataccttagtgcatcgctaagtgctggttctctgattcacataagaaatgtgatacaaatggttagttcaatcaatgcagaaaagttcaatcaaataaaatgggcccactgcagtcaattaacaggcattcaataggattcacattcctgggcttctatatatggaagtttgcatacaatgttttggaaataaaatgaaatataaattgcttgcatttagtgtaagttaatactcgctcccttctcgaatatttgtcgtccgctagttcatttttgaactaaaacatgataaataaaaaaacggaaggagtacatgtttgtaacaggagagcccatgaatacttgcttgtaacaggtggagcgctaagtatgcttaggagaactttaggcaacttgtattctttagcacttcgacgcagtttgtatggtaatatctactgatagacagaatcctggttttggatttttaatttttcctgcttttggttacacctctacagtcccatactcgcagtccaatagtacatggtctgataataaaccaattaagaaggactcatgtctcagtcattatgacttgagcataggagttgagatcaagaaatatttgagttgcagcttaaggtccagagaggaaatccccatacacttgcttgtaacggtatgaatgtatgatcatttttttttcaaggtaacattttctagcatcttcacctgtctacttgactgaatgcagtatatattagttgtaataactactggccttctgctgtgcacaaaaggcgggtattaccacttgcagaaatttgtcgggtaaaggtaattgccagttaccttgtgttcttcccttgatcaggaacacctggaggaggatgcgctgtggttgaaccgaagccctgtgagcgaagtactgatgacagaaagagcggaagataagataagaaaggaacccttgcgcggcaaggcctggtgacatagaggtagtgcgaggctcataccgccgccgctggcaggttccaggcctgtgcttttcttgccctgtatccccagtctatacttctgcgcacatcagacgagcctcagtgtttcggcacagtggtgcaacagaaaaggagagtgctggtaggtaacgctgaggcggtgaagaaagagaggtcagacggacctggaggtggctttttaactggtaaagagtgaggtctttcatgcccatcaatctgagcaccgacttgggtgttgctcctgttcgcaggaagcacaagaaatggtcagtactccacagcgtaggcatgtcggtggtgtgttggaggaggcaagattcagatgattattatatgagctcgaaaagctagagaatggatgttcagacttgagagctctgatttgagagraattgcacttgtcgttttcccaaggcgacgcggcctttttccagaggttttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacgggaggaaggaaggaaaacgagagcgaggactcactgtccggtccgcccagcttggtgacggcgtcgacgaagcgctggtggaggtccggcgtccagcgcagccgcggcttggggtcccgtgacgccgccccgtcgtagccgtagctcccctgcatcgccgttccttcctggcgatcgccgctccctagctatccggtggccaaagacacggctagtggtaggctcgagcgagacgagctcttgctgaagagagaatgaatgtagcgttaccgcctcctggtcgtaggggtgtgggtatgtgaggacaagaggaggagcgagaggaggagcgcagagcgtggcggggaaggagggcgtcatgtgtgcgaggaatctaggacgacttgttggcagctgggccggggtgcgtgcgagatgcaatgcaagaacaaagcggacgggcatctcgctcggccacgcttccaagtccatccggggggcgccactcggccgccgctcattgaggcccaggcgccaagacggcggctccacccacgtcacaattggcaacaagaagcacacggctggggctgggacgcgtcgaatttttcaccagaaaataccgtctgatcctggcgtttcgtgaacggcaaaacctagcagcagcagcagcattccacgggtcggatgacatatcatatcctcgtgcggagcggactcaacggcgagtccagctgtggctgcggaatattccggcggaagcgcggggagagcgacggcggcctccggtgggacccggggcgagcgggagatgcggcgaagatgttcggcgctgatgtcgctggaatattcgcgccagctgtggctgccggtgcgacctgctgaccagacgaccagtggcaatggccaccgcctctccatccaacctccatcacagattggcggacgattagccgagactaatcgctattctcaacactttaaaaaccgtgcgtgcagaatgctaagcctgctagattcgagcatctgcgtgactctactttggctcttctcgtacgatgcgacctgacgatgcatttgg

SEQ ID NO: 225 - DNA, 2855 bp, Zea mays
  misc_feature (1601)..(1748): n is unknown
tcgcatctgcagcttcttttgcacctgattacagacataagcacttgtagcgtttatggaagaaagttttggagtgcagatctcatgacaatgatgtaaatctatcttgcctcagtttgttcttgtagtttcctttggacttgaatttgataccttagtgcatcgctaagtgctatttctctgattcacataagaaatgtgatacaaatggttagttcaatcaatgcagaaaagttcaatcaaataaaatgggcccactgcagtcaattaacaggcattcaataggattcacattcctgggcttctatatatggaagtttgcatacaatgttttggaaataaaatgaaatataaattgcttgcatttagtgtaagttaatactcgctcccttctcgaatatttgtcgtccgctagttcatttttgaactaaaacatgataaataaaaaaacggaaggagtacatgtttgtaacaggagagcccatgaatacttgcttgtaacaggtggagcgctaagtatgcttaggagaactttaggcaacttgtattctttagcacttcgacgcagtttgtatggtaatatctactgatagacagaatcctggttttggatttttaatttttcctgcttttggttacacctctacagtcccatactcgcagtccaatagtacatggtctgataataaaccaattaagaaggactcatgtctcagtcattatgacttgagcataggagttgagatcaagaaatatttgagttgcagcttaaggtccagagaggaaatccccatacacttgcttgtaacggtatgaatgtatgatcatttttttttcaaggtaacattttctagcatcttcacctgtctacttgactgaatgcagtatatattagttgtaataactactggccttctgctgtgcacaaaaggcgggtattaccacttgcagaaatttgtcgggtaaaggtaattgccagttaccttgtgttcttcccttgatcaggaacacctggaggaggatgcgctgtggttgaaccgaagccctgtgagcgaagtactgatgacagaaagagcggaagataagataagaaaggaacccttgcgcggcaaggcctggtgacatagaggtagtgcgaggctcataccgccgccgctggcaggttccaggcctgtgcttttcttgccctgtatccccagtctatacttctgcgcacatcagacgagcctcagtgtttcggcacagtggtgcaacagaaaaggagagtgctggtaggtaacgctgaggcggtgaagaaagagaggtcagacggacctggaggtggctttttaactggtaaagagtgaggtctttcatgcccatcaatctgagcaccgacttgggtgttgctcctgttcgcaggaagcacaagaaatggtcagtactccacagcgtaggcatgtcggtggtgtgttggaggaggcaagattcagatgattattatatgagctcgaaaagctagagaatggatgttcagacttgagagctctgatttgagagraattgcacttgtcgttttcccaaggcgacgcggccttttccagaggttttttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgaaaaaaaaatgcacaccgacatgctctgtagcacaagcaccataccggcgaactggagaggtctcggctcatcaagcaatcgccttggtgtcggacggggtcatcaagacaaggcgactagaggagcactacatctacacggggtgaacggacgggagcagtggcggacccaggaactgatgacagccttggcgagaatacggtgtgatccccacgcctgtgctcgtgccacgtgctgcttgcttccgtgcactgtgctcgtgccttgcccattgcagccggcgagccagctcaggccaccgcctgcggtgcctggtgagtccgcccctggacgggaggaaggaaggaaaacgagagcgaggactcactgtccggtccgcccagcttggtgacggcgtcgacgaagcgctggtggaggtccggcgtccagcgcagccgcggcttggggtcccgtgacgccgccccgtcgtagccgtagctcccctgcatcgccgttccttcctggcgatcgccgctccctagctatccggtggccaaagacacggctagtggtaggctcgagcgagacgagctcttgctgaagagagaatgaatgtagcgttaccgcctcctggtcgtaggggtgtgggtatgtgaggacaagaggaggagcgagaggaggagcgcagagcgtggcggggaaggagggcgtcatgtgtgcgaggaatctaggacgacttgttggcagctgggccggggtgcgtgcgagatgcaatgcaagaacaaagcggacgggcatctcgctcggccacgcttccaagtccatccggggggcgccactcggccgccgctcattgaggcccaggcgccaagacggcggctccacccacgtcacaattggcaacaagaagcacacggctggggctgggacgcgtcgaatttttcaccagaaaataccgtctgatcctggcgtttcgtgaacggcaaaacctagcagcagcagcagcattccacgggtcggatgacatatcatatcctcgtgcggagcggactcaacggcgagtccagctgtggnnncggaatattccggcggaagcgcggggagagcgacggcggcctccggtgg

SEQ ID NO: 226 - DNA, 3212 bp, Zea mays
  misc_feature (1601)..(1805): n is unknown
tcgcatctgcagcttcttttgcacctgattacagacataagcacttgtagcgtttatggaagaaagttttggagtgcagatctcatgacaatgatgtaaatctatcttgcctcagtttgttcttgtagtttcctttggacttgaatttgataccttagtgcatcgctaagtgctatttctctgattcacataagaaatgtgatacaaatggttagttcaatcaatgcagaaaagttcaatcaaataaaatgggcccactgcagtcaattaacaggcattcaataggattcacattcctgggcttctatatatggaagtttgcatacaatgttttggaaataaaatgaaatataaattgcttgcatttagtgtaagttaatactcgctcccttctcgaatatttgtcgtccgctagttcatttttgaactaaaacatgataaataaaaaaacggaaggagtacatgtttgtaacaggagagcccatgaatacttgcttgtaacaggtggagcgctaagtatgcttaggagaactttaggcaacttgtattctttagcacttcgacgcagtttgtatggtaatatctactgatagacagaatcctggttttggatttttaatttttcctgcttttggttacacctctacagtcccatactcgcagtccaatagtacatggtctgataataaaccaattaagaaggactcatgtctcagtcattatgacttgagcataggagttgagatcaagaaatatttgagttgcagcttaaggtccagagaggaaatccccatacacttgcttgtaacggtatgaatgtatgatcatttttttttcaaggtaacattttctagcatcttcacctgtctacttgactgaatgcagtatatattagttgtaataactactggccttctgctgtgcacaaaaggcgggtattaccacttgcagaaatttgtcgggtaaaggtaattgccagttaccttgtgttcttcccttgatcaggaacacctggaggaggatgcgctgtggttgaaccgaagccctgtgagcgaagtactgatgacagaaagagcggaagataagataagaaaggaacccttgcgcggcaaggcctggtgacatagaggtagtgcgaggctcataccgccgccgctggcaggttccaggcctgtgcttttcttgccctgtatccccagtctatacttctgcgcacatcagacgagcctcagtgtttcggcacagtggtgcaacagaaaaggagagtgctggtaggtaacgctgaggcggtgaagaaagagaggtcagacggacctggaggtggctttttaactggtaaagagtgaggtctttcatgcccatcaatctgagcaccgacttgggtgttgctcctgttcgcaggaagcacaagaaatggtcagtactccacagcgtaggcatgtcggtggtgtgttggaggaggcaagattcagatgattattatatgagctcgaaaagctagagaatggatgttcagacttgagagctctgatttgagagraattgcacttgtcgttttcccaaggcgacgcggcctttttccagaggtttttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggagaggtctcggctcatcaagcaatcgccttggtgtcggacggggtcatcaagacaagrcgactagaggagcactacatctacacggggtgaacggacgggagcagtggcggacccaggaactgatgacagccttggcgagaatacggtgtgatccccacgcctgtgctcgtgccacgtgctgcttgcttccgtgcactgtgctcgtgccttgcccattgcagccggcgagccagctcaggccaccgcctgcggtgcctggtgagtccgcccctggacgggaggaaggaaggaaaacgagagcgaggactcactgtccggtccgcccagcttggtgacggcgtcgacgaagcgctggtggaggtccggcgtccagcgcagccgcggcttggggtcccgtgacgccgccccgtcgtagccgtagctcccctgcatcgccgttccttcctggcgatcgccgctccctagctatccggtggccaaagacacggctagtggtaggctcgagcgagacgagctcttgctgaagagagaatgaatgtagcgttaccgcctcctggtcgtaggggtgtgggtatgtgaggacaagaggaggagcgagaggaggagcgcagagcgtggcggggaaggagggcgtcatgtgtgcgaggaatctaggacgacttgttggcagctgggccggggtgcgtgcgagatgcaatgcaagaacaaagcggacgggcatctcgctcggccacgcttccaagtccatccggggggcgccactcggccgccgctcattgaggcccaggcgccaagacggcggctccacccacgtcacaattggcaacaagaagcacacggctggggctgggacgcgtcgaatttttcaccagaaaataccgtctgatcctggcgtttcgtgaacggcaaaacctagcagcagcagcagcattccacgggtcggatgacatatcatatcctcgtgcggagcggactcaacggcgagtccagctgtggctgcggaatattccggcggaagcgcggggagagcgacggcggcctccggtgggacccggggcgagcgggagatgcggcgaagatgttcggcgctgatgtcgctggaatattcgcgccagctgtggctgccggtgcgacctgctgaccagacgaccagtggcaatggccaccgcctctccatccaacctccatcacagattggcggacgattagccgagactaatcgctattctcaacactttaaaaaccgtgcgtgcagaatgctaagcctgctagattcgagcatctgcgtgactctactttggctcttctcgtacgatgcgacctgacgatgcatttgggcnnncctntagcgtcactttcctgattagtcccccggaaacgcaactctaccactatcagccgccg

SEQ ID NO: 227 - DNA, 3315 bp, Zea mays
  misc_feature (1601)..(1748): n is unknown
tcgcatctgcagcttcttttgcacctgattacagacataagcacttgtagcgtttatggaagaaagttttggagtgcagatctcatgacaatgatgtaaatctatcttgcctcagtttgttcttgtagtttcctttggacttgaatttgataccttagtgcatcgctaagtgctatttctctgattcacataagaaatgtgatacaaatggttagttcaatcaatgcagaaaagttcaatcaaataaaatgggcccactgcagtcaattaacaggcattcaataggattcacattcctgggcttctatatatggaagtttgcatacaatgttttggaaataaaatgaaatataaattgcttgcatttagtgtaagttaatactcgctcccttctcgaatatttgtcgtccgctagttcatttttgaactaaaacatgataaataaaaaaacggaaggagtacatgtttgtaacaggagagcccatgaatacttgcttgtaacaggtggagcgctaagtatgcttaggagaactttaggcaacttgtattctttagcacttcgacgcagtttgtatggtaatatctactgatagacagaatcctggttttggatttttaatttttcctgcttttggttacacctctacagtcccatactcgcagtccaatagtacatggtctgataataaaccaattaagaaggactcatgtctcagtcattatgacttgagcataggagttgagatcaagaaatatttgagttgcagcttaaggtccagagaggaaatccccatacacttgcttgtaacggtatgaatgtatgatcatttttttttcaaggtaacattttctagcatcttcacctgtctacttgactgaatgcagtatatattagttgtaataactactggccttctgctgtgcacaaaaggcgggtattaccacttgcagaaatttgtcgggtaaaggtaattgccagttaccttgtgttcttcccttgatcaggaacacctggaggaggatgcgctgtggttgaaccgaagccctgtgagcgaagtactgatgacagaaagagcggaagataagataagaaaggaacccttgcgcggcaaggcctggtgacatagaggtagtgcgaggctcataccgccgccgctggcaggttccaggcctgtgcttttcttgccctgtatccccagtctatacttctgcgcacatcagacgagcctcagtgtttcggcacagtggtgcaacagaaaaggagagtgctggtaggtaacgctgaggcggtgaagaaagagaggtcagacggacctggaggtggctttttaactggtaaagagtgaggtctttcatgcccatcaatctgagcaccgacttgggtgttgctcctgttcgcaggaagcacaagaaatggtcagtactccacagcgtaggcatgtcggtggtgtgttggaggaggcaagattcagatgattattatatgagctcgaaaagctagagaatggatgttcagacttgagagctctgatttgagaggaattgcacttgtcgttttcccaaggcgacgcggccttttccagaggttttttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgaaaaaaaaatgcacaccgacatgctctgtagcacaagcaccataccggcgaactggagaggtctcggctcatcaagcaatcgccttggtgtcggacggggtcatcaagacaaggcgactagaggagcactacatctacacggggtgaacggacgggagcagtggcggacccaggaactgatgacagccttggcgagaatacggtgtgatccccacgcctgtgctcgtgccacgtgctgcttgcttccgtgcactgtgctcgcgccttgcccattgcagccggcgagccagctcaggccaccgcctgcggtgcctggtgagtccgcccctggacgggaggaaggaaggaaaacgagagcgaggactcactgtccggtccgcccagcttggtgacggcgtcgacgaagcgctggtggaggtccggcgtccagcgcagccgcggcttggggtcccgtgacgccgccccgtcgtagccgtagctcccctgcatcgccgttccttcctggcgatcgccgctccctagctatccggtggccaaagacacggctagtggtaggctcgagcgagacgagctcttgctgaagagagaatgaatgtagcgttaccgcctcctggtcgtaggggtgtgggtatgtgaggacaagaggaggagcgagaggaggagcgcagagcgtggcggggaaggagggcgtcatgtgtgtgaggaatctaggacgacttgttggcagctgggccggggtgcgtgcgagatgcaatgcaagaacaaagcggacgggcatcacgcctccaggtccaacccgggggcgccactcggccgccgctcattgaggcccaggcgccaagacggcggctccacccacgtcacaattggcaataagaagcacacggctggggctgggacgcgtcgaatttttcaccagaaaataccgtctgatcctggcgtttcgtcagatgctatgctacgtgaacggcaaaacctagcagcagcagcagcactcagactggacaagaggagggaaatctttgcgtgggaaccaaactgaacgcgaatcgcacgagtcggatgacatatcctcgtccggagcggactcgaccgcgagtccagctgtggctgcggaatattccggcggaagcgcggggagaacgacggcggcctccggtgggacccggggcgagcgggagatgcggcgaagatgttcggcgctgatgtcgctggaatattcgcgccagctgtggctgccggtgtgacctgctgaccagacgaccagtggcagtggccaccgcctctccatcacagattcgcggacgattagccgagactaatcgctattctcaacacttttaaaaccgtgcgtgcagaatgctaagggcgcgttcgtttgcacagcaatagacatggatttatttcagctcatcaaaatttatataaattaaagaagtaatccggctagaaattaatccggagcttcaatccctaacaaccgaacagggtctaagcctgctagattcgagcatctgcgtgactctactttggctcttctcgtacgatgcgacttgacgatgcat

SEQ ID NO: 228 - DNA, 3271 bp, Zea mays
  misc_feature (1605)..(1751): n is unknown
tcgcatctgcagcttcttttgcacctgattacagacataagcacttgtagcgtttatggaagaaagttttggagtgcagatctcatgacaatgatgtaaatctatcttgcctcagtttgttcttgtagtttcctttggacttgaatttgataccttagtgcatcgctaagtgctatttctctgattcacataagaaatgtgatacaaatggttagttcaatcaatgcagaaaagttcaatcaaataaaatgggcccactgcagtcaattaacaggcattcaataggattcacattcctgggcttctatatatggaagtttgcatacaatgttttggaaataaaatgaaatataaattgcttgcatttagtgtaagttaatactcgctcccttctcgaatatttgtcgtccgctagttcatttttgaactaaaacatgataaataaaaaaacggaaggagtacatgtttgtaacaggagagcccatgaatacttgcttgtaacaggtggagcgctaagtatgcttaggagaactttaggcaacttgtattctttagcacttcgacgcagtttgtatggtaatatctactgatagacagaatcctggttttggatttttaatttttcctgcttttggttacacctctacagtcccatactcgcagtccaatagtacatggtctgataataaaccaattaagaaggactcatgtctcagtcattatgacttgagcataggagttgagatcaagaaatatttgagttgcagcttaaggtccagagaggaaatccccatacacttgcttgtaacggtatgaatgtatgatcatttttttttcaaggtaacattttctagcatcttcacctgtctacttgactgaatgcagtatatattagttgtaataactactggccttctgctgtgcacaaaaggcgggtattaccacttgcagaaatttgtcgggtaaaggtaattgccagttaccttgtgttcttcccttgatcaggaacacctggaggaggatgcgctgtggttgaaccgaagccctgtgagcgaagtactgatgacagaaagagcggaagataagataagaaaggaacccttgcgcggcaaggcctggtgacatagaggtagtggcgaggctcataccgccgccgctggcaggttccaggcctgtgcttttcttgccctgtatccccagtctatacttctgcgcacatcagacgagcctcagtgtttcggcacagtggtgcaacagaaaaggagagtgctggtaggtaacgctgaggcggtgaagaaagagaggtcagacggacctggaggtggctttttaactggtaaagagtgaggtctttcatgcccatcaatctgagcaccgacttgggtgttgctcctgttcgcaggaagcacaagaaatggtcagtactccacagcgtaggcatgtcggtggtgtgttggaggaggcaagattcagatgattattatatgagctcgaaaagctagagaatggatgttcagacttgagagctctgatttgagaggaattgcacttgtcgttttcccaaggcgacgcggccttttccagaggtttttttttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaaaaaaatgcacaccgacatgctctgtagcacaagcaccataccggcgaactggagaggtctcggctcatcaagcaatcgccttggtgtcggacggggtcatcaagacaaggcgactagaggagcactacatctacacggggtgaacggacgggagcagtggcggacccaggaactgatgacagccttggcgagaatacggtgtgatccccacgcctgtgctcgtgccacgtgctgcttgcttccgtgcactgtgctcgcgccttgcccattgcagccggcgagccagctcaggccaccgcctgcggtgcctggtgagtccgcccctggacgggaggaaggaaggaaaacgagagcgaggactcactgtccggtccgcccagcttggtgacggcgtcgacgaagcgctggtggaggtccggcgtccagcgcagccgcggcttggggtcccgtgacgccgccccgtcgtagccgtagctcccctgcatcgccgttccttcctggcgatcgccgctccctagctatccggtggccaaagacacggctagtggtaggctcgagcgagacgagctcttgctgaagagagaatgaatgtagcgttaccgcctcctggtcgtaggggtgtgggtatgtgaggacaagaggaggagcgagaggaggagcgcagagcgtggcggggaaggagggcgtcatgtgtgtgaggaatctaggacgacttgttggcagctgggccggggtgcgtgcgagatgcaatgcaagaacaaagcggacgggcatcacgcctccaggtccaacccgggggcgccactcggccgccgctcattgaggcccaggcgccaagacggcggctccacccacgtcacaattggcaataagaagcacacggctggggctgggacgcgtcgaatttttcaccagaaaataccgtctgatcctggcgtttcgtcagatgctatgctacgtgaacggcaaaacctagcagcagcagcactcagactggacaagaggagggaaatctttgcgtgggaaccaaactgaacgcgaatcgcacgggtcggatgacatatcatatcctcgtgcggagcggactcaacggcgagtccagctgtggctgcggaatattccggcggaagcgcggggagagcgacggcggcctccggtgggacccggggcgagcgggagatgcggcgaagatgttcggcgctgatgtcgctggaatattcgcgccagctgtggctgccggtgcgacctgctgaccagacgaccagtggcaatggccaccgcctctccatccaacctccatcacagattggcggacgattagccgagactaatcgctattctcaacactttaaaaaccgtgcgtgcagaatgctaagcctgctagattcgagcatctgcgtgactctactttggctcttctcgtacgatgcgacctgacgatgcatttgggcgttcctgtagcgtcactttcctgattagtcccccggaaacgcaactctaccactatcagccgccg

SEQ ID NO: 229 - DNA, 3251 bp, Zea mays
  misc_feature (1599)..(1745): n is unknown
tcgcatctgcagcttcttttgcacctgattacagacataagcacttgtagcgtttatggaagaaagttttggagtgcagatctcatgacaatgatgtaaatctatcttgcctcagtttgttcttgtagtttcctttggacttgaatttgataccttagtgcatcgctaagtgctatttctctgattcacataagaaatgtgatacaaatggttagttcaatcaatgcagaaaagttcaatcaaataaaatgggcccactgcagtcaattaacaggcattcaataggattcacattcctgggcttctatatatggaagtttgcatacaatgttttggaaataaaatgaaatataaattgcttgcatttagtgtaagttaatactcgctcccttctcgaatatttgtcgtccgctagttcatttttgaactaaaacatgataaataaaaaaacggaaggagtacatgtttgtaacaggagagcccatgaatacttgcttgtaacaggtggagcgctaagtatgcttaggagaactttaggcaacttgtattctttagcacttcgacgcagtttgtatggtaatatctactgatagacagaatcctggttttggatttttaatttttcctgcttttggttacacctctacagtcccatactcgcagtccaatagtacatggtctgataataaaccaattaagaaggactcatgtctcagtcattatgacttgagcataggagttgagatcaagaaatatttgagttgcagcttaaggtccagagaggaaatccccatacacttgcttgtaacggtatgaatgtatgatcatttttttttcaaggtaacattttctagcatcttcacctgtctacttgactgaatgcagtatatattagttgtaataactactggccttctgctgtgcacaaaaggcgggtattaccacttgcagaaatttgtcgggtaaaggtaattgccagttaccttgtgttcttcccttgatcaggaacacctggaggaggatgcgctgtggttgaaccgaagccctgtgagcgaagtactgatgacagaaagagcggaagataagataagaaaggaacccttgcgcggcaaggcctggtgacatagaggtagtgcgaggctcataccgccgccgctggcaggttccaggcctgtgcttttcttgccctgtatccccagtctatacttctgcgcacatcagacgagcctcagtgtttcggcacagtggtgcaacagaaaaggagagtgctggtaggtaacgctgaggcggtgaagaaagagaggtcagacggacctggaggtggctttttaactggtaaagagtgaggtctttcatgcccatcaatctgagcaccgacttgggtgttgctcctgttcgcaggaagcacaagaaatggtcagtactccacagcgtaggcatgtcggtggtgtgttggaggaggcaagattcagatgattattatatgagctcgaaaagctagagaatggatgttcagacttgagagctctgatttgatatgaattgcacttgtcgttttcccaaggcgacacggcctttttccagagttttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaaaaaaaaaagaatgcacaccgacatgctctgtagcacaagcaccatactggcgaactggagaggtctcggctcatcaagcaatcgccttggtgtcggacggggtcatcaagacaagacgactagacgagcactacatatagacgggaacgtacgggaggaaggaaggaaaacgagagcgaggactcactgtccggtccgcccagcttggtgacggcgtcgacgaagcgctggtggaggtccggcgtccagcgcagccgcggcttggggtcccgtgacgccgccccgtcgtagccgtagctcccctgcatcgtcgttccttcctggcgatcgccgcttcctagctatccggtggccaaagacacggctagtggtaggctcgagtgagacgagctcttgctgaagagagaatgaatgtaacgttaccgcctcctggtcgtaggtgtaataagttgtaacgcgagcgtcgttagcaagcacaggggtttgtgtatgtgaggacaagaggaggagcgagaggaggagcgcagagcgtggcggggaaggagggcgtcatgtgtgcgaggaatctaggacgacttgttggcacttggcagctgggccggggtgcgtgcgagatgcaatgcaagaacaaagcggacgggcatcacgcctccaggtccaacccgggggcgccactcggccgccgctcattgaggcccaggcgccaagacggcggctccacccacatcacaattggcaacaagaagcacacggctggggttgggacgcgtcgaatttttcaccagaaaataccgtctgatcctggcgtttcgtcagatgctatgctacgtgaacggcaaaacctagcagcagcagcagcactcagactggacaagaggagggaaatctttgcgtgggaaccaaactgaacgcgaatcgcacgagtcggatgacatatcctcgtccggagcggactcgaccgcgagtccagctgtggctgcggaatattccggcggaagcgcggggagaacgacggcggcctccggtgggacccggggcgagcgggagatgcggcgaagatgttcggcgctgatgtcgctggaatattcgcgccagctgtggctgccggtgtgacctgctgaccagacgaccagtggcagtggccaccgcctctccatcacagattcgcggacgattagccgagactaatcgctattctcaacacttttaaaaccgtgcgtgcagaatgctaagggcgcgttcgtttgcacagcaatagacatggatttatttcagctcatcaaaatctatataaattaaagaagtaatccggctagaaattaatccggagcttcaatccctaacaaccgaacagggtctaagcctgctagattcgagcatctgcgtgactctactttggctcttctcgtacgatgcgacttgacgatgcattngggncnnccnntagcgacactctcctgattagtcccacggaaacgcaactctaccactatcagccgccg

SEQ ID NO: 230 - DNA, 3102 bp, Zea mays
  misc_feature (1599)..(1743): n is unknown
tcgcatctgcagcttcttttgcacctgattacagacataagcacttgtagcgtttatggaagaaagttttggagtgcagatctcatgacaatgatgtaaatctatcttgcctcagtttgttcttgtagtttcctttggacttgaatttgataccttagtgcatcgctaagtgctatttctctgattcacataagaaatgtgatacaaatggttagttcaatcaatgcagaaaagttcaatcaaataaaatgggcccactgcagtcaattaacaggcattcaataggattcacattcctgggcttctatatatggaagtttgcatacaatgttttggaaataaaatgaaatataaattgcttgcatttagtgtaagttaatactcgctcccttctcgaatatttgtcgtccgctagttcatttttgaactaaaacatgataaataaaaaaacggaaggagtacatgtttgtaacaggagagcccatgaatacttgcttgtaacaggtggagcgctaagtatgcttaggagaactttaggcaacttgtattctttagcacttcgacgcagtttgtatggtaatatctactgatagacagaatcctggttttggatttttaatttttcctgcttttggttacacctctacagtcccatactcgcagtccaatagtacatggtctgataataaaccaattaagaaggactcatgtctcagtcattatgacttgagcataggagttgagatcaagaaatatttgagttgcagcttaaggtccagagaggaaatccccatacacttgcttgtaacggtatgaatgtatgatcatttttttttcaaggtaacattttctagcatcttcacctgtctacttgactgaatgcagtatatattagttgtaataactactggccttctgctgtgcacaaaaggcgggtattaccacttgcagaaatttgtcgggtaaaggtaattgccagttaccttgtgttcttcccttgatcaggaacacctggaggaggatgcgctgtggttgaaccgaagccctgtgagcgaagtactgatgacagaaagagcggaagataagataagaaaggaacccttgcgcggcaaggcctggtgacatagaggtagtgcgaggctcataccgccgccgctggcaggttccaggcctgtgcttttcttgccctgtatccccagtctatacttctgcgcacatcagacgagcctcagtgtttcggcacagtggtgcaacagaaaaggagagtgctggtaggtaacgctgaggcggtgaagaaagagaggtcagacggacctggaggtggctttttaactggtaaagagtgaggtctttcatgcccatcaatctgagcaccgacttgggtgttgctcctgttcgcaggaagcacaagaaatggtcagtactccacagcgtaggcatgtcggtggtgtgttggaggaggcaagattcagatgattattatatgagctcgaaaagctagagaatggatgttcagacttgagagctctgatttgagaggaattgcacttgtcgttttcccaaggcgacgcggcctttttccagaggcttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaaaaaaaaaaaagaatgcagaccgacatgctctgtagcacaagcaccatactggcgaactggagaggtctcggctcatcaagcaatcgccttggtgtcggacggggtcatcaagacaagacgactagacgagcactacatatagacgggaacgtacgggaggaaggaaggaaaacgagagcgaggactcactgtccggtccgcccagcttggtgacggcgtcgacgaagcgctggtggaggtccggcgtccagcgcagccgcggcttggggtcccgtgacgccgccccgtcgtagccgtagctcccctgcatcgtcgttccttcctggcgatcgccgcttcctagctatccggtggccaaagacacggctagtggtaggctcgagtgagacgagctcttgctgaagagagaatgaatgtaacgttaccgcctcctggtcgtaggtgtaataagttgtaacgcgagcgtcgttagcaagcacaggggtttgtgtatgtgaggacaagaggaggagcgagaggaggagcgcagagcgtggcggggaaggagggcgtcatgtgtgcgaggaatctaggacgacttgttggcacttggcagctgggccggggtgcgtgcgagatgcaatgcaagaacaaagcggacgggcatcacgcctccaggtccaacccgggggcgccactcggccgccgctcattgaggcccaggcgccaagacggcggctccacccacatcacaattggcaacaagaagcacacggctggggttgggacgcgtcgaatttttcaccagaaaataccgtcggcgtttcgtcagatgctatgctacgtgaacggcaaaacctagcagcagcagcactcagactggacgagaggagggaaatctttgcgtgggaaccaaactgaacgcgaatcgcacgagtcggatgacatatcctcgtccggagcggactcgaccgcgagtccagctgtggctgcggaatattccggcggaagcgcggggagaacgacggcggcctccggtgggaaccggggcgagcgggagatgcggcgaagatgttcggcgctgatgtcgctggaatattcgcgccagctgtggctgccggtgtgacctgctgaccagtggcagtggccaccgcctctccatcacagattggcggacgattagccgagactaatcgctattctcaacacttttaaaaccgtgcgtgcagaatgataaccctgctagattcgagcatctgcgtgactctactctggctcttctcgtacgatgcgacttgacgatgcatttgcgcgcctttagcgtcactttcctgattagtcccacggaaacgcaactctaccactatcagccgcca

SEQ ID NO: 231 - DNA, 3251 bp, Zea mays
  misc_feature (1598)..(1740): n is unknown
tcgcatctgcagcttcttttgcacctgattacagacataagcacttgtagcgtttatggaagaaagttttggagtgcagatctcatgacaatgatgtaaatctatcttgcctcagtttgttcttgtagtttcctttggacttgaatttgataccttagtgcatcgctaagtgctatttctctgattcacataagaaatgtgatacaaatggttagttcaatcaatgcagaaaagttcaatcaaataaaatgggcccactgcagtcaattaacaggcattcaataggattcacattcctgggcttctatatatggaagtttgcatacaatgttttggaaataaaatgaaatataaattgcttgcatttagtgtaagttaatactcgctcccttctcgaatatttgtcgtccgctagttcatttttgaactaaaacatgataaataaaaaaacggaaggagtacatgtttgtaacaggagagcccatgaatacttgcttgtaacaggtggagcgctaagtatgcttaggagaactttaggcaacttgtattctttagcacttcgacgcagtttgtatggtaatatctactgatagacagaatcctggttttggatttttaatttttcctgcttttggttacacctctacagtcccatactcgcagtccaatagtacatggtctgataataaaccaattaagaaggactcatgtctcagtcattatgacttgagcataggagttgagatcaagaaatatttgagttgcagcttaaggtccagagaggaaatccccatacacttgcttgtaacggtatgaatgtatgatcatttttttttcaaggtaacattttctagcatcttcacctgtctacttgactgaatgcagtatatattagttgtaataactactggccttctgctgtgcacaaaaggcgggtattaccacttgcagaaatttgtcgggtaaaggtaattgccagttaccttgtgttcttcccttgatcaggaacacctggaggaggatgcgctgtggttgaaccgaagccctgtgagcgaagtactgatgacagaaagagcggaagataagataagaaaggaacccttgcgcggcaaggcctggtgacatagaggtagtgcgaggctcataccgccgccgctggcaggttccaggcctgtgcttttcttgccctgtatccccagtctatacttctgcgcacatcagacgagcctcagtgtttcggcacagtggtgcaacagaaaaggagagtgctggtaggtaacgctgaggcggtgaagaaagagaggtcagacggacctggaggtggctttttaactggtaaagagtgaggtctttcatgcccatcaatctgagcaccgacttgggtgttgctcctgttcgcaggaagcacaagaaatggtcagtactccacagcgtaggcatgtcggtggtgtgttggaggaggcaagattcagatgattattatatgagctcgaaaagctagagaatggatgttcagacttgagagctctgatttgagaggaattgcacttgtcgttttcccaaggcgacgcggcctttttccagagtttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaaaaaaaaaaaaaaagaatgcacaccgacatgctctgtagcacaagcaccatactggcgaactggagaggtctcggctcatcaagcaatcgccttggtgtcggacggggtcatcaagacaagacgactagacgagcactacatatagacgggaacgtacgggaggaaggaaggaaaacgagagcgaggactcactgtccggtccgcccagcttggtgacggcgtcgacgaagcgctggtggaggtccggcgtccagcgcagccgcggcttggggtcccgtgacgccgccccgtcgtagccgtagctcccctgcatcgtcgttccttcctggcgatcgccgcttcctagctatccggtggccaaagacacggctagtggtaggctcgagtgagacgagctcttgctgaagagagaatgaatgtaacgttaccgcctcctggtcgtaggtgtaataagttgtaacgcgagcgtcgttagcaagcacaggggtttgtgtatgtgaggacaagaggaggagcgagaggaggagcgcagagcgtggcggggaaggagggcgtcatgtgtgcgaggaatctaggacgacttgttggcacttggcagctgggccggggtgcgtgcgagatgcaatgcaagaacaaagcggacgggcatcacgcctccaggtccaacccgggggcgccactcggccgccgctcattgaggcccaggcgccaagacggcggctccacccacatcacaattggcaacaagaagcacacggctggggttgggacgcgtcgaatttttcaccagaaaataccgtctgatcctggcgtttcgtcagatgctatgctacgtgaacggcaaaacctagcagcagcagcagcactcagactggacaagaggagggaaatctttgcgtgggaaccaaactgaacgcgaatcgcacgagtcggatgacatatcctcgtccggagcggactcgaccgcgagtccagctgtggctgcggaatattccggcggaagcgcggggagaacgacggcggcctccggtgggacccggggcgagcgggagatgcggcgaagatgttcggcgctgatgtcgctggaatattcgcgccagctgtggctgccggtgtgacctgctgaccagacgaccagtggcagtggccaccgcctctccatcacagattcgcggacgattagccgagactaatcgctattctcaacacttttaaaaccgtgcgtgcagaatgctaagggcgcgttcgtttgcacagcaatagacatggatttatttcagctcatcaaaatctatataaattaaagaagtaatccggctagaaattaatccggagcttcaatccctaacaaccgaacagggtctaagcctgctagattcgagcatctgcgtgactctactttggctcttctcgtacgatgcgacttgacgatgcatttgggnnnnnnngtagcgacactctcctgattagtcccacggaaacgcaactctaccactatcagccgccg

SEQ ID NO: 232 - DNA, 3034 bp, Zea mays
  misc_feature (1599)..(1742): n is unknown
tcgcatctgcagcttcttttgcacctgattacagacataagcacttgtagcgtttatggaagaaagttttggagtgcagatctcatgacaatgatgtaaatctatcttgcctcagtttgttcttgtagtttcctttggacttgaatttgataccttagtgcatcgctaagtgctatttctctgattcacataagaaatgtgatacaaatggttagttcaatcaatgcagaaaagttcaatcaaataaaatgggcccactgcagtcaattaacaggcattcaataggattcacattcctgggcttctatatatggaagtttgcatacaatgttttggaaataaaatgaaatataaattgcttgcatttagtgtaagttaatactcgctcccttctcgaatatttgtcgtccgctagttcatttttgaactaaaacatgataaataaaaaaacggaaggagtacatgtttgtaacaggagagcccatgaatacttgcttgtaacaggtggagcgctaagtatgcttaggagaactttaggcaacttgtattctttagcacttcgacgcagtttgtatggtaatatctactgatagacagaatcctggttttggatttttaatttttcctgcttttggttacacctctacagtcccatactcgcagtccaatagtacatggtctgataataaaccaattaagaaggactcatgtctcagtcattatgacttgagcataggagttgagatcaagaaatatttgagttgcagcttaaggtccagagaggaaatccccatacacttgcttgtaacggtatgaatgtatgatcatttttttttcaaggtaacattttctagcatcttcacctgtctacttgactgaatgcagtatatattagttgtaataactactggccttctgctgtgcacaaaaggcgggtattaccacttgcagaaatttgtcgggtaaaggtaattgccagttaccttgtgttcttcccttgatcaggaacacctggaggaggatgcgctgtggttgaaccgaagccctgtgagcgaagtactgatgacagaaagagcggaagataagataagaaaggaacccttgcgcggcaaggcctggtgacatagaggtagtgcgaggctcataccgccgccgctggcaggttccaggcctgtgcttttcttgccctgtatccccagtctatacttctgcgcacatcagacgagcctcagtgtttcggcacagtggtgcaacagaaaaggagagtgctggtaggtaacgctgaggcggtgaagaaagagaggtcagacggacctggaggtggctttttaactggtaaagagtgaggtctttcatgcccatcaatctgagcaccgacttgggtgttgctcctgttcgcaggaagcacaagaaatggtcagtactccacagcgtaggcatgtcggtggtgtgttggaggaggcaagattcagatgattattatatgagctcgaaaagctagagaatggatgttcagacttgagagctctgatttgagaggaattgcacttgtcgttttcccaaggcgacgcggcctttttccagaggcttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaaaaaaaaaaaaagaatgcagaccgacatgctctgtagcacaagcaccatactggcgaactggagaggtctcggctcatcaagcaatcgccttggtgtcggacggggtcatcaagacaagacgactagacgagcactacatatagacgggaacgtacgggaggaaggaaggaaaacgagagcgaggactcactgtccggtccgcccagcttggtgacggcgtcgacgaagcgctggtggaggtccggcgtccagcgcagccgcggcttggggtcccgtgacgccgccccgtcgtagccgtagctcccctgcatcgtcgttccttcctggcgatcgccgcttcctagctatccggtggccaaagacacggctagtggtaggctcgagtgagacgagctcttgctgaagagagaatgaatgtaacgttaccgcctcctggtcgtaggtgtaataagttgtaacgcgagcgtcgttagcaagcacaggggtttgtgtatgtgaggacaagaggaggagcgagaggaggagcgcagagcgtggcggggaaggagggcgtcatgtgtgcgaggaatctaggacgacttgttggcacttggcagctgggccggggtgcgtgcgagatgcaatgcaagaacaaagcggacgggcatcacgcctccaggtccaacccgggggcgccactcggccgccgctcattgaggcccaggcgccaagacggcggctccacccacatcacaattggcaacaagaagcacacggctggggttgggacgcgtcgaatttttcaccagaaaataccgtcggcgtttcgtcagatgctatgctacgtgaacggcaaaacctagcagcagcagcactcagactggacgagaggagggaaatctttgcgtgggaaccaaactgaacgcgaatcgcacgagtcggatgacatatcctcgtccggagcggactcgaccgcgagtccagctgtggctgcggaatattccggcggaagcgcggggagaacgacggcggcctccggtgggaaccggggcgagcgggagatgcggcgaagatgttcggcgctgatgtcgctggaatattcgcgccagctgtggctgccggtgtgacctgctgaccagtggcagtggccaccgcctctccatcacagattggcggacgattagccgagactaatcgctattctcaacacttttaaaaccgtgcgtgcagaatgataaccctgctagattcgagcatctgcgtgactctactctggctcttctcgtacgatgcgacttgacgatgcatt

SEQ ID NO: 233 - DNA, 3139 bp, Zea mays
  misc_feature (1722)..(1744): n is unknown
tcgcatctgcagcttcttttgcacctgattacagacataagcacttgtagcgtttatggaagaaagttttggagtgcagatctcatgacaatgatgtaaatctatcttgcctcagtttgttcttgtagtttcctttggacttgaatttgataccttagtgcatcgctaagtgctatttctctgattcacataagaaatgtgatacaaatggttagttcaatcaatgcagaaaagttcaatcaaataaaatgggcccactgcagtcaattaacaggcattcaataggattcacattcctgggcttctatatatggaagtttgcatacaatgttttggaaataaaatgaaatataaattgcttgcatttagtgtaagttaatactcgctcccttctcgaatatttgtcgtccgctagttcatttttgaactaaaacatgataaataaaaaaacggaaggagtacatgtttgtaacaggagagcccatgaatacttgcttgtaacaggtggagcgctaagtatgcttaggagaactttaggcaacttgtattctttagcacttcgacgcagtttgtatggtaatatctactgatagacagaatcctggttttggatttttaatttttcctgcttttggttacacctctacagtcccatactcgcagtccaatagtacatggtctgataataaaccaattaagaaggactcatgtctcagtcattatgacttgagcataggagttgagatcaagaaatatttgagttgcagcttaaggtccagagaggaaatccccatacacttgcttgtaacggtatgaatgtatgatcatttttttttcaaggtaacattttctagcatcttcacctgtctacttgactgaatgcagtatatattagttgtaataactactggccttctgctgtgcacaaaaggcgggtattaccacttgcagaaatttgtcgggtaaaggtaattgccagttaccttgtgttcttcccttgatcaggaacacctggaggaggatgcgctgtggttgaaccgaagccctgtgagcgaagtactgatgacagaaagagcggaagataagataagaaaggaacccttgcgcggcaaggcctggtgacatagaggtagtgcgaggctcataccgccgccgctggcaggttcsaggcctgtgcttttcttgccctgtatccccagtctatacttctgcgcacatcagacgagcgtcagtgtttcggcacagtggtgcaacagaaaaggagagtgctggtaggtaacgctgaggcggtgaagaaagagaggtcagacggacctggaggtggctttttaactggtaaagagtgaggtctttcatgcccatcaatctgagcaccgacttgggtgttgcttctgttcgcaggaagcacaagaaatggtcagtactccacagcgtaggcatgtcggtgtgttcgaggaggcaagattcagatgattattatatgagctcgaaaagctagagaatggatgttcagacttgagatctctgatttgagaggaattgcacttgtcgttttcccagggcgacgcggcctttttccagaggcattttttttcaactgccttttggtcatgtcaacggaactgccttttcctctgactgcatgctatagacttggcaatggcagaagcgcaaagccaggcagcgaaggattcggactgcaactggccgtcgttttacaannnnnnnnnnnnnnnnnnnnnnnaaaaaaaaaagaatgcagaccgacatgctctgtagcacaagcaccatacttgcgaactgcagaggtgtcgggtcatcaagcaatcgccttggtgtcggacggggtggggtcatcaagacaagacgactagaggagcactacatctacacgggggggaacggacgggaggaaggaaggaaaacgagagcgaggactcactgtccggtccgcccagcttggtgacggcgtcgacgaagcgctggtggaggaccggcgtccagcgcagccgcggcttggggtcccgtgacgccgccccgtcgtagccgtagctcccctgcatcgtcgttccttcctggcgatcgccgctccctagctatccggtggccaaagacacggctagtgctgaagagagaatgaatgtaacgttaccgcctcctggtcgtaggtgtaataagttgtaacgcgagtgtcgttagaagcacaggggtgtgtgtatgtgaggacaagaggaggagcgagaggaggagcgcagagcgtggcggggaaggagggcgtcatgtgtgtgaggaatctaggacgacttgttggcagctgggccggggtgcgtgcgagatgcaatgcaagaacaaagcatcacgcctccaagtccaaccggggggcgccactcggccgccgctcattgaggcccaggcgccaagacggcggctccacccacatcacaattggcaacaagaagcacacggctggggctgggacgcgtcgaatttttcaccagaaaataccgtcggcgtttcgtcagatgctatgctacgtgaacggcaaaacctagcagcagcagcagcagcactcagactggacaagaggagggaaatctttgcgtgggaaccaaactgaacgcgaatcgcacgagtcggatgacatatcctcgtccggagcggactcggccgcgagtccagctgtggctgcggaatattccggcggaagcgcggggagaacgacggcggcctccggtgggacccggggcgagcgggagatgcggcgaagatgttcggcgctgatgtcgctggaatattcgcgccagctgtggctgccggtgtgacctgctgaccagacgaccagtggcagtggccaccgcctctccatcacagattcgcggacgattagccgagactaatcgctattctcaacacttttaaaaccgtgcgtgcagaatgctaagggcgcgttcgtttgcacagcaatagacattgatttatttcagctcatcaaaatctatataaattaaagaagtaatccggctagaaattaatccggagcttcaatccctaacaaccgaacagggtctaagcctgctagattcgagcatctgcgtgactctactttggctcttctcgtacgatgcgacttgacgatgca

SEQ ID NO: 234 - DNA, 3052 bp, Zea mays
  misc_feature (1563)..(1708): n is unknown
tcgcatctgcagcttcttttgcacctgattacagacataagcacttgtagcgtttatggaagaaagttttggagtgcagatctcatgacaatgatgtaaatctatcttgcctcagtttgttcttgtagtttcctttggacttgaatttgataccttaatgcatcgctaagtgctatttctctgattcacataagaaatgcgatacaaatggttagttcaatcaatgcagaaaagttcaaccaaataaaatgggcccactgcagtcaattaacaggcattcaataggattcacattcctgggcttctatatatggaagtttgcatacaaagttttggaaataaaatggaatagaaattgcttgcatttagtgtaagttaatacccgctagttcattttttaactaaaacacgacaaataaaaaaatggaggagtacatctttgtaacaggtgagcctgaatacttgtttgtagcaggtggggcgctaagtatgcttaggagaagtttaggcaacttgtattctgtagcatttcgacgccgtttgtatggtaatatctactgataggcagaatcctggttggattttttttcctgcttttgtttacacctatacagtcccatactcgcagtcgaataatacatggtctgatgataaaccaattaagaaggactcatgtctcagtcattatgacttgagcataggagttgagatcaagaaatatttgagttgcagcttaaggtccagagaggaaatccccatacacttgcttgtaacggtatgaatgtatgatcatttttttttcaaggtaacattttctagcatcttcacctgtctacttgactgaatgcagtatatattagttgtaataactactggccttctgctgtgcacaaaaggcgggtattaccacttgcagaaatttgtcgggtaaaggtaattgccagttaccttgtgttcttcccttgatcaggaacacctggaggaggatgcgctgtggttgaaccgaagccctgtgagcgaagtactgatgacagaaagagcggaagataagataagaaaggaacccttgcgcggcaaggcctggtgacatagaggtagtgcgaggctcataccgccgccgctggcaggttccaggcctgtgcttttcttgccctgtatccccagtctatacttctgcgcacatcagacgagcctcagtgtttcggcacagtggtgcaacagaaaaggagagtgctggtaggtaacgctgaggcggtgaagaaagagaggtcagacggacctggaggtggctttttaactggtaaagagtgaggtctttcatgcccatcaatctgagcaccgacttgggtgttgctcctgttcgcaggaagcacaagaaatggtcagtactccacagcgtaggcatgtcggtggtgtgttggaggaggcaagattcagatgattattatatgagctcgaaaagctagagaatggatgttcagacttgagagctctgatttgagaggaattgcacttgtcgttttcccaaggcgacgcggcctttttccagagtttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaaaaaaaaaaagaatgcagaccgacatgctctgtagcacaagcaccatactggcgaactggagaggtctcggctcatcaagcaatcgccttggtgtcggacggggtcatcaagacaagacgactagacgagcactacatatagacgggaacgtacgggaggaaggaaggaaaacgagagcgaggactcactgtccggtccgcccagcttggtgacggcgtcgacgaagcgctggtggaggtccggcgtccagcgcagccgcggcttggggtcccgtgacgccgccccgtcgtagccgtagctcccctgcatcgccgttccttcctggcgatcgccgctccctagctatccggtggccaaagacacggctagtggtaggctcgagcgagacgagctcttggtgaagagagaatgaatgtaacgttaccgcctcctggtcgtaggggtgtgtgtatgtgaggacaagaggaggagcgagaggaggagcgcagagcgtggcggggaaggagggcgtcatgtgtgtgaggaatctaggacgacttgttggcagctgggccggggtgcgtgcgagatgcaatgcaagaacaaagcggacgggcatcacgcctccaggtccaacccgggggcgccactcggccgccgctcattgaggcccaggcgccaagacggcggctccacccacgtcacaattggcaataagaagcacacggctggggctgggacgcgtcgaatttttcaccagaaaataccgtctgatcctggcgtttcgtcagatgctatgctacgtgaacggcaaaacctagcagcagcagcactcagactggacaagaggagggaaatctttgcgtgggaaccaaactgaacgcgaatcgcacgggtcggatgacatatcatatcctcgtgcggagcggactcaacggcgagtccagctgtggctgcggaatattccggcggaagcgcggggagagcgacggcggcctccggtgggacccggggcgagcgggagatgcggcgaagatgttcggcgctgatgtcgctggaatattcgcgccagctgtggctgccggtgcgacctgctgaccagacgaccagtggcaatggccaccgcctctccatccaacctccatcacagattggcggacgattagccgagactaatcgctattctcaacactttaaaaaccgtgcgtgcagaatgctaagcctgctagattcgagcatctgcgtgactctactttggctcttctcgtacgatgcgacctgacgatgcatttgggnnnncctntagcgtcactttcctgattagtcccccggaaacgcaactctaccactatcagccgccg

SEQ ID NO: 235 - DNA, 3219 bp, Zea mays
  misc_feature (1554)..(1701): n is unknown
tcgcatctgcagcttcttttgcacctgattacagacataagcacttgtagcgtttatggaagaaagttttggagtgcagatctcatgacaatgatgtaaatctatcttgcctcagtttgttcttgtagtttcctttggacttgaatttgataccttagtgcatcgctaagtgctatttctctgattcacataagaaatgtgatacaaatggttagctcaatcaatgcagaaaagttcaaccaaataaaatgggcccactgcagtcaattaacaggcattcaataggattcacattcctgggcttctatatatggaagtttgcatacaaagttttggaaataaaatggaatagaaattgcttgcatttagtgtaagttaatacccgctagttcattttttaactaaaacacgacaaataaaaaaatggaggagtacatctttgtaacaggtgagcctgaatacttgtttgtagcaggtggggcgctaagtatgcttaggagaagtttaggcaacttgtattctgtagcatttcgacgccgtttgtatggtaatatctactgataggcagaatcctggttggattttttttcctgcttttgtttacacctatacagtcccatactcgcagtcgaataatacatggtctgatgataaaccaattaagaaggactcatgtctcagtcattatgacttgagcataggagttcagatcgagaaatatttgagttgcagcttaaggttcagagaggaaatcccatacacttgcttgtaacgatatgatcattttttttcaaggtaacattttctagcatcttcagctgtctacttgactgaatgcagtatatattagttgtaataaatactgcccttctgctgtgcacaaaaggcgggtattaccacttgcagaaatttgtcgggtaaaggtaattgccagttaccttgtgttcttcccttcatcaggaacacctggaggaggatgcgctgtggttgaactgaagccctgcgagagaagtactgatgacagaaagagcggaagataagataagaaaggaaacccttgcgcggcaaggcctggtgacatagaggtagtgcgaggctcataccgccgctggcaggttccaggcctgtgcttttcttgccctgtatccccagtctatacttctgcgcacatcagacgagcctcagtgtttcggcacagtggtgcaacagaaaaggagagtgctggtaggtaacgctgaggcggtgaagaaagagaggtcagacggacctggaggtggctttttaactggtaaagagtgaggtctttcatgcccatcaatctgagcaccgacttgggtgttgctcctgttcgcaggaagcacaagaaatggtcagtactccacagcgtaggcatgtcggtggtgtgttggaggaggcaagattcagatgattattatatgagctcgaaaagctagagaatggatgttcagacttgagagctctgatttgagaggaattgcacttgtcgttttcccaaggcgacgcggccttttccagaggttttttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgaaaaaaaaatgcacaccgacatgctctgtagcacaagcaccataccggcgaactggagaggtctcggctcatcaagcaatcgcccttggtgtcggacggggtcatcaagacaaggcgactagaggagcactacatctacacggggtgaacggacgggagcagtggcggacccaggaactgatgacagccttggcgagaatacggtgtgatccccacgcctgtgctcgtgccacgtgctgcttgcttccgtgcactgtgctcgcgccttgcccattgcagccggcgagccagctcaggccaccgcctgcggtgcctggtgagtccgcccctggacgggaggaaggaaggaaaacgagagcgaggactcactgtccggtccgcccagcttggtgacggcgtcgacgaagcgctggtggaggtccggcgtccagcgcagccgcggcttggggtcccgtgacgccgccccgtcgtagccgtagctcccctgcatcgccgttccttcctggcgatcgccgctccctagctatccggtggccaaagacacggctagtggtaggctcgagcgagacgagctcttgctgaagagagaatgaatgtagcgttaccgcctcctggtcgtaggggtgtgggtatgtgaggacaagaggaggagcgagaggaggagcgcagagcgtggcggggaaggagggcgtcatgtgtgtgaggaatctaggacgacttgttggcagctgggccggggtgcgtgcgagatgcaatgcaagaacaaagcggacgggcatcacgcctccaggtccaacccgggggcgccactcgatcggccgccgctcattgaggcccaggcgccaagacggcggctccacccacgtcacaattggcaataagaagcacacggctggggctgggacgcgtcgaatttttcaccagaaaataccgtctgatcctggcgtttcgtcagatgctatgctacgtgaacggcaaaacctagcagcagcagcactcagactggacaagaggagggaaatctttgcgtgggaaccaaactgaacgcgaatcgcacgggtcggatgacatatcatatcctcgtgcggagcggactcaacggcgagtccagctgtggctgcggaatattccggcggaagcgcggggagagcgacggcggcctccggtgggacccggggcgagcgggagatgcggcgaagatgttcggcgctgatgtcgctggaatattcgcgccagctgtggctgccggtgcgacctgctgaccagtggcaatggccaccgcctctccatccaacctccatcacagattggcggacgattagccgagactaatcgctattctcaacactttaaaaaccgtgcgtgcagaatgctaagcctgctagattcgagcatctgcgtgactctactttggctcttctcgtacgatgcgacctgacgatgcatttgggnnncctntagcgtcactttcctgattagtcccccggaaacgcaactctaccactatcagccgccg

SEQ ID NO: 236 - DNA, 3099 bp, Zea mays
  misc_feature (1555)..(1701): n is unknown
tcgcatctgcagcttcttttgcacctgattacagacataagcacttgtagcgtttatggaagaaagttttggagtgcagatctcatgacaatgatgtaaatctatcttgcctcagtttgttcttgtagtttcctttggacttgaatttgataccttaatgcatcgctaagtgctatttctctgattcacataagaaatgcgatacaaatggttagttcaatcaatgcagaaaagttcaaccaaataaaatgggcccactgcagtcaattaacaggcattcaataggattcacattcctgggcttctatatatggaagtttgcatacaaagttttggaaataaaatggaatagaaattgcttgcatttagtgtaagttaatacccgctagttcattttttaactaaaacacgacaaataaaaaaatggaggagtacatctttgtaacaggtgagcctgaatacttgtttgtagcaggtggggcgctaagtatgcttaggagaagtttaggcaacttgtattctgtagcatttcgacgccgtttgtatggtaatatctactgataggcagaatcctggttggattttttttcctgcttttgtttacacctatacagtcccatactcgcagtcgaataatacatggtctgatgataaaccaattaagaaggactcatgtctcagtcattatgacttgagcataggagttcagatcgagaaatatttgagttgcagcttaaggttcagagaggaaatcccatacacttgcttgtaacgatatgatcattttttttcaaggtaacattttctagcatcttcagctgtctacttgactgaatgcagtatatattagttgtaataaatactgcccttctgctgtgcacaaaaggcgggtattaccacttgcagaaatttgtcgggtaaaggtaattgccagttaccttgtgttcttcccttcatcaggaacacctggaggaggatgcgctgtggttgaactgaagccctgcgagagaagtactgatgacagaaagagcggaagataagataagaaaggaaacccttgcgcggcaaggcctggtgacatagaggtagtgcgaggctcataccgccgctggcaggttccaggcctgtgcttttcttgccctgtatccccagtctatacttctgcgcacatcagacgagcctcagtgtttcggcacagtggtgcaacagaaaaaggagagtgctggactgctggtaacgctgaggcggtgaagaaagagaggtcagacggacctggaggtggctttttaactggtaaagagtgaggtctttcatgcccatcaatctgagcaccgacttgggtgttgcttctgttcgcaggaagcacaagaaatggtcagtactccacagcgtaggcatgtcggtgtgttcgaggaggcaagattcagatgattattatatgagctcgaaaagctagagaatggatgttcagacttgagatctctgatttgagaggaattgcacttgtcgttttcccarggcgacgcggcctttttccagaggcattttttttcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntnnnnnnngaaaaaaaaaagaatgcagaccgacatgctctgtagcacaagcaccatacttgcgaactgcagaggtgtcgggtcatcaagcaatcgccttggtgtcggacggggtggggtcatcaagacaagacgactagaggagcactacatctacacgggggggaacggacgggaggaaggaaggaaaacgagagcgaggactcactgtccggtccgcccagcttggtgacggcgtcgacgaagcgctggtggaggaccggcgtccagcgcagccgcggcttggggtcccgtgacgccgccccgtcgtagccgtagctcccctgcatcgtcgttccttcctggcgatcgccgctccctagctatccggtggccaaagacacggctagtgctgaagagagaatgaatgtaacgttaccgcctcctggtcgtaggtgtaataagttgtaacgcgagtgtcgttagaagcacaggggtgtgtgtatgtgaggacaagaggaggagcgagaggaggagcgcagagcgtggcggggaaggagggcgtcatgtgtgtgaggaatctaggacgacttgttggcagctgggccggggtgcgtgcgagatgcaatgcaagaacaaagcatcacgcctccaagtccaaccggggggcgccactcggccgccgctcattgaggcccaggcgccaagacggcggctccacccacatcacaattggcaacaagaagcacacggctggggctgggacgcgtcgaatttttcaccagaaaataccgtcggcgtttcgtcagatgctatgctacgtgaacggcaaaacctagcagcagcagcagcactcagactggacaagaggagggaaatctttgcgtgggaaccaaactgaacgcgaatcgcacgagtcggatgacatatcctcgtccggagcggactcggccgcgagtccagctgtggctgcggaatattccggcggaagcgcggggagaacgacggcggcctccggtgggacccggggcgagcgggagatgcggcgaagatgttcggcgctgatgtcgctggaatattcgcgccagctgtggctgccggtgtgacctgctgaccagacgaccagtggcagtggccaccgcctctccatcacagattcgcggacgattagccgagactaatcgctattctcaacacttttaaaaccgtgcgtgcagaatgctaagggcgcgttcgtttgcacagcaatagacatggatttatttcagctcatcaaaatctatataaattaaagaagtaatccggctagaaattaatccggagcttcaatccctaacaaccgaacagggtctaagcctgctagattcgagcatctgcgtgactctactttggctcttctcgtacgatgcgacttgacgatgcatttgg

(19) United States
(12) Patent Application Publication
(10) Pub. No.: US 2010/0325750 A1
(43) Pub. Date: Dec. 23, 2010

(54) Major QTLS Conferring Resistance Of Corn To Fijivirus

(76) Inventors: Teresita Martin, Pergamino (AR); Jose Alejandro Franchino, Pergamino (AR); Enrique Domingo Kreff, Pergamino (AR); Ana María Procopiuk, Pergamino (AR); Adriana Tomas, Newark, DE (US); Stanley D. Luck, Wilmington, DE (US); Guoping G. Shu, Johnston, IA (US)

Correspondence Address: POTTER ANDERSON & CORROON LLP;ATTN: JANET E. REED, PH.D., P.O. BOX 951, WILMINGTON, DE, 19899-0951, US

(21) Appl. No.: 12/740,140

(22) PCT Filed: Oct. 31, 2008
(86) PCT No.: PCT/US08/12327

§ 371 Date: Sep. 7, 2010

Related U.S. Application Data
(60) Provisional application No. 61/001,455, filed on Nov. 1, 2007.

(51) Int. Cl. A01H 1/02; C12Q 1/68; A01H 5/00; C12N 15/09

(52) U.S. Cl. 800/265; 435/6; 800/320.1; 800/280

(57) ABSTRACT

The invention relates to methods and compositions for identifying maize plants that have newly conferred resistance or enhanced resistance to, or are susceptible to, a Fijivirus, particularly Mal de Río Cuarto Virus (MRCV) and/or Maize Rough Dwarf Virus (MRDV). The methods use molecular genetic markers to identify, select and/or construct resistant plants or identify and counter-select susceptible plants. Maize plants that display newly conferred resistance or enhanced resistance to a Fijivirus that are generated by the methods of the invention are also a feature of the invention.

### CROSS-REFERENCE TO RELATED APPLICATION

This application claims the benefit of U.S. Provisional Application No. 61/001,455, filed Nov. 1, 2007, which is incorporated by reference in its entirety.

### FIELD OF THE INVENTION

The present disclosure relates to compositions and methods useful in creating or enhancing Fijivirus, particularly Mal de Río Cuarto Virus and/or Maize Rough Dwarf Virus, resistance in plants. Additionally, the invention relates to plants that have been genetically transformed with the compositions of the invention.

### BACKGROUND OF THE INVENTION

The disease caused by Mal de Río Cuarto Virus (MRCV) is a major corn disease in Argentina, accounting for yield losses of greater than 70% in years of severe outbreak (Rodriguez P E et al. (1998) Plant Dis. 82:149-52). The disease is a member of Serogroup 2 of Fijivirus, which includes other viruses such as maize rough dwarf virus, rice black streaked dwarf virus, and pangola stunt virus (Uyeda I & Milne R G (1995) Semin. Virol. 6:85-88). The main vector for MRCV is Delphacodes kuscheli, but other Delphacodes species, such as D. haywardi and D. tigrinus, and Toya propinqua have been shown to carry the virus. The virus does not appear to be transmitted to progeny via seeds. Distéfano et al., Arch. Virol. 147:1699-1709 (2002), analyzed the MRCV sequence and proposed that it is a new Fijivirus species related to MRDV (Maize Rough Dwarf Virus). MRDV is found in several European countries (e.g., the Czech Republic, France, Italy, Norway, Spain, Sweden) and in China, while MRCV has been also detected in Uruguay (Ornaghi J. A., Beviacqua J. E., Aguirrezabala D. A., March G. J. and Lenardón S. L. 1999. Detection of Mal de Río Cuarto virus in Uruguay. Fitopatologia Brasileira 24: 471).

MRCV infection causes abnormal maize development and significantly reduces crop yields. The susceptible phenotype includes stunting, shortening of internodes, multiple ears with scattered grain, deformed tassel with no anthers, presence of small enations in the back of the leaves, reduced roots, cut and reduced leaves. Plants symptoms depend on phenological stage of the plant, plant genotype, and environment (Lenardón et al., “Virus del Mal de Río Cuarto en maíz”, in Proyecto de Investigaciones en Fitovirología (Lenardón ed.), 2:10 (1999). Most severe symptoms occur when infected at the coleoptile—first leaf stage.

In the severe MRCV outbreak of 1996-1997, over 300,000 hectares of maize in Argentina were affected, resulting in losses totaling approximately $120 million. Increased populations of Delphacodes kuscheli in 2006 apparently led to a reoccurrence of the viral disease in Argentinean corn plants, which significantly affected the 2007 harvesting. Susceptible genotypes were strongly affected by MRCV at the endemic region (Córdoba Province) and moderately affected at other maize regions.

The development of molecular genetic markers has facilitated mapping and selection of agriculturally important traits in maize. Markers tightly linked to disease resistant genes are an asset in the rapid identification of resistant maize lines on the basis of genotype by the use of marker assisted selection (MAS). Introgressing disease resistance genes into a desired cultivar would also be facilitated by using suitable DNA markers.

### Molecular Markers and Marker Assisted Selection

A genetic map is a graphical representation of a genome (or a portion of a genome such as a single chromosome) where the distances between landmarks on the chromosome are measured by the recombination frequencies between the landmarks. A genetic landmark can be any of a variety of known polymorphic markers, for example but not limited to, molecular markers such as SSR markers, RFLP markers, FLP markers, or SNP markers. Furthermore, SSR markers can be derived from genomic or expressed nucleic acids (e.g., ESTs). The nature of these physical landmarks and the methods used to detect them vary, but all of these markers are physically distinguishable from each other (as well as from the plurality of alleles of any one particular marker) on the basis of polynucleotide length and/or sequence.

Although specific DNA sequences which encode proteins are generally well-conserved across a species, other regions of DNA (typically non-coding) tend to accumulate polymorphism, and therefore, can be variable between individuals of the same species. Such regions provide the basis for numerous molecular genetic markers. In general, any differentially inherited polymorphic trait (including nucleic acid polymorphism) that segregates among progeny is a potential marker. The genomic variability can be of any origin, for example, insertions, deletions, duplications, repetitive elements, point mutations, recombination events, or the presence and sequence of transposable elements. A large number of maize molecular markers are known in the art, and are published or available from various sources, such as the MaizeGDB internet resource. Similarly, numerous methods for detecting molecular markers are also well-established.

The primary motivation for developing molecular marker technologies from the point of view of plant breeders has been the possibility to increase breeding efficiency through marker assisted selection (MAS). A molecular marker allele that demonstrates linkage disequilibrium with a desired phenotypic trait (e.g., a quantitative trait locus, or QTL, such as resistance to a particular disease) provides a useful tool for the selection of a desired trait in a plant population. The key components to the implementation of this approach are: (i) the creation of a dense genetic map of molecular markers, (ii) the detection of QTL based on statistical associations between marker and phenotypic variability, (iii) the definition of a set of desirable marker alleles based on the results of the QTL analysis, and (iv) the use and/or extrapolation of this information to the current set of breeding germplasm to enable marker-based selection decisions to be made.

The availability of integrated linkage maps of the maize genome containing increasing densities of public maize markers has facilitated maize genetic mapping and MAS. See, e.g., the MaizeGDB resource on the world wide web.

Two types of markers are frequently used in marker assisted selection protocols, namely simple sequence repeat (SSR, also known as microsatellite) markers and single nucleotide polymorphism (SNP) markers. The term SSR refers generally to any type of molecular heterogeneity that results in length variability, and most typically is a short (up to several hundred base pairs) segment of DNA that consists of multiple tandem repeats of a two or three base-pair sequence. These repeated sequences result in highly polymorphic DNA regions of variable length due to poor replication fidelity, e.g., caused by polymerase slippage. SSRs appear to be randomly dispersed through the genome and are generally flanked by conserved regions. SSR markers can also be derived from RNA sequences (in the form of a cDNA, a partial cDNA or an EST) as well as genomic material.

The characteristics of SSR heterogeneity make them well suited for use as molecular genetic markers; namely, SSR genomic variability is inherited, is multiallelic, codominant and is reproducibly detectable. The proliferation of increasingly sophisticated amplification-based detection techniques (e.g., PCR-based) provides a variety of sensitive methods for the detection of nucleotide sequence heterogeneity. Primers (or other types of probes) are designed to hybridize to conserved regions that flank the SSR domain, resulting in the amplification of the variable SSR region. The different sized amplicons generated from an SSR region have characteristic and reproducible sizes. The different sized SSR amplicons observed from two homologous chromosomes in an individual, or from different individuals in the plant population, are generally termed “marker alleles”. As long as there exists at least two SSR alleles that produce PCR products with at least two different sizes, the SSRs can be employed as a marker.

Maize markers that rely on single nucleotide polymorphisms (SNPs) are also well known in the art. Various techniques have been developed for the detection of SNPs, including allele specific hybridization (ASH; see, e.g., Shattuck-Eidens et al., (1991) “Rapid detection of maize DNA sequence variation”, Genet. Anal. Tech. Appl. 8:240-245). Additional types of molecular markers are also widely used, including but not limited to expressed sequence tags (ESTs) and SSR markers derived from EST sequences, restriction fragment length polymorphism (RFLP), amplified fragment length polymorphism (AFLP), randomly amplified polymorphic DNA (RAPD) and isozyme markers. A wide range of protocols are known to one of skill in the art for detecting this variability, and these protocols are frequently specific for the type of polymorphism they are designed to detect. For example, PCR amplification, single-strand conformation polymorphisms (SSCP) and self-sustained sequence replication (3SR; see Chan and Fox, “NASBA and other transcription-based amplification methods for research and diagnostic microbiology”, Reviews in Medical Microbiology 10:185-196 (1999)).

Various types of FLP markers can also be generated. Most commonly, amplification primers are used to generate fragment length polymorphisms. Such FLP markers are in many ways similar to SSR markers, except that the region amplified by the primers is not typically a highly repetitive region. Still, the amplified region, or amplicon, will have sufficient variability among germplasm, often due to insertions or deletions, such that the fragments generated by the amplification primers can be distinguished among polymorphic individuals, and such indels are known to occur frequently in maize (Bhattramakki et al. (2002) Plant Mol Biol 48, 539-547; Rafalski (2002) Plant Sci 162:329-333). The term “indel” refers to an insertion or deletion, wherein one line may be referred to as having an insertion relative to a second line, or the second line may be referred to as having a deletion relative to the first line. The MZA markers disclosed herein are examples of amplified FLP markers that have been selected because they are in close proximity to the major chromosome 2 QTL.

Linkage of one molecular marker to another molecular marker is measured as a recombination frequency. In general, the closer two loci (e.g., two SSR markers) are on the genetic map, the closer they lie to each other on the physical map. A relative genetic distance (determined by crossing over frequencies, measured in centimorgans; cM) is generally proportional to the physical distance (measured in base pairs, e.g., kilobase pairs (kb) or megabasepairs (Mbp)) that two linked loci are separated from each other on a chromosome. A lack of precise proportionality between cM and physical distance can result from variation in recombination frequencies for different chromosomal regions, e.g., some chromosomal regions are recombinational “hot spots”, while others regions do not show any recombination, or only demonstrate rare recombination events. In general, the closer one marker is to another marker, whether measured in terms of recombination or physical distance, the more strongly they are linked. In some aspects, the closer a molecular marker is to a gene that encodes a polypeptide that imparts a particular phenotype (disease resistance), whether measured in terms of recombination or physical distance, the better that marker serves to tag the desired phenotypic trait.

Genetic mapping variability can also be observed between different populations of the same crop species, including maize. In spite of this variability in the genetic map that may occur between populations, genetic map and marker information derived from one population generally remains useful across multiple populations in identification of plants with desired traits, counter-selection of plants with undesirable traits and in guiding MAS.

### QTL Mapping

It is the goal of the plant breeder to select plants and enrich the plant population for individuals that have desired traits, for example, pathogen resistance, leading ultimately to increased agricultural productivity. It has been recognized for quite some time that specific chromosomal loci (or intervals) can be mapped in an organism's genome that correlate with particular quantitative phenotypes. Such loci are termed quantitative trait loci, or QTL. The plant breeder can advantageously use molecular markers to identify desired individuals by identifying marker alleles that show a statistically significant probability of co-segregation with a desired phenotype (e.g., pathogen resistance), manifested as linkage disequilibrium. By identifying a molecular marker or clusters of molecular markers that co-segregate with a quantitative trait, the breeder is thus identifying a QTL. By identifying and selecting a marker allele (or desired alleles from multiple markers) that associates with the desired phenotype, the plant breeder is able to rapidly select a desired phenotype by selecting for the proper molecular marker allele (a process called marker-assisted selection, or MAS). The more molecular markers that are placed on the genetic map, the more potentially useful that map becomes for conducting MAS.

Multiple experimental paradigms have been developed to identify and analyze QTL (see, e.g., Jansen (1996) Trends Plant Sci 1:89). The majority of published reports on QTL mapping in crop species have been based on the use of the bi-parental cross (Lynch and Walsh (1997) Genetics and Analysis of Quantitative Traits, Sinauer Associates, Sunderland). Typically, these paradigms involve crossing one or more parental pairs, which can be, for example, a single pair derived from two inbred strains, or multiple related or unrelated parents of different inbred strains or lines, which each exhibit different characteristics relative to the phenotypic trait of interest. Typically, this experimental protocol involves deriving 100 to 300 segregating progeny from a single cross of two divergent inbred lines (e.g., selected to maximize phenotypic and molecular marker differences between the lines). The parents and segregating progeny are genotyped for multiple marker loci and evaluated for one to several quantitative traits (e.g., disease resistance). QTL are then identified as significant statistical associations between genotypic values and phenotypic variability among the segregating progeny. The strength of this experimental protocol comes from the utilization of the inbred cross, because the resulting F1 parents all have the same linkage phase. Thus, after selfing of the F1 plants, all segregating progeny (F2) are informative and linkage disequilibrium is maximized, the linkage phase is known, there are only two QTL alleles, and, except for backcross progeny, the frequency of each QTL allele is 0.5.

Numerous statistical methods for determining whether markers are genetically linked to a QTL (or to another marker) are known to those of skill in the art and include, e.g., standard linear models, such as ANOVA or regression mapping (Haley and Knott (1992) Heredity 69:315), maximum likelihood methods such as expectation-maximization algorithms, (e.g., Lander and Botstein (1989) “Mapping Mendelian factors underlying quantitative traits using RFLP linkage maps”, Genetics 121:185-199; Jansen (1992) “A general mixture model for mapping quantitative trait loci by using molecular markers”, Theor. Appl. Genet., 85:252-260; Jansen (1993) “Maximum likelihood in a generalized linear finite mixture model by using the EM algorithm”, Biometrics 49:227-231; Jansen (1994) “Mapping of quantitative trait loci by using genetic markers: an overview of biometrical models”, In J. W. van Ooijen and J. Jansen (eds.), Biometrics in Plant breeding: applications of molecular markers, pp. 116-124, CPRO-DLO Metherlands; Jansen (1996) “A general Monte Carlo method for mapping multiple quantitative trait loci”, Genetics 142:305-311; and Jansen and Stam (1994) “High Resolution of quantitative trait into multiple loci via interval mapping”, Genetics 136:1447-1455). Exemplary statistical methods include single point marker analysis, interval mapping (Lander and Botstein (1989) Genetics 121:185), composite interval mapping, penalized regression analysis, complex pedigree analysis, MCMC analysis, MQM analysis (Jansen (1994) Genetics 138:871), HAPLO-IM+ analysis, HAPLO-MQM analysis, HAPLO-MQM+ analysis, Bayesian MCMC, ridge regression, identity-by-descent analysis, Haseman-Elston regression, any of which are suitable in the context of the present invention. In addition, additional details regarding alternative statistical methods applicable to complex breeding populations which can be used to identify and localize QTLs are described in U.S. Pat. No. 6,399,855 and WO0149104. Any of these approaches are computationally intensive and are usually performed with the assistance of a computer based system and specialized software. Appropriate statistical packages are available from a variety of public and commercial sources, and are known to those of skill in the art.

Sala et al., in Argentinean Patent Application No. ARP20030100125, disclose a combination of three QTL alleles related to resistance of maize plants to MRCV. The loci are linked to favorable resistance alleles of markers bnlg1017, bnlg2277, bnlg125, bnlg381, and bnlg180, where resistance is found when all of the alleles conferring the resistance are present.

There is a need in the art for improved maize strains that are resistant to fijivirus, particularly MRCV and MRDV, and their causative agents, e.g. Delphacodes kuscheli infection. There is a need in the art for methods that identify maize plants or populations (germplasm) that display resistance to fijivirus. What is needed in the art is to identify molecular genetic markers that are linked to fijivirus resistance loci in order to facilitate MAS, and also to facilitate gene discovery and cloning of gene alleles that impart fijivirus resistance. Such markers can be used to select individual plants and plant populations that show favorable marker alleles in maize populations and then employed to select the resistant phenotype, or alternatively, be used to counterselect plants or plant populations that show a fijivirus susceptibility phenotype. The present invention provides these and other advantages.

### SUMMARY OF THE INVENTION

Compositions and methods for identifying maize plants or germplasm with newly conferred or enhanced resistance to fijivirus are provided. Methods of making maize plants or germplasm that are resistant to fijivirus, e.g., through introgression of desired resistance marker alleles and/or by transgenic production methods, as well as plants and germplasm made by these methods, are also provided. Systems and kits for selecting resistant plants and germplasm are also a feature of the invention.

In some aspects, the invention provides methods for identifying a first maize plant or germplasm (e.g., a line or variety) that has newly conferred resistance, enhanced resistance, or susceptibility to MRCV. In the methods, at least one allele of one or more marker locus (e.g., a plurality of marker loci) that is associated with the newly conferred resistance, enhanced resistance, or susceptibility are detected in the first maize plant or germplasm. The marker loci can be selected from the loci provided in Tables 1 and 2, including MZA625, MZA16656, MZA15451, MZA15490, MZA2038, MZA11826, and MZA9105, as well as any other marker that is linked to these QTL markers (e.g., within about 10 cM of these loci). Tables 1 and 2 show maize markers demonstrating linkage disequilibrium with the MRCV resistance phenotype as determined by association mapping analysis and QTL interval mapping (including single marker regression analysis) methods. The table indicates the genomic-SSR or EST-SSR marker type (all simple sequence repeats) or SNP or MZA markers, the chromosome on which the marker is located and its approximate genetic map position relative to other known markers, given in cM, with position zero being the first (most distal) marker on the chromosome. Also shown are the maize populations used in the analysis and the statistical probability of random segregation of the marker and the resistance/susceptibility phenotype given as an adjusted probability taking into account the variability and false positives of multiple tests. Probability values from single marker regression are also shown.

The invention also provides chromosomal QTL intervals that correlate with MRCV. These intervals are located on linkage group 2. Any marker located within these intervals also finds use as a marker for MRCV resistance and is also a feature of the invention. These intervals include:
(i) MZA8381 and MZA18180;
(ii) MZA4305 and MZA2803;
(iii) MZA15490 and MZA2038;
(iv) bnlg1458b and umc1261a;
(v) bnlg1458b and umc1262a;
(vi) bnlg1327 and umc1261a; and
(viii) bnlg1327 and umc1262a.

A plurality of marker loci can be selected in the same plant. Which QTL markers are selected in combination is not particularly limited. The QTL markers used in combinations can be any of the markers listed in Tables 1 and 2, any other marker that is linked to the markers in Tables 1 and 2 (e.g., the linked markers determined from the MaizeGDB resource), or any marker within the QTL intervals described herein.

TABLE 1

| | | | | Adjusted | | | | |
|---|---|---|---|---|---|---|---|---|
| | | | | Probability | | | | |
| | | | | Structured association | | | Not structured association | |
| | Relative | | | | Association | Association | | Association |
| | Map | | | Association | analysis | analysis | | analysis |
| | Position | | Gene Pool | analysis | I | II | Association | SNPs at |
| | (cM). | | Analyzed/ | Myriad | Myriad | Myriad | analyisis set | MRCV1. |
| | PHD | Method of | Mapping | Argentine | SS | SS | 1 (SS) | Argentine |
| Marker | v1.4 | Identification | Population | inbreds | inbreds | inbreds | inbreds | inbreds |
| MZA7588 | 63.17 | Association | Broad | 0.12 | 0.341 | 0.742 | 0.000676917 | |
| | | analysis, identity | Pioneer | | | | | |
| | | by descent | germplasm | | | | | |
| MZA8381 | 63.47 | Association | Broad | 0.002 | 0.0037 | 0.0044 | 0.000198191 | less than |
| | | analysis, identity | Pioneer | | | | | 0.001 |
| | | by descent | germplasm | | | | | |
| MZA3105 | 63.55 | Association | Broad | 0.0412 | | | | 0.064 |
| | | analysis, identity | Pioneer | | | | | |
| | | by descent | germplasm | | | | | |
| MZA482 | 63.64 | Association | Broad | 0.551 | 0.0958 | 0.197 | 0.002172499 | |
| | | analysis, identity | Pioneer | | | | | |
| | | by descent | germplasm | | | | | |
| MZA16531 | 63.83 | Association | Broad | | 0.174 | 0.0282 | 0.055088894 | |
| | | analysis, identity | Pioneer | | | | | |
| | | by descent | germplasm | | | | | |
| MZA14553 | 64.1 | Association | Broad | | | | | |
| | | analysis, identity | Pioneer | | | | | |
| | | by descent | germplasm | | | | | |
| MZA4305 | 64.1 | Association | Broad | 0.644 | 0.0394 | 0.066 | 0.331615457 | |
| | | analysis, identity | Pioneer | | | | | |
| | | by descent | germplasm | | | | | |
| MZA625 | 64.1 | Association | Broad | 0.0476 | 0.685 | 0.74 | 0.000136376 | |
| | | analysis, identity | Pioneer | | | | | |
| | | by descent, QTL | germplasm | | | | | |
| | | mapping | | | | | | |
| MZA625-30-A | 64.1 | Identity by | Broad | | | | | less than |
| | | descent, QTL | Pioneer | | | | | 0.001 |
| | | mapping | germplasm | | | | | |
| MZA625-29-A | 64.1 | Identity by | Broad | | | | | less than |
| | | descent, QTL | Pioneer | | | | | 0.001 |
| | | mapping | germplasm | | | | | |
| MZA15451 | 65.3 | Association | Broad | 0.0105 | 0.0438 | 0.0612 | 0.51165696 | |
| | | analysis, identity | Pioneer | | | | | |
| | | by descent | germplasm | | | | | |
| MZA9105 | 65.4 | Association | Broad | 0.0226 | 0.436 | 0.453 | 0.003621576 | |
| | | analysis, identity | Pioneer | | | | | |
| | | by descent | germplasm | | | | | |
| MZA9105-8-A | 65.4 | Identity by | Broad | | | | | less than |
| | | descent, QTL | Pioneer | | | | | 0.001 |
| | | mapping | germplasm | | | | | |
| MZA9105-6-A | 65.4 | Identity by | Broad | | | | | 0.066 |
| | | descent, QTL | Pioneer | | | | | |
| | | mapping | germplasm | | | | | |
| MZA11826 | 66.0 | Association | Broad | 0.0201 | 0.16 | 0.486 | 1.79182E−06 | |
| | | analysis, identity | Pioneer | | | | | |
| | | by descent, QTL | germplasm | | | | | |
| | | mapping | | | | | | |
| MZA11826- | 66.0 | Identity by | Broad | | | | | 0.014 |
| 803-A | | descent, QTL | Pioneer | | | | | |
| | | mapping | germplasm | | | | | |
| MZA11826- | 66.0 | Identity by | Broad | | | | | 0.034 |
| 801-A | | descent, QTL | Pioneer | | | | | |
| | | mapping | germplasm | | | | | |
| MZA11826- | 66.0 | Identity by | Broad | | | | | 0.04 |
| 27-A | | descent, QTL | Pioneer | | | | | |
| | | mapping | germplasm | | | | | |
| MZA15490 | 66.0 | Association | Broad | 0.0079 | 0.186 | 0.523 | 0.4067326 | |
| | | analysis, identity | Pioneer | | | | | |
| | | by descent | germplasm | | | | | |
| MZA15490- | 66.0 | Identity by | Broad | | | | | less than |
| 801-A | | descent, QTL | Pioneer | | | | | 0.001 |
| | | mapping | germplasm | | | | | |
| MZA15490- | 66.0 | Identity by | Broad | | | | | less than |
| 138-A | | descent, QTL | Pioneer | | | | | 0.001 |
| | | mapping | germplasm | | | | | |
| MZA15490- | 66.0 | Identity by | Broad | | | | | less than |
| 137-A | | descent, QTL | Pioneer | | | | | 0.001 |
| | | mapping | germplasm | | | | | |
| MZA16656 | 66.0 | Association | Broad | 0.000194 | 0.452 | 0.474 | 0.011514162 | |
| | | analysis, identity | Pioneer | | | | | |
| | | by descent, QTL | germplasm | | | | | |
| | | mapping | | | | | | |
| MZA16656- | 66.0 | Identity by | Broad | | | | | less than |
| 8-A | | descent, QTL | Pioneer | | | | | 0.001 |
| | | mapping | germplasm | | | | | |
| MZA16656- | 66.0 | Identity by | Broad | | | | | less than |
| 19-A | | descent, QTL | Pioneer | | | | | 0.001 |
| | | mapping | germplasm | | | | | |
| MZA2038 | 66.0 | Association | Broad | 0.0035 | 0.104 | 0.391 | 2.66345E−06 | |
| | | analysis, identity | Pioneer | | | | | |
| | | by descent | germplasm | | | | | |
| MZA2038- | 66.0 | Identity by | Broad | | | | | 0.161 |
| 76-A | | descent, QTL | Pioneer | | | | | |
| | | mapping | germplasm | | | | | |
| MZA2038- | 66.0 | Identity by | Broad | | | | | 0.298 |
| 71-A | | descent, QTL | Pioneer | | | | | |
| | | mapping | germplasm | | | | | |
| MZA2803 | 66.0 | Association | Broad | 0.404 | 0.0728 | 0.0916 | 0.116318398 | |
| | | analysis, identity | Pioneer | | | | | |
| | | by descent | germplasm | | | | | |
| MZA18224 | 68.8 | Association | Broad | 0.000066 | 0.039 | 0.041 | 0.003921924 | |
| | | analysis, identity | Pioneer | | | | | |
| | | by descent, QTL | germplasm | | | | | |
| | | mapping | | | | | | |
| MZA18224- | 68.8 | Identity by | Broad | | | | | 0.052 |
| 801-A | | descent, QTL | Pioneer | | | | | |
| | | mapping | germplasm | | | | | |
| MZA2349 | 68.8 | Association | Broad | 0.0498 | 0.238 | 0.185 | 0.001262359 | 0.277 |
| | | analysis, identity | Pioneer | | | | | |
| | | by descent | germplasm | | | | | |
| MZA564 | 68.8 | Association | Broad | 0.756 | 0.167 | 0.0524 | 0.000254878 | |
| | | analysis, identity | Pioneer | | | | | |
| | | by descent | germplasm | | | | | |
| MZA11066 | 70.7 | Association | Broad | 0.617 | 0.819 | 0.786 | 0.330400979 | |
| | | analysis, identity | Pioneer | | | | | |
| | | by descent | germplasm | | | | | |
| MZA18180 | 71.3 | Association | Broad | 0.0272 | 0.0201 | 0.0204 | 0.091180064 | 0.005 |
| | | analysis, identity | Pioneer | | | | | |
| | | by descent | germplasm | | | | | |
| MZA8442 | 71.4 | Association | Broad | 0.000234 | 0.0358 | 0.0402 | 0.000598737 | |
| | | analysis, identity | Pioneer | | | | | |
| | | by descent | germplasm | | | | | |
| MZA15563 | 71.5 | Association | Broad | 0.0754 | 0.0079 | 0.0079 | 0.114427854 | 0.524 |
| | | analysis, identity | Pioneer | | | | | |
| | | by descent | germplasm | | | | | |
| MZA18036 | 71.8 | Association | Broad | 0.000138 | 0.112 | 0.0474 | 0.008370189 | 0.007 |
| | | analysis, identity | Pioneer | | | | | |
| | | by descent | germplasm | | | | | |
| MZA15264 | 71.9 | Association | Broad | 0.794 | 0.608 | 0.664 | 0.207135606 | |
| | | analysis, identity | Pioneer | | | | | |
| | | by descent | germplasm | | | | | |
| MZA10384 | 72.2 | Association | Broad | 0.706 | 0.133 | 0.0442 | 0.001530899 | |
| | | analysis, identity | Pioneer | | | | | |
| | | by descent | germplasm | | | | | |
| MZA12874 | 72.3 | Association | Broad | 0.829 | 0.141 | 0.215 | 0.009463312 | 0.059 |
| | | analysis, identity | Pioneer | | | | | |
| | | by descent | germplasm | | | | | |
| MZA12454 | 72.4 | Association | Broad | 0.000064 | 0.126 | 0.088 | 5.75703E−05 | |
| | | analysis, identity | Pioneer | | | | | |
| | | by descent | germplasm | | | | | |
| MZA8926 | 72.9 | Association | Broad | 0.0089 | | | 0.641842316 | |
| | | analysis, identity | Pioneer | | | | | |
| | | by descent | germplasm | | | | | |
| MZA5057 | 73.0 | Association | Broad | 4.5231E−05 | 0.0246 | 0.0098 | 0.050959299 | less than |
| | | analysis, identity | Pioneer | | | | | 0.001 |
| | | by descent | germplasm | | | | | |
| BNLG1327 | 66.9 | Link between | Extrapolation | | | | | |
| | | Pioneer and | by map | | | | | |
| | | public maps | position | | | | | |
| BNLG1458B | | Link between | Extrapolation | | | | | |
| | | Pioneer and | by map | | | | | |
| | | public maps | position | | | | | |
| UMC1261 | 70.0 | Link between | Extrapolation | | | | | |
| | | Pioneer and | by map | | | | | |
| | | public maps | position | | | | | |
| UMC1262 | 70.2 | Link between | Extrapolation | | | | | |
| | | Pioneer and | by map | | | | | |
| | | public maps | position | | | | | |

TABLE 2

| | QTL mapping | | | | |
|---|---|---|---|---|---|
| | | | | MEPS | |
| | | | | populations | |
| | PH7WTxPH3DT | PH9TJxPH890 | PH7WTxPH3DT | (adjusted | |
| Marker | mapping pop | mapping pop | BC3F3 by MAS | probability) | Notes |
| MZA625 | QTL position | QTL position | QTL position | | QTL position by |
| MZA15451 | extrapolated from | extrapolated from | corresponding to | | using the |
| MZA9105 | LOD score peak. | LOD score peak. | the highest | | information |
| MZA11826 | LOD score peak: | LOD score peak: | associated | | across different |
| MZA15490 | >6 Position 65.8; | >20; Position | markers. LOD | | association |
| MZA16656 | flanking markers | 65.99-68.8; | score peak: >10 | less than 0.05 | analysis, QTL |
| MZA2038 | umc1756- | flanking markers | | | mapping studies |
| MZA2803 | umc1518 | MZA625- | | | and Identity by |
| | | MZA18224 | | | descent |
| | | | | | infotmation. |
| BNLG1327 | | | | | Markers to |
| BNLG1458B | | | | | extrapolate the |
| UMC1261 | | | | | QTL position to |
| UMC1262 | | | | | public maps |

The markers that are linked to the QTL markers of Tables 1 and 2 can be closely linked, for example, within about 10 cM from the Tables 1 and 2 QTL markers. In some embodiments, the linked locus displays a genetic recombination distance of 9 centiMorgans, 8, 7, 6, 5, 4, 3, 2, 1, 0.75, 0.5 or 0.25, or less from the QTL marker.

In some embodiments, preferred QTL markers are selected from MZA625, MZA16656, MZA15451, MZA15490, MZA2038, MZA11826, and MZA9105. Most preferred are QTL markers selected from MZA15490 and MZA2038.

In some embodiments, the germplasm is a maize line or variety. In some aspects, the newly conferred resistance, enhanced resistance, or susceptibility of a maize plant to MRCV can be quantitated using any suitable means, for example 1 to 9 scale (MRCV score), where 1, represents a highly susceptible genotype and 9, a completely resistant genotype; 4 represents a genotype with the minimum level of resistance to generate a commercial hybrid.

A second way of evaluating MRCV resistance is by evaluating the percentage of highly susceptible plants on a specific genotype. For example, a field experiment where the genotypes are arranged on a randomly completely block design and each experimental unit is represented by a field row of 4 meters and approximately 20 plants are planted on each row. The MRCV enhanced resistance is evaluated by observing each experimental unit and assiging a field score (1 to 9 scale). At the same time, the percentage of highly susceptible plants on each experimental unit is assayed. See FIG. 3A for examples of maize showing MRCV symptoms and FIG. 3B for examples of maize showing susceptibility to MRCV.

Any of a variety of techniques can be used to identify a marker allele. It is not intended that the method of allele detection be limited in any way. Methods for allele detection typically include molecular identification methods such as amplification and detection of the marker amplicon. For example, an allelic form of a polymorphic simple sequence repeat (SSR) or of a single nucleotide polymorphism (SNP) can be detected, e.g., by an amplification based technology. In these and other amplification based detection methods, the marker locus or a portion of the marker locus is amplified (e.g., via PCR, LCR or transcription using a nucleic acid isolated from a maize plant of interest as a template), and the resulting amplified marker amplicon is detected. In one example of such an approach, an amplification primer or amplification primer pair is admixed with genomic nucleic acid isolated from the first maize plant or germplasm, wherein the primer or primer pair is complementary or partially complementary to at least a portion of the marker locus, and is capable of initiating DNA polymerization by a DNA polymerase using the maize genomic nucleic acid as a template. The primer or primer pair (e.g., a primer pair provided in Table 3) is extended in a DNA polymerization reaction having a DNA polymerase and a template genomic nucleic acid to generate at least one amplicon.

TABLE 3

| Marker | | | | |
|---|---|---|---|---|
| Name | Left Primer Sequence | Right Primer Sequence | Repeat | Also Known As (AKA) |
| BNLG1327 | SEQ ID NO: 49 | SEQ ID NO: 50 | CT(25) | bmc1327, A4615G09, |
| | | | | p-bnlg1327, |
| | | | | A4615G10, bnlg1327, |
| | | | | LGI456705 |
| BNLG1458B | SEQ ID NO: 51 | SEQ ID NO: 52 | — | bnlg1458, p- |
| | | | | bnlg1458, A4651C06, |
| | | | | bmc1458, A4651C05 |
| UMC1261 | SEQ ID NO: 53 | SEQ ID NO: 54 | (TG)8 | AI987278 |
| UMC1262 | SEQ ID NO: 55 | SEQ ID NO: 56 | (GTC)4 | AI987278 |

Table 3 lists genomic and SSR markers, including those markers that demonstrated linkage disequilibrium with the MRCV resistance phenotype (directly or by extrapolation from the genetic map). Table 3 provides the sequences of the left and right PCR primers used in the SSR marker locus genotyping analysis. Also shown is the pigtail sequence used on the 5′ end of the right primer, and the number of nucleotides in the tandem repeating element in the SSR.

In any case, data representing the detected allele(s) can be transmitted (e.g., electronically or via infrared, wireless or optical transmission) to a computer or computer readable medium for analysis or storage. In some embodiments, plant RNA is the template for the amplification reaction. In other embodiments, plant genomic DNA is the template for the amplification reaction. In some embodiments, the QTL marker is a SNP type marker, and the detected allele is a SNP allele (see, e.g., Table 4 (showing SNP markers at QTL position and the specific PH7WT (=630=PH14J) and PH9TJ haplotypes)), and the method of detection is allele specific hybridization (ASH).

TABLE 4

| QTL | MRCV1 | | | | |
|---|---|---|---|---|---|
| STARS | PASS | PASS | PASS | PASS | PASS |
| Ctg Pos | 745 | 745 | 897 | 897 | |
| Ctg | 203 | 203 | 203 | 203 | |
| PHD | 64.1 | 64.1 | 66.0 | 66.0 | 66.0 |
| Chromosome | 2 | 2 | 2 | 2 | 2 |
| Sample Name | MZA-625-29-A | MZA625-30-A | MZA16656-8-A | MZA16656-19-A | MZA15490-137-A |
| PH7WT | C | T | C | G | C |
| PH9TJ | C | T | T | A | A |

| QTL | MRCV1 | | | | |
|---|---|---|---|---|---|
| STARS | PASS | PASS | PASS | PASS | PASS |
| Ctg Pos | | | 897 | 930 | 930 |
| Ctg | | | 203 | 203 | 203 |
| PHD | 66.0 | 66.0 | 66.0 | 66.0 | 66.0 |
| Chromosome | 2 | 2 | 2 | 2 | 2 |
| Sample Name | MZA15490-138-A | MZA15490-801-A | MZA2038-71-A | MZA2038-76-A | C00081-01-A |
| PH7WT | G | G | A | T | P |
| PH9TJ | C | C | T | C | X |

| QTL | MRCV1 | | | | |
|---|---|---|---|---|---|
| STARS | PASS | PASS | PASS | PASS | PASS |
| Ctg Pos | 930 | 930 | 930 | 1018 | 1018 |
| Ctg | 203 | 203 | 203 | 203 | 203 |
| PHD | 66.0 | 66.0 | 66.0 | 65.4 | 65.4 |
| Chromosome | 2 | 2 | 2 | 2 | 2 |
| Sample Name | MZA11826-27-A | MZA11826-801-A | MZA11826-803-A | MZA9105-6-A | MZA9105-8-A |
| PH7WT | C | A | C | G | A |
| PH9TJ | T | G | T | G | A |

In some embodiments, the allele that is detected is a favorable allele that positively correlates with newly conferred resistance or enhanced resistance. Alternatively, the allele that is detected can be an allele that correlates with disease susceptibility or reduced disease resistance, and that allele is counter-selected. For example, alleles that can be selected for (favorable alleles, e.g., PH7VVT and PH9TJ (see Table 5)) or against (unfavorable alleles, e.g., PH3DT, PH890, and PH6 KW (see Table 5)).

TABLE 5

| QTL | | MRCV1 | | | | |
|---|---|---|---|---|---|---|
| STARS | | PASS | PASS | PASS | PASS | PASS |
| Ctg Pos | | 745 | 745 | 897 | 897 | |
| Ctg | | 203 | 203 | 203 | 203 | |
| PHD | | 64.1 | 64.1 | 66.0 | 66.0 | 66.0 |
| Chromosome | | 2 | 2 | 2 | 2 | 2 |
| Sample Name | MRCV1 | MZA-625-29-A | MZA625-30-A | MZA16656-8-A | MZA16656-19-A | MZA15490-137-A |
| PH7WT | Positive | C | T | C | G | C |
| | Effect | | | | | |
| PH9TJ | Positive | C | T | T | A | A |
| | Effect | | | | | |
| PH3DT | Negative | T | C | T | A | A |
| | Effect | | | | | |
| PH890 | Negative | T | C | C | A | A |
| | Effect | | | | | |
| PH6KW | Negative | T | C | T | A | A |
| | Effect | | | | | |

| QTL | | MRCV1 | | | | |
|---|---|---|---|---|---|---|
| STARS | | PASS | PASS | PASS | PASS | PASS |
| Ctg Pos | | | | 897 | 930 | 930 |
| Ctg | | | | 203 | 203 | 203 |
| PHD | | 66.0 | 66.0 | 66.0 | 66.0 | 66.0 |
| Chromosome | | 2 | 2 | 2 | 2 | 2 |
| Sample Name | MRCV1 | MZA15490-138-A | MZA15490-801-A | MZA2038-71-A | MZA2038-76-A | C00081-01-A |
| PH7WT | Positive | G | G | A | T | P |
| | Effect | | | | | |
| PH9TJ | Positive | C | C | T | C | X |
| | Effect | | | | | |
| PH3DT | Negative | C | C | T | C | X |
| | Effect | | | | | |
| PH890 | Negative | C | C | T | C | X |
| | Effect | | | | | |
| PH6KW | Negative | C | C | A | T | P |
| | Effect | | | | | |

| QTL | | MRCV1 | | | | |
|---|---|---|---|---|---|---|
| STARS | | PASS | PASS | PASS | PASS | PASS |
| Ctg Pos | | 930 | 930 | 930 | 1018 | 1018 |
| Ctg | | 203 | 203 | 203 | 203 | 203 |
| PHD | | 66.0 | 66.0 | 66.0 | 65.4 | 65.4 |
| Chromosome | | 2 | 2 | 2 | 2 | 2 |
| Sample Name | MRCV1 | MZA11826-27-A | MZA11826-801-A | MZA11826-803-A | MZA9105-6-A | MZA9105-8-A |
| PH7WT | Positive | C | A | C | G | A |
| | Effect | | | | | |
| PH9TJ | Positive | T | G | T | G | A |
| | Effect | | | | | |
| PH3DT | Negative | T | G | T | A | G |
| | Effect | | | | | |
| PH890 | Negative | T | G | T | A | G |
| | Effect | | | | | |
| PH6KW | Negative | C | A | C | G | A |
| | Effect | | | | | |

In the case where more than one marker is selected, an allele is selected for each of the markers; thus, two or more alleles are selected. In some embodiments, it can be the case that a marker locus will have more than one advantageous allele, and in that case, either allele can be selected.

It will be appreciated that the ability to identify QTL marker loci that correlate with newly conferred resistance, enhanced resistance, or susceptibility to MRCV provides a method for selecting plants that have favorable marker loci as well. That is, any plant that is identified as comprising a desired marker locus (e.g., a marker allele that positively correlates with resistance) can be selected for, while plants that lack the locus, or that have a locus that negatively correlates with resistance, can be selected against. Thus, in one method, subsequent to identification of a marker locus, the methods include selecting (e.g., isolating) the first maize plant or germplasm, or selecting a progeny of the first plant or germplasm. In some embodiments, the resulting selected first maize plant or germplasm can be crossed with a second maize plant or germplasm (e.g., an elite or exotic maize, depending on characteristics that are desired in the progeny).

Similarly, in other embodiments, if an allele is correlated with newly conferred resistance or enhanced resistance to MRCV, the method can include introgressing the allele into a second maize plant or germplasm to produce an introgressed maize plant or germplasm. In some embodiments, the second maize plant or germplasm will typically display reduced resistance to MRCV as compared to the first maize plant or germplasm, while the introgressed maize plant or germplasm will display an increased resistance to MRCV as compared to the second maize plant or germplasm. An introgressed maize plant or germplasm produced by these methods is also a feature of the invention. (In some embodiments, the favorable introgressed allele is PH7WT/PH9TJ, see Table 5).

In other aspects, various mapping populations are used to determine the linked markers of the invention. In one embodiment, the mapping population used is the population derived from the cross PH7WT×PH3DT or PH9TJ×PH890. In other embodiments, other populations can be used. In other aspects, various software is used in determining linked marker loci. For example, TASSEL, MapManager-QTX, and GeneFlow all find use with the invention. In some embodiments, such as when software is used in the linkage analysis, the detected allele information (i.e., the data) is electronically transmitted or electronically stored, for example, in a computer readable medium.

In other aspects, various mapping populations are used to determine the linked markers that find use in constructing the transgenic plant. In one embodiment, the mapping population used is the population derived from the cross PH7WT×PH3DT or PH9TJ×PH890. In other embodiments, other populations can be used. In other aspects, various software is used in determining linked marker loci used to construct the transgenic plant. For example, TASSEL, MapManager-QTX, and GeneFlow all find use with the invention.

Systems for identifying a maize plant predicted to have newly conferred resistance or enhanced resistance to MRCV are also a feature of the invention. Typically, the systems include a set of marker primers and/or probes configured to detect at least one favorable allele of one or more marker locus associated with newly conferred resistance or enhanced resistance to MRCV, wherein the marker locus or loci are selected from: MZA7588, MZA8381, MZA3105, MZA482, MZA16531, MZA14553, MZA4305, MZA625, MZA15451, MZA9105, MZA11826, MZA15490, MZA16656, MZA2038, MZA2803, MZA18224, MZA2349, MZA564, MZA11066, MZA18180, MZA8442, MZA15563, MZA18036, MZA15264, MZA10384, MZA12874, MZA12454, MZA8926, and MZA5057, as well as any other marker that is linked (or in some embodiments, closely linked, e.g., demonstrating not more than 10% recombination frequency) to these QTL markers; and furthermore, any marker locus that is located within the chromosomal QTL intervals including:
(i) MZA8381 and MZA18180;
(ii) MZA4305 and MZA2803;
(iii) MZA15490 and MZA2038;
(iv) bnlg1458b and umc1261a;
(v) bnlg1458b and umc1262a;
(vi) bnlg1327 and umc1261a; and
(viii) bnlg1327 and umc1262a.
   In some embodiments, preferred QTL markers used are selected from MZA625, MZA16656, MZA15451, MZA15490, MZA2038, MZA11826, and MZA9105.

Where a system that performs marker detection or correlation is desired, the system can also include a detector that is configured to detect one or more signal outputs from the set of marker probes or primers, or amplicon thereof, thereby identifying the presence or absence of the allele and/or system instructions that correlate the presence or absence of the favorable allele with the predicted resistance. The precise configuration of the detector will depend on the type of label used to detect the marker allele. Typical embodiments include light detectors, radioactivity detectors, and the like. Detection of the light emission or other probe label is indicative of the presence or absence of a marker allele. Similarly, the precise form of the instructions can vary depending on the components of the system, e.g., they can be present as system software in one or more integrated unit of the system, or can be present in one or more computers or computer readable media operably coupled to the detector. In one typical embodiment, the system instructions include at least one look-up table that includes a correlation between the presence or absence of the favorable allele and predicted newly conferred resistance, enhanced resistance, or susceptibility.

In some embodiments, the system can be comprised of separate elements or can be integrated into a single unit for convenient detection of markers alleles and for performing marker-resistance trait correlations. In some embodiments, the system can also include a sample, for example, genomic DNA, amplified genomic DNA, cDNA, amplified cDNA, RNA, or amplified RNA from maize or from a selected maize plant tissue.

Kits are also a feature of the invention. For example, a kit can include appropriate primers or probes for detecting resistance-associated marker loci and instructions in using the primers or probes for detecting the marker loci and correlating the loci with predicted MRCV resistance. The kits can further include packaging materials for packaging the probes, primers or instructions, controls such as control amplification reactions that include probes, primers or template nucleic acids for amplifications, molecular size markers, or the like.

### DEFINITIONS

Before describing the present invention in detail, it is to be understood that this invention is not limited to particular embodiments, which can, of course, vary. It is also to be understood that the terminology used herein is for the purpose of describing particular embodiments only, and is not intended to be limiting. As used in this specification and the appended claims, terms in the singular and the singular forms “a”, “an” and “the”, for example, include plural referents unless the content clearly dictates otherwise. Thus, for example, reference to “plant”, “the plant” or “a plant” also includes a plurality of plants; also, depending on the context, use of the term “plant” can also include genetically similar or identical progeny of that plant; use of the term “a nucleic acid” optionally includes, as a practical matter, many copies of that nucleic acid molecule; similarly, the term “probe” optionally (and typically) encompasses many similar or identical probe molecules.

Unless otherwise indicated, nucleic acids are written left to right in 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

A “plant” can be a whole plant, any part thereof, or a cell or tissue culture derived from a plant. Thus, the term “plant” can refer to any of: whole plants, plant components or organs (e.g., leaves, stems, roots, etc.), plant tissues, seeds, plant cells, and/or progeny of the same. A plant cell is a cell of a plant, taken from a plant, or derived through culture from a cell taken from a plant. Thus, the term “maize plant” includes whole maize plants, maize plant cells, maize plant protoplast, maize plant cell or maize tissue culture from which maize plants can be regenerated, maize plant calli, maize plant clumps and maize plant cells that are intact in maize plants or parts of maize plants, such as maize seeds, maize cobs, maize flowers, maize cotyledons, maize leaves, maize stems, maize buds, maize roots, maize root tips and the like.

“Germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leafs, stems, pollen, or cells, that can be cultured into a whole plant.

The term “allele” refers to one of two or more different nucleotide sequences that occur at a specific locus. For example, a first allele can occur on one chromosome, while a second allele occurs on a second homologous chromosome, e.g., as occurs for different chromosomes of a heterozygous individual, or between different homozygous or heterozygous individuals in a population. A “favorable allele” is the allele at a particular locus that confers, or contributes to, an agronomically desirable phenotype, e.g., resistance to MRCV, or alternatively, is an allele that allows the identification of susceptible plants that can be removed from a breeding program or planting. A favorable allele of a marker is a marker allele that segregates with the favorable phenotype, or alternatively, segregates with susceptible plant phenotype, therefore providing the benefit of identifying disease-prone plants. A favorable allelic form of a chromosome segment is a chromosome segment that includes a nucleotide sequence that contributes to superior agronomic performance at one or more genetic loci physically located on the chromosome segment. “Allele frequency” refers to the frequency (proportion or percentage) at which an allele is present at a locus within an individual, within a line, or within a population of lines. For example, for an allele “A”, diploid individuals of genotype “AA”, “Aa”, or “aa” have allele frequencies of 1.0, 0.5, or 0.0, respectively. One can estimate the allele frequency within a line by averaging the allele frequencies of a sample of individuals from that line. Similarly, one can calculate the allele frequency within a population of lines by averaging the allele frequencies of lines that make up the population. For a population with a finite number of individuals or lines, an allele frequency can be expressed as a count of individuals or lines (or any other specified grouping) containing the allele.

An allele “positively” correlates with a trait when it is linked to it and when presence of the allele is an indictor that the desired trait or trait form will occur in a plant comprising the allele. An allele negatively correlates with a trait when it is linked to it and when presence of the allele is an indicator that a desired trait or trait form will not occur in a plant comprising the allele.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

A “locus” is a chromosomal region where a polymorphic nucleic acid, trait determinant, gene or marker is located. Thus, for example, a “gene locus” is a specific chromosome location in the genome of a species where a specific gene can be found.

The term “quantitative trait locus” or “QTL” refers to a polymorphic genetic locus with at least one allele that correlates with the differential expression of a phenotypic trait in at least one genetic background, e.g., in at least one breeding population or progeny. A QTL can act through a single gene mechanism or by a polygenic mechanism.

The terms “marker”, “molecular marker”, “marker nucleic acid”, and “marker locus” refer to a nucleotide sequence or encoded product thereof (e.g., a protein) used as a point of reference when identifying a linked locus. A marker can be derived from genomic nucleotide sequence or from expressed nucleotide sequences (e.g., from a spliced RNA or a cDNA), or from an encoded polypeptide. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. A “marker locus” is a locus that can be used to track the presence of a second linked locus, e.g., a linked locus that encodes or contributes to expression of a phenotypic trait. For example, a marker locus can be used to monitor segregation of alleles at a locus, such as a QTL, that are genetically or physically linked to the marker locus. Thus, a “marker allele”, alternatively an “allele of a marker locus”, is one of a plurality of polymorphic nucleotide sequences found at a marker locus in a population that is polymorphic for the marker locus. In some aspects, the present invention provides marker loci correlating with resistance to MRCV in maize. Each of the identified markers is expected to be in close physical and genetic proximity (resulting in physical and/or genetic linkage) to a genetic element, e.g., a QTL, that contributes to resistance.

“Genetic markers” are nucleic acids that are polymorphic in a population and where the alleles of which can be detected and distinguished by one or more analytic methods, e.g., RFLP, AFLP, isozyme, SNP, SSR, and the like. The term also refers to nucleic acid sequences complementary to the genomic sequences, such as nucleic acids used as probes.

Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also know for the detection of expressed sequence tags (ESTs) and SSR markers derived from EST sequences and randomly amplified polymorphic DNA (RAPD).

A “genetic map” is a description of genetic linkage relationships among loci on one or more chromosomes (or linkage groups) within a given species, generally depicted in a diagrammatic or tabular form. “Genetic mapping” is the process of defining the linkage relationships of loci through the use of genetic markers, populations segregating for the markers, and standard genetic principles of recombination frequency. A “genetic map location” is a location on a genetic map relative to surrounding genetic markers on the same linkage group where a specified marker can be found within a given species. In contrast, a “physical map” of the genome refers to absolute distances (for example, measured in base pairs or isolated and overlapping contiguous genetic fragments, e.g., contigs). A physical map of the genome does not take into account the genetic behavior (e.g., recombination frequencies) between different points on the physical map.

A “genetic recombination frequency” is the frequency of a crossing over event (recombination) between two genetic loci. Recombination frequency can be observed by following the segregation of markers and/or traits following meiosis. A genetic recombination frequency can be expressed in centimorgans (cM), where one cM is the distance between two genetic markers that show a 1% recombination frequency (i.e., a crossing-over event occurs between those two markers once in every 100 cell divisions).

As used herein, the term “linkage” is used to describe the degree with which one marker locus is “associated with” another marker locus or some other locus (for example, a resistance locus).

As used herein, “linkage equilibrium” describes a situation where two markers independently segregate, i.e., sort among progeny randomly. Markers that show linkage equilibrium are considered unlinked (whether or not they lie on the same chromosome).

As used herein, “linkage disequilibrium” describes a situation where two markers segregate in a non-random manner, i.e., have a recombination frequency of less than 50% (and by definition, are separated by less than 50 cM on the same linkage group). Markers that show linkage disequilibrium are considered linked. Linkage occurs when the marker locus and a linked locus are found together in progeny plants more frequently than not together in the progeny plants. As used herein, linkage can be between two markers, or alternatively between a marker and a phenotype. A marker locus can be associated with (linked to) a trait, e.g., a marker locus can be associated with newly conferred resistance or enhanced resistance to a plant pathogen when the marker locus is in linkage disequilibrium with the resistance trait. The degree of linkage of a molecular marker to a phenotypic trait is measured, e.g., as a statistical probability of co-segregation of that molecular marker with the phenotype.

As used herein, the linkage relationship between a molecular marker and a phenotype is given as a “probability” or “adjusted probability”. The probability value is the statistical likelihood that the particular combination of a phenotype and the presence or absence of a particular marker allele is random. Thus, the lower the probability score, the greater the likelihood that a phenotype and a particular marker will co-segregate. In some aspects, the probability score is considered “significant” or “nonsignificant”. In some embodiments, a probability score of 0.05 (p=0.05, or a 5% probability) of random assortment is considered a significant indication of co-segregation. However, the present invention is not limited to this particular standard, and an acceptable probability can be any probability of less than 50% (p=0.5). For example, a significant probability can be less than 0.25, less than 0.20, less than 0.15, or less than 0.1.

The term “physically linked” is sometimes used to indicate that two loci, e.g., two marker loci, are physically present on the same chromosome.

Advantageously, the two linked loci are located in close proximity such that recombination between homologous chromosome pairs does not occur between the two loci during meiosis with high frequency, e.g., such that linked loci co-segregate at least about 90% of the time, e.g., 91%, 92%, 93%, 94%, 95%, 96%, 97%, 98%, 99%, 99.5%, 99.75%, or more of the time.

The phrase “closely linked”, in the present application, means that recombination between two linked loci occurs with a frequency of equal to or less than about 10% (i.e., are separated on a genetic map by not more than 10 cM). Put another way, the closely linked loci co-segregate at least 90% of the time. Marker loci are especially useful in the present invention when they demonstrate a significant probability of co-segregation (linkage) with a desired trait (e.g., pathogenic resistance). For example, in some aspects, these markers can be termed linked QTL markers. In other aspects, especially useful molecular markers are those markers that are linked or closely linked.

In some aspects, linkage can be expressed as any desired limit or range. For example, in some embodiments, two linked loci are two loci that are separated by less than 50 cM map units. In other embodiments, linked loci are two loci that are separated by less than 40 cM. In other embodiments, two linked loci are two loci that are separated by less than 30 cM. In other embodiments, two linked loci are two loci that are separated by less than 25 cM. In other embodiments, two linked loci are two loci that are separated by less than 20 cM. In other embodiments, two linked loci are two loci that are separated by less than 15 cM. In some aspects, it is advantageous to define a bracketed range of linkage, for example, between 10 and 20 cM, or between 10 and 30 cM, or between 10 and 40 cM.

The more closely a marker is linked to a second locus, the better an indicator for the second locus that marker becomes. Thus, in one embodiment, closely linked loci such as a marker locus and a second locus display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination a frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “proximal to” each other. In some cases, two different markers can have the same genetic map coordinates. In that case, the two markers are in such close proximity to each other that recombination occurs between them with such low frequency that it is undetectable.

When referring to the relationship between two genetic elements, such as a genetic element contributing to resistance and a proximal marker, “coupling” phase linkage indicates the state where the “favorable” allele at the resistance locus is physically associated on the same chromosome strand as the “favorable” allele of the respective linked marker locus. In coupling phase, both favorable alleles are inherited together by progeny that inherit that chromosome strand. In “repulsion” phase linkage, the “favorable” allele at the locus of interest is physically linked with an “unfavorable” allele at the proximal marker locus, and the two “favorable” alleles are not inherited together (i.e., the two loci are “out of phase” with each other).

As used herein, the terms “chromosome interval” or “chromosome segment” designate a contiguous linear span of genomic DNA that resides in planta on a single chromosome. The genetic elements or genes located on a single chromosome interval are physically linked. The size of a chromosome interval is not particularly limited.

In some aspects, for example in the context of the present invention, generally the genetic elements located within a single chromosome interval are also genetically linked, typically within a genetic recombination distance of, for example, less than or equal to 20 cM, or alternatively, less than or equal to 10 cM. That is, two genetic elements within a single chromosome interval undergo recombination at a frequency of less than or equal to 20% or 10%.

In one aspect, any marker of the invention is linked (genetically and physically) to any other marker that is at or less than 50 cM distant. In another aspect, any marker of the invention is closely linked (genetically and physically) to any other marker that is in close proximity, e.g., at or less than 10 cM distant. Two closely linked markers on the same chromosome can be positioned 9, 8, 7, 6, 5, 4, 3, 2, 1, 0.75, 0.5 or 0.25 cM or less from each other.

The phrase “disease caused by Mal de Río Cuarto Virus” or “disease caused by MRCV” refers to the plant disease caused by an infection of the plant with MRCV.

“Newly conferred resistance” or “enhanced resistance” in a maize plant to MRCV is an indication that the maize plant is less affected with respect to yield and/or survivability or other relevant agronomic measures, upon introduction of the causative agents of that disease. Resistance is a relative term, indicating that the infected plant produces better yield of maize than another, similarly treated, more susceptible plant. That is, the conditions cause a reduced decrease in maize survival and/or yield in a resistant maize plant, as compared to a susceptible maize plant.

One of skill will appreciate that maize plant resistance to MRCV varies widely, can represent a spectrum of more resistant or less resistant phenotypes, and can vary depending on the severity of the infection. However, by simple observation, one of skill can determine the relative resistance or susceptibility of different plants, plant lines or plant families to MRCV, and furthermore, will also recognize the phenotypic gradations of “resistant” (an exemplary scoring system is presented in Example 7 below). As used in the art, “resistance” is sometimes referred to as “general resistance”, “rate-reducing resistance”, or “partial resistance”.

The term “crossed” or “cross” in the context of this invention means the fusion of gametes via pollination to produce progeny (e.g., cells, seeds or plants). The term encompasses both sexual crosses (the pollination of one plant by another) and selfing (self-pollination, e.g., when the pollen and ovule are from the same plant).

The term “introgression” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a QTL, a transgene, or the like. In any case, offspring comprising the desired allele can be repeatedly backcrossed to a line having a desired genetic background and selected for the desired allele, to result in the allele becoming fixed in a selected genetic background.

A “line” or “strain” is a group of individuals of identical parentage that are generally inbred to some degree and that are generally homozygous and homogeneous at most loci (isogenic or near isogenic). A “subline” refers to an inbred subset of descendents that are genetically distinct from other similarly inbred subsets descended from the same progenitor.

An “ancestral line” is a parent line used as a source of genes e.g., for the development of elite lines. An “ancestral population” is a group of ancestors that have contributed the bulk of the genetic variation that was used to develop elite lines. “Descendants” are the progeny of ancestors, and may be separated from their ancestors by many generations of breeding. For example, elite lines are the descendants of their ancestors. A “pedigree structure” defines the relationship between a descendant and each ancestor that gave rise to that descendant. A pedigree structure can span one or more generations, describing relationships between the descendant and it's parents, grand parents, great-grand parents, etc.

An “elite line” or “elite strain” is an agronomically superior line that has resulted from many cycles of breeding and selection for superior agronomic performance. Numerous elite lines are available and known to those of skill in the art of maize breeding. An “elite population” is an assortment of elite individuals or lines that can be used to represent the state of the art in terms of agronomically superior genotypes of a given crop species, such as maize. Similarly, an “elite germplasm” or elite strain of germplasm is an agronomically superior germplasm, typically derived from and/or capable of giving rise to a plant with superior agronomic performance, such as an existing or newly developed elite line of maize.

In contrast, an “exotic maize strain” or an “exotic maize germplasm” is a strain or germplasm derived from a maize not belonging to an available elite maize line or strain of germplasm. In the context of a cross between two maize plants or strains of germplasm, an exotic germplasm is not closely related by descent to the elite germplasm with which it is crossed. Most commonly, the exotic germplasm is not derived from any known elite line of maize, but rather is selected to introduce novel genetic elements (typically novel alleles) into a breeding program.

The term “amplifying” in the context of nucleic acid amplification is any process whereby additional copies of a selected nucleic acid (or a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods. An “amplicon” is an amplified nucleic acid, e.g., a nucleic acid that is produced by amplifying a template nucleic acid by any available amplification method (e.g., PCR, LCR, transcription, or the like).

A “genomic nucleic acid” is a nucleic acid that corresponds in sequence to a heritable nucleic acid in a cell. Common examples include nuclear genomic DNA and amplicons thereof. A genomic nucleic acid is, in some cases, different from a spliced RNA, or a corresponding cDNA, in that the spliced RNA or cDNA is processed, e.g., by the splicing machinery, to remove introns. Genomic nucleic acids optionally comprise non-transcribed (e.g., chromosome structural sequences, promoter regions, or enhancer regions) and/or non-translated sequences (e.g., introns), whereas spliced RNA/cDNA typically do not have non-transcribed sequences or introns. A “template nucleic acid” is a nucleic acid that serves as a template in an amplification reaction (e.g., a polymerase based amplification reaction such as PCR, a ligase mediated amplification reaction such as LCR, a transcription reaction, or the like). A template nucleic acid can be genomic in origin, or alternatively, can be derived from expressed sequences, e.g., a cDNA or an EST.

An “exogenous nucleic acid” is a nucleic acid that is not native to a specified system (e.g., a germplasm, plant, or variety), with respect to sequence, genomic position, or both. As used herein, the terms “exogenous” or “heterologous” as applied to polynucleotides or polypeptides typically refers to molecules that have been artificially supplied to a biological system (e.g., a plant cell, a plant gene, a particular plant species or variety or a plant chromosome under study) and are not native to that particular biological system. The terms can indicate that the relevant material originated from a source other than a naturally occurring source, or can refer to molecules having a non-natural configuration, genetic location or arrangement of parts.

In contrast, for example, a “native” or “endogenous” gene is a gene that does not contain nucleic acid elements encoded by sources other than the chromosome or other genetic element on which it is normally found in nature. An endogenous gene, transcript or polypeptide is encoded by its natural chromosomal locus, and not artificially supplied to the cell.

The term “recombinant” in reference to a nucleic acid or polypeptide indicates that the material (e.g., a recombinant nucleic acid, gene, polynucleotide, or polypeptide) has been altered by human intervention. Generally, the arrangement of parts of a recombinant molecule is not a native configuration, or the primary sequence of the recombinant polynucleotide or polypeptide has in some way been manipulated. The alteration to yield the recombinant material can be performed on the material within or removed from its natural environment or state. For example, a naturally occurring nucleic acid becomes a recombinant nucleic acid if it is altered, or if it is transcribed from DNA which has been altered, by means of human intervention performed within the cell from which it originates. A gene sequence open reading frame is recombinant if that nucleotide sequence has been removed from its natural context and cloned into any type of artificial nucleic acid vector. Protocols and reagents to produce recombinant molecules, especially recombinant nucleic acids, are common and routine in the art. In one embodiment, an artificial chromosome can be created and inserted into maize plants by any method known in the art (e.g., direct transfer processes, such as, e.g., PEG-induced DNA uptake, protoplast fusion, microinjection, electroporation, and microprojectile bombardment). An artificial chromosome is a piece of DNA that can stably replicate and segregate alongside endogenous chromosomes. It has the capacity to accommodate and express heterologous genes inserted therein. Integration of heterologous DNA into the megareplicator region (primary replication initiation site of centromeres) or in close proximity thereto, initiates a large-scale amplification of megabase-size chromosomal segments, which leads to de novo chromosome formation. See, e.g., U.S. Pat. No. 6,077,697, incorporated herein by reference.

The term recombinant can also refer to an organism that harbors recombinant material, e.g., a plant that comprises a recombinant nucleic acid is considered a recombinant plant. In some embodiments, a recombinant organism is a transgenic organism.

The term “introduced” when referring to translocating a heterologous or exogenous nucleic acid into a cell refers to the incorporation of the nucleic acid into the cell using any methodology. The term encompasses such nucleic acid introduction methods as “transfection”, “transformation”, and “transduction”.

As used herein, the term “vector” is used in reference to polynucleotide or other molecules that transfer nucleic acid segment(s) into a cell. The term “vehicle” is sometimes used interchangeably with “vector”. A vector optionally comprises parts which mediate vector maintenance and enable its intended use (e.g., sequences necessary for replication, genes imparting drug or antibiotic resistance, a multiple cloning site, or operably linked promoter/enhancer elements which enable the expression of a cloned gene). Vectors are often derived from plasmids, bacteriophages, or plant or animal viruses. A “cloning vector” or “shuttle vector” or “subcloning vector” contains operably linked parts that facilitate subcloning steps (e.g., a multiple cloning site containing multiple restriction endonuclease sites).

The term “expression vector” as used herein refers to a vector comprising operably linked polynucleotide sequences that facilitate expression of a coding sequence in a particular host organism (e.g., a bacterial expression vector or a plant expression vector). Polynucleotide sequences that facilitate expression in prokaryotes typically include, e.g., a promoter, an operator (optional), and a ribosome binding site, often along with other sequences. Eukaryotic cells can use promoters, enhancers, termination and polyadenylation signals and other sequences that are generally different from those used by prokaryotes.

The term “transgenic plant” refers to a plant that comprises within its cells a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to refer to any cell, cell line, callus, tissue, plant part or plant, the genotype of which has been altered by the presence of heterologous nucleic acid including those transgenic organisms or cells initially so altered, as well as those created by crosses or asexual propagation from the initial transgenic organism or cell. The term “transgenic” as used herein does not encompass the alteration of the genome (chromosomal or extra-chromosomal) by conventional plant breeding methods (e.g., crosses) or by naturally occurring events such as random cross-fertilization, non-recombinant viral infection, non-recombinant bacterial transformation, non-recombinant transposition, or spontaneous mutation.

“Positional cloning” is a cloning procedure in which a target nucleic acid is identified and isolated by its genomic proximity to marker nucleic acid. For example, a genomic nucleic acid clone can include part or all of two more chromosomal regions that are proximal to one another. If a marker can be used to identify the genomic nucleic acid clone from a genomic library, standard methods such as sub-cloning or sequencing can be used to identify and/or isolate subsequences of the clone that are located near the marker.

A specified nucleic acid is “derived from” a given nucleic acid when it is constructed using the given nucleic acid's sequence, or when the specified nucleic acid is constructed using the given nucleic acid. For example, a cDNA or EST is derived from an expressed mRNA.

The term “genetic element” or “gene” refers to a heritable sequence of DNA, i.e., a genomic sequence, with functional significance. The term “gene” can also be used to refer to, e.g., a cDNA and/or a mRNA encoded by a genomic sequence, as well as to that genomic sequence.

The term “genotype” is the genetic constitution of an individual (or group of individuals) at one or more genetic loci, as contrasted with the observable trait (the phenotype). Genotype is defined by the allele(s) of one or more known loci that the individual has inherited from its parents. The term genotype can be used to refer to an individual's genetic constitution at a single locus, at multiple loci, or, more generally, the term genotype can be used to refer to an individual's genetic make-up for all the genes in its genome. A “haplotype” is the genotype of an individual at a plurality of genetic loci. Typically, the genetic loci described by a haplotype are physically and genetically linked, i.e., on the same chromosome segment.

The terms “phenotype”, or “phenotypic trait” or “trait” refers to one or more trait of an organism. The phenotype can be observable to the naked eye, or by any other means of evaluation known in the art, e.g., microscopy, biochemical analysis, genomic analysis, or an assay for a particular disease resistance. In some cases, a phenotype is directly controlled by a single gene or genetic locus, i.e., a “single gene trait”. In other cases, a phenotype is the result of several genes.

A “molecular phenotype” is a phenotype detectable at the level of a population of (one or more) molecules. Such molecules can be nucleic acids such as genomic DNA or RNA, proteins, or metabolites. For example, a molecular phenotype can be an expression profile for one or more gene products, e.g., at a specific stage of plant development, in response to an environmental condition or stress, etc. Expression profiles are typically evaluated at the level of RNA or protein, e.g., on a nucleic acid array or “chip” or using antibodies or other binding proteins.

The term “yield” refers to the productivity per unit area of a particular plant product of commercial value. For example, yield of maize is commonly measured in bushels of seed per acre or metric tons of seed per hectare per season. Yield is affected by both genetic and environmental factors. “Agronomics”, “agronomic traits”, and “agronomic performance” refer to the traits (and underlying genetic elements) of a given plant variety that contribute to yield over the course of growing season. Individual agronomic traits include emergence vigor, vegetative vigor, stress tolerance, disease resistance or tolerance, herbicide resistance, branching, flowering, seed set, seed size, seed density, standability, threshability and the like. Yield is, therefore, the final culmination of all agronomic traits.

A “set” of markers or probes refers to a collection or group of markers or probes, or the data derived therefrom, used for a common purpose, e.g., identifying maize plants with a desired trait (e.g., resistance to MRCV). Frequently, data corresponding to the markers or probes, or data derived from their use, is stored in an electronic medium. While each of the members of a set possess utility with respect to the specified purpose, individual markers selected from the set as well as subsets including some, but not all, of the markers are also effective in achieving the specified purpose.

A “look up table” is a table that correlates one form of data to another, or one or more forms of data with a predicted outcome that the data is relevant to. For example, a look up table can include a correlation between allele data and a predicted trait that a plant comprising a given allele is likely to display. These tables can be, and typically are, multidimensional, e.g., taking multiple alleles into account simultaneously, and, optionally, taking other factors into account as well, such as genetic background, e.g., in making a trait prediction.

A “computer readable medium” is an information storage media that can be accessed by a computer using an available or custom interface. Examples include memory (e.g., ROM, RAM, or flash memory), optical storage media (e.g., CD-ROM), magnetic storage media (computer hard drives, floppy disks, etc.), punch cards, and many others that are commercially available. Information can be transmitted between a system of interest and the computer, or to or from the computer and the computer readable medium for storage or access of stored information. This transmission can be an electrical transmission, or can be made by other available methods, such as an IR link, a wireless connection, or the like.

“System instructions” are instruction sets that can be partially or fully executed by the system. Typically, the instruction sets are present as system software.

### BRIEF DESCRIPTION OF THE DRAWINGS AND SEQUENCES

FIG. 1A shows a structured association analysis of an Argentinean group. Note: significant region (p-value: less than 0.00005) from position 65.99 to 85.84. X axis: Distance expressed on cM from the extreme of Chr 2. Y axis: probability value. FIG. 1B shows a structured association analysis for an SS group. Note: main significant marker at MRCV1, MZA1525 at position 54.62 and MZA11826 at position 65.99. X axis: Distance expressed on cM from the extreme of chromosome 2. Y axis: probability value. FIG. 1C shows a structured association analysis for another SS group. Note: The highest associated marker on the short arm of chromosome 2 was MZA12899 at position 53.83 (p=0.000298). X axis: Distance expressed on cM from the extreme of chromosome 2. Y axis: probability value.

FIG. 2 shows an interval mapping for the PH3DT×PH7WT cross. Chromosome 2, LOD score peak: position 65.89, 46% of phenotypic variation.

FIG. 3A shows photographs of maize displaying MRCV symptoms. FIG. 3B shows photographs of maize having MRCV susceptibility.

FIG. 4A shows a graphic of genotypes at the QTL region and averaged phenotypes (MRCVSC) for a group of recombinants of the high resolution mapping BC5F3 population from the cross PH3DT×PH7WT. The piece of the resistant parent into the susceptible background and the region of recombination is shown. The region includes the recombinants located between MZA1525-98-A and MZA10094-9-A. FIG. 4B shows a graphic of genotypes at the QTL region and averaged phenotypes (MRCVSC) for a group of recombinants of the high resolution mapping BC5F3 population from the cross PH3DT×PH7WT. The piece of the resistant parent into the susceptible background and the region of recombination is shown. The region includes the recombinants located between MZA15490 and MZA18224. It also includes three recombinants in the interval MZA11826 to MZA9105 genetically characterized. Phenotype is indicated by the circles at the right of the graphic (black circles: susceptible; white circles: resistant; diagonal lined circle: mix of resistant and susceptible; gray circles: unknown).

FIG. 5 shows an interval mapping for the PH3DT×PH7WT cross. Chromosome 2, LOD score peak: position 65.99 (MZA2038). MZA11826 and MZA9105 were not included in the analysis because there were not recombinants respects to MZA2038 in this specific population. Note: the genetic map was adapted to permit interval mapping at 65.99 position; markers MZA16656, MZA15490 and MZA2038 are highly linked on distances below 0.5 cM, but they were artificially positioned at distances of 0.5 cM for this specific analysis.

FIG. 6 shows an interval mapping analysis for the PH9TJ×PH890 cross on specific QTL regions on Chr 2 and Chr 5. Chromosome 2, LOD score peak: position 65.99-68.8. There were no recombinants between the preferred markers and markers at position 68.8; thus, only MZA9105 was included as representative of preferred markers for this analysis.

FIG. 7 shows a picture of a plant severely affected by MRCV; it corresponds to a isoline at preferred markers harboring the susceptible haplotype from the cross PH3DT×PH7WT. FIG. 7B shows a picture of two rows, side by side, planted in the endemic region of Río Cuarto, Argentina where the row to the left correspond to the isoline harboring susceptible haplotype and the row to the right corresponds to the isoline harboring the resistant allele at preferred markers. These isolines were derived from a single BC5F2 plant heterozygous at preferred markers from the cross PH3DT×PH7WT.

FIG. 8 shows the chromosome 2 QTL region between markers MZA15490 and MZA2038.

FIG. 9 shows a graphic of the region at the MZA15490 to MZA2038 interval where the position of specific sequenced fragments in a group of representative susceptible and resistant inbreds is indicated.

FIG. 10 shows a graphic description of a recombinant at the MZA15490 to MZA2038 interval. The point of recombination was located inside PCO644442, generating a quimeric gene from resistant (PH7WT) and susceptible (PH3DT) parents. The position of SNPs and indels is indicated in the sequenced region.

FIG. 11 shows the performance (MRDV score) of maize hybrids under MRDV infection across genotypic classes for the region of preferred markers. “−2”, “0” and “2” in the X coordinate (genotypic class) represent the genotypic classes of susceptible haplotype, heterozygous haplotype and homozygous resistant haplotype, respectively.

FIG. 12 is an interval map of mean phenotypic scores across three crop seasons for the PH7WT×PH3DT mapping population. Note that the LOD score peak is close to umc1756.

FIG. 13 is a composite interval map of mean phenotypic scores across three crop seasons for the PH7WT×PH3DT mapping population. Note that the LOD score peak is close to the umc1756-umc1518 interval.

FIG. 14 is a composite interval map of the PH9TJ×PH890 mapping population. The LOD score peak for the MRCV1 QTL was located at position 65.99-68.8.

FIG. 15 is a ClustalW sequence alignment between SEQ ID NO:211 (pco644442 promoter from PH7WT) and SEQ ID NO:212 (pco644442 promoter from PH3DT).

FIG. 16 is a ClustalW sequence alignment between SEQ ID NOs: 213-236.

The following sequence descriptions summarize the Sequence Listing attached hereto. The Sequence Listing contains one letter codes for nucleotide sequence characters and the single and three letter codes for amino acids as defined in the IUPAC-IUB standards described in Nucleic Acids Research 13:3021-3030 (1985) and in the Biochemical Journal 219(2):345-373 (1984).

SEQ ID NOs: 1-5, 8-11, 14, 15, 18, 21, 25, 29, 30, 32, 34-37, 39, and 42-48 are consensus sequences for the MZA markers found in Table 6.

SEQ ID NOs: 6, 7, 12, 13, 16, 17, 19, 20, 22-24, 26-28, 31, 33, 38, 40, and 41 are SNP consensus sequences for the SNP markers found in Table 7.

SEQ ID NOs: 49-56 are left and right primer sequences for the public markers found in Table 3.

SEQ ID NOs: 57-172 are forward external, forward internal, reverse internal, and reverse external primers for the MZA markers found in Table 6.

SEQ ID NOs: 173-210 are forward and reverse primers for the SNP markers found in Table 7.

SEQ ID NO:211 is the PCO644442 promoter region of maize inbred line PH7WT.

SEQ ID NO:212 is the PCO644442 promoter region of maize inbred line PH3DT.

SEQ ID NO:213 is the sequence region including MRQV8381 and MRQV10673 for maize inbred line PH3DT.

SEQ ID NO:214 is the sequence region including MRQV8381 and MRQV10673 for maize inbred line AP19506160.

SEQ ID NO:215 is the sequence region including MRQV8381 and MRQV10673 for maize inbred line AP19506157.

SEQ ID NO:216 is the sequence region including MRQV8381 and MRQV10673 for maize inbred line AP19506156.

SEQ ID NO:217 is the sequence region including MRQV8381 and MRQV10673 for maize inbred line PH7WT.

SEQ ID NO:218 is the sequence region including MRQV8381 and MRQV10673 for maize inbred line 630.

SEQ ID NO:219 is the sequence region including MRQV8381 and MRQV10673 for maize inbred line PHG63.

SEQ ID NO:220 is the sequence region including MRQV8381 and MRQV10673 for maize inbred line PHK09.

SEQ ID NO:221 is the sequence region including MRQV8381 and MRQV10673 for maize inbred line PHR33.

SEQ ID NO:222 is the sequence region including MRQV8381 and MRQV10673 for maize inbred line 501.

SEQ ID NO:223 is the sequence region including MRQV8381 and MRQV10673 for maize inbred line 157.

SEQ ID NO:224 is the sequence region including MRQV8381 and MRQV10673 for maize inbred line PHK56.

SEQ ID NO:225 is the sequence region including MRQV8381 and MRQV10673 for maize inbred line 661.

SEQ ID NO:226 is the sequence region including MRQV8381 and MRQV10673 for maize inbred line PHR03.

SEQ ID NO:227 is the sequence region including MRQV8381 and MRQV10673 for maize inbred line 1047.

SEQ ID NO:228 is the sequence region including MRQV8381 and MRQV10673 for maize inbred line PHJ40.

SEQ ID NO:229 is the sequence region including MRQV8381 and MRQV10673 for maize inbred line 274.

SEQ ID NO:230 is the sequence region including MRQV8381 and MRQV10673 for maize inbred line 165.

SEQ ID NO:231 is the sequence region including MRQV8381 and MRQV10673 for maize inbred line B73.

SEQ ID NO:232 is the sequence region including MRQV8381 and MRQV10673 for maize inbred line PHN47.

SEQ ID NO:233 is the sequence region including MRQV8381 and MRQV10673 for maize inbred line PH26N.

SEQ ID NO:234 is the sequence region including MRQV8381 and MRQV10673 for maize inbred line PHDG9.

SEQ ID NO:235 is the sequence region including MRQV8381 and MRQV10673 for maize inbred line ST10H60.

SEQ ID NO:236 is the sequence region including MRQV8381 and MRQV10673 for maize inbred line PHKP5.

### DETAILED DESCRIPTION OF THE INVENTION

The identification and selection of maize plants that show resistance to MRCV using MAS can provide an effective and environmentally friendly approach to overcoming losses caused by this disease. The present invention provides maize marker loci that demonstrate statistically significant co-segregation with MRCV resistance. Detection of these loci or additional linked loci can be used in marker assisted maize breeding programs to produce resistant plants, or plants with improved resistance to MRCV or a related fijivirus. The linked SSR and SNP markers identified herein are provided in Tables 1 and 2. These markers include MZA625, MZA16656, MZA15451, MZA15490, MZA2038, MZA11826, and MZA9105.

Each of the SSR-type markers display a plurality of alleles that can be visualized as different sized PCR amplicons. The PCR primers that are used to generate the SSR-marker amplicons are provided in Table 3. The alleles of SNP-type markers are determined using an allele-specific hybridization protocol, as known in the art. The PCR primers used to amplify the SNP domain, and the allele-specific probes used to genotype the locus, are provided in Tables 6 and 7.

TABLE 6

| | MZA primers | | | | |
|---|---|---|---|---|---|
| MZA Marker | Forward/external | Forward/internal | Reverse/internal | Reverse/external | MZA consensus |
| MZA7588 | SEQ ID NO: 57 | SEQ ID NO: 58 | SEQ ID NO: 59 | SEQ ID NO: 60 | SEQ ID NO: 1 |
| MZA8381 | SEQ ID NO: 61 | SEQ ID NO: 62 | SEQ ID NO: 63 | SEQ ID NO: 64 | SEQ ID NO: 2 |
| MZA3105 | SEQ ID NO: 65 | SEQ ID NO: 66 | SEQ ID NO: 67 | SEQ ID NO: 68 | SEQ ID NO: 3 |
| MZA482 | SEQ ID NO: 69 | SEQ ID NO: 70 | SEQ ID NO: 71 | SEQ ID NO: 72 | SEQ ID NO: 4 |
| MZA16531 | SEQ ID NO: 73 | SEQ ID NO: 74 | SEQ ID NO: 75 | SEQ ID NO: 76 | SEQ ID NO: 5 |
| MZA625 | SEQ ID NO: 77 | SEQ ID NO: 78 | SEQ ID NO: 79 | SEQ ID NO: 80 | SEQ ID NO: 8 |
| MZA4305 | SEQ ID NO: 81 | SEQ ID NO: 82 | SEQ ID NO: 83 | SEQ ID NO: 84 | SEQ ID NO: 9 |
| MZA14553 | SEQ ID NO: 85 | SEQ ID NO: 86 | SEQ ID NO: 87 | SEQ ID NO: 88 | SEQ ID NO: 10 |
| MZA15451 | SEQ ID NO: 89 | SEQ ID NO: 90 | SEQ ID NO: 91 | SEQ ID NO: 92 | SEQ ID NO: 11 |
| MZA9105 | SEQ ID NO: 93 | SEQ ID NO: 94 | SEQ ID NO: 95 | SEQ ID NO: 96 | SEQ ID NO: 14 |
| MZA2803 | SEQ ID NO: 97 | SEQ ID NO: 98 | SEQ ID NO: 99 | SEQ ID NO: 100 | SEQ ID NO: 15 |
| MZA2038 | SEQ ID NO: 101 | SEQ ID NO: 102 | SEQ ID NO: 103 | SEQ ID NO: 104 | SEQ ID NO: 18 |
| MZA16656 | SEQ ID NO: 105 | SEQ ID NO: 106 | SEQ ID NO: 107 | SEQ ID NO: 108 | SEQ ID NO: 21 |
| MZA15490 | SEQ ID NO: 109 | SEQ ID NO: 110 | SEQ ID NO: 111 | SEQ ID NO: 112 | SEQ ID NO: 25 |
| MZA11826 | SEQ ID NO: 113 | SEQ ID NO: 114 | SEQ ID NO: 115 | SEQ ID NO: 116 | SEQ ID NO: 29 |
| MZA564 | SEQ ID NO: 117 | SEQ ID NO: 118 | SEQ ID NO: 119 | SEQ ID NO: 120 | SEQ ID NO: 30 |
| MZA2349 | SEQ ID NO: 121 | SEQ ID NO: 122 | SEQ ID NO: 123 | SEQ ID NO: 124 | SEQ ID NO: 32 |
| MZA18224 | SEQ ID NO: 125 | SEQ ID NO: 126 | SEQ ID NO: 127 | SEQ ID NO: 128 | SEQ ID NO: 34 |
| MZA11066 | SEQ ID NO: 129 | SEQ ID NO: 130 | SEQ ID NO: 131 | SEQ ID NO: 132 | SEQ ID NO: 35 |
| MZA18180 | SEQ ID NO: 133 | SEQ ID NO: 134 | SEQ ID NO: 135 | SEQ ID NO: 136 | SEQ ID NO: 36 |
| MZA8442 | SEQ ID NO: 137 | SEQ ID NO: 138 | SEQ ID NO: 139 | SEQ ID NO: 140 | SEQ ID NO: 37 |
| MZA15563 | SEQ ID NO: 141 | SEQ ID NO: 142 | SEQ ID NO: 143 | SEQ ID NO: 144 | SEQ ID NO: 39 |
| MZA18036 | SEQ ID NO: 145 | SEQ ID NO: 146 | SEQ ID NO: 147 | SEQ ID NO: 148 | SEQ ID NO: 42 |
| MZA15264 | SEQ ID NO: 149 | SEQ ID NO: 150 | SEQ ID NO: 151 | SEQ ID NO: 152 | SEQ ID NO: 43 |
| MZA10384 | SEQ ID NO: 153 | SEQ ID NO: 154 | SEQ ID NO: 155 | SEQ ID NO: 156 | SEQ ID NO: 44 |
| MZA12874 | SEQ ID NO: 157 | SEQ ID NO: 158 | SEQ ID NO: 159 | SEQ ID NO: 160 | SEQ ID NO: 45 |
| MZA12454 | SEQ ID NO: 161 | SEQ ID NO: 162 | SEQ ID NO: 163 | SEQ ID NO: 164 | SEQ ID NO: 46 |
| MZA8926 | SEQ ID NO: 165 | SEQ ID NO: 166 | SEQ ID NO: 167 | SEQ ID NO: 168 | SEQ ID NO: 47 |
| MZA5057 | SEQ ID NO: 169 | SEQ ID NO: 170 | SEQ ID NO: 171 | SEQ ID NO: 172 | SEQ ID NO: 48 |

TABLE 7

| | SNP primers | | SNP alleles | |
|---|---|---|---|---|
| SNP Marker | Forward | Reverse | SNP | SNP consensus |
| MZA625-30-A | SEQ ID NO: 173 | SEQ ID NO: 174 | T/C | SEQ ID NO: 6 (SNP at position 186) |
| MZA625-29-A | SEQ ID NO: 175 | SEQ ID NO: 176 | T/C | SEQ ID NO: 7 (SNP at position 165) |
| MZA9105-8-A | SEQ ID NO: 177 | SEQ ID NO: 178 | G/A | SEQ ID NO: 12 (SNP at position 123) |
| MZA9105-6-A | SEQ ID NO: 179 | SEQ ID NO: 180 | G/A | SEQ ID NO: 13 (SNP at position 98) |
| MZA2038-76-A | SEQ ID NO: 181 | SEQ ID NO: 182 | T/C | SEQ ID NO: 16 (SNP at position 277) |
| MZA2038-71-A | SEQ ID NO: 183 | SEQ ID NO: 184 | T/A | SEQ ID NO: 17 (SNP at position 258) |
| MZA16656-8-A | SEQ ID NO: 185 | SEQ ID NO: 186 | T/C | SEQ ID NO: 19 (SNP at position 85) |
| MZA16656-19-A | SEQ ID NO: 187 | SEQ ID NO: 188 | G/A | SEQ ID NO: 20 (SNP at position 218) |
| MZA15490-801-A | SEQ ID NO: 189 | SEQ ID NO: 190 | G/C | SEQ ID NO: 22 (SNP at position 84) |
| MZA15490-138-A | SEQ ID NO: 191 | SEQ ID NO: 192 | G/C | SEQ ID NO: 23 (SNP at position 96) |
| MZA15490-137-A | SEQ ID NO: 193 | SEQ ID NO: 194 | C/A | SEQ ID NO: 24 (SNP at position 84) |
| MZA11826-803-A | SEQ ID NO: 195 | SEQ ID NO: 196 | C/T | SEQ ID NO: 26 (SNP at position 802) |
| MZA11826-801-A | SEQ ID NO: 197 | SEQ ID NO: 198 | A/G | SEQ ID NO: 27 (SNP at position 89) |
| MZA11826-27-A | SEQ ID NO: 199 | SEQ ID NO: 200 | T/C | SEQ ID NO: 28 (SNP at position 222) |
| MZA2349-71-A | SEQ ID NO: 201 | SEQ ID NO: 202 | T/C | SEQ ID NO: 31 (SNP at position 133) |
| MZA18224-801-A | SEQ ID NO: 203 | SEQ ID NO: 204 | A/G | SEQ ID NO: 33 (SNP at position 188) |
| MZA15563-12-A | SEQ ID NO: 205 | SEQ ID NO: 206 | T/A | SEQ ID NO: 38 (SNP at position 601) |
| MZA18036-9-A | SEQ ID NO: 207 | SEQ ID NO: 208 | A/G | SEQ ID NO: 40 (SNP at position 90) |
| MZA18036-23-A | SEQ ID NO: 209 | SEQ ID NO: 210 | A/G | SEQ ID NO: 41 (SNP at position 285) |

Tables 6 and 7 list the SNP markers that demonstrated linkage disequilibrium with the MRCV resistance phenotype. These tables provide the sequences of the PCR primers used to generate a SNP-containing amplicon, and the allele-specific probes that were used to identify the SNP allele in an allele-specific hybridization assay (ASH assay).

As recognized in the art, any other marker that is linked to a QTL marker (e.g., a disease resistance marker) also finds use for that same purpose. Examples of additional markers that are linked to the disease resistance markers recited herein are provided. For example, a linked marker can be determined from the closely linked markers provided in Table 8.

TABLE 8
Linked Markers

pco061820a, pco116928a, sog0930a, pco102443, sog5467ac, cl7211_1l,
k4-14p, pco135612a, pco101521, si687005h09c, si707023g07c,
cl15901_1a, pco134907, si660032f12i, cl7048_1b,
cl2578_1, cl5312_1a, pco094715, sog5829a,
cl30_1e, pco125905, sog0690, cl36282_1b, pco118508, gpm636,
pco066747a, pco083425q, sog5844av, bnlg1458b, si606065e12a,
cl22018_1, pco091058, si946053g10, sog1265, sog0743c,
cl9862_1, pco114887, bnlg1327, sog5587a, cl1488_-4a,
pco085208a, sog1295c, sog5609b, sog0912a, tel7sc1ah, si66060d11b,
cl10933_1d, cl37019_1a, sog1856ae, pco117007l,
cl40761_1a, siaf099388e, pco137067a, sog2274m, cl31185_3a,
pco098939a, pco151039r, cl11825_1a, pco122145b, cl24291_1a,
si618065b03a, si707029g03a, sog1495a, IDP4006, umc1262a,
umc1261a, sog5758o

It is not intended, however, that linked markers finding use with the invention be limited to those recited in Table 8.

The invention also provides chromosomal QTL intervals that correlate with MRCV resistance. These intervals are located on linkage group 2. Any marker located within these intervals finds use as a marker for MRCV resistance. These intervals include:
(i) MZA8381 and MZA18180;
(ii) MZA4305 and MZA2803;
(iii) MZA15490 and MZA2038;
(iv) bnlg1458b and umc1261a;
(v) bnlg1458b and umc1262a;
(vi) bnlg1327 and umc1261a; and
(viii) bnlg1327 and umc1262a.

Methods for identifying maize plants or germplasm that carry preferred alleles of resistance marker loci are a feature of the invention. In these methods, any of a variety of marker detection protocols are used to identify marker loci, depending on the type of marker loci. Typical methods for marker detection include amplification and detection of the resulting amplified markers, e.g., by PCR, LCR, transcription based amplification methods, or the like. These include ASH, SSR detection, RFLP analysis and many others.

Although particular marker alleles can show co-segregation with a disease resistance or susceptibility phenotype, it is important to note that the marker locus is not necessarily part of the QTL locus responsible for the resistance or susceptibility. For example, it is not a requirement that the marker polynucleotide sequence be part of a gene that imparts disease resistance (for example, be part of the gene open reading frame). The association between a specific marker allele with the resistance or susceptibility phenotype is due to the original “coupling” linkage phase between the marker allele and the QTL resistance or susceptibility allele in the ancestral maize line from which the resistance or susceptibility allele originated. Eventually, with repeated recombination, crossing over events between the marker and QTL locus can change this orientation. For this reason, the favorable marker allele may change depending on the linkage phase that exists within the resistant parent used to create segregating populations. This does not change the fact that the genetic marker can be used to monitor segregation of the phenotype. It only changes which marker allele is considered favorable in a given segregating population.

Identification of maize plants or germplasm that include a marker locus or marker loci linked to a resistance trait or traits provides a basis for performing marker assisted selection of maize. Maize plants that comprise favorable markers or favorable alleles are selected for, while maize plants that comprise markers or alleles that are negatively correlated with resistance can be selected against. Desired markers and/or alleles can be introgressed into maize having a desired (e.g., elite or exotic) genetic background to produce an introgressed resistant maize plant or germplasm. In some aspects, it is contemplated that a plurality of resistance markers are sequentially or simultaneous selected and/or introgressed. The combinations of resistance markers that are selected for in a single plant is not limited, and can include any combination of markers recited in Tables 1 and 2, any markers linked to the markers recited in Tables 1 and 2, or any markers located within the QTL intervals defined herein.

As an alternative to standard breeding methods of introducing traits of interest into maize (e.g., introgression), transgenic approaches can also be used. In these methods, exogenous nucleic acids that encode traits linked to markers are introduced into target plants or germplasm. For example, a nucleic acid that codes for a resistance trait is cloned, e.g., via positional cloning and introduced into a target plant or germplasm.

Verification of resistance can be performed by available resistance protocols (see, e.g., Example 10). Resistance assays are useful to verify that the resistance trait still segregates with the marker in any particular plant or population, and, of course, to measure the degree of resistance improvement achieved by introgressing or recombinantly introducing the trait into a desired background.

Systems, including automated systems for selecting plants that comprise a marker of interest and/or for correlating presence of the marker with resistance are also a feature of the invention. These systems can include probes relevant to marker locus detection, detectors for detecting labels on the probes, appropriate fluid handling elements and temperature controllers that mix probes and templates and/or amplify templates, and systems instructions that correlate label detection to the presence of a particular marker locus or allele.

Kits are also a feature of the invention. For example, a kit can include appropriate primers or probes for detecting resistance-associated marker loci and instructions in using the primers or probes for detecting the marker loci and correlating the loci with predicted MRCV resistance. The kits can further include packaging materials for packaging the probes, primers or instructions, controls such as control amplification reactions that include probes, primers or template nucleic acids for amplifications, molecular size markers, or the like.

### Resistance Markers and Favorable Alleles

In traditional linkage analysis, no direct knowledge of the physical relationship of genes on a chromosome is required. Mendel's first law is that factors of pairs of characters are segregated, meaning that alleles of a diploid trait separate into two gametes and then into different offspring. Classical linkage analysis can be thought of as a statistical description of the relative frequencies of cosegregation of different traits. Linkage analysis is the well characterized descriptive framework of how traits are grouped together based upon the frequency with which they segregate together. That is, if two non-allelic traits are inherited together with a greater than random frequency, they are said to be “linked”. The frequency with which the traits are inherited together is the primary measure of how tightly the traits are linked, i.e., traits which are inherited together with a higher frequency are more closely linked than traits which are inherited together with lower (but still above random) frequency. Traits are linked because the genes which underlie the traits reside on the same chromosome. The further apart on a chromosome the genes reside, the less likely they are to segregate together, because homologous chromosomes recombine during meiosis. Thus, the further apart on a chromosome the genes reside, the more likely it is that there will be a crossing over event during meiosis that will result in two genes segregating separately into progeny.

A common measure of linkage is the frequency with which traits cosegregate. This can be expressed as a percentage of cosegregation (recombination frequency) or, also commonly, in centiMorgans (cM). The cM is named after the pioneering geneticist Thomas Hunt Morgan and is a unit of measure of genetic recombination frequency. One cM is equal to a 1% chance that a trait at one genetic locus will be separated from a trait at another locus due to crossing over in a single generation (meaning the traits segregate together 99% of the time). Because chromosomal distance is approximately proportional to the frequency of crossing over events between traits, there is an approximate physical distance that correlates with recombination frequency. For example, in maize, 1 cM correlates, on average, to about 2,140,000 base pairs (2.14 Mbp).

Marker loci are themselves traits and can be assessed according to standard linkage analysis by tracking the marker loci during segregation. Thus, in the context of the present invention, one cM is equal to a 1% chance that a marker locus will be separated from another locus (which can be any other trait, e.g., another marker locus, or another trait locus that encodes a QTL), due to crossing over in a single generation. The markers herein, as described in Tables 1 and 2, e.g., MZA625, MZA16656, MZA15451, MZA15490, MZA2038, MZA11826, and MZA9105, as well as any of the chromosome intervals
(i) MZA8381 and MZA18180;
(ii) MZA4305 and MZA2803;
(iii) MZA15490 and MZA2038;
(iv) bnlg1458b and umc1261a;
(v) bnlg1458b and umc1262a;
(vi) bnlg1327 and umc1261a; and
(viii) bnlg1327 and umc1262a;
   have been found to correlate with newly conferred resistance, enhanced resistance, or susceptibility to MRCV in maize. This means that the markers are sufficiently proximal to a resistance trait that they can be used as a predictor for the resistance trait. This is extremely useful in the context of marker assisted selection (MAS), discussed in more detail herein. In brief, maize plants or germplasm can be selected for markers or marker alleles that positively correlate with resistance, without actually raising maize and measuring for newly conferred resistance or enhanced resistance (or, contrarily, maize plants can be selected against if they possess markers that negatively correlate with newly conferred resistance or enhanced resistance). MAS is a powerful shortcut to selecting for desired phenotypes and for introgressing desired traits into cultivars of maize (e.g., introgressing desired traits into elite lines). MAS is easily adapted to high throughput molecular analysis methods that can quickly screen large numbers of plant or germplasm genetic material for the markers of interest and is much more cost effective than raising and observing plants for visible traits.

In some embodiments, the most preferred QTL markers are a subset of the markers provided in Tables 1 and 2. For example, the most preferred markers are MZA15490 and MZA2038.

When referring to the relationship between two genetic elements, such as a genetic element contributing to resistance and a proximal marker, “coupling” phase linkage indicates the state where the “favorable” allele at the resistance locus is physically associated on the same chromosome strand as the “favorable” allele of the respective linked marker locus. In coupling phase, both favorable alleles are inherited together by progeny that inherit that chromosome strand. In “repulsion” phase linkage, the “favorable” allele at the locus of interest (e.g., a QTL for resistance) is physically linked with an “unfavorable” allele at the proximal marker locus, and the two “favorable” alleles are not inherited together (i.e., the two loci are “out of phase” with each other).

A favorable allele of a marker is that allele of the marker that co-segregates with a desired phenotype (e.g., disease resistance). As used herein, a QTL marker has a minimum of one favorable allele, although it is possible that the marker might have two or more favorable alleles found in the population. Any favorable allele of that marker can be used advantageously for the identification and construction of resistant maize lines. Optionally, one, two, three or more favorable allele(s) of different markers are identified in, or introgressed into a plant, and can be selected for or against during MAS. Desirably, plants or germplasm are identified that have at least one such favorable allele that positively correlates with newly conferred or enhanced resistance.

Alternatively, a marker allele that co-segregates with disease susceptibility also finds use with the invention, since that allele can be used to identify and counter select disease-susceptible plants. Such an allele can be used for exclusionary purposes during breeding to identify alleles that negatively correlate with resistance, to eliminate susceptible plants or germplasm from subsequent rounds of breeding.

In some embodiments of the invention, a plurality of marker alleles are simultaneously selected for in a single plant or a population of plants. In these methods, plants are selected that contain favorable alleles from more than one resistance marker, or alternatively, favorable alleles from more than one resistance marker are introgressed into a desired maize germplasm. One of skill in the art recognizes that the simultaneous selection of favorable alleles from more than one disease resistance marker in the same plant is likely to result in an additive (or even synergistic) protective effect for the plant.

One of skill recognizes that the identification of favorable marker alleles is germplasm-specific. The determination of which marker alleles correlate with resistance (or susceptibility) is determined for the particular germplasm under study. One of skill recognizes that methods for identifying the favorable alleles are routine and well known in the art, and furthermore, that the identification and use of such favorable alleles is well within the scope of the invention. Furthermore still, identification of favorable marker alleles in maize populations other than the populations used or described herein is well within the scope of the invention.

Amplification primers for amplifying SSR-type marker loci are a feature of the invention. Another feature of the invention is primers specific for the amplification of SNP domains (SNP markers), and the probes that are used to genotype the SNP sequences. Tables 6 and 7 provide specific primers for marker locus amplification and probes for detecting amplified marker loci. However, one of skill will immediately recognize that other sequences to either side of the given primers can be used in place of the given primers, so long as the primers can amplify a region that includes the allele to be detected. Further, it will be appreciated that the precise probe to be used for detection can vary, e.g., any probe that can identify the region of a marker amplicon to be detected can be substituted for those examples provided herein. Further, the configuration of the amplification primers and detection probes can, of course, vary. Thus, the invention is not limited to the primers and probes specifically recited herein.

In some aspects, methods of the invention utilize an amplification step to detect/genotype a marker locus. However, it will be appreciated that amplification is not a requirement for marker detection—for example, one can directly detect unamplified genomic DNA simply by performing a Southern blot on a sample of genomic DNA. Procedures for performing Southern blotting, amplification (PCR, LCR, or the like) and many other nucleic acid detection methods are well established and are taught, e.g., in Sambrook et al., Molecular Cloning—A Laboratory Manual (3^{rd} Ed.), Vol. 1-3, Cold Spring Harbor Laboratory, Cold Spring Harbor, N.Y., 2000 (“Sambrook”); Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”) and PCR Protocols A Guide to Methods and Applications (Innis et al. eds) Academic Press Inc. San Diego, Calif. (1990) (“Innis”). Additional details regarding detection of nucleic acids in plants can also be found, e.g., in Plant Molecular Biology (1993) Croy (ed.) BIOS Scientific Publishers, Inc. (“Croy”).

Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

Typically, molecular markers are detected by any established method available in the art, including, without limitation, allele specific hybridization (ASH) or other methods for detecting single nucleotide polymorphisms (SNP), amplified fragment length polymorphism (AFLP) detection, amplified variable sequence detection, randomly amplified polymorphic DNA (RAPD) detection, restriction fragment length polymorphism (RFLP) detection, self-sustained sequence replication detection, simple sequence repeat (SSR) detection, single-strand conformation polymorphisms (SSCP) detection, isozyme markers detection, or the like. While the exemplary markers provided in the figures and tables herein are either SSR or SNP (ASH) markers, any of the aforementioned marker types can be employed in the context of the invention to identify chromosome segments encompassing genetic element that contribute to superior agronomic performance (e.g., newly conferred resistance or enhanced resistance).

### QTL Chromosome Intervals

In some aspects, the invention provides QTL chromosome intervals, where a QTL (or multiple QTL) that segregate with MRCV resistance are contained in those intervals. A variety of methods well known in the art are available for identifying chromosome intervals (also as described in detail in Examples 1 and 2). The boundaries of such chromosome intervals are drawn to encompass markers that will be linked to one or more QTL. In other words, the chromosome interval is drawn such that any marker that lies within that interval (including the terminal markers that define the boundaries of the interval) can be used as markers for disease resistance. Each interval comprises at least one QTL, and furthermore, may indeed comprise more than one QTL. Close proximity of multiple QTL in the same interval may obfuscate the correlation of a particular marker with a particular QTL, as one marker may demonstrate linkage to more than one QTL. Conversely, e.g., if two markers in close proximity show co-segregation with the desired phenotypic trait, it is sometimes unclear if each of those markers identify the same QTL or two different QTL. Regardless, knowledge of how many QTL are in a particular interval is not necessary to make or practice the invention.

The present invention provides maize chromosome intervals, where the markers within that interval demonstrate co-segregation with resistance to MRCV. Thus, each of these intervals comprises at least one MRCV resistance QTL as shown in Table 9.

TABLE 9

| | Method(s) of |
|---|---|
| Flanking Markers | Identification |
| MZA8381 and | Association |
| MZA1810 | analysis, identity |
| | by descent |
| MZA4305 and | Association |
| MZA2803 | analysis, identity |
| | by descent |
| MZA15490 and | Association |
| MZA2038 | analysis, identity |
| | by descent |
| bnlg1458b and | Linkage to a |
| umc1261a | preferred marker |
| bnlg1458b and | Linkage to a |
| umc1262a | preferred marker |
| bnlg1327 and | Linkage to a |
| umc1261a | preferred marker |
| bnlg1327 and | Linkage to a |
| umc1262a | preferred marker |

Each of the intervals described above shows a clustering of markers that co-segregate with MRCV resistance. This clustering of markers occurs in relatively small domains on the linkage groups, indicating the presence of one or more QTL in those chromosome regions. QTL intervals were drawn to encompass the markers that co-segregate with resistance. The intervals are defined by the markers on their termini, where the interval encompasses all the markers that map within the interval as well as the markers that define the termini.

In some cases, an interval can be drawn where the interval is defined by linkage to a preferred marker. For example, an interval on chromosome 2 is defined where any marker that is linked to the marker MZA16656 is a member of that interval. For example, as used here, linkage is defined as any marker that is within 25 cM from MZA16656. This interval on chromosome 2 is further illustrated in Table 8. The markers that are linked to MZA16656 (e.g., within 5 cM of MZA16656) as determined by any suitable genetic linkage map (for example, the IBM2 2005 Neighbors Frame 2 map found on the MaizeGDB website). These markers are shown in genetic order. Each of the markers listed, including the terminal markers pco061820a and sog5758o, are members of the interval. The pco061820a and sog5758o markers are known in the art.

As described above, an interval (e.g., a chromosome interval or a QTL interval) need not depend on an absolute measure of interval size such as a centimorgans value. An interval can be described by the terminal markers that define the endpoints of the interval, and typically the interval will include the terminal markers that define the extent of the interval. An interval can include any marker localizing within that chromosome domain, whether those markers are currently known or unknown. The invention provides a variety of means for defining a chromosome interval, for example, in the lists of linked markers of Table 8, and in references cited herein.

### Genetic Maps

As one of skill in the art will recognize, recombination frequencies (and as a result, genetic map positions) in any particular population are not static. The genetic distances separating two markers (or a marker and a QTL) can vary depending on how the map positions are determined. For example, variables such as the parental mapping populations used, the software used in the marker mapping or QTL mapping, and the parameters input by the user of the mapping software can contribute to the QTL/marker genetic map relationships. However, it is not intended that the invention be limited to any particular mapping populations, use of any particular software, or any particular set of software parameters to determine linkage of a particular marker or chromosome interval with the MRCV resistance phenotype. It is well within the ability of one of ordinary skill in the art to extrapolate the novel features described herein to any maize gene pool or population of interest, and using any particular software and software parameters. Indeed, observations regarding resistance markers and chromosome intervals in populations in additions to those described herein are readily made using the teaching of the present disclosure.

### Mapping Software

A variety of commercial software is available for genetic mapping and marker association studies (e.g., QTL mapping). This software includes but is not limited to those listed in Table 10.

TABLE 10

| Software | Description/References |
|---|---|
| Windows QTL | Wang S., C. J. Basten, and Z.-B. Zeng (2007). Windows QTL |
| Cartographer | Cartographer 2.5. Department of Statistics, North Carolina |
| Version 2.5 | State University, Raleigh, NC. |
| JoinMap ® | VanOoijen, and Voorrips (2001) “JoinMap 3.0 software for the |
| | calculation of genetic linkage maps”, Plant Research |
| | International, Wageningen, the Netherlands; and, Stam |
| | “Construction of integrated genetic linkage maps by means of |
| | a new computer package: JoinMap”, The Plant Journal |
| | 3(5): 739-744 (1993) |
| MapQTL ® | J. W. vanOoijen, “Software for the mapping of quantitative trait |
| | loci in experimental populations”, Kyazma B. V., Wageningen, |
| | Netherlands |
| MapManager QT | Manly and Olson, “Overview of QTL mapping software and |
| | introduction to Map Manager QT”, Mamm. Genome 10: 327-334 |
| | (1999) |
| MapManager QTX | Manly, Cudmore and Meer, “MapManager QTX, cross-platform |
| | software for genetic mapping”, Mamm. Genome 12: 930-932 |
| | (2001) |
| GeneFlow ® and | GENEFLOW, Inc. (Alexandria, VA) |
| QTLocate ™ | |
| TASSEL | (Trait Analysis by aSSociation, Evolution, and Linkage) by |
| | Edward Buckler, and information about the program can be |
| | found on the Buckler Lab web page at the Institute for |
| | Genomic Diversity at Cornell University. |

### Unified Genetic Maps

“Unified”, “consensus” or “integrated” genetic maps have been created that incorporate mapping data from two or more sources, including sources that used different mapping populations and different modes of statistical analysis. The merging of genetic map information increases the marker density on the map, as well as improving map resolution. These improved maps can be advantageously used in marker assisted selection, map-based cloning, provide an improved framework for positioning newly identified molecular markers and aid in the identification of QTL chromosome intervals and clusters of advantageously-linked markers.

In some aspects, a consensus map is derived by simply overlaying one map on top of another. In other aspects, various algorithms, e.g., JoinMap® analysis, allows the combination of genetic mapping data from multiple sources, and reconciles discrepancies between mapping data from the original sources. See, Van Ooijen and Voorrips (2001) “JoinMap 3.0 software for the calculation of genetic linkage maps”, Plant Research International, Wageningen, the Netherlands; Stam (1993) “Construction of integrated genetic linkage maps by means of a new computer package: JoinMap”, The Plant Journal 3(5):739-744.

### Linked Markers

From the present disclosure and widely recognized in the art, it is clear that any genetic marker that has a significant probability of co-segregation with a phenotypic trait of interest (e.g., in the present case, a newly conferred resistance or enhanced resistance trait) can be used as a marker for that trait. A list of useful QTL markers provided by the present invention is provided in Tables 1 and 2.

In addition to the QTL markers noted in Tables 1 and 2, additional markers linked to (showing linkage disequilibrium with) the QTL markers can also be used to predict the newly conferred resistance or enhanced resistance trait in a maize plant. In other words, any other marker showing less than 50% recombination frequency (separated by a genetic distance less than 50 cM) with a QTL marker of the invention (e.g., the markers provided in Tables 1 and 2) is also a feature of the invention. Any marker that is linked to a QTL marker can also be used advantageously in marker-assisted selection for the particular trait.

Genetic markers that are linked to QTL markers (e.g., QTL markers provided in Tables 1 and 2) are particularly useful when they are sufficiently proximal (e.g., closely linked) to a given QTL marker so that the genetic marker and the QTL marker display a low recombination frequency. In the present invention, such closely linked markers are a feature of the invention. As defined herein, closely linked markers display a recombination frequency of about 10% or less (the given marker is within 10 cM of the QTL). Put another way, these closely linked loci co-segregate at least 90% of the time. Indeed, the closer a marker is to a QTL marker, the more effective and advantageous that marker becomes as an indicator for the desired trait.

Thus, in other embodiments, closely linked loci such as a QTL marker locus and a second locus display an inter-locus cross-over frequency of about 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci (e.g., a marker locus and a target locus such as a QTL) display a recombination a frequency of about 1% or less, e.g., about 0.75% or less, more preferably about 0.5% or less, or yet more preferably about 0.25% or less. Thus, the loci are about 10 cM, 9 cM, 8 cM, 7 cM, 6 cM, 5 cM, 4 cM, 3 cM, 2 cM, 1 cM, 0.75 cM, 0.5 cM or 0.25 cM or less apart. Put another way, two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are said to be “proximal to” each other.

In some aspects, linked markers (including closely linked markers) of the invention are determined by review of a genetic map, for example, the integrated genetic maps found on the MaizeGDB website. For example, it is shown herein that the linkage group 2 markers MZA625, MZA16656, MZA15451, MZA15490, MZA2038, MZA11826, and MZA9105 correlate with at least one MRCV resistance QTL. Markers that are linked to MZA625, MZA16656, MZA15451, MZA15490, MZA2038, MZA11826, and MZA9105 can be determined from the list provided in Table 8 (see also Table 11, which shows Rice Locus and Working Maize Gene ID of genetic markers between MZA625 and MZA9105).

TABLE 11

| | PHD | UC7 PCO | | | | |
|---|---|---|---|---|---|---|
| PHD | Map | Vs. Myriad | Locus | | | |
| Chr | Pos | Amplicons | Order | Rice Locus | Working Maize Gene ID | Annotation Summary |
| 2 | 64.05 | MZA625 | Loc_029 | LOC_Os04g51320 | AC191302_5part | Transcription Factor |
| | | | Loc_028 | LOC_Os04g51310 | AC191302_3 | Putrescine-binding protein; |
| | | | | | | Hypothetical protein |
| | | | Loc_027 | LOC_Os04g51300 | pco600856 | Putative L-ascorbate |
| | | | | | | peroxidase |
| | | | Loc_025 | LOC_Os04g51280 | pco530474 | Plastid development protein; |
| | | | | | | DAG |
| | | | Loc_024 | LOC_Os04g51270 | pco593067 | Hypothetical protein; Vacuolar |
| | | | | | | ATP synthase subunit? |
| | | | Loc_023 | LOC_Os04g51260 | AC191302_6 | Hypothetical protein |
| | | | Loc_022 | LOC_Os04g51250 | Inferred by rice and sorghum | Hypothetical protein |
| | | | Loc_021 | LOC_Os04g51240 | pco641713 | Hypothetical protein |
| | | | Loc_016 | LOC_Os04g51190 | pco591841 | Growth regulating factor |
| | | | Loc_015 | LOC_Os04g51180 | Genomic_PCO622600_PCO666161 | G protein-coupled receptor |
| | | | | | | 89C (Homo sapiens) |
| 2 | 65.99 | MZA166656 | Loc_014 | LOC_Os04g51172 | pco638426 | Major intrinsic protein; NIP; |
| | | | | | | BREVIS RADIX like 1 |
| | | | Loc_013 | LOC_Os04g51166 | pco514627 | Hypothetical protein |
| 2 | 65.30 | MZA15451 | Loc_012 | LOC_Os04g51160 | pco588936 | Alternative oxidase AOX3 |
| | | | | LOC_Os04g51150 | | |
| | | | Loc_010 | LOC_Os04g51140 | Inferred by rice and sorghum | Hypothetical protein |
| | | | Loc_009 | LOC_Os04g51130 | pco644442 | Myb-like; 2-component |
| | | | | | | response regulator |
| 2 | 65.99 | MZA2038 | Loc_008 | LOC_Os04g51120 | pco641455 | Clathrin interactor; Epsin; |
| | | | | | | Hypothetical protein |
| | | | Loc_007 | LOC_Os04g51110 | pco640541 | CDC20 WD-repeat protein |
| | | | Loc_006 | LOC_Os04g51100 | pco651091 | Cobalamin synthesis protein |
| | | | Loc_005 | LOC_Os04g51090 | pco571541 | Hypothetical protein |
| | | | Loc_004 | LOC_Os04g51080 | pco525409 | Scramblase |
| | | | Loc_003 | LOC_Os04g51070 | pco553755 | Hypothetical protein |
| | | | Loc_002 | LOC_Os04g51060 | pco644099 | Hypothetical protein |
| 2 | 65.44 | MZA9105 | Loc_001 | LOC_Os04g51050 | pco588179 | Receptor protein kinase |

For example, markers on linkage group 2 that are linked to MZA625, MZA16656, MZA15451, MZA15490, MZA2038, MZA11826, and MZA9105 include those listed in Table 12.

TABLE 12

| | Map |
|---|---|
| Marker | Position |
| pco061820a | 148.07 |
| pco116928a | 148.07 |
| sog0930a | 148.07 |
| pco102443 | 148.07 |
| pco133385a | 148.07 |
| sog5467ac | 148.07 |
| cl7211_1l | 148.08 |
| K4-14p | 148.08 |
| pco135612a | 148.08 |
| si687005h09c | 148.08 |
| si707023g07c | 148.08 |
| cl15901_1a | 148.08 |
| pco134907 | 148.08 |
| si660032f12i | 148.08 |
| cl7048_1b | 148.08 |
| cl2578_1 | 148.09 |
| cl5312_1a | 148.09 |
| pco094715 | 148.09 |
| sog5829a | 148.09 |
| cl30_1e | 148.09 |
| pco125905 | 148.09 |
| sog0690 | 148.09 |
| cl36282_1b | 148.09 |
| pco118508 | 148.09 |
| gpm636 | 148.09 |
| pco066747a | 148.09 |
| pco083425q | 148.09 |
| sog5844av | 148.09 |
| bnlg1458b | 148.09 |
| si606065e12a | 148.09 |
| cl22018_1 | 148.09 |
| pco091058 | 148.09 |
| si946053g10 | 148.10 |
| sog1265 | 148.10 |
| sog0743c | 148.10 |
| cl9862_1 | 148.10 |
| pco114887 | 148.10 |
| bnlg1327 | 148.10 |
| sog5587a | 148.10 |
| cl1488_-4a | 148.11 |
| pco085208a | 148.11 |
| sog1295c | 148.11 |
| sog5609b | 148.11 |
| sog0912a | 148.11 |
| tel7sc1ah | 148.11 |
| si660060d11b | 148.11 |
| cl10933_1d | 148.11 |
| cl37019_1a | 148.11 |
| sog1856ae | 148.11 |
| pco117007l | 148.11 |
| cl40761_1a | 148.11 |
| siaf099388e | 148.11 |
| pco137067a | 148.11 |
| sog2274m | 148.11 |
| cl31185_3a | 148.11 |
| pco098939a | 148.11 |
| pco150139r | 148.11 |
| cl11825_1a | 148.11 |
| pco122145b | 148.11 |
| cl24291_1a | 148.11 |
| si618065g03a | 148.11 |
| si707029g03a | 148.11 |
| sog1495a | 148.75 |
| umc1262a | 153.10 |
| umc1261a | 154.60 |
| sog5758o | 154.71 |

Similarly, linked markers (including closely linked markers) of the invention can be determined by review of any suitable maize genetic map. For example, integrated genetic maps can be found on the MaizeGDB website resource.

It is not intended that the determination of linked or closely linked markers be limited to the use of any particular maize genetic map. Indeed, a large number of maize genetic maps is available and are well known to one of skill in the art. Alternatively, the determination of linked and closely linked markers can be made by the generation of an experimental dataset and linkage analysis.

It is also not intended that the identification of markers that are linked (e.g., within about 50 cM or within about 10 cM) to the MRCV resistance QTL markers identified herein be limited to any particular map or methodology. The integrated genetic maps provided on the MaizeGDB website serve only as example for identifying linked markers. Indeed, linked markers as defined herein can be determined from any genetic map known in the art (an experimental map or an integrated map), or alternatively, can be determined from any new mapping dataset.

It is noted that lists of linked and closely linked markers may vary between maps and methodologies due to various factors. First, the markers that are placed on any two maps may not be identical, and furthermore, some maps may have a greater marker density than another map. Also, the mapping populations, methodologies and algorithms used to construct genetic maps can differ. One of skill in the art recognizes that one genetic map is not necessarily more or less accurate than another, and furthermore, recognizes that any maize genetic map can be used to determine markers that are linked and closely linked to the QTL markers of the present invention.

### Techniques for Marker Detection

The invention provides molecular markers that have a significant probability of co-segregation with QTL that impart an MRCV resistance phenotype. These QTL markers find use in marker assisted selection for desired traits (newly conferred resistance or enhanced resistance), and also have other uses. It is not intended that the invention be limited to any particular method for the detection of these markers.

Markers corresponding to genetic polymorphisms between members of a population can be detected by numerous methods well-established in the art (e.g., PCR-based sequence specific amplification, restriction fragment length polymorphisms (RFLPs), isozyme markers, allele specific hybridization (ASH), amplified variable sequences of the plant genome, self-sustained sequence replication, simple sequence repeat (SSR), single nucleotide polymorphism (SNP), random amplified polymorphic DNA (“RAPD”) or amplified fragment length polymorphisms (AFLP)). In one additional embodiment, the presence or absence of a molecular marker is determined simply through nucleotide sequencing of the polymorphic marker region. This method is readily adapted to high throughput analysis as are the other methods noted above, e.g., using available high throughput sequencing methods such as sequencing by hybridization.

In general, the majority of genetic markers rely on one or more property of nucleic acids for their detection. For example, some techniques for detecting genetic markers utilize hybridization of a probe nucleic acid to nucleic acids corresponding to the genetic marker (e.g., amplified nucleic acids produced using genomic maize DNA as a template). Hybridization formats, including but not limited to solution phase, solid phase, mixed phase, or in situ hybridization assays, are useful for allele detection. An extensive guide to the hybridization of nucleic acids is found in Tijssen (1993) Laboratory Techniques in Biochemistry and Molecular Biology—Hybridization with Nucleic Acid Probes Elsevier, N.Y., as well as in Sambrook and Ausubel (herein); and Berger and Kimmel, Guide to Molecular Cloning Techniques, Methods in Enzymology volume 152 Academic Press, Inc., San Diego, Calif. (“Berger”).

For example, markers that comprise restriction fragment length polymorphisms (RFLP) are detected, e.g., by hybridizing a probe which is typically a sub-fragment (or a synthetic oligonucleotide corresponding to a sub-fragment) of the nucleic acid to be detected to restriction digested genomic DNA. The restriction enzyme is selected to provide restriction fragments of at least two alternative (or polymorphic) lengths in different individuals or populations. Determining one or more restriction enzyme that produces informative fragments for each cross is a simple procedure, well known in the art. After separation by length in an appropriate matrix (e.g., agarose or polyacrylamide) and transfer to a membrane (e.g., nitrocellulose, nylon, etc.), the labeled probe is hybridized under conditions which result in equilibrium binding of the probe to the target followed by removal of excess probe by washing.

Nucleic acid probes to the marker loci can be cloned and/or synthesized. Any suitable label can be used with a probe of the invention. Detectable labels suitable for use with nucleic acid probes include, for example, any composition detectable by spectroscopic, radioisotopic, photochemical, biochemical, immunochemical, electrical, optical or chemical means. Useful labels include biotin for staining with labeled streptavidin conjugate, magnetic beads, fluorescent dyes, radiolabels, enzymes, and colorimetric labels. Other labels include ligands which bind to antibodies labeled with fluorophores, chemiluminescent agents, and enzymes. A probe can also constitute radiolabelled PCR primers that are used to generate a radiolabelled amplicon. Labeling strategies for labeling nucleic acids and corresponding detection strategies can be found, e.g., in Haugland (1996) Handbook of Fluorescent Probes and Research Chemicals Sixth Edition by Molecular Probes, Inc. (Eugene Oreg.); or Haugland (2001) Handbook of Fluorescent Probes and Research Chemicals Eighth Edition by Molecular Probes, Inc. (Eugene Oreg.) (Available on CD ROM).

### Amplification-Based Detection Methods

PCR, RT-PCR and LCR are in particularly broad use as amplification and amplification-detection methods for amplifying nucleic acids of interest (e.g., those comprising marker loci), facilitating detection of the markers. Details regarding the use of these and other amplification methods can be found in any of a variety of standard texts, including, e.g., Sambrook, Ausubel, Berger and Croy, herein. Many available biology texts also have extended discussions regarding PCR and related amplification methods. One of skill will appreciate that essentially any RNA can be converted into a double stranded DNA suitable for restriction digestion, PCR expansion and sequencing using reverse transcriptase and a polymerase (“Reverse Transcription-PCR, or “RT-PCR”). See also, Ausubel, Sambrook and Berger, above.

### Real Time Amplification/Detection Methods

In one aspect, real time PCR or LCR is performed on the amplification mixtures described herein, e.g., using molecular beacons or TaqMan™ probes. A molecular beacon (MB) is an oligonucleotide or PNA which, under appropriate hybridization conditions, self-hybridizes to form a stem and loop structure. The MB has a label and a quencher at the termini of the oligonucleotide or PNA; thus, under conditions that permit intra-molecular hybridization, the label is typically quenched (or at least altered in its fluorescence) by the quencher. Under conditions where the MB does not display intra-molecular hybridization (e.g., when bound to a target nucleic acid, e.g., to a region of an amplicon during amplification), the MB label is unquenched. Details regarding standard methods of making and using MBs are well established in the literature, and MBs are available from a number of commercial reagent sources. See also, e.g., Leone et al. (1995) “Molecular beacon probes combined with amplification by NASBA enable homogenous real-time detection of RNA”, Nucleic Acids Res. 26:2150-2155; Tyagi and Kramer (1996) “Molecular beacons: probes that fluoresce upon hybridization” Nature Biotechnology 14:303-308; Blok and Kramer (1997) “Amplifiable hybridization probes containing a molecular switch” Mol Cell Probes 11:187-194; Hsuih et al. (1997) “Novel, ligation-dependent PCR assay for detection of hepatitis C in serum” J Clin Microbiol 34:501-507; Kostrikis et al. (1998) “Molecular beacons: spectral genotyping of human alleles” Science 279:1228-1229; Sokol et al. (1998) “Real time detection of DNA:RNA hybridization in living cells” Proc. Natl. Acad. Sci. U.S.A. 95:11538-11543; Tyagi et al. (1998) “Multicolor molecular beacons for allele discrimination” Nature Biotechnology 16:49-53; Bonnet et al. (1999) “Thermodynamic basis of the chemical specificity of structured DNA probes” Proc. Natl. Acad. Sci. U.S.A. 96:6171-6176; Fang et al. (1999) “Designing a novel molecular beacon for surface-immobilized DNA hybridization studies” J. Am. Chem. Soc. 121:2921-2922; Marras et al. (1999) “Multiplex detection of single-nucleotide variation using molecular beacons” Genet. Anal. Biomol. Eng. 14:151-156; and Vet et al. (1999) “Multiplex detection of four pathogenic retroviruses using molecular beacons” Proc. Natl. Acad. Sci. U.S.A. 96:6394-6399. Additional details regarding MB construction and use is found in the patent literature, e.g., U.S. Pat. Nos. 5,925,517, 6,150,097, and 6,037,130.

PCR detection and quantification using dual-labeled fluorogenic oligonucleotide probes, commonly referred to as “TaqMan™” probes, can also be performed according to the present invention. These probes are composed of short (e.g., 20-25 base) oligodeoxynucleotides that are labeled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. Accordingly, TaqMan™ probes are oligonucleotides that have a label and a quencher, where the label is released during amplification by the exonuclease action of the polymerase used in amplification. This provides a real time measure of amplification during synthesis. A variety of TaqMan™ reagents are commercially available, e.g., from Applied Biosystems (Division Headquarters in Foster City, Calif.) as well as from a variety of specialty vendors such as Biosearch Technologies (e.g., black hole quencher probes).

### Additional Details Regarding Amplified Variable Sequences, SSR, AFLP, ASH, SNPs and Isozyme Markers

Amplified variable sequences refer to amplified sequences of the plant genome which exhibit high nucleic acid residue variability between members of the same species. All organisms have variable genomic sequences and each organism (with the exception of a clone) has a different set of variable sequences. Once identified, the presence of specific variable sequence can be used to predict phenotypic traits. Preferably, DNA from the plant serves as a template for amplification with primers that flank a variable sequence of DNA. The variable sequence is amplified and then sequenced.

Alternatively, self-sustained sequence replication can be used to identify genetic markers. Self-sustained sequence replication refers to a method of nucleic acid amplification using target nucleic acid sequences which are replicated exponentially in vitro under substantially isothermal conditions by using three enzymatic activities involved in retroviral replication: (1) reverse transcriptase, (2) RNase H, and (3) a DNA-dependent RNA polymerase (Guatelli et al. (1990)Proc Natl Acad Sci USA 87:1874). By mimicking the retroviral strategy of RNA replication by means of cDNA intermediates, this reaction accumulates cDNA and RNA copies of the original target.

Amplified fragment length polymorphisms (AFLP) can also be used as genetic markers (Vos et al. (1995) Nucleic Acids Res 23:4407). The phrase “amplified fragment length polymorphism” refers to selected restriction fragments which are amplified before or after cleavage by a restriction endonuclease. The amplification step allows easier detection of specific restriction fragments. AFLP allows the detection large numbers of polymorphic markers and has been used for genetic mapping of plants (Becker et al. (1995) Mol Gen Genet 249:65; and Meksem et al. (1995) Mol Gen Genet 249:74).

Allele-specific hybridization (ASH) can be used to identify the genetic markers of the invention. ASH technology is based on the stable annealing of a short, single-stranded, oligonucleotide probe to a completely complementary single-strand target nucleic acid. Detection is via an isotopic or non-isotopic label attached to the probe.

For each polymorphism, two or more different ASH probes are designed to have identical DNA sequences except at the polymorphic nucleotides. Each probe will have exact homology with one allele sequence so that the range of probes can distinguish all the known alternative allele sequences. Each probe is hybridized to the target DNA. With appropriate probe design and hybridization conditions, a single-base mismatch between the probe and target DNA will prevent hybridization. In this manner, only one of the alternative probes will hybridize to a target sample that is homozygous or homogenous for an allele. Samples that are heterozygous or heterogeneous for two alleles will hybridize to both of two alternative probes.

ASH markers are used as dominant markers where the presence or absence of only one allele is determined from hybridization or lack of hybridization by only one probe. The alternative allele may be inferred from the lack of hybridization. ASH probe and target molecules are optionally RNA or DNA; the target molecules are any length of nucleotides beyond the sequence that is complementary to the probe; the probe is designed to hybridize with either strand of a DNA target; the probe ranges in size to conform to variously stringent hybridization conditions, etc.

PCR allows the target sequence for ASH to be amplified from low concentrations of nucleic acid in relatively small volumes. Otherwise, the target sequence from genomic DNA is digested with a restriction endonuclease and size separated by gel electrophoresis. Hybridizations typically occur with the target sequence bound to the surface of a membrane or, as described in U.S. Pat. No. 5,468,613, the ASH probe sequence may be bound to a membrane.

In one embodiment, ASH data are typically obtained by amplifying nucleic acid fragments (amplicons) from genomic DNA using PCR, transferring the amplicon target DNA to a membrane in a dot-blot format, hybridizing a labeled oligonucleotide probe to the amplicon target, and observing the hybridization dots by autoradiography.

Single nucleotide polymorphisms (SNP) are markers that consist of a shared sequence differentiated on the basis of a single nucleotide. Typically, this distinction is detected by differential migration patterns of an amplicon comprising the SNP on, e.g., an acrylamide gel. However, alternative modes of detection, such as hybridization, e.g., ASH, or RFLP analysis are also appropriate.

Isozyme markers can be employed as genetic markers, e.g., to track markers other than the resistance markers herein, or to track isozyme markers linked to the markers herein. Isozymes are multiple forms of enzymes that differ from one another in their amino acid sequences, and therefore their nucleic acid sequences. Some isozymes are multimeric enzymes containing slightly different subunits. Other isozymes are either multimeric or monomeric but have been cleaved from the proenzyme at different sites in the amino acid sequence. Isozymes can be characterized and analyzed at the protein level, or alternatively, isozymes which differ at the nucleic acid level can be determined. In such cases any of the nucleic acid based methods described herein can be used to analyze isozyme markers.

### Additional Details Regarding Nucleic Acid Amplification

As noted, nucleic acid amplification techniques such as PCR and LCR are well known in the art and can be applied to the present invention to amplify and/or detect nucleic acids of interest, such as nucleic acids comprising marker loci. Examples of techniques sufficient to direct persons of skill through such in vitro methods, including the polymerase chain reaction (PCR), the ligase chain reaction (LCR), Qβ β-replicase amplification and other RNA polymerase mediated techniques (e.g., NASBA), are found in the references noted above, e.g., Innis, Sambrook, Ausubel, Berger and Croy. Additional details are found in Mullis et al. (1987) U.S. Pat. No. 4,683,202; Arnheim & Levinson (Oct. 1, 1990) C&EN 36-47; The Journal Of NIH Research (1991) 3:81-94; Kwoh et al. (1989) Proc. Natl. Acad. Sci. USA 86:1173; Guatelli et al. (1990) Proc. Natl. Acad. Sci. USA 87:1874; Lomeli et al. (1989) J. Clin. Chem 35:1826; Landegren et al. (1988) Science 241:1077-1080; Van Brunt (1990) Biotechnology 8:291-294; Wu and Wallace (1989) Gene 4: 560; Barringer et al. (1990) Gene 89:117; and Sooknanan and Malek (1995) Biotechnology 13:563-564. Improved methods of amplifying large nucleic acids by PCR, which is useful in the context of positional cloning, are further summarized in Cheng et al. (1994) Nature 369:684, and the references therein, in which PCR amplicons of up to 40 kb are generated.

### Detection of Markers For Positional Cloning

In some embodiments, a nucleic acid probe is used to detect a nucleic acid that comprises a marker sequence. Such probes can be used, for example, in positional cloning to isolate nucleotide sequences linked to the marker nucleotide sequence. It is not intended that the nucleic acid probes of the invention be limited to any particular size. In some embodiments, a nucleic acid probe is at least 20 nucleotides in length, or alternatively, at least 50 nucleotides in length, or alternatively, at least 100 nucleotides in length, or alternatively, at least 200 nucleotides in length.

A hybridized probe is detected using autoradiography, fluorography or other similar detection techniques depending on the label to be detected. Examples of specific hybridization protocols are widely available in the art, see, e.g., Berger, Sambrook, and Ausubel, all herein.

### Probe/Primer Synthesis Methods

In general, synthetic methods for making oligonucleotides, including probes, primers, molecular beacons, PNAs, LNAs (locked nucleic acids), etc., are well known. For example, oligonucleotides can be synthesized chemically according to the solid phase phosphoramidite triester method described by Beaucage and Caruthers (1981), Tetrahedron Letts. 22(20):1859-1862, e.g., using a commercially available automated synthesizer, e.g., as described in Needham-VanDevanter et al. (1984) Nucleic Acids Res. 12:6159-6168. Oligonucleotides, including modified oligonucleotides can also be ordered from a variety of commercial sources known to persons of skill. There are many commercial providers of oligo synthesis services, and thus this is a broadly accessible technology. Any nucleic acid can be custom ordered from any of a variety of commercial sources, such as The Midland Certified Reagent Company, The Great American Gene Company, ExpressGen Inc., Operon Technologies Inc. (Alameda, Calif.) and many others. Similarly, PNAs can be custom ordered from any of a variety of sources, such as PeptidoGenic, HTI Bio-Products, Inc., BMA Biomedicals Ltd (U.K.), Bio-Synthesis, Inc., and many others.

### In Silico Marker Detection

In alternative embodiments, in silico methods can be used to detect the marker loci of interest. For example, the sequence of a nucleic acid comprising the marker locus of interest can be stored in a computer. The desired marker locus sequence or its homolog can be identified using an appropriate nucleic acid search algorithm as provided by, for example, in such readily available programs as BLAST, or even simple word processors.

### Amplification Primers for Marker Detection

In some preferred embodiments, the molecular markers of the invention are detected using a suitable PCR-based detection method, where the size or sequence of the PCR amplicon is indicative of the absence or presence of the marker (e.g., a particular marker allele). In these types of methods, PCR primers are hybridized to the conserved regions flanking the polymorphic marker region. As used in the art, PCR primers used to amplify a molecular marker are sometimes termed “PCR markers” or simply “markers”.

It will be appreciated that, although many specific examples of primers are provided herein (see, Table 3), suitable primers to be used with the invention can be designed using any suitable method. It is not intended that the invention be limited to any particular primer or primer pair. For example, primers can be designed using any suitable software program, such as LASERGENE®.

In some embodiments, the primers of the invention are radiolabelled, or labeled by any suitable means (e.g., using a non-radioactive fluorescent tag), to allow for rapid visualization of the different size amplicons following an amplification reaction without any additional labeling step or visualization step. In some embodiments, the primers are not labeled, and the amplicons are visualized following their size resolution, e.g., following agarose gel electrophoresis. In some embodiments, ethidium bromide staining of the PCR amplicons following size resolution allows visualization of the different size amplicons.

It is not intended that the primers of the invention be limited to generating an amplicon of any particular size. For example, the primers used to amplify the marker loci and alleles herein are not limited to amplifying the entire region of the relevant locus. The primers can generate an amplicon of any suitable length. In some embodiments, marker amplification produces an amplicon at least 20 nucleotides in length, or alternatively, at least 50 nucleotides in length, or alternatively, at least 100 nucleotides in length, or alternatively, at least 200 nucleotides in length.

### Marker Assisted Selection and Breeding of Plants

A primary motivation for development of molecular markers in crop species is the potential for increased efficiency in plant breeding through marker assisted selection (MAS). Genetic markers are used to identify plants that contain a desired genotype at one or more loci, and that are expected to transfer the desired genotype, along with a desired phenotype, to their progeny. Genetic markers can be used to identify plants that contain a desired genotype at one locus, or at several unlinked or linked loci (e.g., a haplotype), and that would be expected to transfer the desired genotype, along with a desired phenotype to their progeny. The present invention provides the means to identify plants, particularly maize plants, that have newly conferred resistance or enhanced resistance to, or are susceptible to, MRCV by identifying plants having a specified allele at one of those loci, e.g., MZA625, MZA16656, MZA15451, MZA15490, MZA2038, MZA11826, or MZA9105. In one embodiment, identified resistant plants have the haplotype: C at MRQV_{—}08351-173, A at MRQV_{—}08351-262, G at MRQV_{—}08351-280, G at MRQV_{—}08351-323, C at MRQV_{—}08351-369, C at MRQV_{—}08351-372.

Similarly, by identifying plants lacking the desired marker locus, susceptible or less resistant plants can be identified and, e.g., eliminated from subsequent crosses. Similarly, these marker loci can be introgressed into any desired genomic background, germplasm, plant, line, variety, etc., as part of an overall MAS breeding program designed to enhance maize yield. In one embodiment, identified susceptible plants have the haplotype: T at MRQV_{—}08351-173, T at MRQV_{—}08351-262, A at MRQV_{—}08351-280, C at MRQV_{—}08351-323, T at MRQV_{—}08351-369, T at MRQV_{—}08351-372.

The invention also provides chromosome QTL intervals that find equal use in MAS to select plants that demonstrate newly conferred or enhanced MRCV resistance. Similarly, the QTL intervals can also be used to counter-select plants that are susceptible or have reduced resistance MRCV. Any marker that maps within the QTL interval (including the termini of the intervals) finds use with the invention. These intervals are defined by the following pairs of markers:
(i) MZA8381 and MZA18180;
(ii) MZA4305 and MZA2803;
(iii) MZA15490 and MZA2038;
(iv) bnlg1458b and umc1261a;
(v) bnlg1458b and umc1262a;
(vi) bnlg1327 and umc1261a; and
(viii) bnlg1327 and umc1262a.

In general, MAS uses polymorphic markers that have been identified as having a significant likelihood of co-segregation with a resistance trait. Such markers are presumed to map near a gene or genes that give the plant its resistance phenotype, and are considered indicators for the desired trait, and are termed QTL markers. Plants are tested for the presence of a desired allele in the QTL marker. The most preferred markers (or marker alleles) are those that have the strongest association with the resistance trait.

Linkage analysis is used to determine which polymorphic marker allele demonstrates a statistical likelihood of co-segregation with the resistance phenotype (thus, a “resistance marker allele”). Following identification of a marker allele for co-segregation with the resistance phenotype, it is possible to use this marker for rapid, accurate screening of plant lines for the resistance allele without the need to grow the plants through their life cycle and await phenotypic evaluations, and furthermore, permits genetic selection for the particular resistance allele even when the molecular identity of the actual resistance QTL is unknown. Tissue samples can be taken, for example, from the first leaf of the plant and screened with the appropriate molecular marker, and it is rapidly determined which progeny will advance. Linked markers also remove the impact of environmental factors that can often influence phenotypic expression.

A polymorphic QTL marker locus can be used to select plants that contain the marker allele (or alleles) that correlate with the desired resistance phenotype, typically called marker-assisted selection (MAS). In brief, a nucleic acid corresponding to the marker nucleic acid allele is detected in a biological sample from a plant to be selected. This detection can take the form of hybridization of a probe nucleic acid to a marker allele or amplicon thereof, e.g., using allele-specific hybridization, Southern analysis, northern analysis, in situ hybridization, hybridization of primers followed by PCR amplification of a region of the marker, or the like. A variety of procedures for detecting markers are described herein, e.g., in the section entitled “TECHNIQUES FOR MARKER DETECTION”. After the presence (or absence) of a particular marker allele in the biological sample is verified, the plant is selected (e.g., used to make progeny plants by selective breeding).

Maize plant breeders desire combinations of resistance loci with genes for high yield and other desirable traits to develop improved maize varieties. Screening large numbers of samples by non-molecular methods (e.g., trait evaluation in maize plants) can be expensive, time consuming, and unreliable. Use of the polymorphic markers described herein, when genetically-linked to resistance loci, provide an effective method for selecting resistant varieties in breeding programs. For example, one advantage of marker-assisted selection over field evaluations for resistance is that MAS can be done at any time of year, regardless of the growing season. Moreover, environmental effects are largely irrelevant to marker-assisted selection.

When a population is segregating for multiple loci affecting one or multiple traits, e.g., multiple loci involved in resistance, or multiple loci each involved in resistance to different diseases, the efficiency of MAS compared to phenotypic screening becomes even greater, because all the loci can be evaluated in the lab together from a single sample of DNA. In the present instance, the MZA625, MZA16656, MZA15451, MZA15490, MZA2038, MZA11826, and MZA9105 markers, as well as any of the chromosome intervals
(i) MZA8381 and MZA18180;
(ii) MZA4305 and MZA2803;
(iii) MZA15490 and MZA2038;
(iv) bnlg1458b and umc1261a;
(v) bnlg1458b and umc1262a;
(vi) bnlg1327 and umc1261a; and
(viii) bnlg1327 and umc1262a;
   can be assayed simultaneously or sequentially from a single sample or a population of samples.

Another use of MAS in plant breeding is to assist the recovery of the recurrent parent genotype by backcross breeding. Backcross breeding is the process of crossing a progeny back to one of its parents or parent lines.

Backcrossing is usually done for the purpose of introgressing one or a few loci from a donor parent (e.g., a parent comprising desirable resistance marker loci) into an otherwise desirable genetic background from the recurrent parent (e.g., an otherwise high yielding maize line). The more cycles of backcrossing that are done, the greater the genetic contribution of the recurrent parent to the resulting introgressed variety. This is often necessary, because resistant plants may be otherwise undesirable, e.g., due to low yield, low fecundity, or the like. In contrast, strains which are the result of intensive breeding programs may have excellent yield, fecundity or the like, merely being deficient in one desired trait such as resistance to MRCV.

The presence and/or absence of a particular genetic marker or allele, e.g., MZA625, MZA16656, MZA15451, MZA15490, MZA2038, MZA11826, and MZA9105 markers, as well as any of the chromosome intervals
(i) MZA8381 and MZA18180;
(ii) MZA4305 and MZA2803;
(iii) MZA15490 and MZA2038;
(iv) bnlg1458b and umc1261a;
(v) bnlg1458b and umc1262a;
(vi) bnlg1327 and umc1261a; and
(viii) bnlg1327 and umc1262a;
   in the genome of a plant is made by any method noted herein. If the nucleic acids from the plant are positive for a desired genetic marker allele, the plant can be self fertilized to create a true breeding line with the same genotype, or it can be crossed with a plant with the same marker or with other desired characteristics to create a sexually crossed hybrid generation.
   Introgression of Favorable Alleles—Efficient Backcrossinq of Resistance Markers into Elite Lines

One application of MAS, in the context of the present invention is to use the newly conferred resistance or enhanced resistance markers to increase the efficiency of an introgression or backcrossing effort aimed at introducing a resistance QTL into a desired (typically high yielding) background. In marker assisted backcrossing of specific markers (and associated QTL) from a donor source, e.g., to an elite or exotic genetic background, one selects among backcross progeny for the donor trait and then uses repeated backcrossing to the elite or exotic line to reconstitute as much of the elite/exotic background's genome as possible.

Thus, the markers and methods of the present invention can be utilized to guide marker assisted selection or breeding of maize varieties with the desired complement (set) of allelic forms of chromosome segments associated with superior agronomic performance (resistance, along with any other available markers for yield, etc.). Any of the disclosed marker alleles can be introduced into a maize line via introgression, by traditional breeding (or introduced via transformation, or both), to yield a maize plant with superior agronomic performance. The number of alleles associated with resistance that can be introduced or be present in a maize plant of the present invention ranges from 1 to the number of alleles disclosed herein, each integer of which is incorporated herein as if explicitly recited.

The present invention also extends to a method of making a progeny maize plant and these progeny maize plants, per se. The method comprises crossing a first parent maize plant with a second maize plant and growing the female maize plant under plant growth conditions to yield maize plant progeny. Methods of crossing and growing maize plants are well within the ability of those of ordinary skill in the art. Such maize plant progeny can be assayed for alleles associated with resistance and, thereby, the desired progeny selected. Such progeny plants or seed can be sold commercially for maize production, used for food, processed to obtain a desired constituent of the maize, or further utilized in subsequent rounds of breeding. At least one of the first or second maize plants is a maize plant of the present invention in that it comprises at least one of the allelic forms of the markers of the present invention, such that the progeny are capable of inheriting the allele.

A method of the present invention can be applied to at least one related maize plant such as from progenitor or descendant lines in the subject maize plant's pedigree such that inheritance of the desired resistance allele can be traced. The number of generations separating the maize plants being subject to the methods of the present invention will generally be from 1 to 20, commonly 1 to 5, and typically 1, 2, or 3 generations of separation, and quite often a direct descendant or parent of the maize plant will be subject to the method (i.e., one generation of separation).

Introgression of Favorable Alleles—Incorporation of “Exotic” Germplasm while Maintaining Breeding Progress

Genetic diversity is important for long term genetic gain in any breeding program. With limited diversity, genetic gain will eventually plateau when all the favorable alleles have been fixed within the elite population. One objective is to incorporate diversity into an elite pool without losing the genetic gain that has already been made and with the minimum possible investment. MAS provide an indication of which genomic regions and which favorable alleles from the original ancestors have been selected for and conserved over time, facilitating efforts to incorporate favorable variation from exotic germplasm sources (parents that are unrelated to the elite gene pool) in the hopes of finding favorable alleles that do not currently exist in the elite gene pool.

For example, the markers of the present invention can be used for MAS in crosses involving elite×exotic maize lines by subjecting the segregating progeny to MAS to maintain major yield alleles, along with the resistance marker alleles herein.

### Positional Cloning

The molecular marker loci and alleles of the present invention, e.g., MZA625, MZA16656, MZA15451, MZA15490, MZA2038, MZA11826, and MZA9105 markers, as well as any of the chromosome intervals
(i) MZA8381 and MZA18180;
(ii) MZA4305 and MZA2803;
(iii) MZA15490 and MZA2038;
(iv) bnlg1458b and umc1261a;
(v) bnlg1458b and umc1262a;
(vi) bnlg1327 and umc1261a; and
(viii) bnlg1327 and umc1262a;
   can be used, as indicated previously, to identify a resistance QTL, which can be cloned by well established procedures, e.g., as described in detail in Ausubel, Berger and Sambrook, herein.

These resistance clones are first identified by their genetic linkage to markers of the present invention. Isolation of a nucleic acid of interest is achieved by any number of methods as discussed in detail in such references as Ausubel, Berger and Sambrook, herein, and Clark, Ed. (1997) Plant Molecular Biology: A Laboratory Manual Springer-Verlag, Berlin.

For example, “positional gene cloning” uses the proximity of a resistance marker to physically define an isolated chromosomal fragment containing a resistance QTL gene. The isolated chromosomal fragment can be produced by such well known methods as digesting chromosomal DNA with one or more restriction enzymes, or by amplifying a chromosomal region in a polymerase chain reaction (PCR), or any suitable alternative amplification reaction. The digested or amplified fragment is typically ligated into a vector suitable for replication and, e.g., expression, of the inserted fragment. Markers that are adjacent to an open reading frame (ORF) associated with a phenotypic trait can hybridize to a DNA clone (e.g., a clone from a genomic DNA library), thereby identifying a clone on which an ORF (or a fragment of an ORF) is located. If the marker is more distant, a fragment containing the ORF is identified by successive rounds of screening and isolation of clones which together comprise a contiguous sequence of DNA, a process termed “chromosome walking”, resulting in a “contig” or “contig map”. Protocols sufficient to guide one of skill through the isolation of clones associated with linked markers are found in, e.g., Berger, Sambrook and Ausubel, all herein.

### Generation of Transgenic Cells and Plants

The present invention also relates to host cells and organisms which are transformed with nucleic acids corresponding to resistance QTL identified according to the invention. For example, such nucleic acids include chromosome intervals (e.g., genomic fragments), ORFs and/or cDNAs that encode a newly conferred resistance or enhanced resistance trait. Additionally, the invention provides for the production of polypeptides that provide newly conferred resistance or enhanced resistance by recombinant techniques.

General texts which describe molecular biological techniques for the cloning and manipulation of nucleic acids and production of encoded polypeptides include Berger, Sambrook, and Ausubel supra. These texts describe mutagenesis, the use of vectors, promoters and many other relevant topics related to, e.g., the generation of clones that comprise nucleic acids of interest, e.g., marker loci, marker probes, QTL that segregate with marker loci, etc.

Host cells are genetically engineered (e.g., transduced, transfected, transformed, etc.) with the vectors of this invention (e.g., vectors, such as expression vectors which comprise an ORF derived from or related to a resistance QTL) which can be, for example, a cloning vector, a shuttle vector or an expression vector. Such vectors are, for example, in the form of a plasmid, a phagemid, an agrobacterium, a virus, a naked polynucleotide (linear or circular), or a conjugated polynucleotide. Vectors can be introduced into bacteria, especially for the purpose of propagation and expansion. The vectors are also introduced into plant tissues, cultured plant cells or plant protoplasts by a variety of standard methods known in the art, including but not limited to electroporation (From et al. (1985) Proc. Natl. Acad. Sci. USA 82:5824), infection by viral vectors such as cauliflower mosaic virus (CaMV) (Hohn et al. (1982) Molecular Biology of Plant Tumors (Academic Press, New York), pp. 549-560; U.S. Pat. No. 4,407,956), high velocity ballistic penetration by small particles with the nucleic acid either within the matrix of small beads or particles, or on the surface (Klein et al. (1987) Nature 327:70), use of pollen as vector (WO85/01856), or use of Agrobacterium tumefaciens or A. rhizogenes carrying a T-DNA plasmid in which DNA fragments are cloned. The T-DNA plasmid is transmitted to plant cells upon infection by Agrobacterium tumefaciens, and a portion is stably integrated into the plant genome (Horsch et al. (1984) Science 233:496; Fraley et al. (1983) Proc. Natl. Acad. Sci. USA 80:4803). Additional details regarding nucleic acid introduction methods are found in Sambrook, Berger and Ausubel, supra. The method of introducing a nucleic acid of the present invention into a host cell is not critical to the instant invention, and it is not intended that the invention be limited to any particular method for introducing exogenous genetic material into a host cell. Thus, any suitable method, e.g., including but not limited to the methods provided herein, which provides for effective introduction of a nucleic acid into a cell or protoplast can be employed and finds use with the invention.

The engineered host cells can be cultured in conventional nutrient media modified as appropriate for such activities as, for example, activating promoters or selecting transformants. These cells can optionally be cultured into transgenic plants. In addition to Sambrook, Berger and Ausubel, supra, plant regeneration from cultured protoplasts is described in Evans et al. (1983) “Protoplast Isolation and Culture”, Handbook of Plant Cell Cultures 1, 124-176 (MacMillan Publishing Co.), New York; Davey (1983) “Recent Developments in the Culture and Regeneration of Plant Protoplasts”, Protoplasts, pp. 12-29, (Birkhauser, Base1); Dale (1983) “Protoplast Culture and Plant Regeneration of Cereals and Other Recalcitrant Crops”, Protoplasts pp. 31-41, (Birkhauser, Base1); Binding (1985) “Regeneration of Plants”, Plant Protoplasts, pp. 21-73, (CRC Press, Boca Raton, Fla.). Additional details regarding plant cell culture and regeneration include Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York) and Plant Molecular Biology (1993) R. R. D. Croy, Ed. Bios Scientific Publishers, Oxford, U.K. ISBN 0 12 198370 6. Cell culture media in general are also set forth in Atlas and Parks (eds) The Handbook of Microbiological Media (1993) CRC Press, Boca Raton, Fla. Additional information for cell culture is found in available commercial literature such as the Life Science Research Cell Culture Catalogue (1998) from Sigma-Aldrich, Inc. (St Louis, Mo.) (“Sigma-LSRCCC”) and, e.g., the Plant Culture Catalogue and supplement (e.g., 1997 or later) also from Sigma-Aldrich, Inc. (St Louis, Mo.) (“Sigma-PCCS”).

The present invention also relates to the production of transgenic organisms, which may be bacteria, yeast, fungi, animals or plants, transduced with the nucleic acids of the invention (e.g., nucleic acids comprising the marker loci and/or QTL noted herein). A thorough discussion of techniques relevant to bacteria, unicellular eukaryotes and cell culture is found in references enumerated herein and are briefly outlined as follows. Several well-known methods of introducing target nucleic acids into bacterial cells are available, any of which may be used in the present invention. These include: fusion of the recipient cells with bacterial protoplasts containing the DNA, treatment of the cells with liposomes containing the DNA, electroporation, projectile bombardment (biolistics), carbon fiber delivery, and infection with viral vectors (discussed further, below), etc. Bacterial cells can be used to amplify the number of plasmids containing DNA constructs of this invention. The bacteria are grown to log phase, and the plasmids within the bacteria can be isolated by a variety of methods known in the art (see, for instance, Sambrook). In addition, a plethora of kits are commercially available for the purification of plasmids from bacteria. For their proper use, follow the manufacturer's instructions (see, for example, EasyPrep™, FlexiPrep™, both from Pharmacia Biotech; StrataClean™, from Stratagene; and, QIAprep™ from Qiagen). The isolated and purified plasmids are then further manipulated to produce other plasmids, used to transfect plant cells or incorporated into Agrobacterium tumefaciens related vectors to infect plants. Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or preferably both. See, Giliman & Smith (1979) Gene 8:81; Roberts et al. (1987) Nature 328:731; Schneider et al. (1995) Protein Expr. Purif. 6:10; Ausubel, Sambrook, Berger (all supra). A catalogue of Bacteria and Bacteriophages useful for cloning is provided, e.g., by the American Type Culture Collection (ATCC), e.g., The ATCC Catalogue of Bacteria and Bacteriophage (1992) Gherna et al. (eds) published by the ATCC. Additional basic procedures for sequencing, cloning and other aspects of molecular biology and underlying theoretical considerations are also found in Watson et al. (1992) Recombinant DNA, Second Edition, Scientific American Books, NY. In addition, essentially any nucleic acid (and virtually any labeled nucleic acid, whether standard or non-standard) can be custom or standard ordered from any of a variety of commercial sources, such as the Midland Certified Reagent Company (Midland, Tex.), The Great American Gene Company (Ramona, Calif.), ExpressGen Inc. (Chicago, Ill.), Operon Technologies Inc. (Alameda, Calif.) and many others.

Introducing Nucleic Acids into Plants.

Embodiments of the present invention pertain to the production of transgenic plants comprising the cloned nucleic acids, e.g., isolated ORFs and cDNAs encoding resistance genes. Techniques for transforming plant cells with nucleic acids are widely available and can be readily adapted to the invention. In addition to Berger, Ausubel and Sambrook, all supra, useful general references for plant cell cloning, culture and regeneration include Jones (ed) (1995) Plant Gene Transfer and Expression Protocols—Methods in Molecular Biology, Volume 49 Humana Press Towata N.J. (“Jones”); Payne et al. (1992) Plant Cell and Tissue Culture in Liquid Systems John Wiley & Sons, Inc. New York, N.Y. (“Payne”); and Gamborg and Phillips (eds) (1995) Plant Cell, Tissue and Organ Culture; Fundamental Methods Springer Lab Manual, Springer-Verlag (Berlin Heidelberg New York) (“Gamborg”). A variety of cell culture media are described in Atlas and Parks (eds) The Handbook of Microbiological Media (1993) CRC Press, Boca Raton, Fla. (“Atlas”). Additional information for plant cell culture is found in available commercial literature such as the Life Science Research Cell Culture Catalogue (1998) from Sigma-Aldrich, Inc. (St Louis, Mo.) (Sigma-LSRCCC) and, e.g., the Plant Culture Catalogue and supplement (1997) also from Sigma-Aldrich, Inc. (St Louis, Mo.) (Sigma-PCCS). Additional details regarding plant cell culture are found in Croy.

The nucleic acid constructs of the invention, e.g., plasmids, cosmids, artificial chromosomes, DNA and RNA polynucleotides, are introduced into plant cells, either in culture or in the organs of a plant by a variety of conventional techniques. Where the sequence is expressed, the sequence is optionally combined with transcriptional and translational initiation regulatory sequences which direct the transcription or translation of the sequence from the exogenous DNA in the intended tissues of the transformed plant.

Isolated nucleic acid acids of the present invention can be introduced into plants according to any of a variety of techniques known in the art. Techniques for transforming a wide variety of higher plant species are also well known and described in widely available technical, scientific, and patent literature. See, for example, Weising et al. (1988) Ann. Rev. Genet. 22:421-477.

The DNA constructs of the invention, for example plasmids, phagemids, cosmids, phage, naked or variously conjugated-DNA polynucleotides (e.g., polylysine-conjugated DNA, peptide-conjugated DNA, liposome-conjugated DNA), or artificial chromosomes, can be introduced directly into the genomic DNA of the plant cell using techniques such as electroporation and microinjection of plant cell protoplasts, or the DNA constructs can be introduced directly to plant cells using ballistic methods, such as DNA particle bombardment.

Microinjection techniques for injecting plant, e.g., cells, embryos, callus and protoplasts, are known in the art and well described in the scientific and patent literature. For example, a number of methods are described in Jones, as well as in the other references noted herein and available in the literature.

For example, the introduction of DNA constructs using polyethylene glycol precipitation is described in Paszkowski et al., EMBO J. 3:2717 (1984). Electroporation techniques are described in Fromm et al., Proc. Natl. Acad. Sci. USA 82:5824 (1985). Ballistic transformation techniques are described in Klein et al., Nature 327:70-73 (1987). Additional details are found in Jones and Gamborg, supra, and in U.S. Pat. No. 5,990,387.

Alternatively, and in some cases preferably, Agrobacterium-mediated transformation is employed to generate transgenic plants. Agrobacterium-mediated transformation techniques, including disarming and use of binary vectors, are also well described in the scientific literature. See, for example, Horsch, et al. (1984) Science 233:496; Fraley et al. (1984) Proc. Natl. Acad. Sci. USA 80:4803: and reviewed in Hansen and Chilton (1998) Curr. Top. Microbiol. Immunol. 240:21 and Das (1998) Subcellular Biochemistry 29: Plant Microbe Interactions, pp. 343-363.

DNA constructs are optionally combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. See, U.S. Pat. No. 5,591,616. Although Agrobacterium is useful primarily in dicots, certain monocots can be transformed by Agrobacterium. For instance, Agrobacterium transformation of maize is described in U.S. Pat. No. 5,550,318.

Other methods of transfection or transformation include (1) Agrobacterium rhizogenes-mediated transformation (see, e.g., Lichtenstein and Fuller (1987) In: Genetic Engineering, vol. 6, P W J Rigby, Ed., London, Academic Press; and Lichtenstein and Draper (1985) In: DNA Cloning, Vol. II, D. M. Glover, Ed., Oxford, IRI Press; WO 88/02405, published Apr. 7, 1988, describes the use of A. rhizogenes strain A4 and its Ri plasmid along with A. tumefaciens vectors pARC8 or pARC16), (2) liposome-mediated DNA uptake (see, e.g., Freeman et al. (1984) Plant Cell Physiol. 25:1353), and (3) the vortexing method (see, e.g., Kindle (1990) Proc. Natl. Acad. Sci. (USA) 87:1228).

DNA can also be introduced into plants by direct DNA transfer into pollen as described by Zhou et al. (1983) Meth. Enzymol. 101:433; D. Hess (1987) Intern Rev. Cytol. 107:367; Luo et al. (1988) Plant Mol. Biol. Rep. 6:165. Expression of polypeptide coding genes can be obtained by injection of the DNA into reproductive organs of a plant as described by Pena et al. (1987) Nature 325:274. DNA can also be injected directly into the cells of immature embryos and the desiccated embryos rehydrated as described by Neuhaus et al. (1987) Theor. Appl. Genet. 75:30; and Benbrook et al. (1986) in Proceedings Bio Expo Butterworth, Stoneham, Mass., pp. 27-54. A variety of plant viruses that can be employed as vectors are known in the art and include cauliflower mosaic virus (CaMV), geminivirus, brome mosaic virus, and tobacco mosaic virus.

### Generation/Regeneration of Transgenic Plants

Transformed plant cells which are derived by any of the above transformation techniques can be cultured to regenerate a whole plant that possesses the transformed genotype and thus the desired phenotype. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Payne; Fundamental Methods Springer Lab Manual, Springer-Verlag (Berlin Heidelberg New York); Evans et al. (1983) Protoplasts Isolation and Culture, Handbook of Plant Cell Culture pp. 124-176, Macmillian Publishing Company, New York; and Binding (1985) Regeneration of Plants, Plant Protoplasts pp. 21-73, CRC Press, Boca Raton. Regeneration can also be obtained from plant callus, explants, somatic embryos (Dandekar et al. (1989) J. Tissue Cult. Meth. 12:145; McGranahan et al. (1990) Plant Cell Rep. 8:512) organs, or parts thereof. Such regeneration techniques are described generally in Klee et al. (1987) Ann. Rev. Plant Phys. 38:467-486. Additional details are found in Payne and Jones, both supra, and Weissbach and Weissbach, eds. (1988) Methods for Plant Molecular Biology Academic Press, Inc., San Diego, Calif. This regeneration and growth process includes the steps of selection of transformant cells and shoots, rooting the transformant shoots, and growth of the plantlets in soil. These methods are adapted to the invention to produce transgenic plants bearing QTL and other genes isolated according to the methods of the invention.

In addition, the regeneration of plants containing polynucleotides of the present invention and introduced by Agrobacterium into cells of leaf explants can be achieved as described by Horsch et al. (1985) Science 227:1229-1231. In this procedure, transformants are grown in the presence of a selection agent and in a medium that induces the regeneration of shoots in the plant species being transformed as described by Fraley et al. (1983) Proc. Natl. Acad. Sci. (U.S.A.) 80:4803. This procedure typically produces shoots within two to four weeks, and these transformant shoots are then transferred to an appropriate root-inducing medium containing the selective agent and an antibiotic to prevent bacterial growth. Transgenic plants of the present invention may be fertile or sterile.

It is not intended that plant transformation and expression of polypeptides that provide disease resistance, as provided by the present invention, be limited to maize species. Indeed, it is contemplated that the polypeptides that provide the desired resistance in maize can also provide such resistance when transformed and expressed in other agronomically and horticulturally important species. For example, such species include: soybean, canola, alfalfa, wheat, sunflower, and sorghum.

In construction of recombinant expression cassettes of the invention, which include, for example, helper plasmids comprising virulence functions, and plasmids or viruses comprising exogenous DNA sequences such as structural genes, a plant promoter fragment is optionally employed which directs expression of a nucleic acid in any or all tissues of a regenerated plant. Examples of constitutive promoters include the cauliflower mosaic virus (CaMV) 35S transcription initiation region, the 1′- or 2′-promoter derived from T-DNA of Agrobacterium tumefaciens, and other transcription initiation regions from various plant genes known to those of skill. Alternatively, the plant promoter may direct expression of the polynucleotide of the invention in a specific tissue (tissue-specific promoters) or may be otherwise under more precise environmental control (inducible promoters). Examples of tissue-specific promoters under developmental control include promoters that initiate transcription only in certain tissues, such as fruit, seeds or flowers.

Any of a number of promoters which direct transcription in plant cells can be suitable. The promoter can be either constitutive or inducible. In addition to the promoters noted above, promoters of bacterial origin that operate in plants include the octopine synthase promoter, the nopaline synthase promoter and other promoters derived from native Ti plasmids. See, Herrara-Estrella et al. (1983) Nature 303:209. Viral promoters include the 35S and 19S RNA promoters of cauliflower mosaic virus. See, Odell et al. (1985) Nature 313:810. Other plant promoters include Kunitz trypsin inhibitor promoter (KTI), SCP1, SUP, UCD3, the ribulose-1,3-bisphosphate carboxylase small subunit promoter and the phaseolin promoter. The promoter sequence from the E8 gene and other genes may also be used. The isolation and sequence of the E8 promoter is described in detail in Deikman and Fischer (1988) EMBO J. 7:3315. Many other promoters are in current use and can be coupled to an exogenous DNA sequence to direct expression of the nucleic acid.

If expression of a polypeptide from a cDNA is desired, a polyadenylation region at the 3′-end of the coding region is typically included. The polyadenylation region can be derived from the natural gene, from a variety of other plant genes, or from, e.g., T-DNA.

The vector comprising the sequences (e.g., promoters or coding regions) from genes encoding expression products and transgenes of the invention will typically include a nucleic acid subsequence, a marker gene which confers a selectable, or alternatively, a screenable, phenotype on plant cells. For example, the marker can encode biocide tolerance, particularly antibiotic tolerance, such as tolerance to kanamycin, G418, bleomycin, hygromycin, or herbicide tolerance, such as tolerance to chiorosulforon, or phosphinothricin (the active ingredient in the herbicides bialaphos or Basta). See, e.g., Padgette et al. (1996) In: Herbicide-Resistant Crops (Duke, ed.), pp 53-84, CRC Lewis Publishers, Boca Raton. For example, crop selectivity to specific herbicides can be conferred by engineering genes into crops that encode appropriate herbicide metabolizing enzymes from other organisms, such as microbes. See, Vasil (1996) In: Herbicide-Resistant Crops (Duke, ed.), pp 85-91, CRC Lewis Publishers, Boca Raton).

One of skill will recognize that after the recombinant expression cassette is stably incorporated in transgenic plants and confirmed to be operable, it can be introduced into other plants by sexual crossing. Any of a number of standard breeding techniques can be used, depending upon the species to be crossed. In vegetatively propagated crops, mature transgenic plants can be propagated by the taking of cuttings or by tissue culture techniques to produce multiple identical plants. Selection of desirable transgenics is made and new varieties are obtained and propagated vegetatively for commercial use. In seed propagated crops, mature transgenic plants can be self crossed to produce a homozygous inbred plant. The inbred plant produces seed containing the newly introduced heterologous nucleic acid. These seeds can be grown to produce plants that would produce the selected phenotype. Parts obtained from the regenerated plant, such as flowers, seeds, leaves, branches, fruit, and the like, are included in the invention, provided that these parts comprise cells comprising the isolated nucleic acid of the present invention. Progeny and variants, and mutants of the regenerated plants, are also included within the scope of the invention, provided that these parts comprise the introduced nucleic acid sequences.

Transgenic or introgressed plants expressing a polynucleotide of the present invention can be screened for transmission of the nucleic acid of the present invention by, for example, standard nucleic acid detection methods or by immunoblot protocols. Expression at the RNA level can be determined to identify and quantitate expression-positive plants. Standard techniques for RNA analysis can be employed and include RT-PCR amplification assays using oligonucleotide primers designed to amplify only heterologous or introgressed RNA templates and solution hybridization assays using marker or linked QTL specific probes. Plants can also be analyzed for protein expression, e.g., by Western immunoblot analysis using antibodies that recognize the encoded polypeptides. In addition, in situ hybridization and immunocytochemistry according to standard protocols can be done using heterologous nucleic acid specific polynucleotide probes and antibodies, respectively, to localize sites of expression within transgenic tissue. Generally, a number of transgenic lines are usually screened for the incorporated nucleic acid to identify and select plants with the most appropriate expression profiles.

One embodiment of the invention is a transgenic plant that is homozygous for the added heterologous nucleic acid; e.g., a transgenic plant that contains two added nucleic acid sequence copies, e.g., a gene at the same locus on each chromosome of a homologous chromosome pair. A homozygous transgenic plant can be obtained by sexually mating (self-fertilizing) a heterozygous transgenic plant that contains a single added heterologous nucleic acid, germinating some of the seed produced and analyzing the resulting plants produced for altered expression of a polynucleotide of the present invention relative to a control plant (e.g., a native, non-transgenic plant). Back-crossing to a parental plant and out-crossing with a non-transgenic plant can be used to introgress the heterologous nucleic acid into a selected background (e.g., an elite or exotic maize line).

### Methods for MRCV Resistant Maize Plants

Experienced plant breeders can recognize resistant maize plants in the field and can select the resistant individuals or populations for breeding purposes or for propagation. In this context, the plant breeder recognizes “resistant” and “non-resistant”, or “susceptible”, maize plants.

Such plant breeding practitioners will appreciate that plant resistance is a phenotypic spectrum consisting of extremes in resistance, susceptibility and a continuum of intermediate resistance phenotypes. Resistance also varies due to environmental effects and the severity of pathogen infection. Evaluation of phenotypes using reproducible assays and resistance scoring methods are of value to scientists who seek to identify genetic loci that impart resistance, conduct marker assisted selection for resistant populations, and for introgression techniques to breed a resistance trait into an elite maize line, for example.

In contrast to fortuitous field observations that classify plants as either “resistant” or “susceptible”, various systems are known for scoring the degree of plant resistance or susceptibility. These techniques can be applied to different fields at different times, and provide approximate resistance scores that can be used to characterize a given strain regardless of growth conditions or location.

### Automated Detection/Correlation Systems of the Invention

In some embodiments, the present invention includes an automated system for detecting markers of the invention and/or correlating the markers with a desired phenotype (e.g., resistance). Thus, a typical system can include a set of marker probes or primers configured to detect at least one favorable allele of one or more marker locus associated with newly conferred resistance or enhanced resistance to MRCV. These probes or primers are configured to detect the marker alleles noted in the tables and examples herein, e.g., using any available allele detection format, e.g., solid or liquid phase array based detection, microfluidic-based sample detection, etc.

For example, in one embodiment, the marker locus is MZA625, MZA16656, MZA15451, MZA15490, MZA2038, MZA11826, and MZA9105, or any combination thereof, as well as any of the chromosome intervals
(i) MZA8381 and MZA18180;
(ii) MZA4305 and MZA2803;
(iii) MZA15490 and MZA2038;
(iv) bnlg1458b and umc1261a;
(v) bnlg1458b and umc1262a;
(vi) bnlg1327 and umc1261a; and
(viii) bnlg1327 and umc1262a; or any combination thereof,
   and the probe set is configured to detect the locus.

The typical system includes a detector that is configured to detect one or more signal outputs from the set of marker probes or primers, or amplicon thereof, thereby identifying the presence or absence of the allele. A wide variety of signal detection apparatus are available, including photo multiplier tubes, spectrophotometers, CCD arrays, arrays and array scanners, scanning detectors, phototubes and photodiodes, microscope stations, galvo-scanns, microfluidic nucleic acid amplification detection appliances and the like. The precise configuration of the detector will depend, in part, on the type of label used to detect the marker allele, as well as the instrumentation that is most conveniently obtained for the user. Detectors that detect fluorescence, phosphorescence, radioactivity, pH, charge, absorbance, luminescence, temperature, magnetism or the like can be used. Typical detector embodiments include light (e.g., fluorescence) detectors or radioactivity detectors. For example, detection of a light emission (e.g., a fluorescence emission) or other probe label is indicative of the presence or absence of a marker allele. Fluorescent detection is especially preferred and is generally used for detection of amplified nucleic acids (however, upstream and/or downstream operations can also be performed on amplicons, which can involve other detection methods). In general, the detector detects one or more label (e.g., light) emission from a probe label, which is indicative of the presence or absence of a marker allele.

The detector(s) optionally monitors one or a plurality of signals from an amplification reaction. For example, the detector can monitor optical signals which correspond to “real time” amplification assay results.

System instructions that correlate the presence or absence of the favorable allele with the predicted resistance are also a feature of the invention. For example, the instructions can include at least one look-up table that includes a correlation between the presence or absence of the favorable alleles and the predicted newly conferred resistance or enhanced resistance. The precise form of the instructions can vary depending on the components of the system, e.g., they can be present as system software in one or more integrated unit of the system (e.g., a microprocessor, computer or computer readable medium), or can be present in one or more units (e.g., computers or computer readable media) operably coupled to the detector. As noted, in one typical embodiment, the system instructions include at least one look-up table that includes a correlation between the presence or absence of the favorable alleles and predicted newly conferred resistance or enhanced resistance. The instructions also typically include instructions providing a user interface with the system, e.g., to permit a user to view results of a sample analysis and to input parameters into the system.

The system typically includes components for storing or transmitting computer readable data representing or designating the alleles detected by the methods of the present invention, e.g., in an automated system. The computer readable media can include cache, main, and storage memory and/or other electronic data storage components (hard drives, floppy drives, storage drives, etc.) for storage of computer code. Data representing alleles detected by the method of the present invention can also be electronically, optically, or magnetically transmitted in a computer data signal embodied in a transmission medium over a network such as an intranet or internet or combinations thereof. The system can also or alternatively transmit data via wireless, IR, or other available transmission alternatives.

During operation, the system typically comprises a sample that is to be analyzed, such as a plant tissue, or material isolated from the tissue such as genomic DNA, amplified genomic DNA, cDNA, amplified cDNA, RNA, amplified RNA, or the like.

The phrase “allele detection/correlation system” in the context of this invention refers to a system in which data entering a computer corresponds to physical objects or processes external to the computer, e.g., a marker allele, and a process that, within a computer, causes a physical transformation of the input signals to different output signals. In other words, the input data, e.g., amplification of a particular marker allele is transformed to output data, e.g., the identification of the allelic form of a chromosome segment. The process within the computer is a set of instructions, or “program”, by which positive amplification or hybridization signals are recognized by the integrated system and attributed to individual samples as a genotype. Additional programs correlate the identity of individual samples with phenotypic values or marker alleles, e.g., statistical methods. In addition there are numerous C/C++ programs for computing, Delphi and/or Java programs for GUI interfaces, and productivity tools (e.g., Microsoft Excel and/or SigmaPlot) for charting or creating look up tables of relevant allele-trait correlations. Other useful software tools in the context of the integrated systems of the invention include statistical packages such as SAS, Genstat, Matlab, Mathematica, and S-Plus and genetic modeling packages such as QU-GENE. Furthermore, additional programming languages such as visual basic are also suitably employed in the integrated systems of the invention.

For example, resistance marker allele values assigned to a population of progeny descending from crosses between elite lines are recorded in a computer readable medium, thereby establishing a database corresponding resistance alleles with unique identifiers for members of the population of progeny. Any file or folder, whether custom-made or commercially available (e.g., from Oracle or Sybase), suitable for recording data in a computer readable medium is acceptable as a database in the context of the present invention. Data regarding genotype for one or more molecular markers, e.g., ASH, SSR, RFLP, RAPD, AFLP, SNP, isozyme markers or other markers as described herein, are similarly recorded in a computer accessible database. Optionally, marker data is obtained using an integrated system that automates one or more aspects of the assay (or assays) used to determine marker(s) genotype. In such a system, input data corresponding to genotypes for molecular markers are relayed from a detector, e.g., an array, a scanner, a CCD, or other detection device directly to files in a computer readable medium accessible to the central processing unit. A set of system instructions (typically embodied in one or more programs) encoding the correlations between resistance and the alleles of the invention is then executed by the computational device to identify correlations between marker alleles and predicted trait phenotypes.

Typically, the system also includes a user input device, such as a keyboard, a mouse, a touchscreen, or the like (for, e.g., selecting files, retrieving data, reviewing tables of maker information), and an output device (e.g., a monitor, a printer) for viewing or recovering the product of the statistical analysis.

Thus, in one aspect, the invention provides an integrated system comprising a computer or computer readable medium comprising a set of files and/or a database with at least one data set that corresponds to the marker alleles herein. The system also includes a user interface allowing a user to selectively view one or more of these databases. In addition, standard text manipulation software such as word processing software (e.g., Microsoft Word™ or Corel WordPerfect™) and database or spreadsheet software (e.g., spreadsheet software such as Microsoft Excel™, Corel Quattro Pro™, or database programs such as Microsoft Access™ or Paradox™) can be used in conjunction with a user interface (e.g., a GUI in a standard operating system such as a Windows, Macintosh, Unix or Linux system) to manipulate strings of characters corresponding to the alleles or other features of the database.

The systems optionally include components for sample manipulation, e.g., incorporating robotic devices. For example, a robotic liquid control armature for transferring solutions (e.g., plant cell extracts) from a source to a destination, e.g., from a microtiter plate to an array substrate, is optionally operably linked to the digital computer (or to an additional computer in the integrated system). An input device for entering data to the digital computer to control high throughput liquid transfer by the robotic liquid control armature and, optionally, to control transfer by the armature to the solid support is commonly a feature of the integrated system. Many such automated robotic fluid handling systems are commercially available. For example, a variety of automated systems are available from Caliper Technologies (Hopkinton, Mass.), which utilize various Zymate systems, which typically include, e.g., robotics and fluid handling modules. Similarly, the common ORCA® robot, which is used in a variety of laboratory systems, e.g., for microtiter tray manipulation, is also commercially available, e.g., from Beckman Coulter, Inc. (Fullerton, Calif.). As an alternative to conventional robotics, microfluidic systems for performing fluid handling and detection are now widely available, e.g., from Caliper Technologies Corp. (Hopkinton, Mass.) and Agilent Technologies (Palo Alto, Calif.).

Systems for molecular marker analysis of the present invention can thus include a digital computer with one or more of high-throughput liquid control software, image analysis software for analyzing data from marker labels, data interpretation software, a robotic liquid control armature for transferring solutions from a source to a destination operably linked to the digital computer, an input device (e.g., a computer keyboard) for entering data to the digital computer to control high throughput liquid transfer by the robotic liquid control armature and, optionally, an image scanner for digitizing label signals from labeled probes hybridized, e.g., to markers on a solid support operably linked to the digital computer. The image scanner interfaces with the image analysis software to provide a measurement of, e.g., nucleic acid probe label intensity upon hybridization to an arrayed sample nucleic acid population (e.g., comprising one or more markers), where the probe label intensity measurement is interpreted by the data interpretation software to show whether, and to what degree, the labeled probe hybridizes to a marker nucleic acid (e.g., an amplified marker allele). The data so derived is then correlated with sample identity, to determine the identity of a plant with a particular genotype(s) for particular markers or alleles, e.g., to facilitate marker assisted selection of maize plants with favorable allelic forms of chromosome segments involved in agronomic performance (e.g., newly conferred resistance or enhanced resistance).

Optical images, e.g., hybridization patterns viewed (and, optionally, recorded) by a camera or other recording device (e.g., a photodiode and data storage device) are optionally further processed in any of the embodiments herein, e.g., by digitizing the image and/or storing and analyzing the image on a computer. A variety of commercially available peripheral equipment and software is available for digitizing, storing and analyzing a digitized video or digitized optical image, e.g., using PC (e.g., Intel x86 or Pentium chip-compatible DOS™, OS2™, WINDOWS™, WINDOWS NT™, WINDOWS 95™, WINDOWS 97™, WINDOWS 2000™, WINDOWS XP™, or WINDOWS VISTA™ based machines), MACINTOSH™, LINUX, or UNIX based (e.g., SUN™ work station) computers.

### EXAMPLES

The following examples are offered to illustrate, but not to limit, the claimed invention. It is understood that the examples and embodiments described herein are for illustrative purposes only, and persons skilled in the art will recognize various reagents or parameters that can be altered without departing from the spirit of the invention or the scope of the appended claims.

The present study was completed by two different association analysis approaches: 1) Population-based Structured association analysis and 2) Pedigree-based association analysis. By identifying such genetic markers, marker assisted selection (MAS) can be used to improve the efficiency of breeding for improved resistance of maize to MRCV infection. Association mapping is known in the art, and is described in various sources, e.g., Jorde (2000) Genome Res. 10:1435-1444; Remington et al. (2001) “Structure of linkage disequilibrium and phenotype associations in the maize genome,” Proc Natl Acad Sci USA 98:11479-11484; Weiss and Clark (2002) Trends Genet. 18:19-24; and Shu et al. (2003) “Detection Power of Random, Case-Control, and Case-Parent Control Designs for Association Tests and Genetic Mapping of Complex Traits,” Proceedings of 15th Annual KSU Conference on Applied Statistics in Agriculture 15:191-204.

### Example 1

### Association Mapping Analysis

An association mapping strategy was undertaken to identify maize genetic markers associated with resistance to MRCV infection, which is the causative agent of “Mal de Río Cuarto”.

### Association Mapping

Understanding the extent and patterns of linkage disequilibrium (LD) in the genome is a prerequisite for developing efficient association approaches to identify and map quantitative trait loci (QTL). Linkage disequilibrium (LD) refers to the non-random association of alleles in a collection of individuals. When LD is observed among alleles at linked loci, it is measured as LD decay across a specific region of a chromosome. The extent of the LD is a reflection of the recombinational history of that region. The average rate of LD decay in a genome can help predict the number and density of markers that are required to undertake a genome-wide association study and provides an estimate of the resolution that can be expected.

Association or LD mapping aims to identify significant genotype-phenotype associations. It has been exploited as a powerful tool for fine mapping in outcrossing species such as humans (Corder et al. (1994) “Protective effect of apolipoprotein-E type-2 allele for late-onset Alzheimer-disease,” Nat Genet 7:180-184; Hastbacka et al. (1992) “Linkage disequilibrium mapping in isolated founder populations: diastrophic dysplasia in Finland,” Nat Genet 2:204-211; Kerem et al. (1989) “Identification of the cystic fibrosis gene: genetic analysis,” Science 245:1073-1080) and maize (Remington et al., (2001) “Structure of linkage disequilibrium and phenotype associations in the maize genome,” Proc Natl Acad Sci USA 98:11479-11484; Thornsberry et al. (2001) “Dwarf8 polymorphisms associate with variation in flowering time,” Nat Genet 28:286-289; reviewed by Flint-Garcia et al. (2003) “Structure of linkage disequilibrium in plants,” Annu Rev Plant Biol. 54:357-374), where recombination among heterozygotes is frequent and results in a rapid decay of LD. In inbreeding species where recombination among homozygous genotypes is not genetically detectable, the extent of LD is greater (i.e., larger blocks of linked markers are inherited together) and this dramatically enhances the detection power of association mapping (Wall and Pritchard (2003) “Haplotype blocks and linkage disequilibrium in the human genome,” Nat Rev Genet 4:587-597).

The recombinational and mutational history of a population is a function of the mating habit as well as the effective size and age of a population. Large population sizes offer enhanced possibilities for detecting recombination, while older populations are generally associated with higher levels of polymorphism, both of which contribute to observably accelerated rates of LD decay. On the other hand, smaller effective population sizes, e.g., those that have experienced a recent genetic bottleneck, tend to show a slower rate of LD decay, resulting in more extensive haplotype conservation (Flint-Garcia et al. (2003) “Structure of linkage disequilibrium in plants,” Annu Rev Plant Biol. 54:357-374).

Elite breeding lines provide a valuable starting point for association analyses. Association analyses use quantitative phenotypic scores (e.g., disease tolerance rated from one to nine for each maize line) in the analysis (as opposed to looking only at tolerant versus resistant allele frequency distributions in intergroup allele distribution types of analysis). The availability of detailed phenotypic performance data collected by breeding programs over multiple years and environments for a large number of elite lines provides a valuable dataset for genetic marker association mapping analyses. This paves the way for a seamless integration between research and application and takes advantage of historically accumulated data sets. However, an understanding of the relationship between polymorphism and recombination is useful in developing appropriate strategies for efficiently extracting maximum information from these resources.

This type of association analysis neither generates nor requires any map data, but rather is independent of map position. This analysis compares the plants' phenotypic score with the genotypes at the various loci. Subsequently, any suitable maize map (for example, a composite map) can optionally be used to help observe distribution of the identified QTL markers and/or QTL marker clustering using previously determined map locations of the markers.

### Maize Lines and Phenotypic Scoring

Maize lines were phenotypically scored based on their degree of resistance to MRCV infection (in contrast to simple categorization of “tolerant” or “susceptible”). The plant varieties used in the analysis were from diverse sources, including elite germplasm, commercially released cultivars and other public varieties. The collections comprised 475 maize lines. The lines used in the study had a broad maturity range varying from CRM (comparative relative maturity) 90 to CRM 140, representing the main inbreds of Pioneer germplasm.

The degree of plant resistance to MRCV infection varied widely, as measured using a scale from one (highly susceptible) to nine (highly resistant). Generally, a score of two (2) indicated the most susceptible strains, a score of four (4) was assigned as the threshold to consider a plant susceptible or resistant (less than 4, susceptible; 4 or higher is resistant) and a score of seven (5-7) was assigned to the most resistant lines. Resistance scores of eight (8) and nine (9) were reserved for resistance levels that are very rare and generally not observed in existing germplasm. If no disease was present in a field, no resistance scoring was done. However, if a disease did occur in a specific field location, all of the lines in that location were scored. Scores for test strains accumulated over multiple locations and multiple years, and an averaged (e.g., consensus) score was ultimately assigned to each line.

Resistance scores for the 475 inbred collection were collected over several growing seasons (394 inbreds were evaluated at the same time in the growing season). Data collection was typically done in one scoring after flowering time.

In assessing the linkage of markers to tolerance, a quantitative approach was used, where a resistance score for each maize line was assessed and incorporated into the association mapping statistical analysis.

### Maize Genotyping

A collection of 475 maize lines was analyzed by DNA sequencing at 4000-10000 genes (genetic loci). SNP variation was used to generate specific haplotypes across inbreds at each loci. This data was used for identifying associations between alleles and MRCV resistance at genome level.

### Statistical Methods

A structure-based association analysis is conducted using standard association mapping methods where the population structure is controlled by using marker data. The model-based cluster analysis software, Structure, developed by Pritchard et al. was used with haplotype data for 880 elite maize inbreds at two hundred markers to estimate admixture coefficients and assign the inbreds to seven subpopulations (J. K. Pritchard, M. Stephens and P. J. Donnelly (2000) “Inference of population structure using multilocus genotype data,” Genetics 155:945-959). This reduces the occurrence of false positives that can arise due to the effect of population structure on association mapping statistics. Kuiper's statistic for testing whether two distributions are the same is used to test a given marker for association between haplotype and phenotype in a given subpopulation (W. H. Press, S. A. Teukolsky, W. T. Vetterling, B. P. Flannery, 2002; Numerical Recipes in C, second edition, Cambridge University Press, NY).

The Pedigree-based association mapping is conducted using GPA Procedure (General Pedigree-Based Association Analysis), developed by Shu et al. (Guoping Shu, Beiyan Zeng, and Oscar Smith, 2003; Detection Power of Random, Case-Control, and Case-Parent Control Designs for Association Tests and Genetic Mapping of Complex Traits. Proceedings of 15th Annual KSU Conference on Applied Statistics in Agriculture. 15: 191-204). The GPA Procedure is a conditional likelihood-based association mapping software implemented in SAS Computer Language Version 9.0 (2001, SAS Institute, Cary, N.C.).

### Results

Tables 1 and 2 provide tables listing the maize markers that demonstrated linkage disequilibrium with the MRCV phenotype using the Association Mapping method, and they were validated on segregating populations. Also indicated in Tables 1 and 2 are the chromosomes on which the markers are located and their approximate map position relative to other known markers, given in cM, with position zero being the first (most distal from centromere) marker known at the beginning of the chromosome. These map positions are not absolute, and represent an estimate of map position. Tables 6 and 7 provide the primer and probe sequences used to type the SNP markers.

The statistical probabilities that the marker allele and disease tolerance phenotype are segregating independently are reflected in the association mapping adjusted probability values in Tables 1 and 2, which is a probability (P) derived from analysis of association between genotype and phenotype. The lower the probability value, the more significant is the association between the marker genotype at that locus and the MRCV infection tolerance phenotype.

Non-structured association analysis for the named SS group revealed the presence of two peaks of probability on chromosome 2, at position 65.99 represented by markers MZA2038 (p=0.00000266) and MZA11826 (p=0.00000179) and at position 127.18-131.13 represented by markers MZA11806 (p=0.000002) and MZA14212 (p=0.00000327). The non-structured analysis also revealed several other associations across the genome. The only consistent association that it was validated by independent approaches corresponded to the position 65.99 on chromosome 2. The non-structured analysis increases the power to evaluate the whole allele variability for a target region but at the same time increase the number of false positive associations because population structure is not corrected by this analysis.

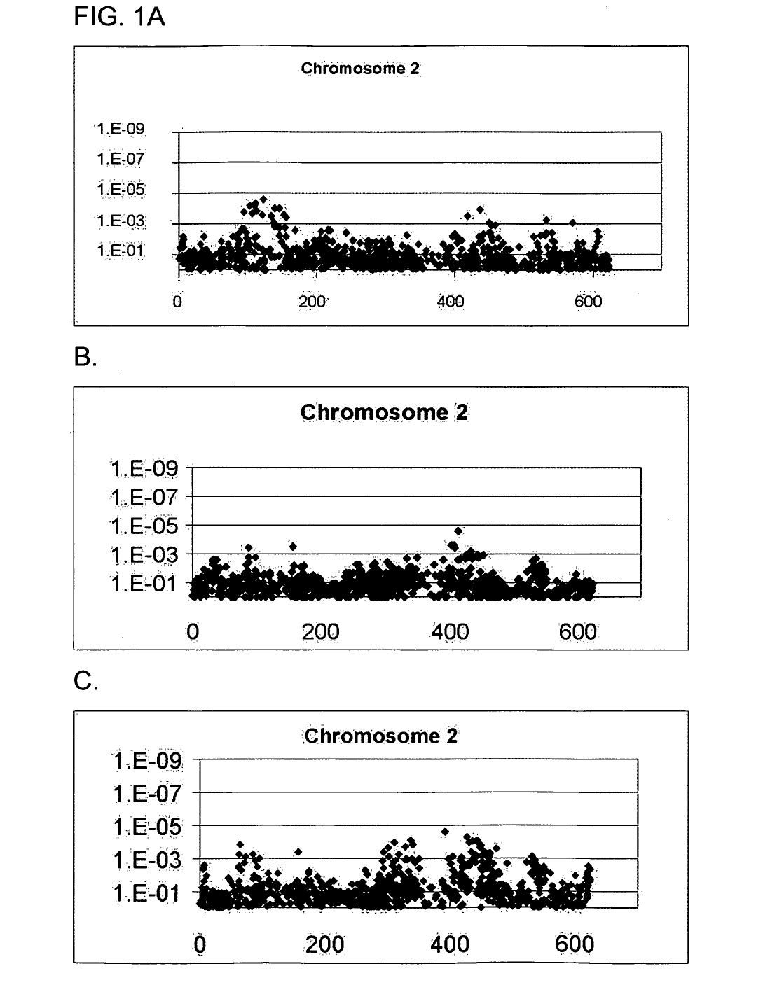

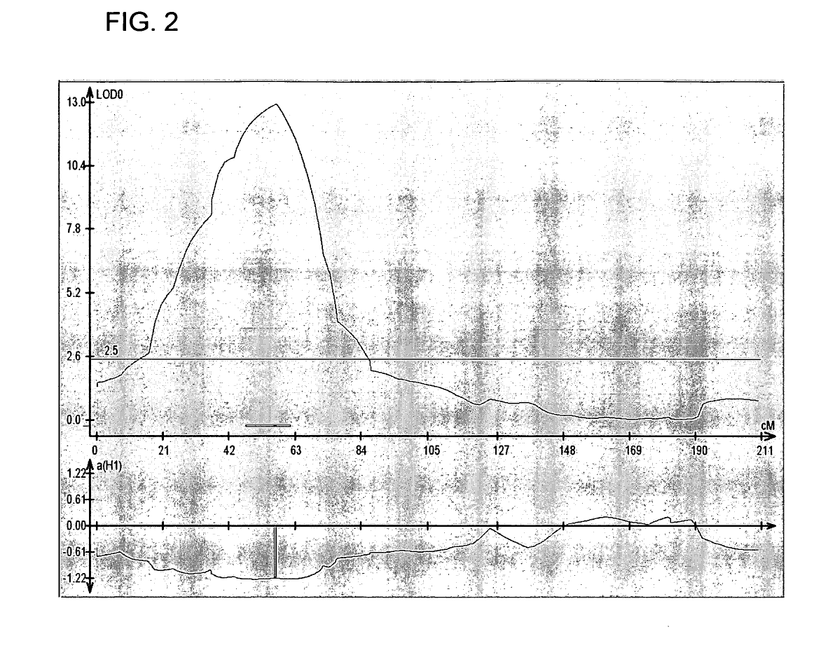

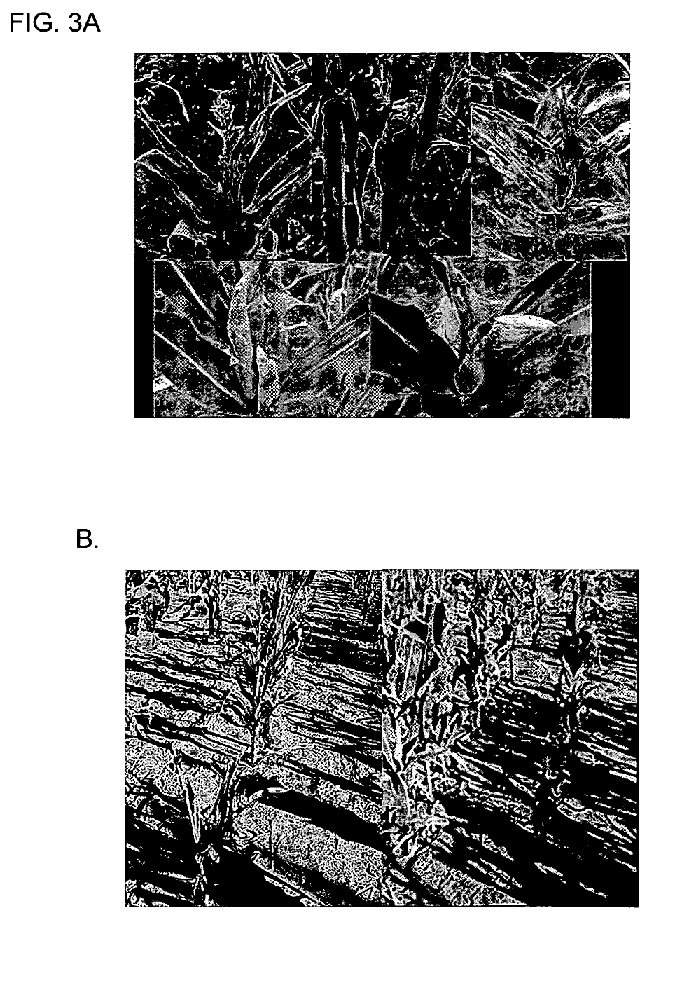

FIG. 1A shows a structured association analysis of a group of argentinian inbreds (or inbreds target for the argentine breeding program) where several markers were significant at 0.0005 p-level at the region from position 65.99 to 85.84, including MZA16656 (P=0.000194), MZA18224 (p=0.000066) and MZA5057 (p=0.000045). FIG. 1B shows a structured association analysis for an SS group where on the short arm of chromosome 2, the most associated markers were MZA1525 at position 54.62 (p=0.00043) and MZA11826 at position 65.99 (p=0.00168). Two additional associations were observed at position 91.19 represented by marker MZA13812 (p=0.000299) and position 154.06 represented by marker MZA10682 (p=0.000024).

FIG. 1C shows a structured association analysis for the SS group with a different set of phenotypic data. The highest associated marker on the short arm of chromosome 2 was MZA12899 at position 53.83 (p=0.000298). There were other associated markers in the long arm of chromosome 2 where the highest associated markers were MZA1067 (Map position: 141.9; p=0.000094) and MZA10832 (Map position: 159.8; p=0.000086).

### Example 2

### QTL Interval Mapping and Single Marker Regression Analysis

A QTL interval mapping and a single marker regression analysis was undertaken to identify maize chromosome intervals and genetic markers (respectively) that are associated with resistance and allow the plant resistance of maize to MRCV infection. QTL mapping and marker regression are widely used methods to identify genetic loci that co-segregate with a desired phenotype. By identifying such genetic loci, marker assisted selection (MAS) can be used to improve the efficiency of breeding for improved maize inbreds and hybrids.

### Maize Lines

Two main mapping populations for MRCV resistance were created from the crosses of inbreds PH7WT (resistant genotype) and PH3DT (highly susceptible genotype), and PH9TJ (resistant genotype) and PH890 (susceptible genotype). The PH7WT×PH3DT population consisted of 120 F5/F7 families and the PH9TJ×PH890 consisted of 212 BC2F4/BC2F5 families.

### Phenotypic Scoring

Phenotypic scoring of each of the lines was based on sets of phenotypic data collected from the field on two (PH890×PH9TJ cross) or three different crop seasons (PH7WT×PH3DT).

### Maize Genotyping

Maize F5 progeny of PH7WT×PH3DT were genotyped using a total of 246 polymorphic and good quality markers and the BC2F4 progeny of PH890×PH9TJ were genotyped with 167 polymorphic and good quality markers. First round of genotyping included SSR markers. A second round of genotyping with a set of 101 polymorphic and good quality markers was performed on F7 PH7WT×PH3DT progeny. A second round of genotyping was performed on PH890×PH9TJ population by using a set of makers at specific genomic regions.

Windows QTL Cartographer (the most up-to-date version of this software was used according the date of QTL mapping) was used for both the marker regression analysis and QTL interval mapping. LOD scores (logarithm of the odds ratio) were estimated across the genome according the standard QTL mapping procedures. The term “likelihood of odds” is used to describe the relative probability of two or more explanations of the sources of variation in a trait. The probability of these two different explanations (models) can be computed, and the most likely model chosen. If model A is 1000 times more probable than model B, then the ratio of the odds are 1000:1 and the logarithm of the odds ratio is 3.

Both the raw data for individual replications and years, and mean scores, were used in QTL interval mapping. The LOD threshold was 2.5. A confidence interval was estimated for each QTL. The positions obtained are then plotted as a histogram overlaying the interval mapping figure.

### Results

### QTL Interval Mapping

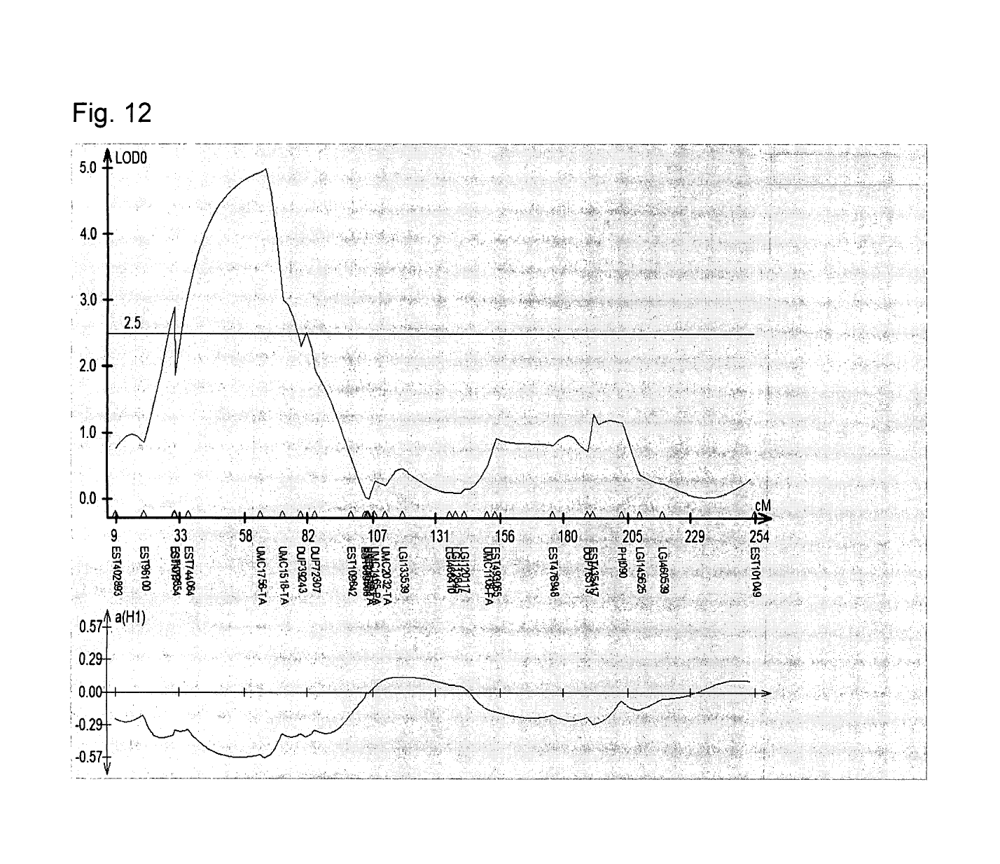

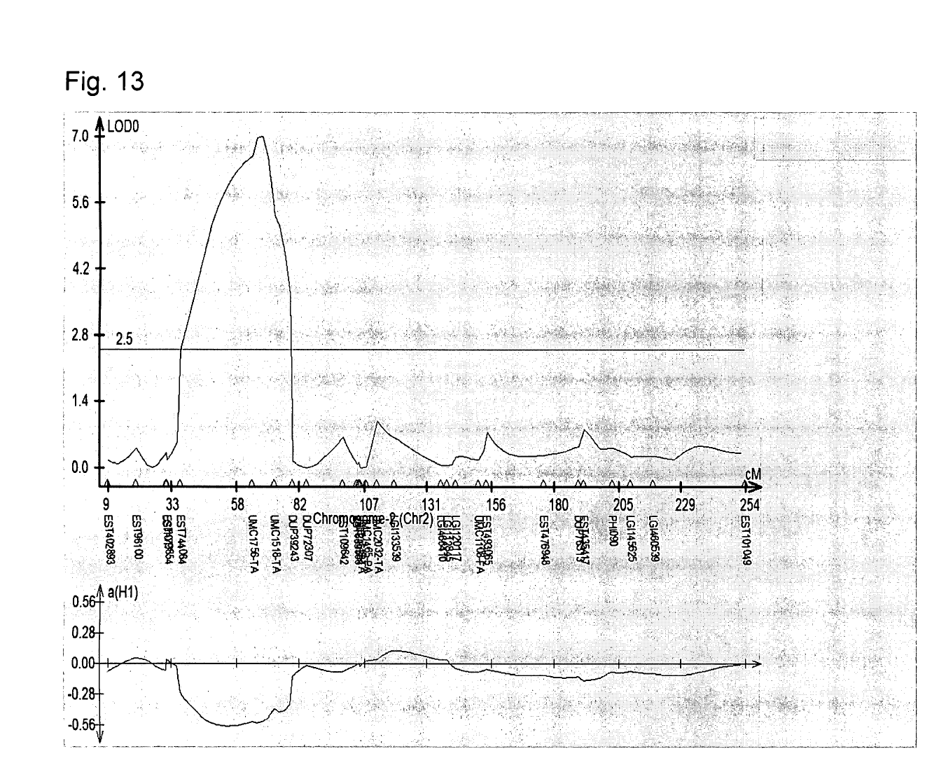

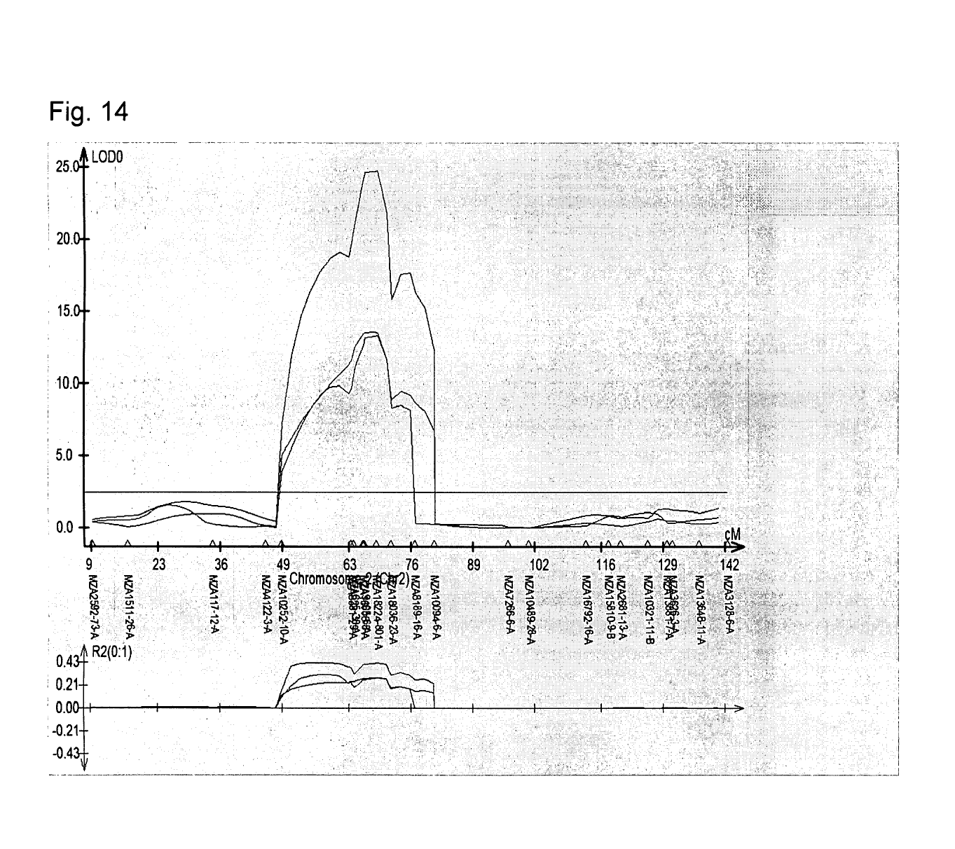

The present study identified various chromosome intervals that correlate with QTLs that associate with resistance/susceptibility to MRCV infection. The QTLs were identified using the field data. One major, significant QTL was located on linkage group 2 on both mapping crosses (see FIGS. 12-14; see also Table 13, which shows a QTL marker regression analysis for the PH890×PH9TJ cross).

TABLE 13

| | | | | | | | | | | | | | | Mean |
|---|---|---|---|---|---|---|---|---|---|---|---|---|---|---|
| Marker | Chrom. | Position | b1 | F(1, n-2) | pr(F) | Rep 1 | b1 | F(1, n-2) | pr(F) | Rep 2 | b1 | F(1, n-2) | pr(F) | Score |
| MZA117-12-A | 2 | 34.53 | −0.245 | 4.691 | 0.032 | * | −0.117 | 1.026 | 0.313 | | −0.242 | 7.452 | 0.007 | ** |
| MZA4122-3-A | 2 | 45.60 | −0.354 | 11.881 | 0.001 | *** | −0.383 | 13.637 | 0 | *** | −0.383 | 23.661 | 0 | **** |
| MZA10252-10-A | 2 | 48.85 | −0.389 | 13.624 | 0 | *** | −0.444 | 17.587 | 0 | **** | −0.380 | 21.621 | 0 | **** |
| MZA8381-29-A | 2 | 63.47 | −0.640 | 45.160 | 0 | **** | −0.716 | 58.110 | 0 | **** | −0.640 | 85.828 | 0 | **** |
| MZA625-30-A | 2 | 64.05 | −0.638 | 50.504 | 0 | **** | −0.686 | 58.803 | 0 | **** | −0.622 | 90.530 | 0 | **** |
| MZA16656-8-A | 2 | 65.99 | −0.719 | 66.790 | 0 | **** | −0.727 | 66.005 | 0 | **** | −0.685 | 117.393 | 0 | **** |
| MZA9105-6-A | 2 | 65.44 | −0.719 | 66.790 | 0 | **** | −0.727 | 66.005 | 0 | **** | −0.685 | 117.393 | 0 | **** |
| MZA9510-8-A | 2 | 65.44 | −0.702 | 64.097 | 0 | **** | −0.720 | 65.830 | 0 | **** | −0.673 | 114.098 | 0 | **** |
| MZA18224-801-A | 2 | 68.80 | −0.739 | 69.538 | 0 | **** | −0.730 | 64.530 | 0 | **** | −0.698 | 119.381 | 0 | **** |
| MZA2349-71-A | 2 | 68.80 | −0.694 | 60.777 | 0 | **** | −0.625 | 44.607 | 0 | **** | −0.651 | 100.026 | 0 | **** |
| MZA18036-23-A | 2 | 71.75 | −0.576 | 41.165 | 0 | **** | −0.531 | 32.632 | 0 | **** | −0.543 | 65.053 | 0 | **** |
| MZA8189-16-A | 2 | 76.80 | −0.542 | 37.689 | 0 | **** | −0.538 | 35.626 | 0 | **** | −0.529 | 64.896 | 0 | **** |
| MZA10094-6-A | 2 | 80.90 | −0.501 | 30.686 | 0 | **** | −0.475 | 26.107 | 0 | **** | −0.477 | 48.187 | 0 | **** |
| MZA7266-6-A | 2 | 96.43 | −0.267 | 5.081 | 0.025 | * | −0.169 | 1.955 | 0.164 | | −0.223 | 5.677 | 0.018 | * |
| MZA15573-12-A | 5 | 144.73 | −0.153 | 1.371 | 0.243 | | −0.332 | 6.482 | 0.012 | * | −0.234 | 5.252 | 0.023 | * |
| MZA7908-20-A | 5 | 152.87 | −0.284 | 7.206 | 0.008 | ** | −0.421 | 16.156 | 0 | **** | −0.336 | 17.077 | 0 | **** |
| MZA8726-9-A | 5 | 154.05 | −0.326 | 10.429 | 0.001 | ** | −0.459 | 21.357 | 0 | **** | −0.375 | 23.713 | 0 | **** |
| MZA4599-24-A | 5 | 167.44 | −0.237 | 5.649 | 0.019 | * | −0.235 | 5.380 | 0.022 | * | −0.293 | 14.552 | 0 | *** |
| MZA8048-8-A | 5 | 168.07 | −0.231 | 5.339 | 0.022 | * | −0.234 | 5.305 | 0.022 | * | −0.291 | 14.201 | 0 | *** |
| MZA3899-10-A | 5 | 175.23 | −0.123 | 1.292 | 0.257 | | −0.225 | 4.264 | 0.040 | * | −0.211 | 6.270 | 0.013 | * |

A second QTL was identified on linkage group 5 at position 150-160 (PH890×PH9TJ pop) and another at position 200-220 on linkage group 5 (PH7WT×PH3DT pop). A third QTL was mapped on PH7WT×PH3DT at position 165-185 on chromosome 2.

### Single Marker Regression

Using single marker regression, there are a number of markers showing association with the resistant phenotype at a confidence level of P=0.05 or better, as shown in Tables 1 and 2. Some of the markers identified in the marker regression analysis show a concordance of observations with the association mapping, where the different approaches identify the same markers. For example, there are markers at the region from 55 to 70 cM on Chr 2 identified by both marker regression and association mapping.

### Discussion/Conclusions

This present study has identified chromosome intervals and individual markers that correlate with MRCV resistance. Markers that lie within these intervals are useful for use in MAS, as well as other purposes.

### Example 3

### QTL Validation by Marker Assisted Selection

A QTL interval mapping and a single marker regression analysis was undertaken to identify maize chromosome intervals and genetic markers (respectively) that are associated with resistance and allow the resistance to MRCV infection. QTL mapping and marker regression are widely used methods to identify genetic loci that co-segregate with a desired phenotype. By identifying such genetic loci, marker assisted selection (MAS) can be used to improve the efficiency of breeding for improved maize inbreds and hybrids.

### Maize Lines

One main population for validation and mapping of MRCV resistance was created from the cross of inbreds PH7WT and PH3DT. Other populations were generated to validate the effect of this QTL across backgrounds. The PH7WT×PH3DT population consisted of 82 BC3F3 families generated by introgress by markers the QTL mapped on chromosome 2 into PH3DT. There were 4 additional BC1F3 populations generated by marker assisted selection that consisted of 24 BC1F3 from the cross PH6KW×PH7WT, 12 BC1F3 from the cross PH6B8×PH7WT, 3 BC1F3 from the cross PHP3P1×PH7WT and 6 BC1F3 from the cross PH6GF×PH7WT. These populations were generated by selfing specific BC3 or BC1 plants and deriving BC3F3 or BC1F3 families with allelic variation at the QTL region.

### Phenotypic Scoring

Phenotypic scoring of each of the BC1F3, BC3F3 and parents was based on sets of phenotypic data collected from the field on one crop season.

### Maize Genotyping

Maize BC1F2 progeny from the different crosses and BC3F3 from the cross PH7WT×PH3DT were genotyped by using polymorphic SNPs at the QTL region. BC3F3 were subjected to background clean at BC3 stage, especially at chromosome 5 QTL. Markers included SNP markers.

Windows QTL Cartographer (up-to-date version according the date of QTL mapping) was used for both the marker regression analysis and QTL interval mapping. LOD scores (logarithm of the odds ratio) were estimated across the genome according the standard QTL mapping procedures.

Both the raw data for individual replications and mean scores were used in QTL interval mapping. The LOD threshold was 2.5. A confidence interval was estimated for each QTL. The positions obtained are then plotted as a histogram overlaying the interval mapping figure.

As these population were generated by marker assisted selection (not random events of recombination), marker regression analysis was considered as powerful as interval mapping analysis.

### Results

### QTL Interval Mapping

The present study identified a single chromosome interval that correlated with QTLs associated with resistance/susceptibility to MRCV infection. The QTL were identified using the field data. One major significant QTL was located on linkage group 2 on the main validation BC3F3 population. The main markers at this QTL in the main validation population when checked on the other BC1F3 progenies confirmed the effect of this QTL on resistance/susceptibility to MRCV infection.

### Single Marker Regression

Using single marker regression, there are a number of markers showing association with the resistant phenotype at a confidence level of P=0.05 or better, as shown in Tables 1 and 2. Some of the markers identified in the marker regression analysis show a concordance of observations with the association mapping, where the different approaches identify the same markers. For example, there are markers at the region from 55 to 70 cM on chromosome 2 identified by both marker regression and association mapping. See FIG. 2 for interval mapping and Table 14 for marker regression analysis for the PH3DT×PH7WT cross. Note that replication #3 was affected by herbicide stress. MRCVSC=MRCV phenotypic score. Inc. Sev. Symp.=frequency of plants with severe symptoms on each experimental unit.

TABLE 14

| | | | | | | | | | | | | | | Mean |
|---|---|---|---|---|---|---|---|---|---|---|---|---|---|---|
| Marker | Chrom. | Position | F(1, n-2) | pr(F) | Rep 1 | F(1, n-2) | pr(F) | Rep 2 | F(1, n-2) | pr(F) | Rep 3 | F(1, n-2) | pr(F) | Score |
| | | | Inc sev symptoms | | | Inc sev symptoms | | | Inc sev symptoms | | | Inc sev symptoms | | |
| MZA2592-73-A | 2 | 9.29 | 3.49 | 0.07 | | 4.22 | 0.04 | | 1.19 | 0.28 | | 6.29 | 0.01 | * |
| MZA225-50-A | 2 | 25.51 | 5.57 | 0.02 | * | 3.10 | 0.08 | | 1.38 | 0.24 | | 6.35 | 0.01 | * |
| MZA3334-4-A | 2 | 33.38 | 9.98 | 0 | ** | 2.15 | 0.015 | | 5.60 | 0.02 | * | 10.58 | 0 | ** |
| MZA4122-3-A | 2 | 45.60 | 26.19 | 0 | **** | 9.49 | 0 | ** | 10.21 | 0 | ** | 31.82 | 0 | **** |
| MZA8067-27-A | 2 | 52.77 | 28.10 | 0 | **** | 9.21 | 0 | ** | 9.57 | 0 | ** | 31.56 | 0 | **** |
| MZA5822-15-A | 2 | 53.53 | 30.53 | 0 | **** | 10.77 | 0 | ** | 10.26 | 0 | ** | 35.66 | 0 | **** |
| MZA1525-98-A | 2 | 54.62 | 32.66 | 0 | **** | 12.89 | 0 | *** | 10.85 | 0 | ** | 40.37 | 0 | **** |
| MZA8381-801-A | 2 | 63.47 | 34.31 | 0 | **** | 14.25 | 0 | *** | 11.56 | 0 | ** | 43.52 | 0 | **** |
| MZA625-29-A | 2 | 64.05 | 34.27 | 0 | **** | 13.94 | 0 | *** | 11.91 | 0 | *** | 44.63 | 0 | **** |
| MZA625-30-A | 2 | 65.99 | 34.88 | 0 | **** | 14.21 | 0 | *** | 11.78 | 0 | *** | 44.21 | 0 | **** |
| MZA16656-19-A | 2 | 65.99 | 32.03 | 0 | **** | 13.17 | 0 | *** | 11.08 | 0 | ** | 40.36 | 0 | **** |
| MZA15490-801-A | 2 | 65.99 | 34.31 | 0 | **** | 14.66 | 0 | *** | 11.46 | 0 | ** | 44.16 | 0 | **** |
| MZA2038-71-A | 2 | 65.99 | 34.31 | 0 | **** | 14.66 | 0 | *** | 11.46 | 0 | ** | 44.16 | 0 | **** |
| MZA11826-803-A | 2 | 65.99 | 34.31 | 0 | **** | 14.66 | 0 | *** | 11.46 | 0 | ** | 44.16 | 0 | **** |
| MZA11826-801-A | 2 | 65.99 | 34.31 | 0 | **** | 14.66 | 0 | *** | 11.46 | 0 | ** | 44.16 | 0 | **** |
| MZA9105-8-A | 2 | 65.44 | 34.30 | 0 | **** | 14.67 | 0 | *** | 11.46 | 0 | ** | 44.16 | 0 | **** |
| MZA18224-801-A | 2 | 68.80 | 34.19 | 0 | **** | 14.76 | 0 | *** | 11.39 | 0 | ** | 44.14 | 0 | **** |
| MZA18036-23-A | 2 | 71.75 | 29.40 | 0 | **** | 11.65 | 0 | ** | 9.41 | 0 | ** | 35.80 | 0 | **** |
| MZA15853-10-A | 2 | 77.72 | 23.81 | 0 | **** | 5.97 | 0.02 | * | 6.02 | 0.02 | * | 22.76 | 0 | **** |
| MZA10094-6-A | 2 | 80.90 | 23.24 | 0 | **** | 5.82 | 0.02 | * | 7.91 | 0.01 | ** | 24.05 | 0 | **** |
| MZA15844-19-A | 2 | 82.87 | 19.48 | 0 | **** | 3.67 | 0.06 | | 8.16 | 0.01 | ** | 19.32 | 0 | **** |
| MZA4425-25-A | 2 | 85.68 | 12.42 | 0 | *** | 1.94 | 0.17 | | 6.37 | 0.01 | * | 12.15 | 0 | *** |
| MZA7964-33-A | 2 | 94.40 | 6.24 | 0.02 | * | 2.08 | 0.15 | | 6.23 | 0.02 | * | 7.90 | 0.01 | ** |
| MZA1962-33-A | 2 | 96.01 | 5.32 | 0.02 | * | 2.01 | 0.16 | | 6.93 | 0.01 | * | 7.36 | 0.01 | ** |
| MZA5581-13-A | 2 | 105.99 | 4.91 | 0.03 | * | 1.47 | 0.23 | | 7.99 | 0.01 | ** | 7.70 | 0.01 | ** |
| MZA3439-8-A | 2 | 128.57 | 4.27 | 0.04 | * | 0.90 | 0.35 | | 7.34 | 0.01 | ** | 6.51 | 0.01 | * |
| MZA4564-49-A | 2 | 142.10 | 5.85 | 0.02 | * | 0.66 | 0.42 | | 8.59 | 0 | ** | 7.49 | 0.01 | ** |
| MZA10883-17-A | 2 | 158.98 | 0.20 | 0.66 | | 0.02 | 0.89 | | 2.26 | 0.14 | | 0.24 | 0.63 | |
| MZA12915-19-A | 2 | 170.53 | 0.00 | 0.98 | | 0.01 | 0.91 | | 0.57 | 0.45 | | 0.04 | 0.84 | |
| MZA10488-21-A | 2 | 177.67 | 0.19 | 0.66 | | 0.01 | 0.91 | | 0.11 | 0.74 | | 0.03 | 0.87 | |
| MZA3152-16-A | 2 | 191.27 | 0.15 | 0.70 | | 0.11 | 0.74 | | 0.74 | 0.39 | | 0.00 | 1.00 | |
| MZA505-250-A | 2 | 201.35 | 1.20 | 0.28 | | 2.22 | 0.14 | | 1.53 | 0.22 | | 3.55 | 0.06 | |
| | | | MRCVSC | | | MRCVSC | | | MRCVSC | | | MRCVSC | | |
| MZA2592-73-A | 2 | 9.29 | 2.87 | 0.09 | | 0.86 | 0.36 | | 0.02 | 0.90 | | 1.37 | 0.25 | |
| MZA225-50-A | 2 | 25.51 | 8.89 | 0 | ** | 3.31 | 0.07 | | 1.15 | 0.29 | | 5.63 | 0.02 | * |
| MZA3334-4-A | 2 | 33.38 | 21.58 | 0 | **** | 4.03 | 0.05 | * | 2.48 | 0.12 | | 11.67 | 0 | ** |
| MZA4122-3-A | 2 | 45.60 | 35.56 | 0 | **** | 6.06 | 0.02 | * | 4.90 | 0.03 | * | 20.90 | 0 | **** |
| MZA8067-27-A | 2 | 52.77 | 48.72 | 0 | **** | 9.52 | 0 | ** | 8.85 | 0 | ** | 32.41 | 0 | **** |
| MZA5822-15-A | 2 | 53.53 | 53.73 | 0 | **** | 9.93 | 0 | ** | 9.06 | 0 | ** | 34.53 | 0 | **** |
| MZA1525-98-A | 2 | 54.62 | 58.15 | 0 | **** | 10.12 | 0 | ** | 8.96 | 0 | ** | 35.86 | 0 | **** |
| MZA8381-801-A | 2 | 63.47 | 58.64 | 0 | **** | 12.09 | 0 | *** | 9.52 | 0 | ** | 39.57 | 0 | **** |
| MZA625-29-A | 2 | 64.05 | 63.88 | 0 | **** | 13.29 | 0 | *** | 10.82 | 0 | ** | 40.92 | 0 | **** |
| MZA625-30-A | 2 | 65.99 | 60.02 | 0 | **** | 12.44 | 0 | *** | 9.81 | 0 | ** | 40.33 | 0 | **** |
| MZA16656-19-A | 2 | 65.99 | 59.49 | 0 | **** | 11.23 | 0 | ** | 10.13 | 0 | ** | 38.87 | 0 | **** |
| MZA15490-801-A | 2 | 65.99 | 62.05 | 0 | **** | 13.09 | 0 | *** | 10.51 | 0 | ** | 41.62 | 0 | **** |
| MZA2038-71-A | 2 | 65.99 | 62.05 | 0 | **** | 13.09 | 0 | *** | 10.51 | 0 | ** | 41.62 | 0 | **** |
| MZA11826-803-A | 2 | 65.99 | 62.05 | 0 | **** | 13.09 | 0 | *** | 10.51 | 0 | ** | 41.62 | 0 | **** |
| MZA11826-801-A | 2 | 65.99 | 62.05 | 0 | **** | 13.09 | 0 | *** | 10.51 | 0 | ** | 41.62 | 0 | **** |
| MZA9105-8-A | 2 | 65.44 | 62.03 | 0 | **** | 13.09 | 0 | *** | 10.50 | 0 | ** | 41.61 | 0 | **** |
| MZA18224-801-A | 2 | 68.80 | 61.77 | 0 | **** | 13.10 | 0 | *** | 10.43 | 0 | ** | 41.50 | 0 | **** |
| MZA18036-23-A | 2 | 71.75 | 47.25 | 0 | **** | 11.83 | 0 | *** | 7.58 | 0.01 | ** | 40.17 | 0 | **** |
| MZA15853-10-A | 2 | 77.72 | 43.32 | 0 | **** | 8.75 | 0 | ** | 6.44 | 0.01 | * | 31.80 | 0 | **** |
| MZA10094-6-A | 2 | 80.90 | 37.87 | 0 | **** | 7.62 | 0.01 | ** | 9.31 | 0 | ** | 31.20 | 0 | **** |
| MZA15844-19-A | 2 | 82.87 | 33.89 | 0 | **** | 4.90 | 0.03 | * | 8.38 | 0.01 | ** | 26.58 | 0 | **** |
| MZA4425-25-A | 2 | 85.68 | 19.92 | 0 | **** | 1.44 | 0.23 | | 5.28 | 0.02 | * | 15.62 | 0 | *** |
| MZA7964-33-A | 2 | 94.40 | 12.94 | 0 | *** | 2.44 | 0.12 | | 7.06 | 0.01 | ** | 13.89 | 0 | *** |
| MZA1962-33-A | 2 | 96.01 | 11.02 | 0 | ** | 2.34 | 0.13 | | 7.63 | 0.01 | ** | 13.26 | 0 | *** |
| MZA5581-13-A | 2 | 105.99 | 8.12 | 0.01 | ** | 1.93 | 0.17 | | 6.48 | 0.01 | * | 14.69 | 0 | *** |
| MZA3439-8-A | 2 | 128.57 | 2.93 | 0.09 | | 1.47 | 0.23 | | 2.82 | 0.10 | | 7.13 | 0.01 | ** |
| MZA4564-49-A | 2 | 142.10 | 2.90 | 0.09 | | 1.47 | 0.23 | | 2.98 | 0.09 | | 6.78 | 0.01 | * |
| MZA10883-17-A | 2 | 158.98 | 0.00 | 0.96 | | 0.86 | 0.36 | | 0.28 | 0.60 | | 1.83 | 0.18 | |
| MZA12915-19-A | 2 | 170.53 | 0.67 | 0.42 | | 0.43 | 0.51 | | 0.16 | 0.69 | | 0.57 | 0.45 | |
| MZA10488-21-A | 2 | 177.67 | 0.21 | 0.65 | | 0.01 | 0.95 | | 0.04 | 0.85 | | 0.01 | 0.91 | |
| MZA3152-16-A | 2 | 191.27 | 0.62 | 0.43 | | 0.32 | 0.57 | | 0.00 | 1.00 | | 0.31 | 0.58 | |
| MZA505-250-A | 2 | 201.35 | 0.66 | 0.42 | | 0.23 | 0.63 | | 0.07 | 0.80 | | 0.80 | 0.37 | |

The effect of MRCV1 allelic variation on several backgrounds was evaluated by the phenotypic data of BC1F3s progeny with allelic variation at MRCV1 region. MRCV1 resistant allele showed a positive effect across another 4 genetic backgrounds (PH6GF, PHP3P1, PH6B8 and PH6 KW inbreds). Table 15 below shows the mean phenotypic score for BC1F3 progeny with allelic variation at MRCV1 region.

TABLE 15

| | Inbreds | | | |
|---|---|---|---|---|
| Marker position 65.99 | PH6GF | PHP3P1 | PH6B8 | PH6KW |
| Inbred score | 3.8 | 2.5 | 3 | 3 |
| BC1F3 susceptible allele (AA) | 5.00 | 4.10 | 4.50 | 4.19 |
| BC1F3 heterozygous allele (AB) | — | 3.53 | 5.17 | 4.26 |
| BC1F3 resistant allele (BB) | 6.11 | 6.17 | 5.47 | 5.00 |
| QTL effect | 1.11 | 2.07 | 0.97 | 0.81 |

### Discussion/Conclusions

This present study has identified chromosome intervals and individual markers that correlate with MRCV resistance. Markers that lie within these intervals are useful for use in MAS, as well as other purposes.

### Example 4

### QTL Validation on DH Breeding Populations

A QTL marker regression analysis was undertaken to identify maize chromosome intervals and genetic markers (respectively) that are associated with resistance and allow the resistance to MRCV infection. QTL mapping and marker regression are widely used methods to identify genetic loci that co-segregate with a desired phenotype. By identifying such genetic loci, marker assisted selection (MAS) can be used to improve the efficiency of breeding for improved maize inbreds and hybrids.

### Maize Lines

Marker enhanced pedigree selection (MEPS) populations means the scheme of breeding population populations for MRCV resistance were created from different crosses of inbreds. The crosses included: a) Crosses with MRCV1 fixed: PHKEF×PHBNA, PHKEF×PHS2G, PHKFD×PHS3J, PHKFA×PHBNA, PHKFA×PHKEF, PHS2Y×PHKEF, and b) Crosses with MRCV1 segregating: PH3DT×PHKEF, PHKEF×PH9PR, PHKEF×PH9PR, PHKEF×PHKDK, PHKDN×PHKFD, PHKDN×PHS3J, PHKFD×PHCOG, PHKDK×PHKFA, PHKDN×PH9PH.

TABLE 16

| Population | # Ind | MRCV1 |
|---|---|---|
| PHKEF/PHBNA | 9 | Fixed |
| PHKEF/PHS2G | 13 | Fixed |
| PHKFD/PHS3J | 12 | Fixed |
| PHKFA/PHBNA | 12 | Fixed |
| PHKFA/PHKEF | 34 | Fixed |
| PHS2Y/PHKEF | 12 | Fixed |
| PH3DT/PHKEF | 11 | Segregating |
| PHKEF/PH9PR | 12 | Segregating |
| PHKEF/PHKDK | 18 | Segregating |
| PHKDN/PHKFD | 30 | Segregating |
| PHKDN/PHS3J | 11 | Segregating |
| PHKFD/PHC0G | 9 | Segregating |
| PHKDK/PHKFA | 15 | Segregating |
| PHKDN/PH9PH | 8 | Segregating |

These populations were generated by the doubled haploids process. The number of individuals characterized for MRCV resistance were included in Table 16. Fingerprint data at the main QTL region and identity by descent information was used to define QTL segregating and QTL fixed populations.

### Phenotypic Scoring

Phenotypic scoring of each of the DH MEPS population was based on sets of phenotypic data collected from the field in one crop season.

### Maize Genotyping

Maize DH progeny from the different crosses were genotyped by using a set of 756 SNPs distributed in the maize genome. The positions obtained are then plotted as a histogram overlaying the interval mapping figure.

### Results

### QTL Marker Analysis

The present study identified a single major chromosome interval that correlated with QTL associated with resistance/susceptibility to MRCV infection when populations from SS crosses Resistant×Susceptible and segregating for MRCV major QTL on chromosome 2 were selected “a priori” 2. The QTL were identified using the field data. One major, significant QTL was located on linkage group 2. Genetic crosses between inbreds harboring the positive allele of the major QTL on chromosome 2 (QTL fixed by parents) showed most of the progenies with a field MRCV score of 4 or higher.

### Single Marker Regression

Using single marker regression, there are a number of markers showing association with the resistant phenotype at a confidence level of P=0.05 or better, as shown in Tables 1 and 2. Some of the markers identified in the marker regression analysis show a concordance of observations with the association mapping, where the different approaches identify the same markers. For example, there are markers at the region from 55 to 70 cM on chromosome 2 identified by both marker regression and association mapping. On a group of SS inbreds, main association was located at the MZA10538 marker (position 54.5). On a group of NSS inbreds, a major association was located at position 72 cM.

### Discussion/Conclusions

This present study has identified chromosome intervals and individual markers that correlate with MRCV resistance. Markers that lie within these intervals are useful for use in MAS, as well as other purposes.

### Example 5

### Main Inbreds Characterization

A set of key argentine genetic materials were phenotypically and genetically characterized to confirm maize genetic marker loci associated with resistance to MRCV. By identifying such genetic markers, marker assisted selection (MAS) can be used to improve the efficiency of breeding for improved resistance of maize to MRCV.

### Maize Lines and Resistance Scoring

The plant varieties used in the analysis were from diverse sources, including elite germplasm, commercially released cultivars and other public lines representing a broad range of germplasm related to the argentine breeding program and including main sources of MRCV resistance.

The groups of maize lines were assembled for the analysis based on their phenotypic responses against MRCV infection, where the plants were sorted into either highly susceptible or highly resistant varieties. The classifications of resistance and susceptible were based solely on observations of fortuitous, naturally occurring disease incidence in field tests over several years. The degree of plant resistance to MRCV infection varied widely, as measured using a scale from one (highly susceptible) to nine (highly tolerant). Generally, a score of two (2) indicated the most susceptible strains, a score of four (4) was assigned as the threshold to consider a plant susceptible or resistant (less than 4, susceptible; 4 or higher is resistant) and a score of seven (5-7) was assigned to the most resistant lines. Resistance scores of eight (8) and nine (9) were reserved for resistance levels that are very rare and generally not observed in existing germplasm. If no disease was present in a field, no resistance scoring was done. However, if a disease did occur in a specific field location, all of the lines in that location were scored. Scores for test strains accumulated over multiple locations and multiple years, and an averaged (e.g., consensus) score was ultimately assigned to each line.

Data collection was typically done in one scoring time. Scoring time is placed after flowering time.

In assessing association of markers to resistance, a comparison by simple regression approach was used. Allele origin was checked by the identity by descent approach. Using this approach, those maize lines that were considered to be representative of either the resistant or susceptible classes were used for assessing association. A list of resistant lines was constructed, where inbreds having a resistance score of 4 or greater were considered “Resistant”. Similarly, maize lines with scores of three or less were collectively considered susceptible. Only lines that could be reliably placed into the two groups were used. Once a line is included in the “Resistant” or “susceptible” group, it was treated as an equal in that group. The actual quantitative ratings were also used for association test. In addition to this test, the identity by descent information was used to confirm the resistant allele origin at the highest associated markers.

In the study, 85 maize lines were identified that were considered resistant in the phenotypic spectrum; these plants formed the “RESISTANT” group. Also, 35 maize lines were identified that were judged to be susceptible to MRCV; these strains formed the “SUSCEPTIBLE” group.

### Maize Genotyping

Each of the tolerant and susceptible lines was genotyped with a set of 63 SNP markers that span the QTL region at Chromosome 2 using techniques well known in the art. The genotyping protocol consisted of collecting young leaf tissue and isolating genomic DNA from pooled tissue of each inbred. The maize genomic DNA was extracted by the CTAB method, as described in Maroof et al. (1984) Proc. Natl. Acad. Sci. (USA) 81:8014-8018.

The isolated genomic DNA was then used in PCR reactions using amplification primers specific for a large number of markers that covered the QTL region. SNP-type markers were genotyped using an ASH protocol.

The underlying logic is that markers with significantly different allele distributions between the resistant and susceptible groups (i.e., non-random distributions) might be associated with the trait and can be used to separate them for purposes of marker assisted selection of maize lines with previously uncharacterized or characterized resistance or susceptibility to MRCV. The present analysis examined one marker locus at a time and determined if the allele distribution within the resistant group is significantly different from the allele distribution within the susceptible group. This analysis compares the plants' phenotypic score with the genotypes at the target loci.

### Results

Tables 1 and 2 list maize markers that demonstrated linkage disequilibrium with the MRCV resistant/susceptibility phenotype. Also indicated in those tables is where the markers are located and their approximate map position relative to other known markers, given in cM, with position zero being the first (most distal) marker known at the beginning of the chromosome. These map positions are not absolute, and represent an estimate of map position. The statistical probabilities that the marker allele and tolerance phenotype are segregating independently are reflected in the adjusted probability values.

Tables 6 and 7 provide the PCR primer sequences that were used to genotype these marker loci.

The non-random distribution of alleles between the resistant and susceptible plant groups at the various marker loci in Tables 1 and 2 is good evidence that a QTL influencing MRCV resistance is linked to these marker loci. Considering that most of the inbreds of this set correspond to a specific breeding program (argentine breeding program), it is expected that Appliants have found linkage disequilibrium with other markers on flanking regions of the gene. The highest associated markers corresponded to the previously considered preferred markers.

As well known in the art, the level of association of target markers to a trait of interest will be determined by the level of linkage disequilibrium at the target region on that specific set of genetic materials. Table 17 below shows the level of association across the target region between the genotypic data of SNPs markers and the response to MRCV.

TABLE 17

| Chr | Pos | Marker | b0 | b1 | F(1, n-2) | pr(F) | MRCV Trait |
|---|---|---|---|---|---|---|---|
| 2 | 64.05 | MZA625-29-A | 1.612 | −0.322 | 95.712 | 0 | **** |
| 2 | 64.05 | MZA625-30-A | 1.602 | −0.326 | 92.373 | 0 | **** |
| 2 | 65.99 | MZA16656-8-A | 1.613 | −0.255 | 44.107 | 0 | **** |
| 2 | 65.99 | MZA16656-19-A | 1.571 | −0.344 | 105.781 | 0 | **** |
| 2 | 65.99 | MZA15490-137-A | 1.724 | −0.189 | 23.834 | 0 | **** |
| 2 | 65.99 | MZA15490-138-A | 1.731 | −0.179 | 20.667 | 0 | **** |
| 2 | 65.99 | MZA15490-801-A | 1.727 | −0.172 | 19.222 | 0 | **** |
| 2 | 65.99 | MZA2038-71-A | 1.702 | −0.045 | 1.095 | 0.298 | |
| 2 | 65.99 | MZA2038-76-A | 1.691 | −0.063 | 1.987 | 0.161 | |
| 2 | 65.99 | MZA11826-801-A | 1.673 | −0.098 | 4.614 | 0.034 | * |
| 2 | 65.99 | MZA11826-27-A | 1.681 | −0.092 | 4.315 | 0.04 | * |
| 2 | 65.99 | MZA11826-803-A | 1.673 | −0.113 | 6.286 | 0.014 | * |
| 2 | 65.44 | MZA9105-8-A | 1.576 | −0.226 | 22.461 | 0 | **** |
| 2 | 65.44 | MZA9105-6-A | 1.681 | −0.081 | 3.452 | 0.066 | |

In order to evaluate the effect of the allelic variation at this QTL at the hybrid level, a set of 371 hybrids (heterogenous genetic backgrounds) was characterized according to the presence of one (heterozygous for the QTL) or two resistant alleles (homozygous for the QTL) from the parent lines. A positive and additive effect of the resistant allele at the major QTL was observed on the hybrid combinations; no maternal effects were observed. Table 18 below shows the field performance of hybrids with different genotypes at the major QTL.

TABLE 18

| | Number of | | |
|---|---|---|---|
| Hybrid genotype at major QTL | hybrids | MRCVSC | Category |
| AA, homozygous susceptible allele | 65 | 3.8 | Susceptible |
| BA, heterozygous, female | 121 | 4.41 | Resistant |
| resistant allele | | | |
| AB, heterozygous, male resistant | 96 | 4.46 | Resistant |
| allele | | | |
| BB, homozygous resistant allele | 89 | 4.76 | Resistant |

### Discussion

There are a number of ways to use the information provided in this analysis for the development of improved maize varieties. One application is to use the associated markers (or more based on a higher probability cutoff value) as candidates for mapping QTL in specific populations that are segregating for plants having tolerance to MRCV infection. In this application, one proceeds with conventional QTL mapping in a segregating population, but focusing on the markers that are associated with MRCV infection tolerance, instead of using markers that span the entire genome. This makes mapping efforts more cost-effective by dramatically reducing lab resources committed to the project. For example, instead of screening segregating populations with a large set of markers that spans the entire genome, one would screen with only those few markers that met some statistical cutoff in the allele association study. This will not only reduce the cost of mapping but will also eliminate false leads that will undoubtedly occur with a large set of markers. In any given cross, it is likely that only a small subset of the associated markers will actually be correlated with tolerance to MRCV infection. Once the few relevant markers are identified in any tolerant parent, future marker assisted selection (MAS) efforts can focus on only those markers that are important for that source of tolerance. By pre-selecting lines that have the allele associated with tolerance via MAS, one can eliminate the undesirable susceptible lines and concentrate the expensive field testing resources on lines that have a higher probability of being resistant to MRCV infection.

### Example 6

### QTL Evaluation on F3 Breeding Populations

Marker associations are widely used methods to identify genetic loci that co-segregate with a desired phenotype. By identifying such genetic loci, marker assisted selection (MAS) can be used to improve the efficiency of breeding for improved maize inbreds and hybrids.

### Maize Lines

Old scheme of breeding was based on the traditional pedigree based method of making F1 crosses and deriving several self generations (F2, F3, F4, etc.). With the goal of checking the importance of the positive and negative alleles at the major QTL for MRCV resistance in a specific set of argentine breeding materials, these steps were followed: a) Selection of resistant parents whose resistance is expected to be based on the major MRCV1; b) Selecting a total of 2372 F3 families originated from multiple breeding crosses; c) Making two groups of F3 families, a first group, based on crosses between parents without the positive alleles of the major QTL and a second group with both parents harboring the positive allele at the major QTL. Fingerprint data at the main QTL region and identity by descent information was used to define QTL segregating and QTL fixed populations (positive/negative). MZA16656 and/or flanking markers were the key markers to define the presence of MRCV1 positive allele.

The total number of individuals located on these groups was 2372. Fingerprint data at main QTL region and identity by descent information was used to define QTL segregating and QTL fixed populations.

### Phenotypic Scoring

Phenotypic scoring of each of the F3 populations was based on sets of phenotypic data collected from the field on one crop season.

### Maize Genotyping

Individual F3 families were not genotyped. Genotype at major QTL on each individual F3 was estimated according to the specific alleles on both parents. If both parents in a specific F3 population harbor the positive allele at MRCV1, all the progenies from that cross were considered as having the positive allele. If both parents in a specific F3 population harbor the negative allele at MRCV1, all the progenies from that cross were considered as having the negative allele. Standard software was used to the marker ANOVA analysis.

### Results

### QTL Marker Analysis

The present study supported the conclusion that a major chromosome interval correlated with QTL associated with resistance/susceptibility to MRCV infection when populations from crosses fixed at MRCV major QTL on chromosome 2 were selected “a priori”.

### Single Marker ANOVA

Using marker ANOVA, there are a number of markers showing association with the tolerance phenotype at a confidence level of P=0.05 or better, as shown in Tables 1 and 2. Some of the markers identified in the marker ANOVA analysis show a concordance of observations with the association mapping, where the different approaches identify the same markers. For example, there are markers at the region from 55 to 70 cM on chromosome 2 identified by both marker regression and association mapping.

### Discussion/Conclusions

This present study has identified chromosome intervals and individual markers that correlate with MRCV resistance. Markers that lie within these intervals are useful for use in MAS, as well as other purposes. In this example, Applicants evaluated the effect on MRCV resistance of the allelic variation at MRCV1, and there was a clear the association between this allelic variation and the expected phenotype on a high number of F3 progenies.

Table 19 below shows the F-test of the model where the Source is QTL and two levels of the source were considered: Level AA: F3 populations with fixed susceptible alleles at target region (position 65.99 or inferred by flanking markers) and Level BB: F3 populations with fixed resistant alleles at target region (position 65.99 or inferred by flanking markers).

TABLE 19
One-Way ANOVA: MRCVSC score versus QTL

| Source | DF | SS | MS | F | P |
|---|---|---|---|---|---|
| QTL | 1 | 1656.41 | 1656.41 | 1383.90 | 0.000 |
| Error | 2370 | 2836.69 | 1.20 | | |
| Total | 2371 | 4493.10 | | | |

S = 1.094;
R-Sq = 36.87%;
R-Sq(adj) = 36.84%
References:
DF. Degree of freedom.
SS. Square Sum.
MS. Mean square.
F. F value.
P. Probability value.

Table 20 below shows a mean test where level AA and level BB represents the allelic variation at target QTL and according to the model included in Table 19. Phenotypic mean for level AA was 3.42 (MRCV susceptible category) and phenotypic mean for level BB was 5.138 (MRCV resistant category).

TABLE 20

| Level | N | Mean | StDev |
|---|---|---|---|
| AA | 1462 | 3.420 | 1.048 |
| BB | 910 | 5.138 | 1.164 |

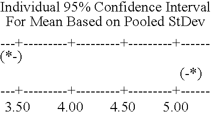

Pooled StDev = 1.094
References:
N: Number of F3 families.
Mean: Phenotypic mean of each level.
StDev: Standard deviation.

### Example 7

### High-Resolution Gene Mapping and Near-Isogenic Lines

High-resolution gene mapping by progeny testing of homozygous recombinant plants was undertaken for high resolution positioning of the MRCV resistance genes. QTL interval mapping and a single marker regression analysis were performed to identify maize chromosome intervals and genetic markers (respectively) that are associated with resistance and enhance resistance to MRCV infection. QTL mapping and marker regression are widely used methods to identify genetic loci that co-segregate with a desired phenotype. By identifying such genetic loci, marker assisted selection (MAS) can be used to improve the efficiency of breeding for improved maize inbreds and hybrids.

### Maize Lines

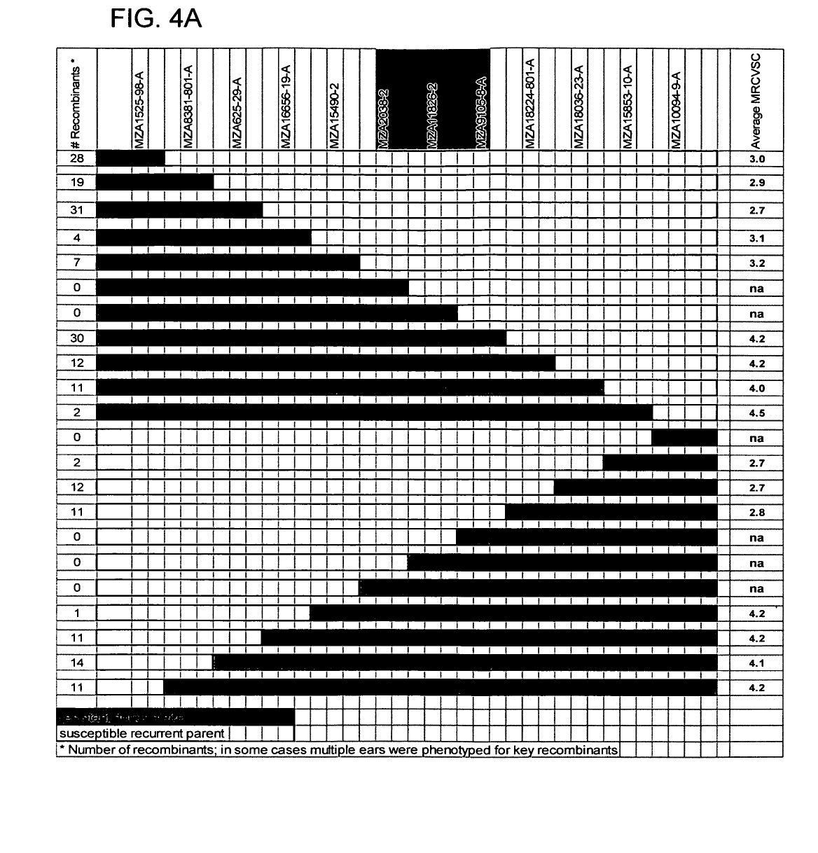

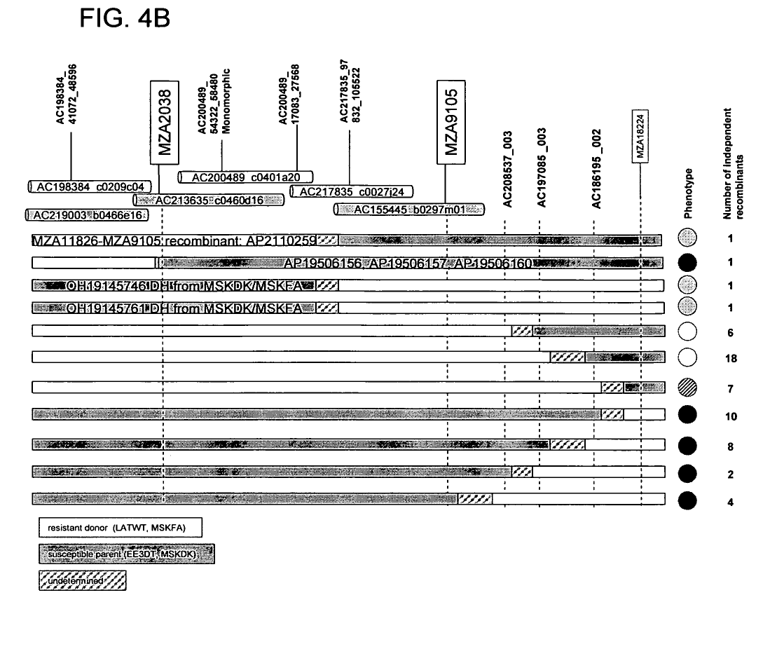

One main population for high-resolution gene mapping of MRCV resistance was created from the cross of inbreds PH7WT and PH3DT. Another population for fine mapping in an independent source was created from the cross of inbreds PH9TJ and PH890. The PH7WT×PH3DT population consisted of 256 BC5F3 families generated by selfing and fixing selected recombinant BC5 plants from a total of 3000 BC5 plants harboring a heterozygous fragment at the region from 50 to 80 cM on chromosome 2. This strategy permitted coverage with recombinants of the whole QTL region (Tables 7 and 8, and FIG. 4A and FIG. 4B). The PH9TJ×PH890 population consisted of 245 BC3F3 families generated by: a) Crossing selected BC2F4 plants homozygous for the major QTL on chromosomes 2 and 5 with PH890 (susceptible parent); b) Making two self generations to advance to fixed BC3F3 families.

Table 21 shows the number of BC5F3s recombinants generated from the cross PH3DT×PH7WT. An expected Kb size for each marker interval is also included. Table 22 shows the number of BC5F3s recombinants generated from the cross PH3DT×PH7WT. A comparison with the first estimation of gene content is included.

TABLE 21

| | | | Estimated size of each | | |
|---|---|---|---|---|---|
| | Pioneer Genetic | | interval (sequence | Total | |
| Marker | Map* | Fingerprint bands** | data) | Recombinants | BC5F3s |
| MZA625 | 64.05 | | | | |
| MZA16656 | 65.99 | 152 | Higher than 100 Kb | 76 | 59 |
| MZA15451 | 65.99 | — | 10 Kb | — | — |
| MZA15490 | 65.99 | — | Less than 100 Kb | 3 | 2 |
| MZA2038 | 65.99 | 0 | Less than 20 Kb | 1 | 1 |
| MZA11826 | 65.99 | 33 | Higher than 20 Kb | 0 | 0 |
| MZA9150-8-A | 65.44 | 88 | Higher than 50 Kb | 0 | 0 |
| MZA18224-801-A | 68.80 | 400 | Higher than 165 Kb | 51 | 44 |

*Marker ordered according to sequencing, physical, and recombination. Pioneer genetic map is included only as reference.
**Number of fingerprint bands between pairs of markers.

TABLE 22

| | PHD | UC7 PCO | | | | |
|---|---|---|---|---|---|---|
| PHD | Map | Vs. Myriad | Locus | | | |
| Chr | Pos | Amplicons | Order | Working Maize Gene ID | Annotation Summary | Recombinants |
| 2 | 64.05 | MZA625 | Loc_029 | AC191302_5part | Transcription Factor | 59 |
| | | | Loc_028 | AC191302_3 | Putrescine-binding | |
| | | | | | protein; Hypothetical | |
| | | | | | protein | |
| | | | Loc_027 | pco600856 | Putative L-ascorbate | |
| | | | | | peroxidase | |
| | | | Loc_025 | pco530474 | Plastid development | |
| | | | | | protein; DAG | |
| | | | Loc_024 | pco593067 | Hypothetical protein; | |
| | | | | | Vacuolar ATP synthase | |
| | | | | | subunit? | |
| | | | Loc_023 | AC191302_6 | Hypothetical protein | |
| | | | Loc_022 | Inferred by rice and sorghum | Hypothetical protein | |
| | | | Loc_021 | pco641713 | Hypothetical protein | |
| | | | Loc_016 | pco591841 | Growth regulating factor | |
| | | | Loc_015 | Genomic_PCO622600_PCO666161 | G protein-coupled | |
| | | | | | receptor 89C (Homo | |
| | | | | | sapiens) | |
| 2 | 65.99 | MZA166656 | Loc_014 | pco638426 | Major intrinsic protein; | |
| | | | | | NIP; BREVIS RADIX | |
| | | | | | like 1 | |
| | | | Loc_013 | pco514627 | Hypothetical protein | 2 |
| 2 | 65.30 | MZA15451 | Loc_012 | pco588936 | Alternative oxidase | |
| | | | | | AOX3 | |
| | 65.99 | MZA15490 | | pco642154 | Alternative oxidase | |
| | | | | | AOX2 | |
| | | | Loc_010 | Inferred by rice and sorghum | Hypothetical protein | 1 |
| | | | Loc_009 | pco644442 | Myb-like; 2-component | |
| | | | | | response regulator | |
| 2 | 65.99 | MZA2038 | Loc_008 | pco641455 | Clathrin interactor; | |
| | | | | | Epsin; Hypothetical | |
| | | | | | protein | |
| | | | Loc_007 | pco640541 | CDC20 WD-repeat | 0 |
| | | | | | protein | |
| | | | Loc_006 | pco651091 | Cobalamin synthesis | |
| | | | | | protein | |
| | | MZA11826 | | | | |
| | | | Loc_005 | pco571541 | Hypothetical protein | |
| | | | Loc_004 | pco525409 | Scramblase | |
| | | | Loc_003 | pco553755 | Hypothetical protein | |
| | | | Loc_002 | pco644099 | Hypothetical protein | |
| 2 | 65.44 | MZA9105 | Loc_001 | pco588179 | Receptor protein kinase | |
| | | AC208537(CAP) | | | | 13 |
| | | AC197085(CAP) | | | | 2 |
| | | MZA18224 | | | | 23 |

BC5F3 near-isogenic lines (NIL) harboring allelic variation at the region of the preferred markers (MZA16656, MZA15451, MZA15490, MZA2038, MZA11826 and MZA9105) were generated by marker assisted selection from the PH7WT×PH3DT cross. The NILs were generated by introgressing the QTL region from PH7WT into PH3DT, cleaning the genetic background, and selecting specific recombinants at the region of the preferred markers. By selfing individual BC5F2 plants harboring a heterozygous fragment at the region of the preferred markers, negative and positive near-isogenic lines were derived, and the QTL was treated as a single Mendelian factor.

### Phenotypic Scoring

Phenotypic scoring of each of the BC5F3 families from PH7WT×PH3DT cross and the 245 BC3F3 families from PH9TJ×PH890 cross and parents was based on sets of phenotypic data collected from the field (field experiments under natural infection, Córdoba Province, Argentina) on one crop season.

In addition to the phenotyping scoring, the specific isolines at the region of preferred markers were characterized by ELISA test for virus in the Buenos Aires Province, Argentina.

### Maize Genotyping

Maize BC5F3 progeny from PH7WT×PH3DT cross and BC3F3 from the PH9TJ×PH890 cross were genotyped by using polymorphic SNPs at the QTL region on chromosome 2 (see Example 2). In addition, two CAPS markers were designed and used to genotype the BC5F3 progenies; these two CAPS markers were positioned to the interval MZA9105 to MZA18224. In the case of the PH9TJ×PH890 cross, additional markers were positioned on the chromosome 5 QTL. The BC5F3s from PH7WT×PH3DT cross were subjected to background cleaning at BC3 stage, especially at chromosome 5 QTL. The BC3F3s from PH9TJ×PH890 cross were subjected to background cleaning at BC2 stage.

Windows QTL Cartographer (up-to-date version according the date of QTL mapping) was used for both the marker regression analysis and QTL interval mapping. LOD scores (logarithm of the odds ratio) were estimated across the target regions according the standard QTL mapping procedures.

Mean scores were used in QTL interval mapping. The LOD threshold was 2.5. A confidence interval was estimated for each QTL. The positions obtained are then plotted as a histogram overlaying the interval mapping figure.

As these population were generated by marker assisted selection (not random events of recombination), marker regression analysis was considered as powerful as interval mapping analysis.

### Results

### QTL Interval Mapping

The present study identified a single chromosome interval that correlated with QTLs associated with resistance/susceptibility to MRCV infection. The QTL were identified using the field data. One major, significant QTL was located on linkage group 2 at the position of “preferred markers” on the high resolution mapping pops from PH7WT×PH3DT and PH9TJ×PH890 crosses. The additional QTL on chromosome 5 from PH9TJ×PH890 cross was not significant in this analysis.

### Single Marker Regression

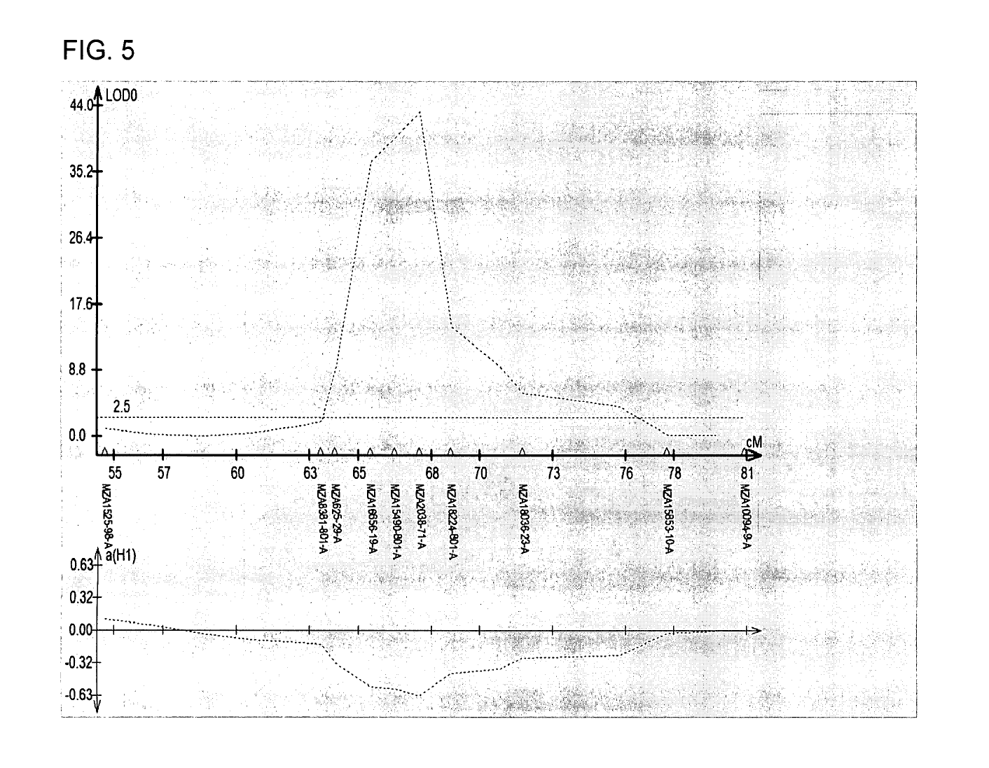

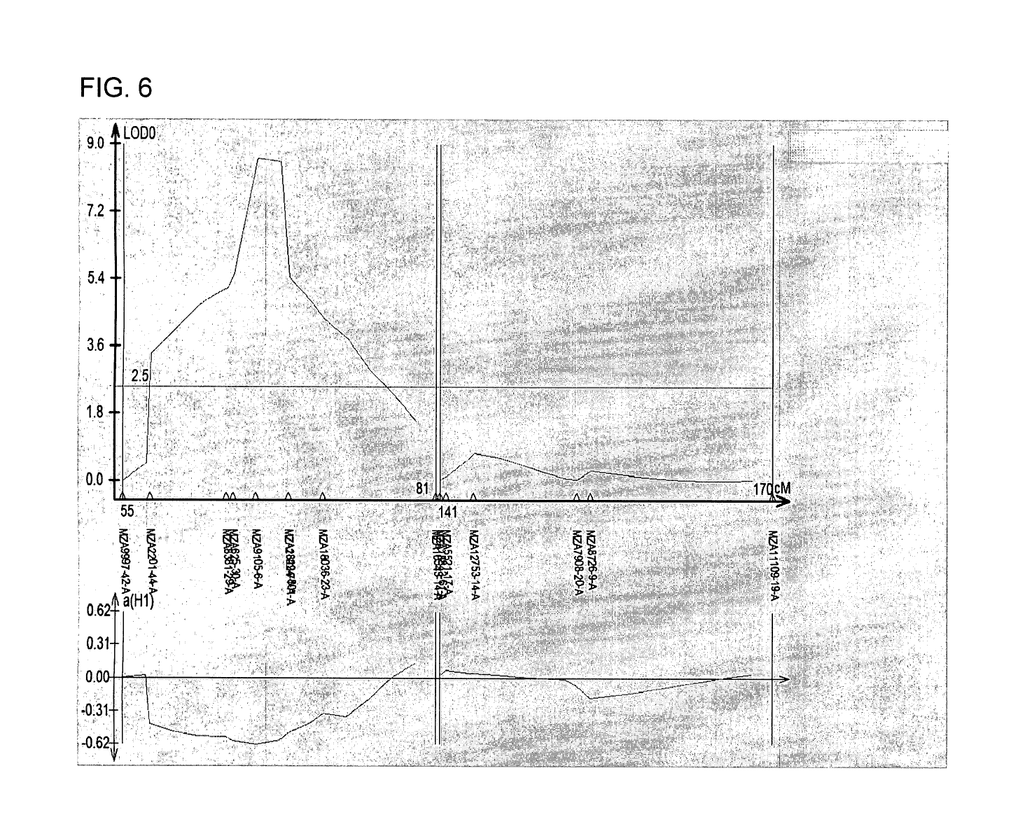

Using single marker regression, there are a number of markers showing association with the resistant phenotype at a confidence level of P=0.05 or better, as shown in Tables 23 and 24. The markers identified in the marker regression analysis show a high resolution gene position for the target QTL, coincident with the position of the preferred markers. See Table 23 for marker regression analysis (MRCVSC=MRCV phenotypic score) and FIG. 5 for interval mapping for the PH7WT×PH3DT cross. See Table 24 for a QTL marker regression analysis for the PH9TJ×PH890 cross on specific QTL at chromosomes 2 and 5 (MRCVSC=MRCV phenotypic score) and FIG. 6 for interval mapping for the PH7WT×PH3DT cross.

TABLE 23

| | Po- | | | | | |
|---|---|---|---|---|---|---|
| Marker | sition | b0 | b1 | F(1, n-2) | pr(F) | MRCVSC |
| MZA1525-98-A | 54.62 | 3.423 | −0.330 | 9.144 | 0.003 | ** |
| MZA8381-801-A | 63.47 | 3.337 | −0.429 | 15.852 | 0 | *** |
| MZA625-29-A | 64.05 | 3.362 | −0.422 | 16.218 | 0 | *** |
| MZA16656-19-A | 65.99 | 3.300 | −0.589 | 38.934 | 0 | **** |
| MZA15490- | 65.99 | 3.331 | −0.597 | 42.193 | 0 | **** |
| 137-A | | | | | | |
| MZA2038-71-A | 65.99 | 3.377 | −0.628 | 51.838 | 0 | **** |
| MZA11826- | 65.99 | 3.377 | −0.628 | 51.838 | 0 | **** |
| 801-A | | | | | | |
| MZA9105-8-A | 65.44 | 3.377 | −0.628 | 51.838 | 0 | **** |
| AC208537_003 | | 3.448 | −0.257 | 5.276 | 0.025 | * |
| AC197085_003 | | 3.472 | −0.135 | 1.331 | 0.253 | |
| MZA18224- | 68.80 | 3.403 | 0.095 | 0.595 | 0.443 | |
| 801-A | | | | | | |

TABLE 24

| Marker | Chr | Pos | b0 | b1 | −2ln(L0/L1) | F(1, n-2) | pr(F) | MRCVSC |
|---|---|---|---|---|---|---|---|---|
| MZA9997-42-A | 2 | 54.56 | 3.891 | −0.460 | 37.209 | 39.855 | 0 | **** |
| MZA2201-44-A | 2 | 56.95 | 3.828 | −0.334 | 24.549 | 25.610 | 0 | **** |
| MZA8381-29-A | 2 | 63.47 | 3.939 | −0.465 | 33.536 | 35.647 | 0 | **** |
| MZA625-30-A | 2 | 64.05 | 3.907 | −0.485 | 38.029 | 40.803 | 0 | **** |
| MZA9105-6-A | 2 | 66.00 | 3.887 | −0.547 | 51.358 | 56.671 | 0 | **** |
| MZA2349-71-A | 2 | 68.80 | 3.907 | −0.534 | 49.417 | 54.307 | 0 | **** |
| MZA18224-801-A | 2 | 68.80 | 3.902 | −0.531 | 48.959 | 53.751 | 0 | **** |
| MZA18036-23-A | 2 | 71.75 | 3.906 | −0.505 | 43.106 | 46.746 | 0 | **** |
| MZA10543-14-A | 2 | 81.45 | 3.934 | −0.054 | 0.332 | 0.320 | 0.572 | |
| MZA18843-61-A | 5 | 141.08 | 3.897 | 0.004 | 0.003 | 0.003 | 0.958 | |
| MZA5521-17-A | 5 | 141.62 | 3.895 | 0.009 | 0.014 | 0.014 | 0.906 | |
| MZA12753-14-A | 5 | 143.95 | 3.886 | 0.044 | 0.333 | 0.331 | 0.566 | |
| MZA7908-20-A | 5 | 152.87 | 3.903 | −0.046 | 0.311 | 0.309 | 0.579 | |
| MZA8726-9-A | 5 | 154.05 | 3.901 | −0.050 | 0.385 | 0.382 | 0.537 | |
| MZA11109-19-A | 5 | 169.77 | 3.895 | 0.036 | 0.156 | 0.155 | 0.694 | |

### Near Isogenic Lines

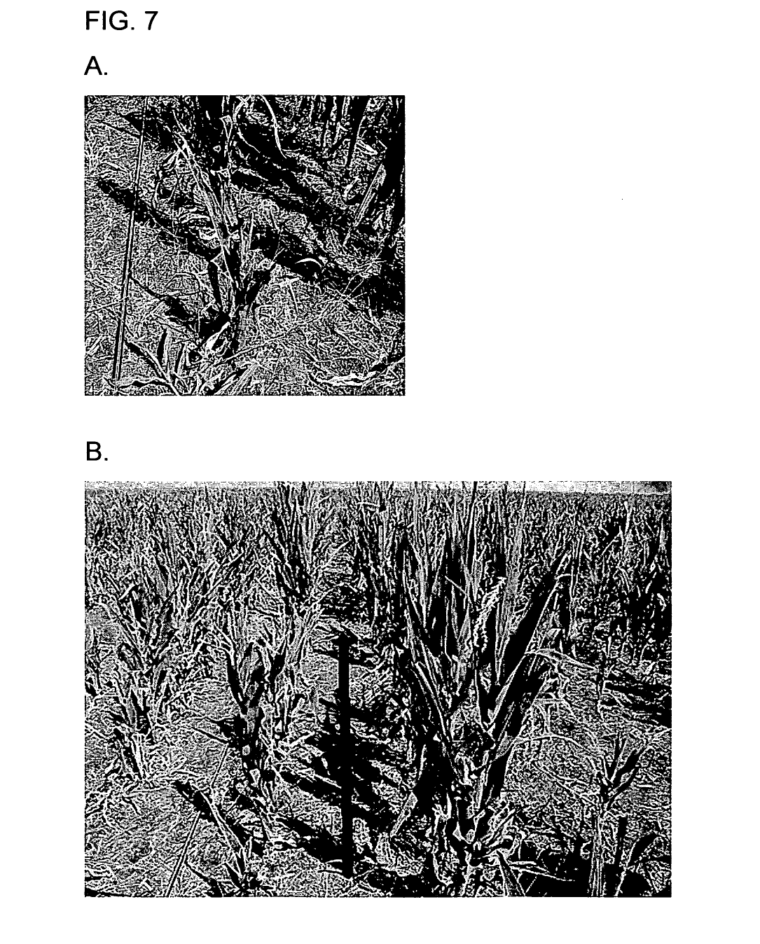

The near isogenic lines harboring allelic variation at the region of preferred markers showed a significant difference in their response to the disease in Córdoba Province (FIG. 7A and FIG. B). Table 25 shows the genotype of SNPs at the region of preferred markers for the near isogenic lines (negative isoline=susceptible haplotype; positive isoline=resistant haplotype); the introgressed fragment is represented by the SNP polymorphics at markers from MZA16656-19-A to MZA9105-8-A while flanking monomorphics markers MZA625-30-A and MZA18224-801-A represent the susceptible haplotype on both near isogenic lines. The ELISA test for virus (samples from Buenos Aires Province) showed 0% of plants positive for virus in the isolines harboring the resistant allele, while 38% of plants were positive for virus in the isolines harboring the susceptibility allele.

TABLE 25

| Isoline | MZA625- | MZA16656- | MZA15490- | MZA15490- | MZA2038- | MZA2038- | MZA11826- | MZA11826- | MZA9105- | MZA18224- |
|---|---|---|---|---|---|---|---|---|---|---|
| | 30-A | 19-A | 801-A | 137-A | 71-A | 76-A | 801-A | 803-A | 8-A | 801-A |
| | 64.05 | 65.99 | 65.99 | 65.99 | 65.99 | 65.99 | 65.99 | 65.99 | 65.44 | 68.8 |
| Neg- | C | A | C | A | T | C | G | T | G | A |
| ative | | | | | | | | | | |
| Positive | C | G | G | C | A | T | A | C | A | A |

Note:
ELISA test was not performed on materials planted in Cordoba Province (the disease pressure was higher than in Buenos Aires Province). However, the presence of enations (a specific symptom of Fijivirus) on both resistant and susceptible materials in Córdoba Province indicates the presence of the virus in the plants.

### Discussion/Conclusions

This present study has identified chromosome intervals and individual markers that correlate with MRCV resistance. Markers that lie within these intervals are useful for use in MAS, as well as other purposes. The high resolution gene position facilitates the cloning of the target QTL.

### Example 8

Gene positioning, sequencing and candidate genes Sequencing, genetic and physical information for the region of the preferred markers was integrated to characterize the target region. Information from independent approaches (recombination data, association analysis and conservative fragments) was used to identify a specific interval for the generation of additional sequencing data.

### Maize Lines and Phenotypic Scoring

Maize lines were phenotypically scored based on their degree of resistance to MRCV infection (in contrast to simple categorization of “tolerant” or “susceptible”). The plant varieties used in the analysis were from diverse sources, including elite germplasm, commercially released cultivars and other public varieties. The collections comprised 883 maize lines. The lines used in the study had a broad maturity range varying from CRM (comparative relative maturity) 90 to CRM 140, representing the main inbreds of Pioneer germplasm.

The degree of plant resistance to MRCV infection varied widely, as measured using a scale from one (highly susceptible) to nine (highly resistant). Generally, a score of two (2) indicated the most susceptible strains, a score of four (4) was assigned as the threshold to consider a plant susceptible or resistant (less than 4, susceptible; 4 or higher is resistant) and a score of seven (5-7) was assigned to the most resistant lines. Resistance scores of eight (8) and nine (9) were reserved for resistance levels that are very rare and generally not observed in existing germplasm. If no disease was present in a field, no resistance scoring was done. However, if a disease did occur in a specific field location, all of the lines in that location were scored. Scores for test strains were accumulated over multiple locations and multiple years, and an averaged (e.g., consensus) score was ultimately assigned to each line.

Resistance scores for part of the 883 inbred collections were collected over several growing seasons (394 inbreds were evaluated at the same time in the growing season). Data collection was typically done in one scoring after flowering time.

### Maize Genotyping

A collection of 883 maize lines was analyzed by DNA sequencing at 4000-10000 genes (genetic loci). SNP variation was used to generate specific haplotypes across inbreds at each locus. This data was used for identifying associations between alleles and MRCV resistance at genome level.

### Maize Pedigree-Resistance Sources

A Pioneer pedigree database was used to understand the relationship between inbreds and haplotypes. This database contains the pedigree relationship between Pioneer inbreds since 1919. In the case of public inbreds, public information about pedigree and origins was incorporated to understand inbred and haplotype relationship. A list of key founders representing sources of resistance and susceptibility to MRCV in Pioneer germplasm (including public lines) was created by using pedigree, phenotypic and genotypic data. Most of the susceptible inbreds trace back to a specific set of haplotypes from U.S. germplasm (Public lines as B37, B73, B14, OH07, C103 and Pioneer inbreds 165 and 938); an exception is PH26N coming from tropical germplasm.

### Gene Positioning

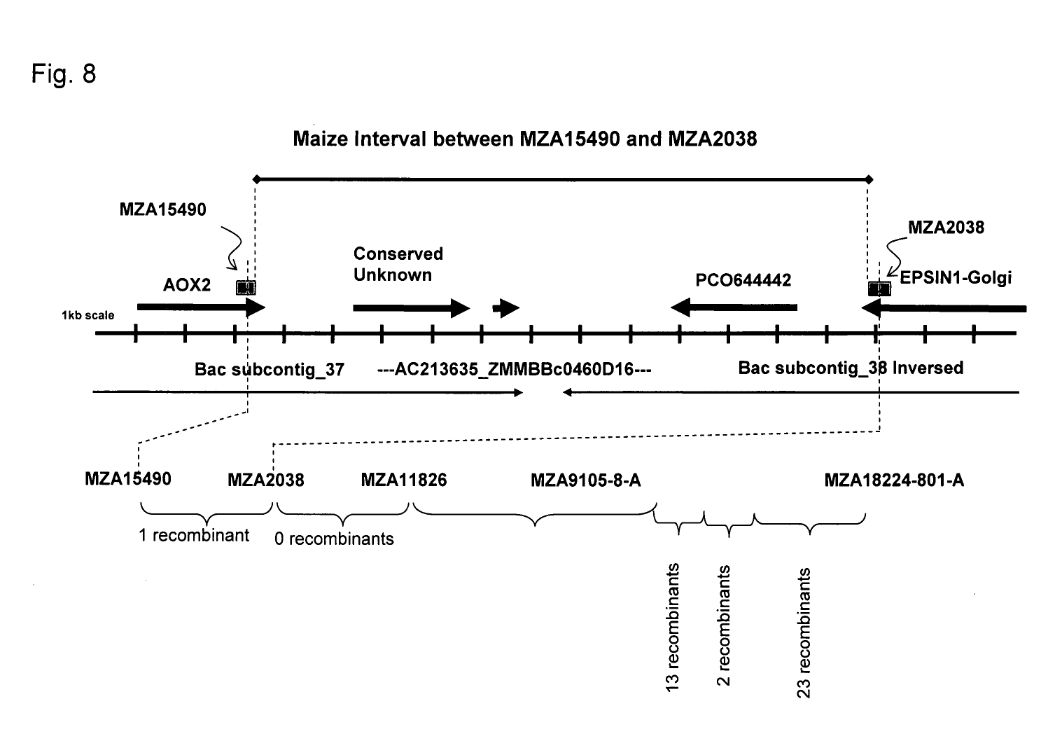

The interval between markers MZA15490 and MZA2038 (FIG. 8) was considered as a candidate region for studying the allelic diversity at the resistance gene region. This region was selected by using information from:
a) Recombinants. The positioning by recombinants was showed in Example 7. The phenotypic data for two recombinants at MZA16656 to MZA15490 interval and one recombinant at MZA15490 to MZA2038 interval was used to delimitate the left side of the gene position. From recombination population, there were no available recombinants at the region MZA2038-MZA11826-MZA9105.
b) Genotypic and phenotypic information across inbreds from Pioneer germplasm was used to detect a conservative fragment across resistant/susceptible inbreds. The detection of a conservative fragment was performed inside specific pedigrees and across multiple independent founders when enough conservative SNPs were available across independent founders.

### Natural Allelic Diversity and Founder Relationship

The interval MZA15490 to MZA2038 was selected for allelic diversity analysis because of the high probability of harboring a candidate gene or the high linkage disequilibrium with a candidate gene. As the full sequence at MZA15490 to MZA2038 interval is available for B73 (B73=274) line, a group of 13 small sequence fragments were targeted for sequencing in a set of tester's lines. The tester's lines (Table 26) included: a) some of the key resistant and susceptible inbreds and haplotypes; b) the resistant and susceptible parents from the mapping populations PH7WT×PH3DT and PH9TJ×PH890; c) key recombinants from the inbred set and recombination population (PHG63 and a recombinant at MZA15490 to MZA2038 interval).

Table 26 shows a list of tester's lines including sources of resistance and susceptibility to MRCV in Pioneer germplasm and a recombinant at MZA15490-MZA2038 interval.

TABLE 26

| | | Expected | |
|---|---|---|---|
| Inbred | Phenotype | haplotype | n |
| PH9TJ | Resistant | PH9TJ | 1 |
| PHJ40 | Resistant | PHJ40 | 5 |
| PHGD3 | — | PHGD3 | 2 |
| 383 | Resistant | — | 1 |
| PHG63 | Resistant | 630 | 14 |
| 630 | Resistant | 630 | 14 |
| PH7WT | Resistant | 630 | 14 |
| PHR33 | Resistant | PHR33 | 1 |
| 501 | — | 501 | — |
| PH467 | Resistant | PH467 | 1 |
| PHDG9 | Resistant | PHDG9 | 1 |
| PHK09 | Resistant | PHK09 | 1 |
| 274 | Susceptible | 274 | 47 |
| 1047 | Susceptible | 1047 | 23 |
| PH26N | Susceptible | PH26N | 1 |
| PH3DT | Susceptible | 274 | 47 |
| PH890 | Susceptible | 1047 | 23 |
| 165 | Susceptible | 165 | 33 |
| 661 | Susceptible | PHAN0 | 93 |
| PHR03 | Susceptible | PHAN0 | 93 |
| PHK56 | Susceptible | PHAN0 | 93 |
| PHN47 | Susceptible | PHN47 | 1 |
| PHNV8 | Susceptible | PHNV8 | 1 |
| ap19506156 | Susceptible | Recombinant | 1 |
| ap19506157 | Susceptible | Recombinant | 1 |
| ap19506160 | Susceptible | Recombinant | 1 |
| 157 | Susceptible | 625 | 7 |
| 625 | Susceptible | 625 | 7 |
| PHKP5 | — | PHKP5 | 1 |

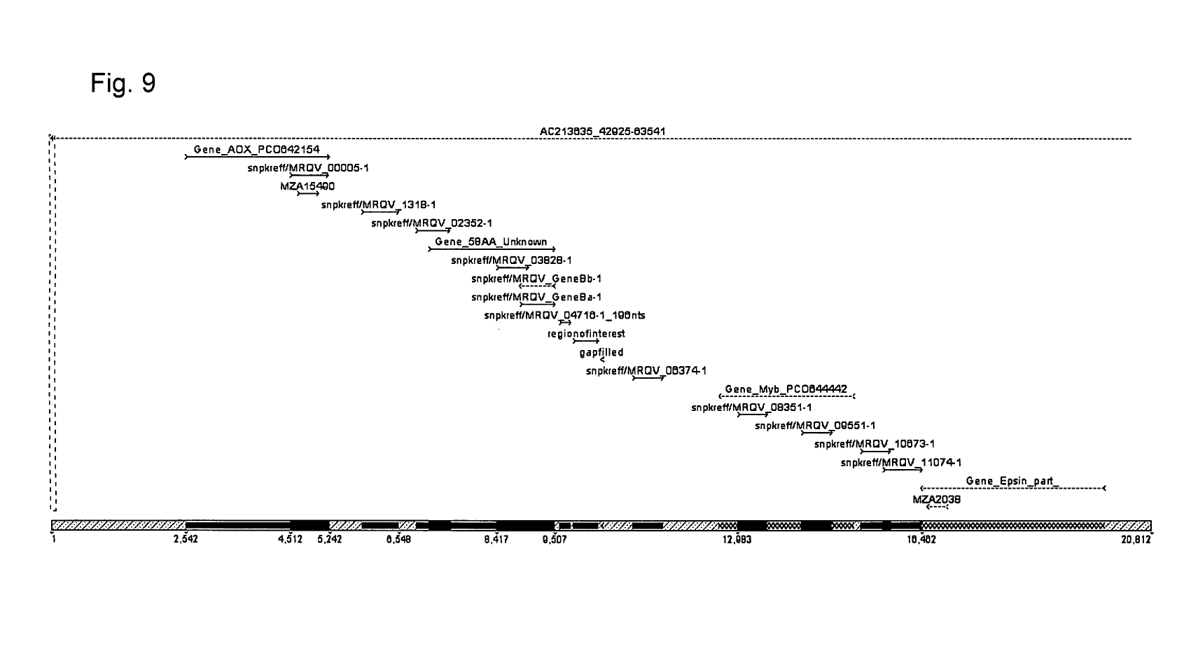

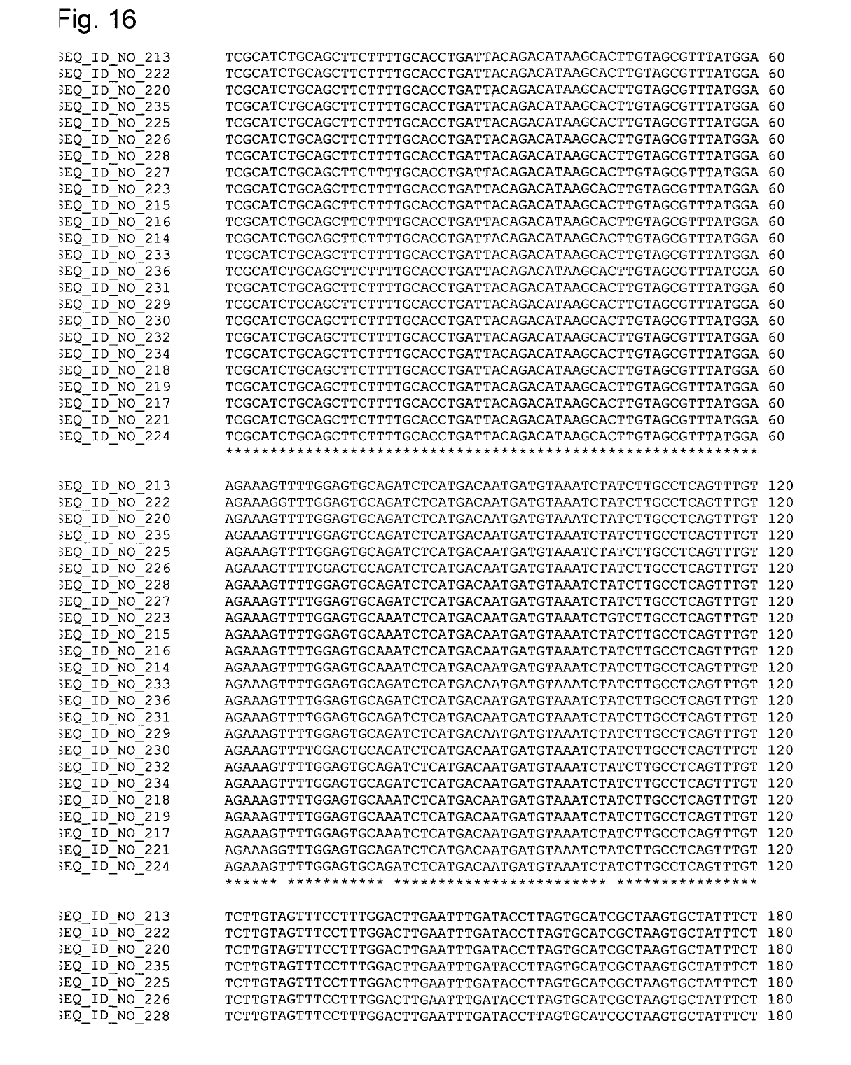

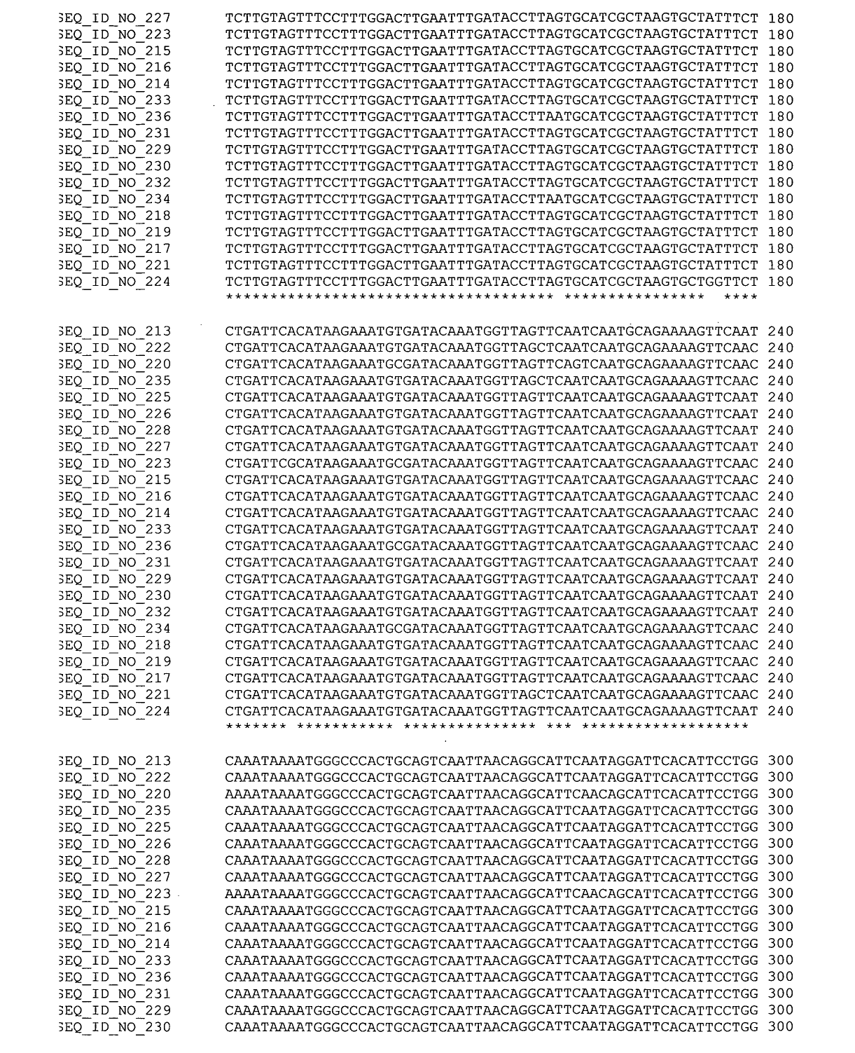

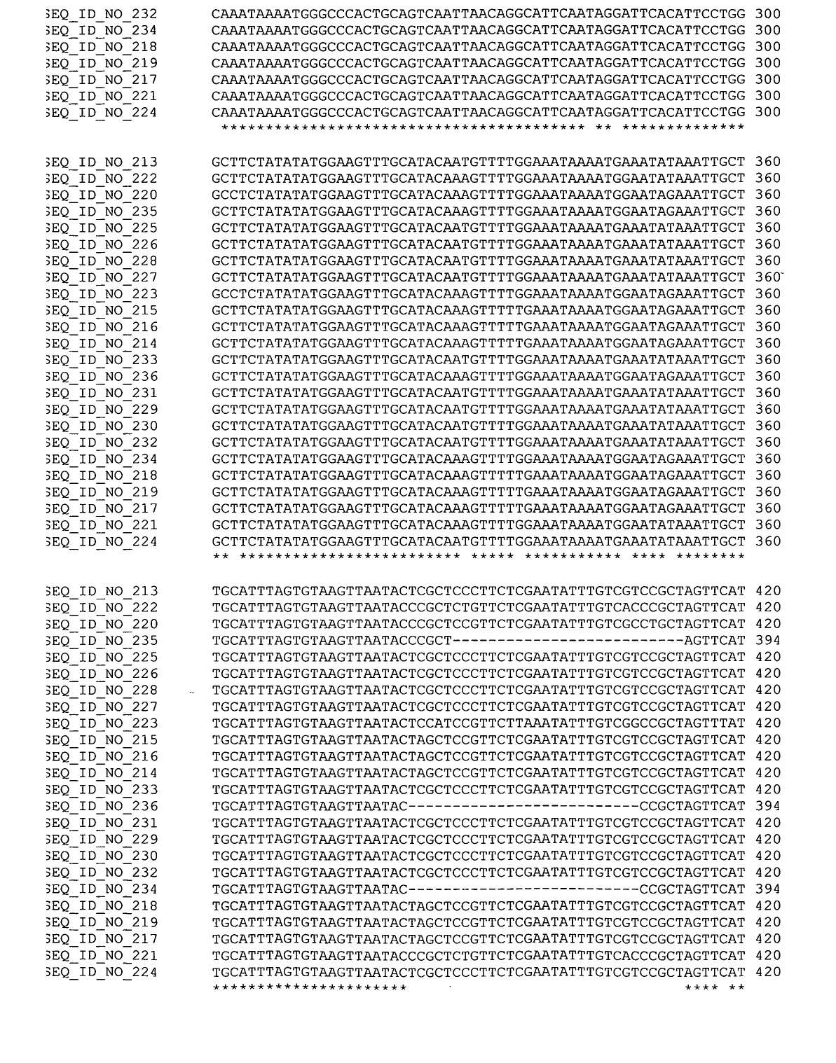

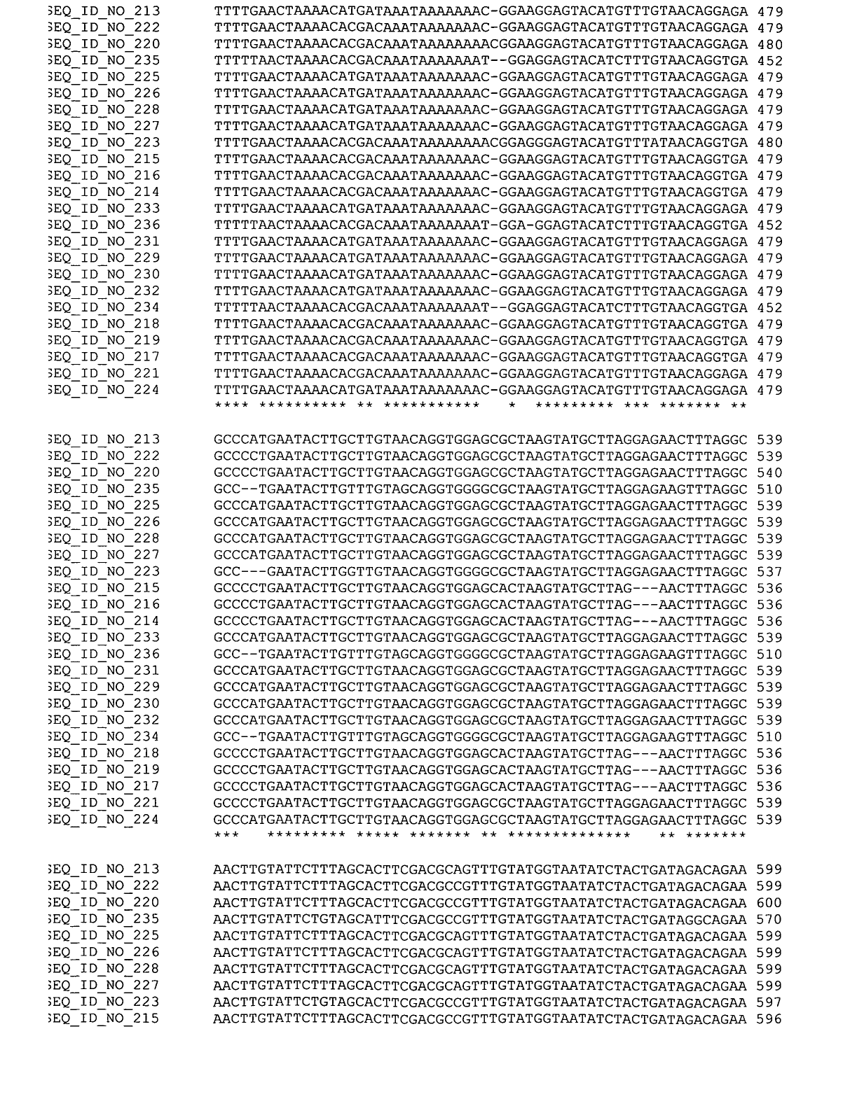

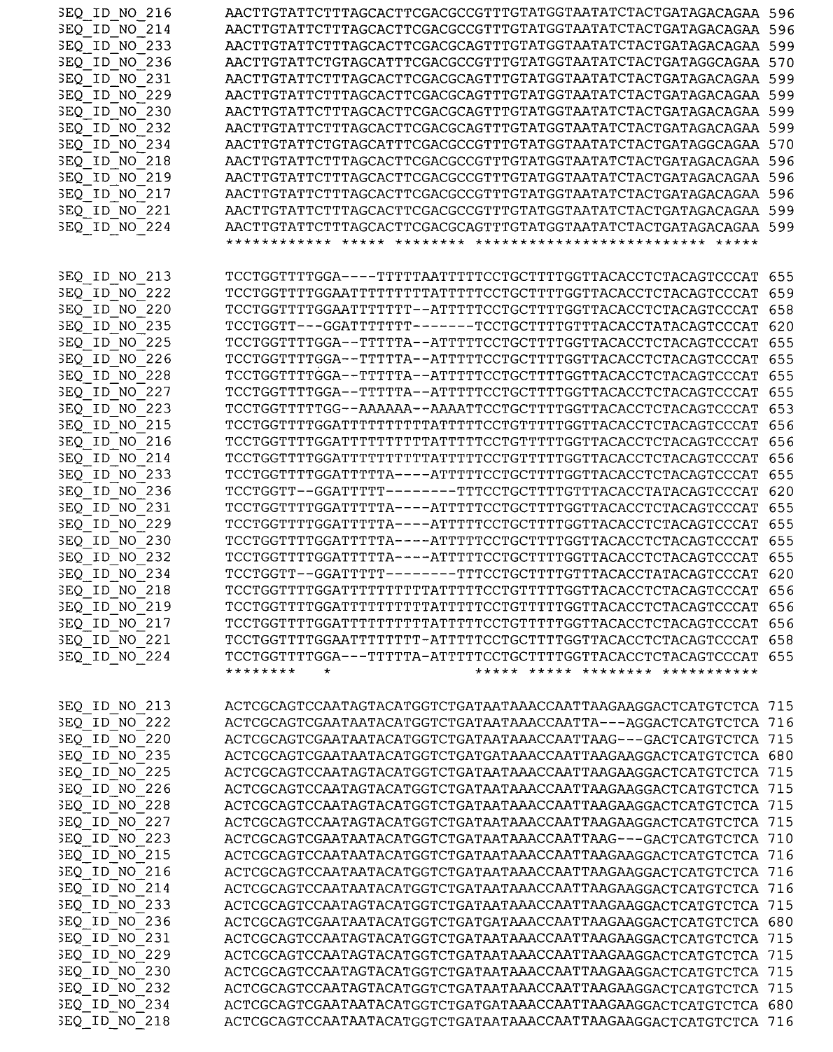

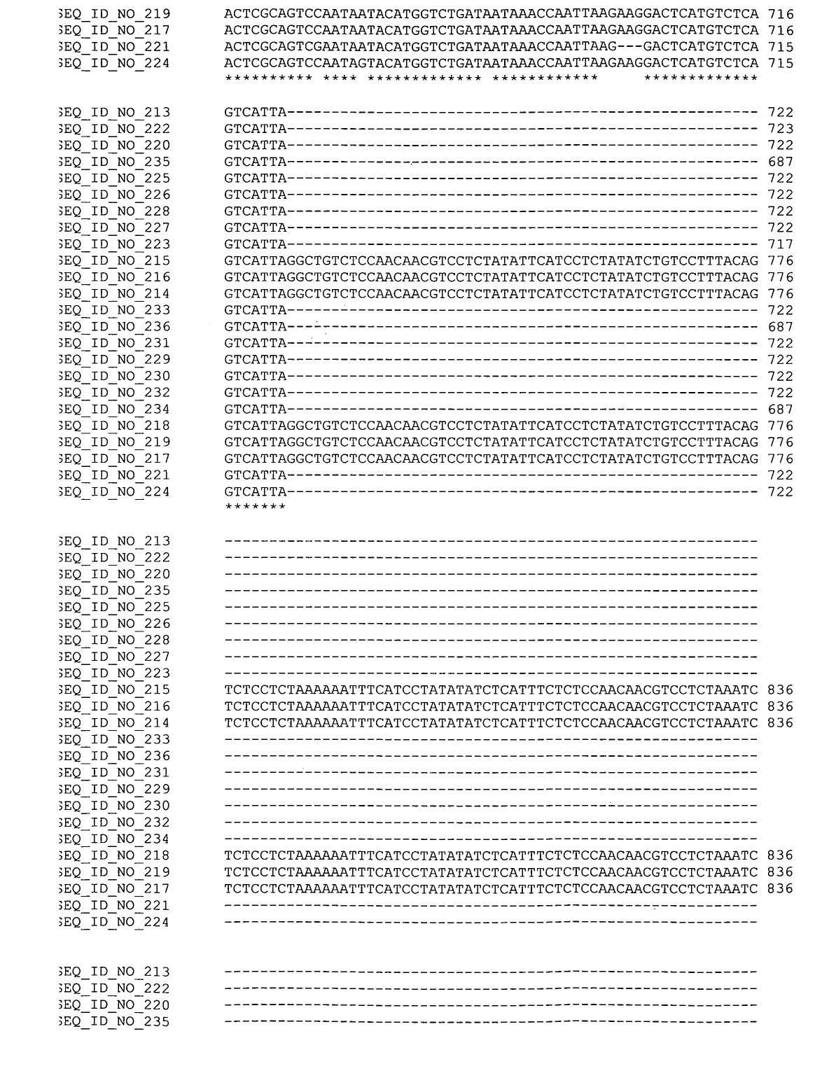

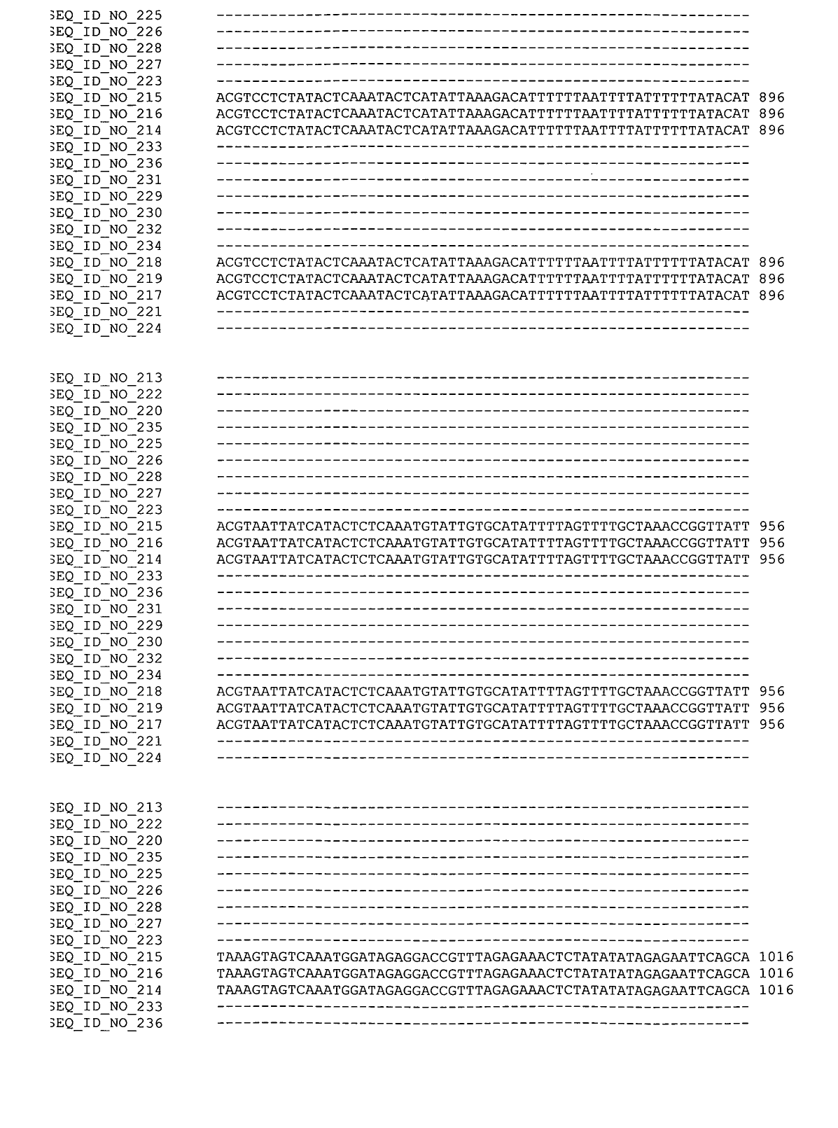

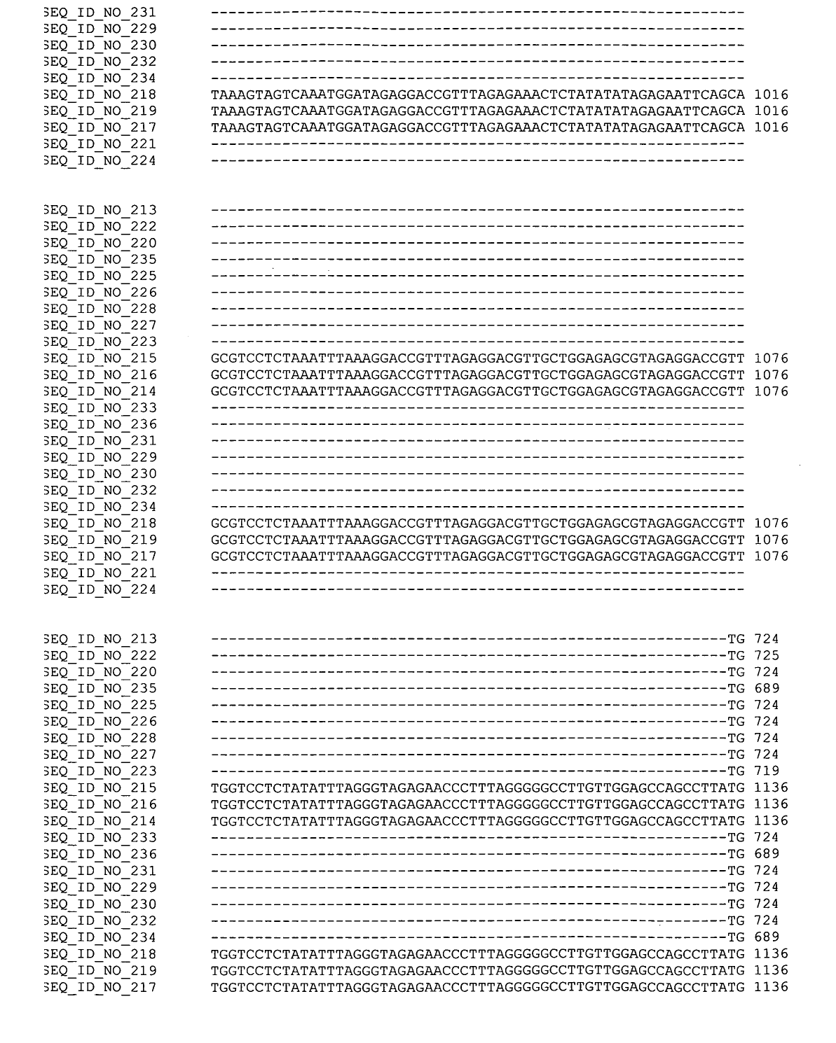

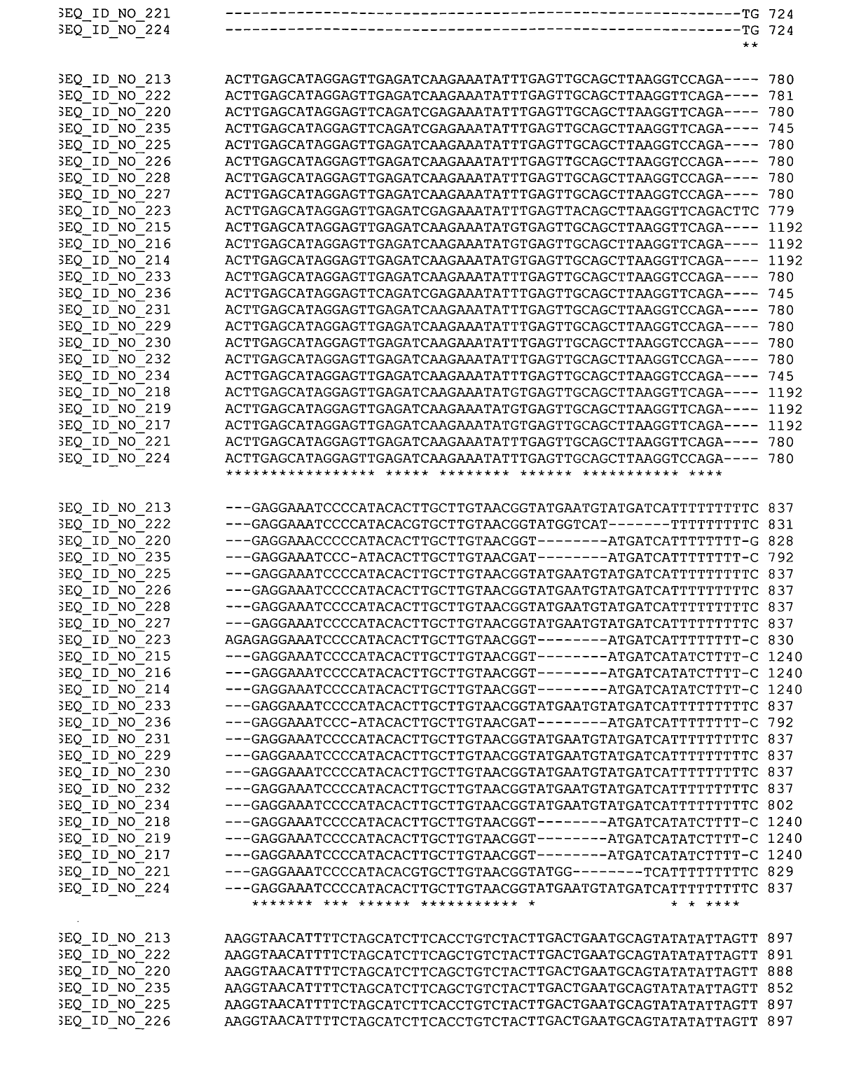

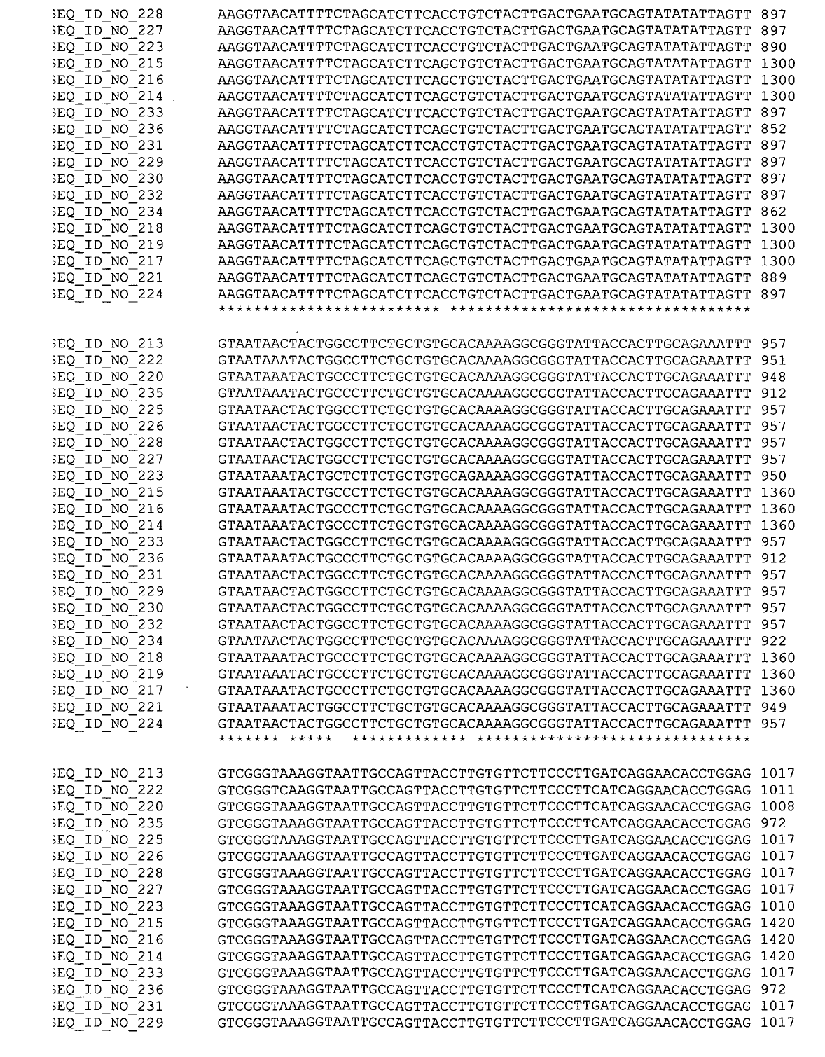

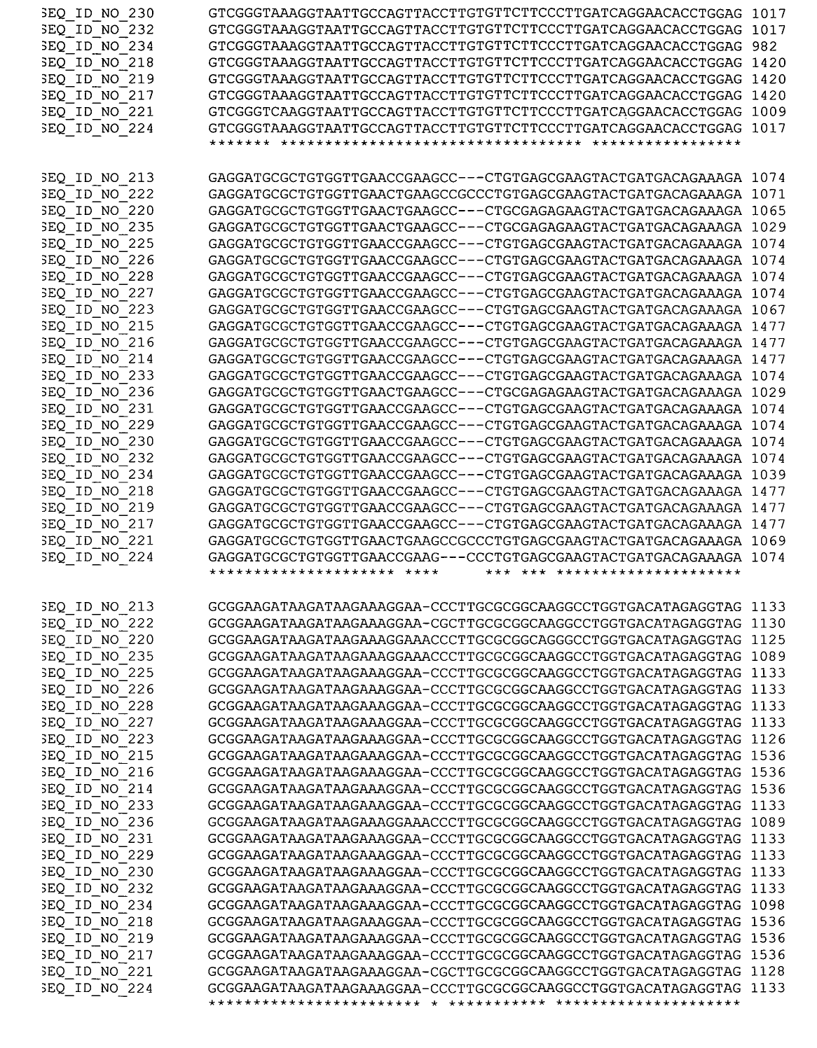

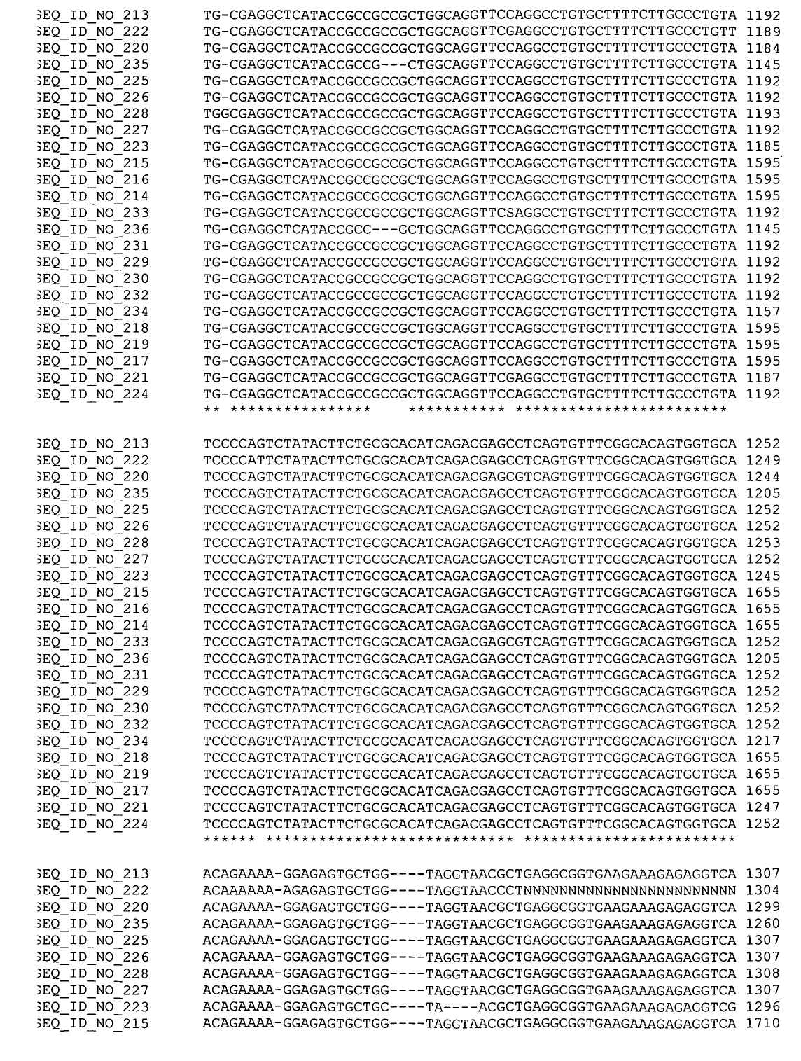

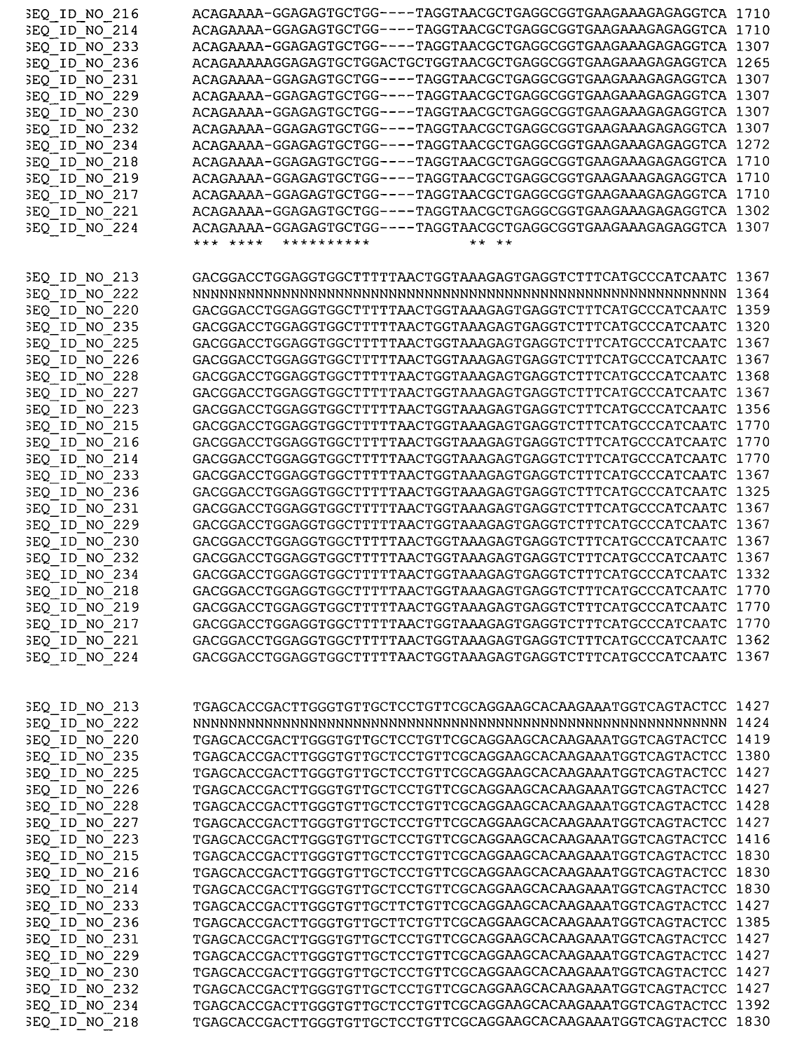

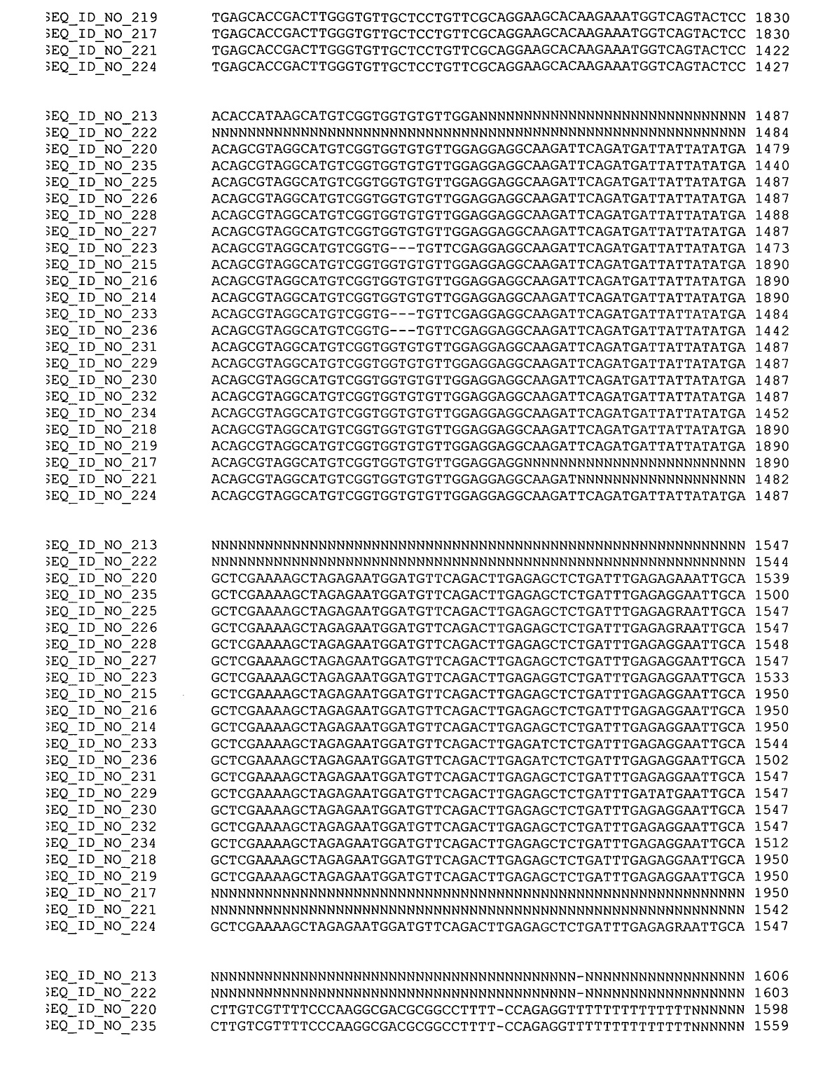

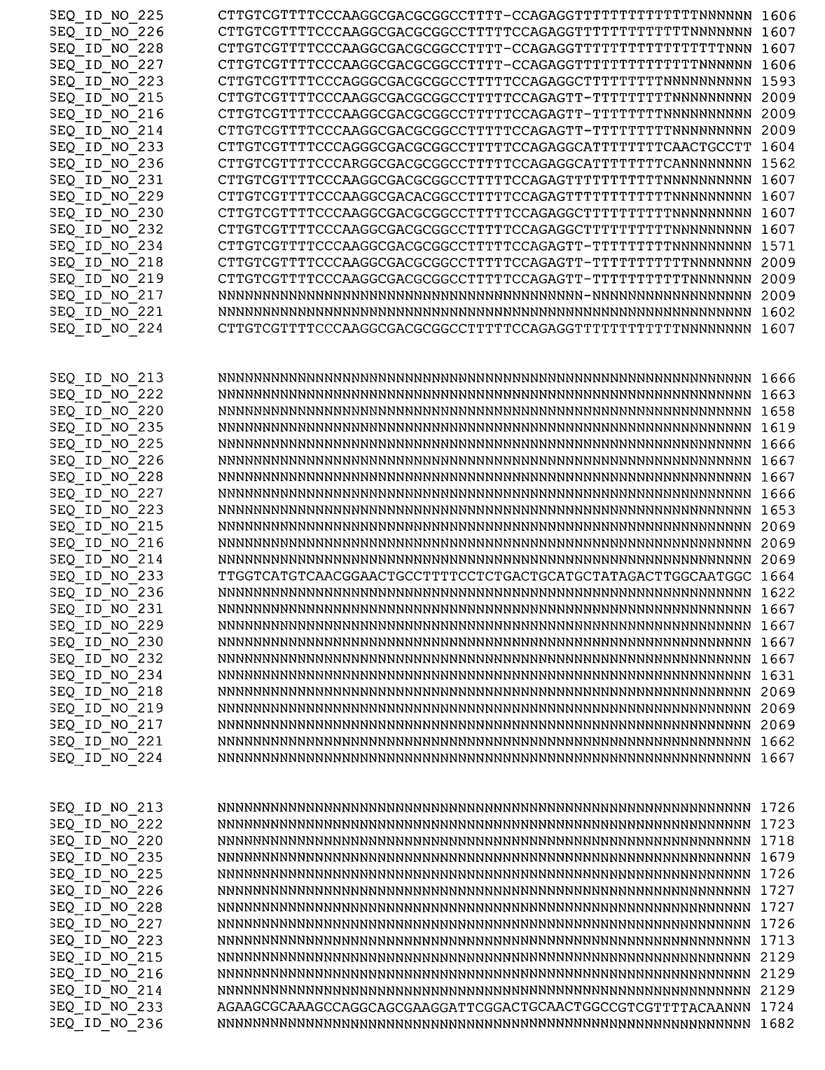

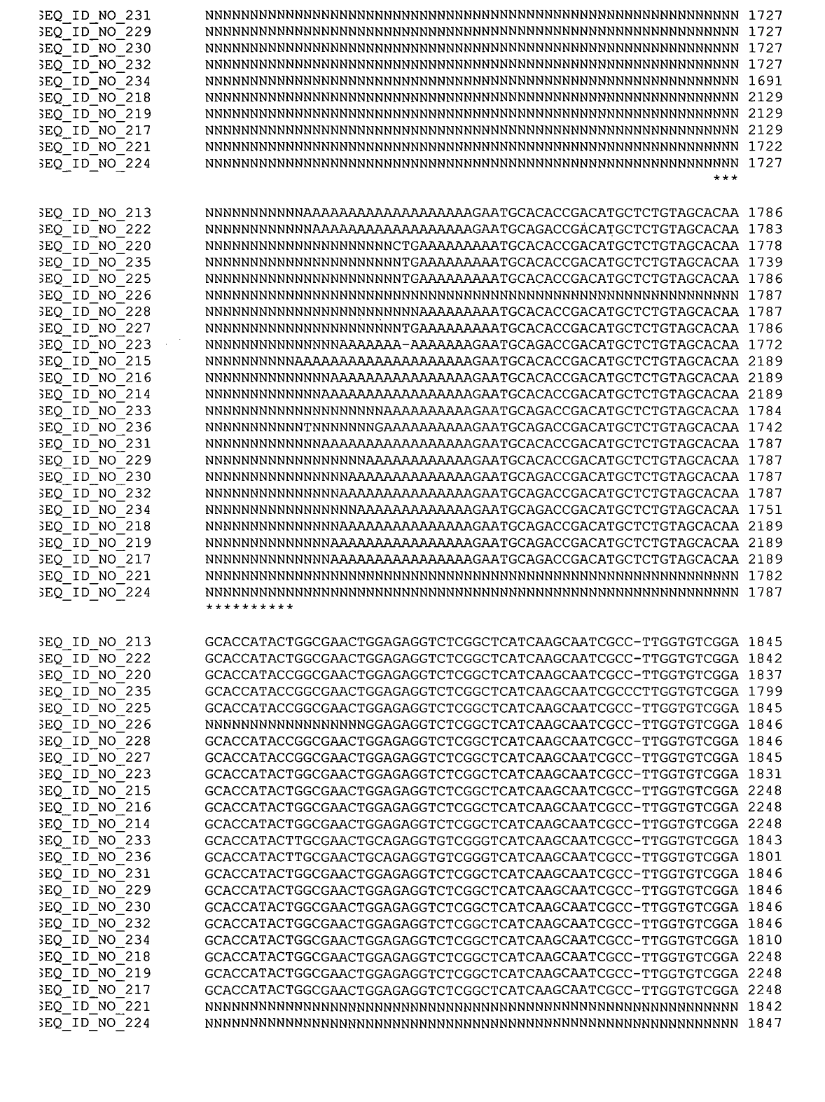

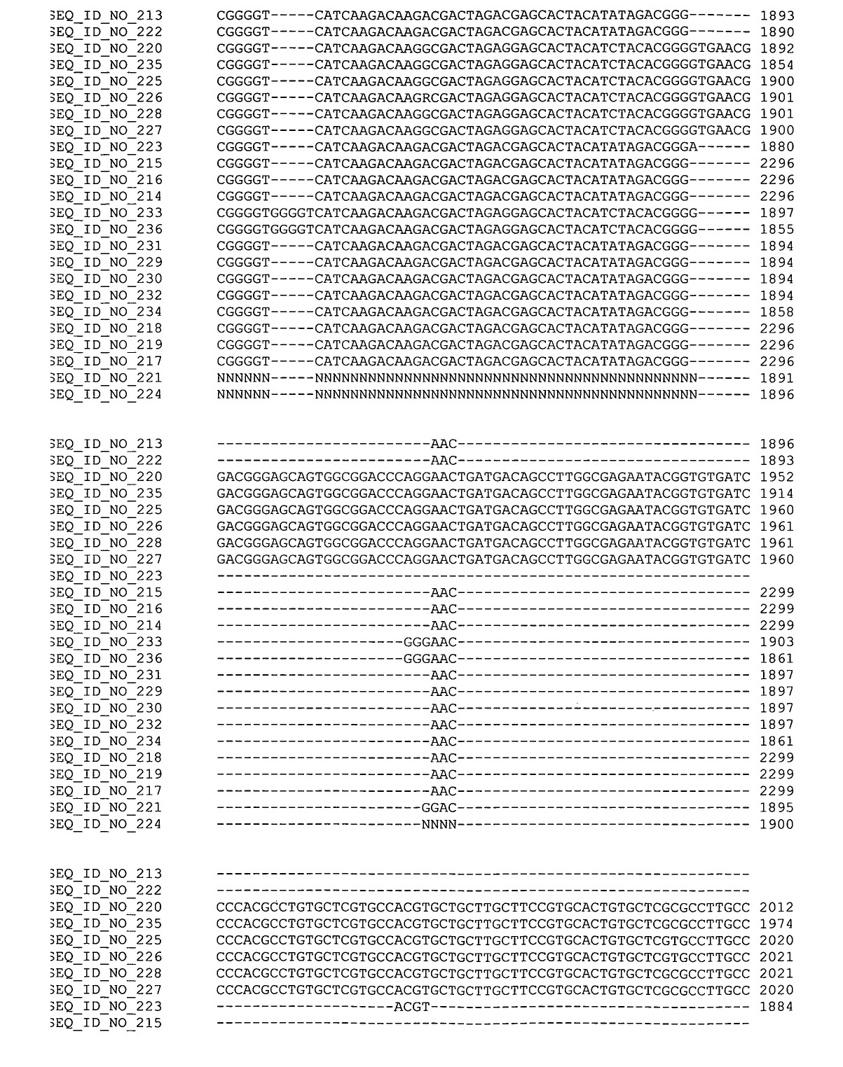

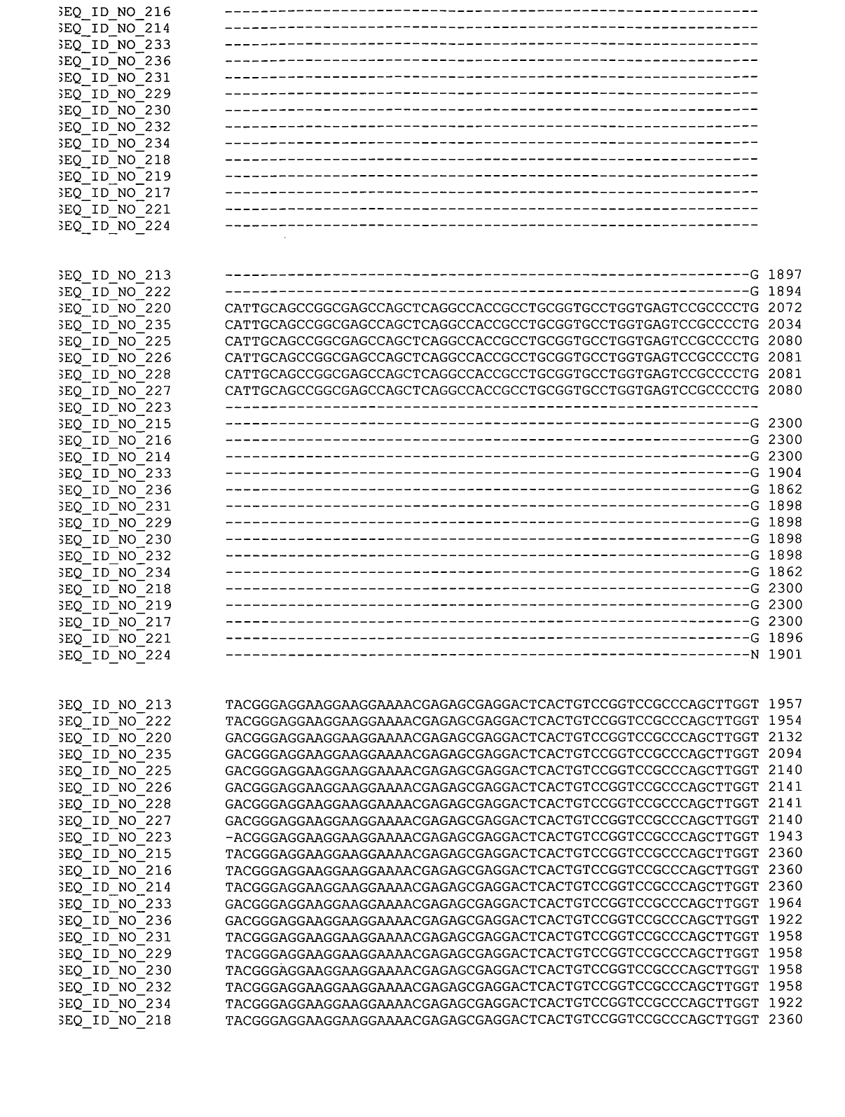

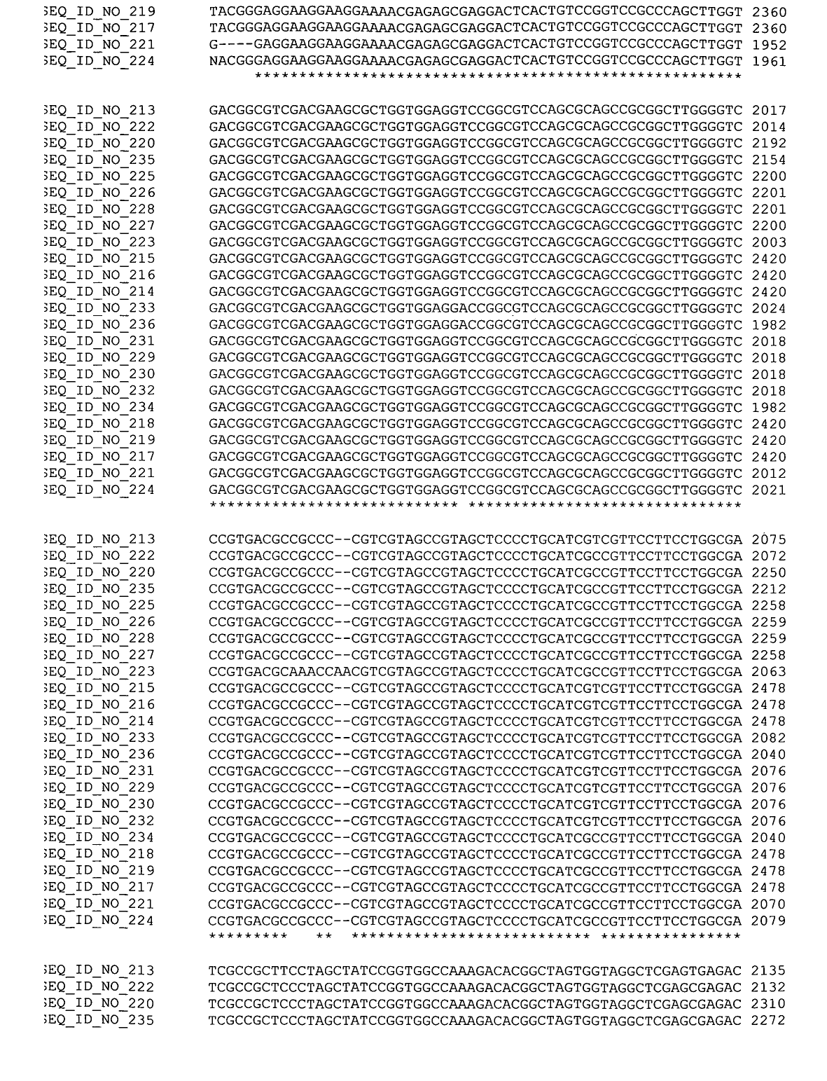

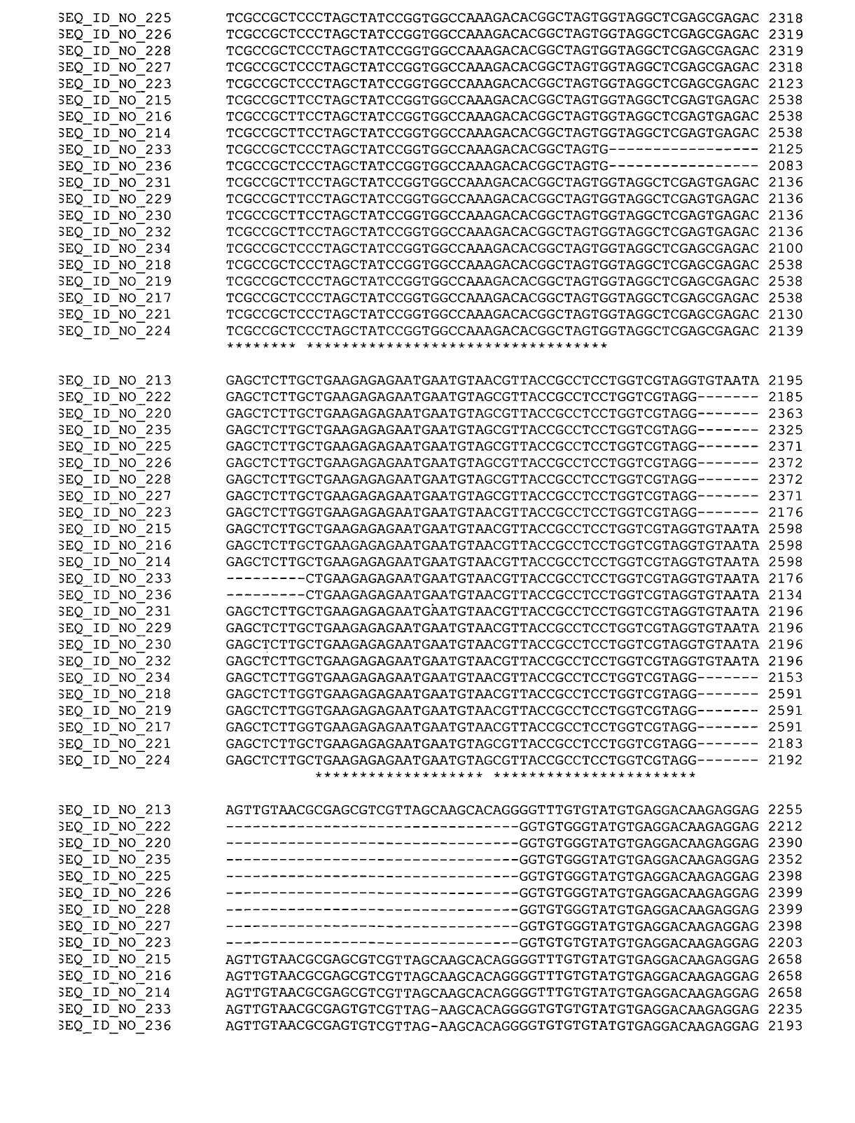

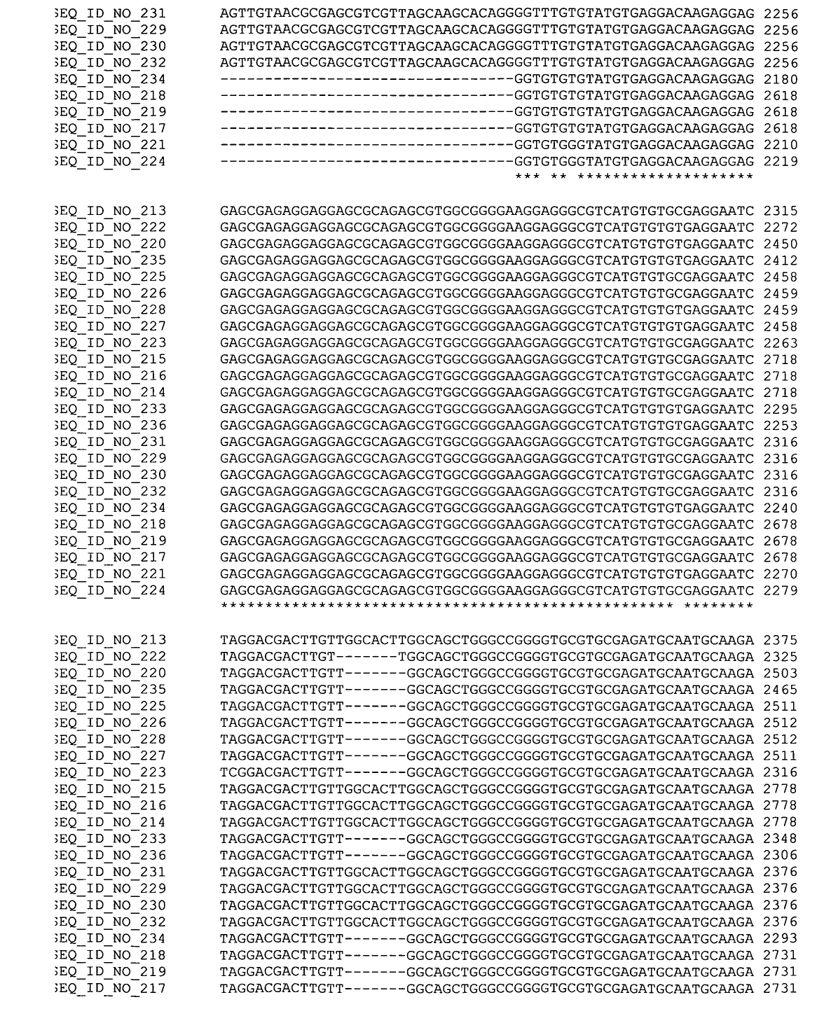

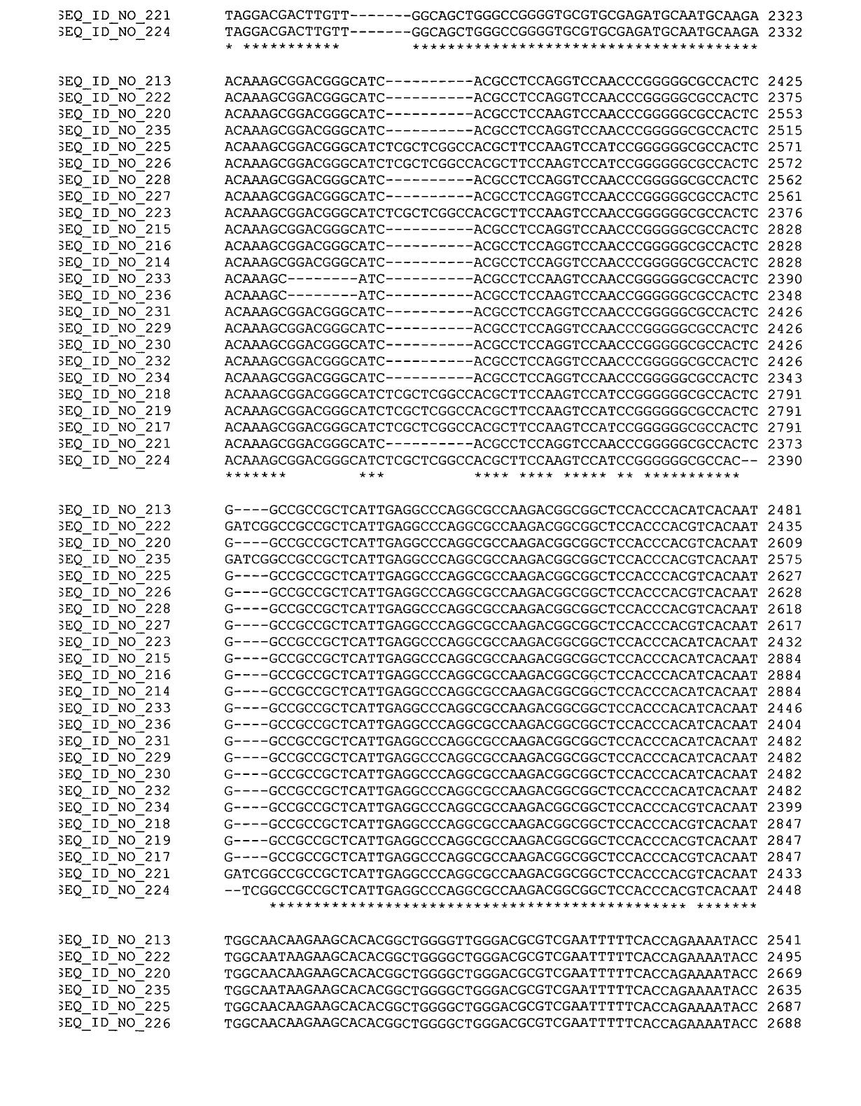

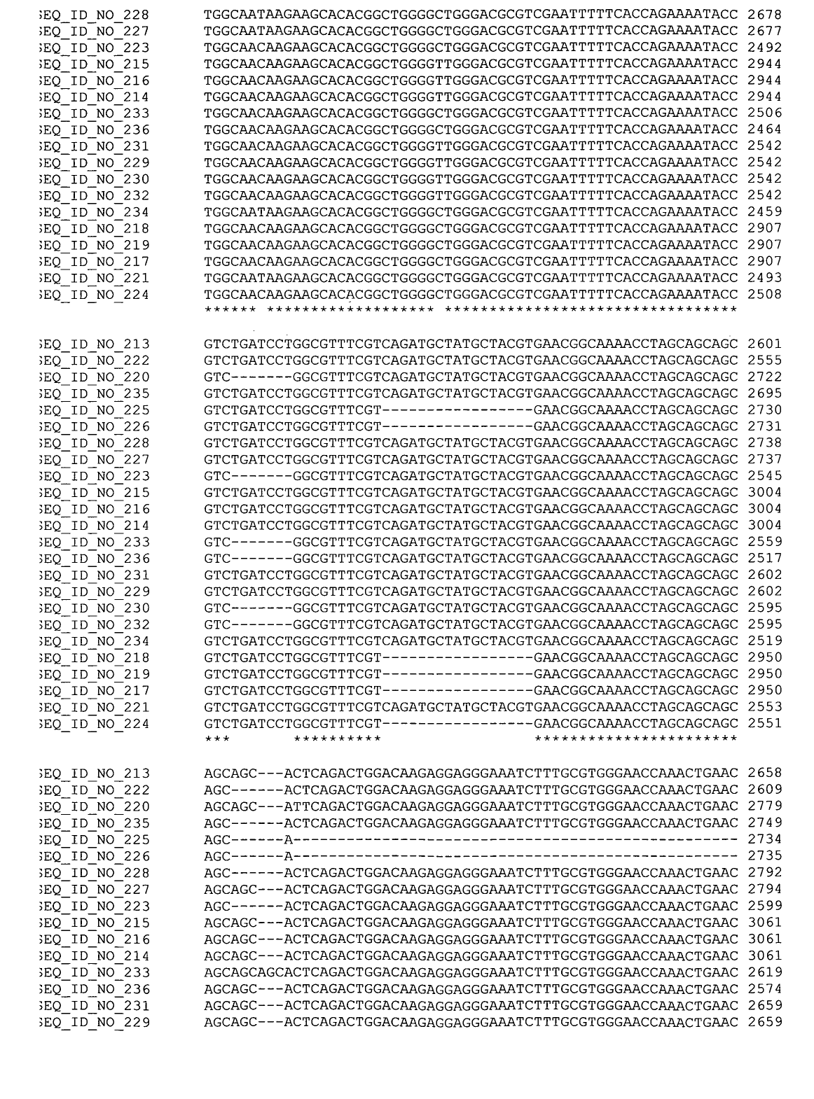

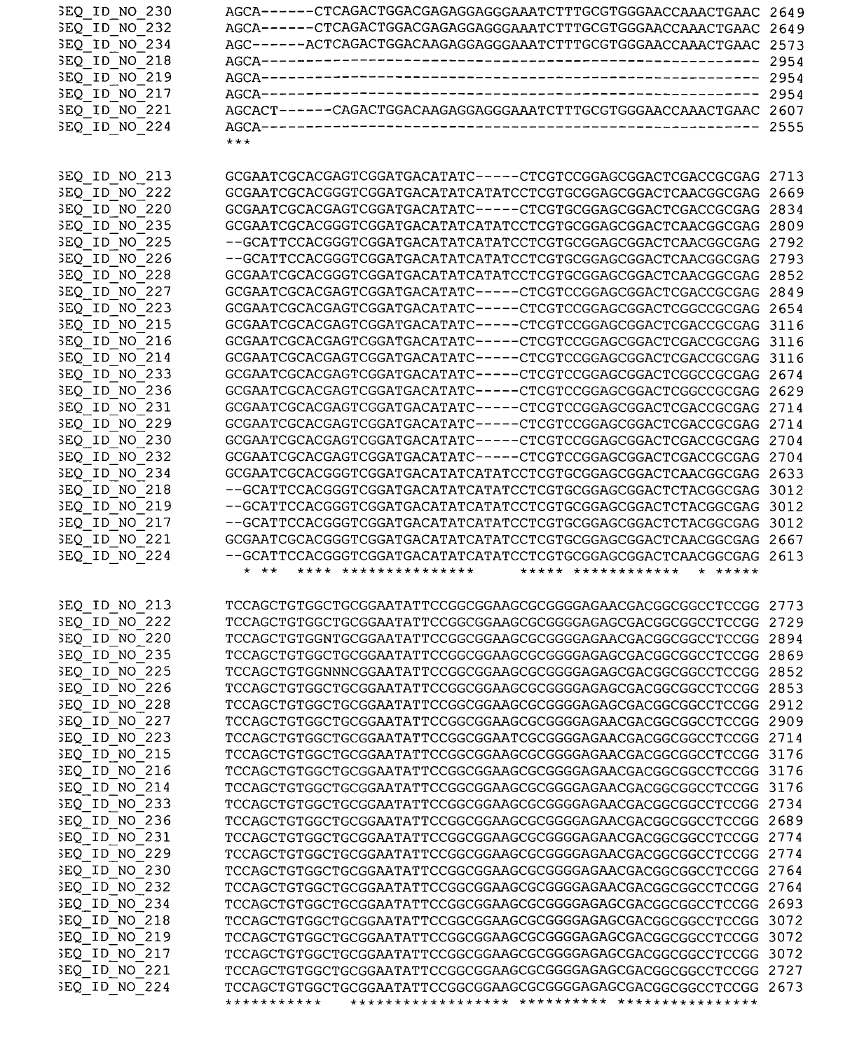

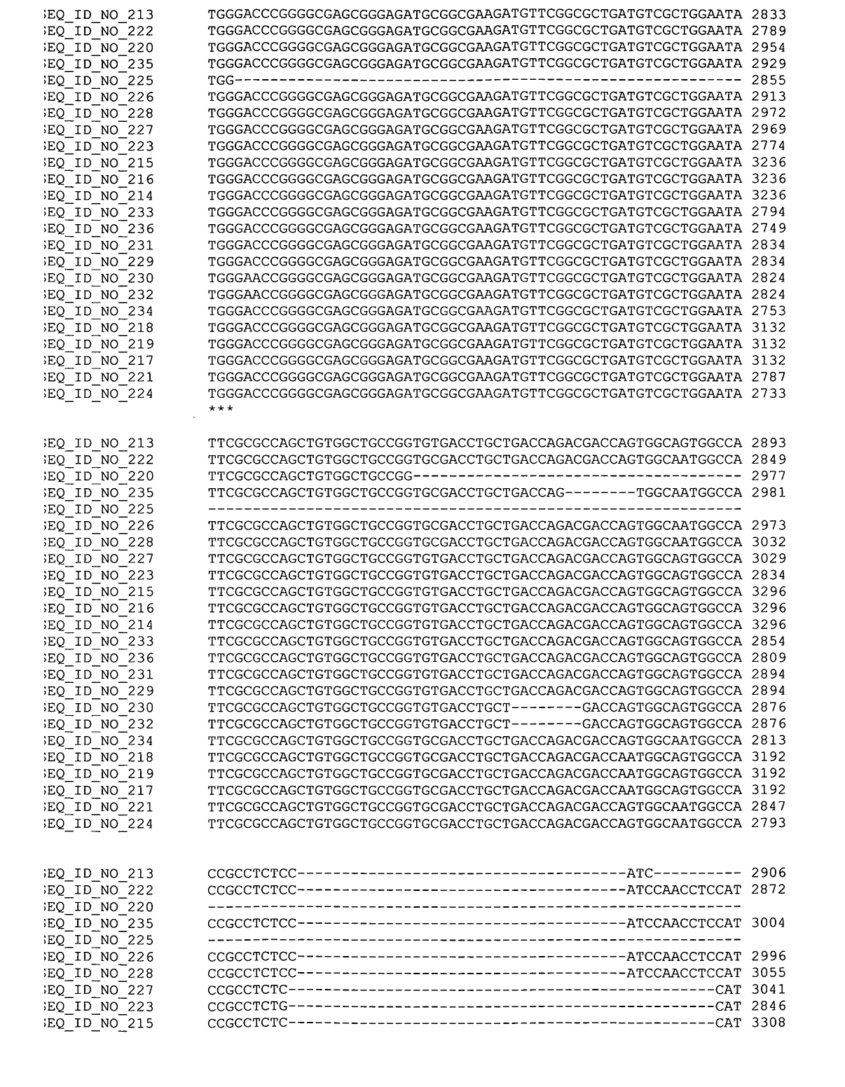

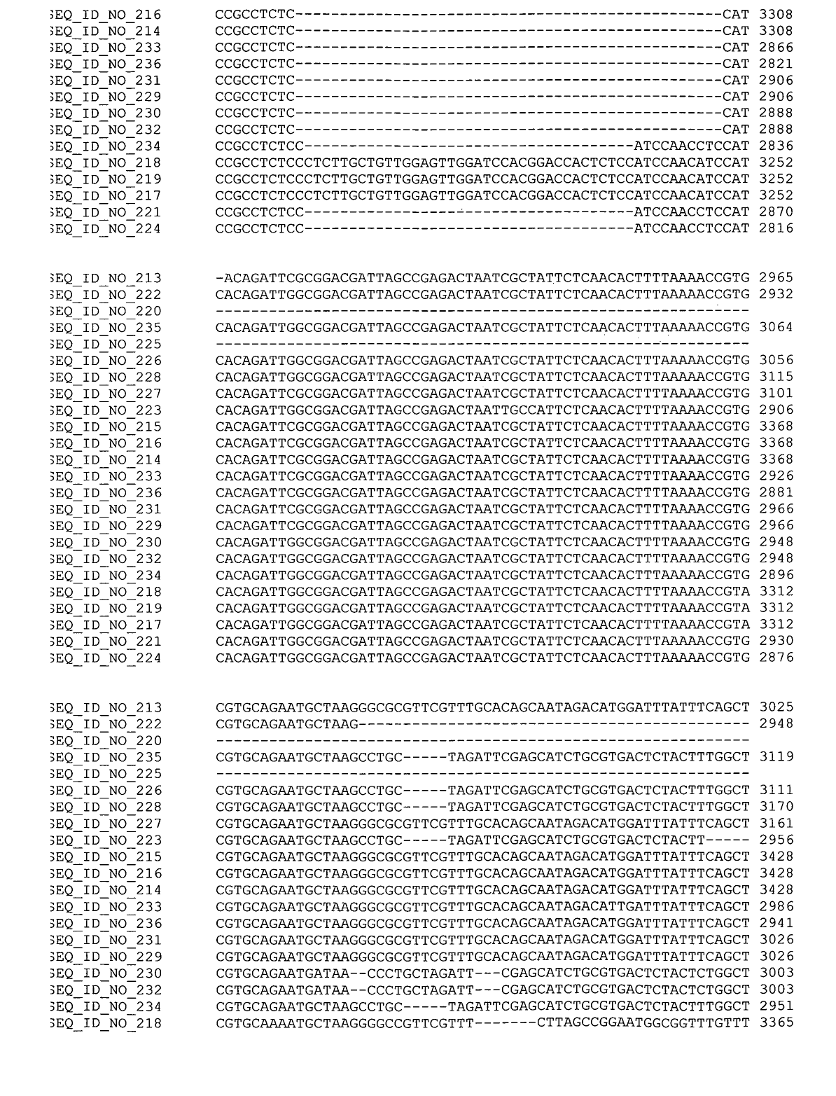

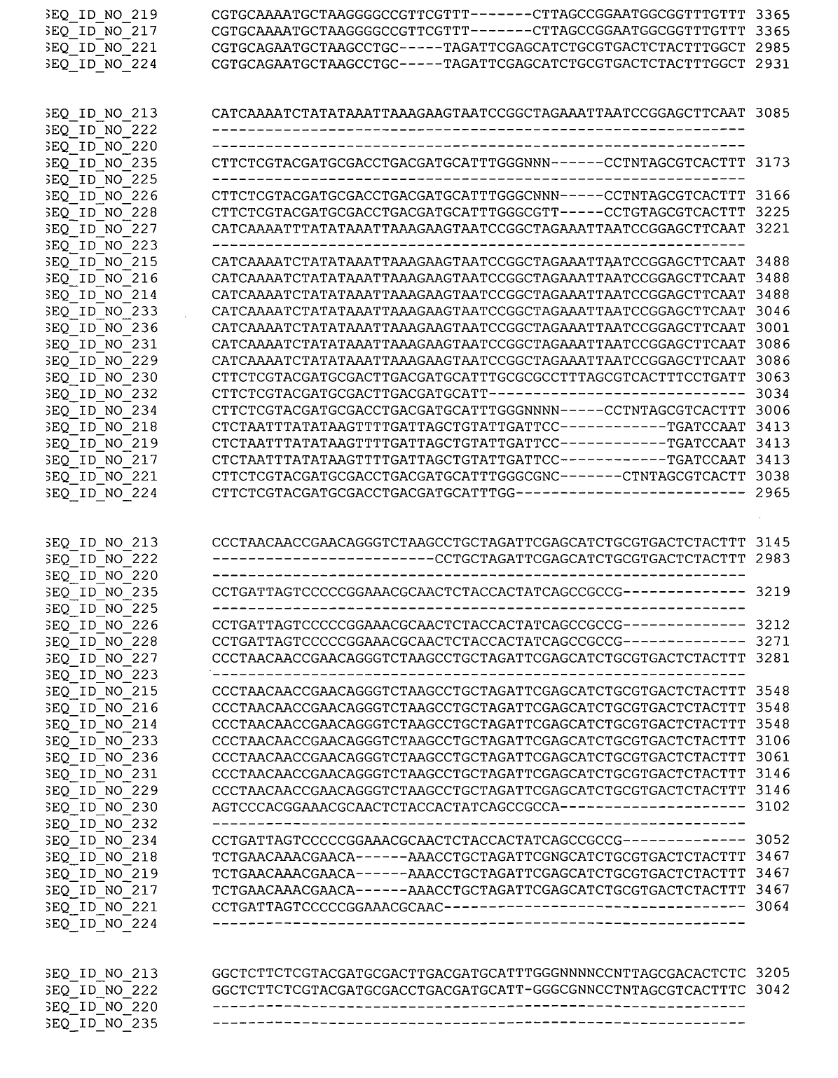

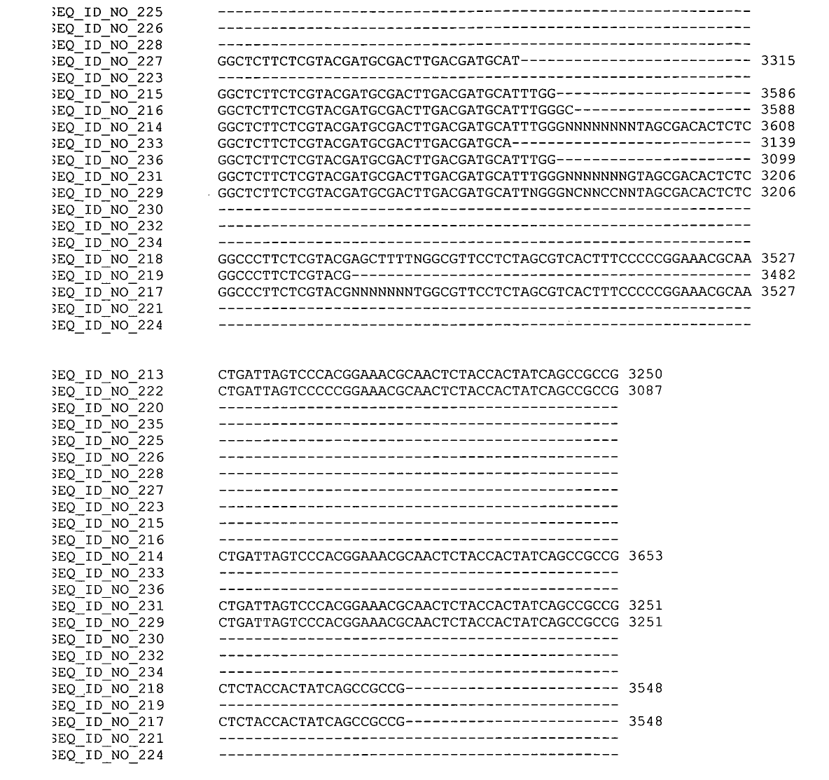

FIG. 9 shows the position of the targeted fragments in the MZA15490 to MZA2038 interval and the position of candidate genes. Sequencing results were obtained for sequences named: MRQV_{—}00005-1; MRQV_{—}1318-1; MRQV_{—}02352-1; MRQV_{—}03828-1; MRQV_{—}06374-1; MRQV_{—}08351-1; MRQV_{—}09551-1-1; MRQV_{—}10673-1 and MRQV_{—}11074-1. The sequences across the group of tester inbreds for the segments MRQV_{—}08351-1 and MRQV_{—}10673-1 are provided herein, including polymorphic SNPs to characterize haploypes (see FIG. 16). The sequence position in the MZA15490 to MZA2038 interval was included in the FIG. 9.

Sequence data was used to identify a putative homologue by descent segments between independent sources of resistance or susceptibility. The region from MRQV_{—}00005-1 to MRQV_{—}08351-1 was shared for most of the independent sources of susceptibility. The data for a key recombinant (from the high resolution mapping population; susceptible to the disease) showed that the recombinant point for this genetic material is located inside a putative Myb transcription factor (PCO644442) and that the sequence variation generating the resistance should be located from the position of this candidate gene towards MZA2038. There was also an expected IBD (identity-by-descent) relationship between independent sources of resistance at the region of or close to PCO644442 as:

a) PHR33 and PH467.

b) PHR33, PH9TJ, PHJ40 and PHDG9.

c) PHK09 showed a specific haplotype.

d) 630 showed a specific haplotype.

There was a group of target SNPs at MRQV_{—}08351-1 very specific for most of resistant sources. However, recombinant data indicates that the target sequence should be located from MRQV_{—}08351-1 (located at PCO644442) towards MZA2038.

Considering the specificity of target SNPs at MRQV_{—}08351-1, this specific fragment was sequenced in a total of 625 inbreds from Pioneer germplasm. A genetic description was developed in relationship to MRCV resistance of part of Pioneer germplasm by using the combined information from: a) flanking markers of this interval (MZA15490 and MZA2038), b) the sequence data for the 625 inbreds and the tester's lines, c) the pedigree relationship between inbreds, and d) the phenotypic data for these inbreds.

A specific group of haplotypes at MRQV_{—}08351-1 or combined with haplotypic information for MRQV_{—}10673-1 and MZA2038 was used to increase the characterization and identity by descent information for the major resistance sources in Pioneer germplasm and to consider putative variants of the target region. Table 27 shows a description of specific haplotypes and the observed and expected response to the disease across materials by haplotype. The representative sources are included as reference.

TABLE 27

| | | | Expected | | | | Segregation |
|---|---|---|---|---|---|---|---|
| MRQV_8381 | MRQV_10673 | MZA2038 link | phenotype | MRCVSC | n | Source | data |
| 1 | 1, 2, 8, 9 | 4, 5, 9, 11 | Susceptible | 3.02 | 247 | PHFV5, 274, PHAN0, | |
| | | | | | | 165, OH7, other | |
| 1 | 3 | 5 | Resistant | 4.60 | 5 | PHJ40 | No |
| 2 | 1 | 12 | Resistant | 4.59 | 22 | PH7WT, 173, 630, | Yes |
| | | | | | | PHB04, PH14J, PHAA4, | |
| | | | | | | PHG64 | |
| 3 | 6 | 1, 14 | Susceptible | 3.21 | 19 | C103, 157, other | |
| 4 | | 4 | Susceptible | 3.31 | 13 | 216, other | |
| 5 | 4 | 4, 11 | Resistant | 5.00 | 3 | PHR33, PH467, 501 | No |
| | | | | | | LACAUNEOP | |
| 7 | 3 | 10, 15 | Resistant | 5.90 | 10 | PHP51, PHDG9, 546, | Yes |
| | | | | | | LACAUNEOP | |
| 9 | 7 | 6 | Resistant | 5.00 | 2 | PHK09, PH884, PHBD6, | Yes |
| | | | | | | PHFCF | |

Using the information for MRQV_{—}08351-1 or combined with flanking sequences (MRQV_{—}10673-1 and MZA2038), Applicants inferred the following:
a) Resistance source 1. The sources PHR33 and PH467 may share a common ancestor at MRQV_{—}08351-1. Shared regions with European materials derived from LACAUNE open pollinated variety support a probable common origin from a single haplotype region.
b) Resistance source 2. The sources PHR33, PH9TJ, PHJ40 and PHDG9 may share a common ancestor at the flanking region of MRQV_{—}08351-1. In addition, PHP51 may be inferred as part of this group. Shared regions with European materials derived from LACAUNE open pollinated variety support a probable common origin from a single haplotype region.
c) Resistance source 3. PHK09 showed a shared haplotype with PHBD6 at MRQV_{—}08351-1, and they should share a common origin from Tuxpen germplasm.
d) Resistance source 4. 630 showed a specific haplotype, and there is not a confirmed IBD relationship with other sources.
   From mapping population results, Applicants thus demonstrate a QTL at the region of preferred markers in these independent sources:

630.

PH9TJ. Allelic to 630.

PHP51. Allelic to 630.

PHBD6. Allelic to 630.

The integration of recombination, sequence, and pedigree analysis and the inference of an expected IBD relationship between independent sources permitted Applicants to consider that four major haplotypes at the region of two of the preferred markers (MZA15490 and MZA2038) can be used to characterize most of the sources of resistance in Pioneer germplasm. These four major haplotypes maybe grouped as these germplasm sources:
(a) Resistance source 1 and 2. Flint SWAN germplasm sharing homologue region with materials from the European flint LACAUNE open pollinated population.
(b) Resistance source 3. Materials from TUXPEN origin.
(c) Resistance source 4. Specific source, 630 is the representative inbred. The development of this inbred included a broad genetic base including TUXPEN and MEXICAN JUNE germplasm.

PCO644442 (FIG. 8, a putative Myb transcription factor) appears to be the likeliest candidate gene for the resistance to MRCV disease. Sequences closely linked to PCO644442 should be also considered as targets for gene cloning, including the putative EPSIN1 and flanking sequences of the interval MZA11826 to MZA9105.

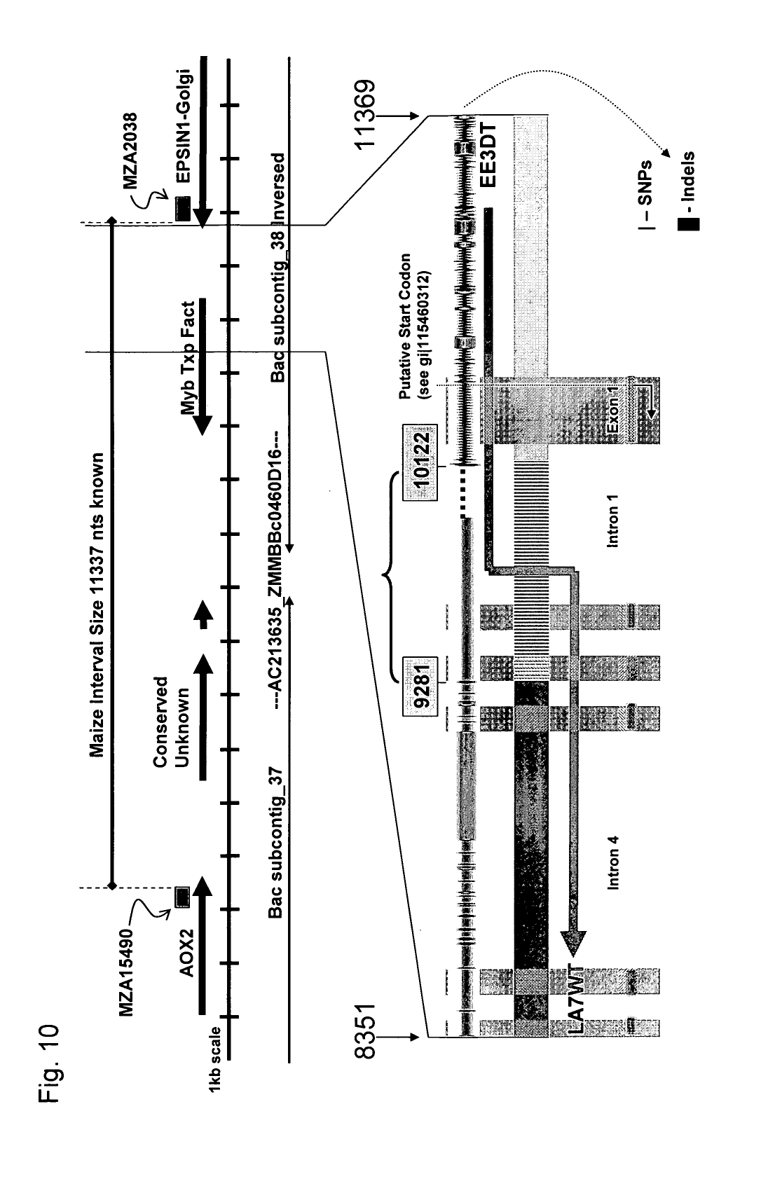

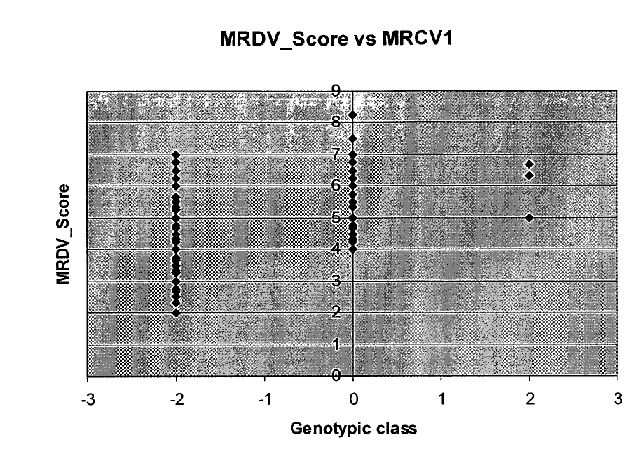

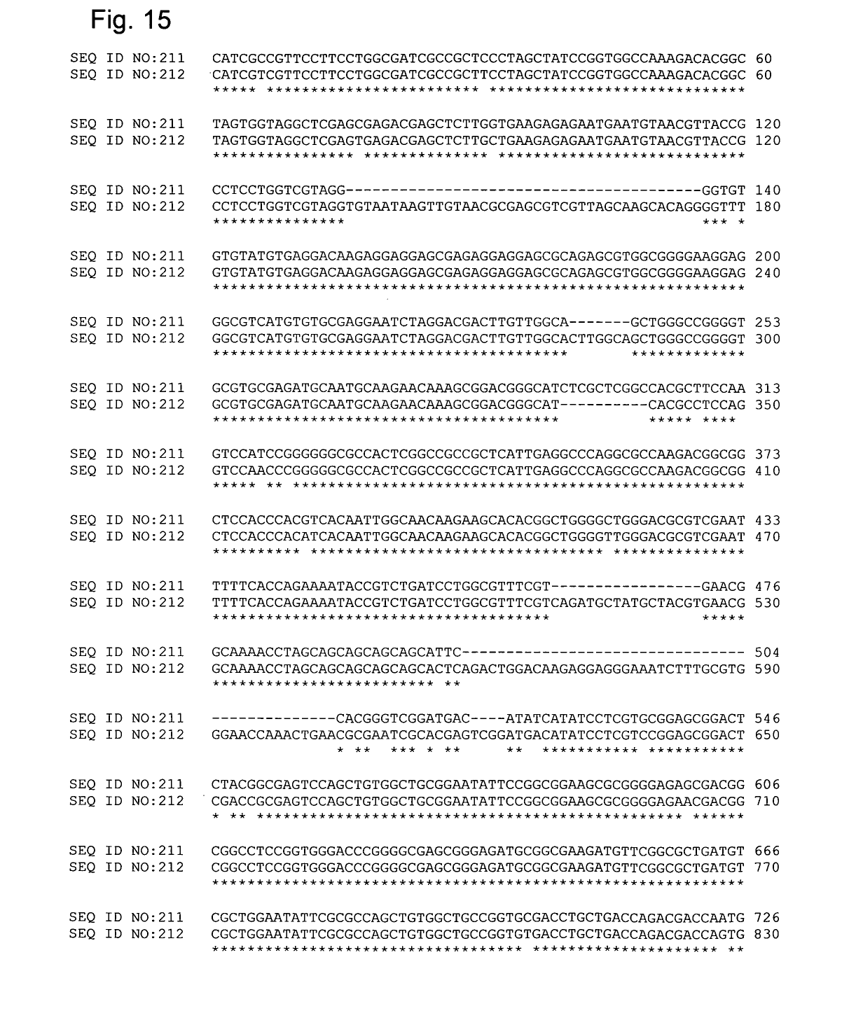

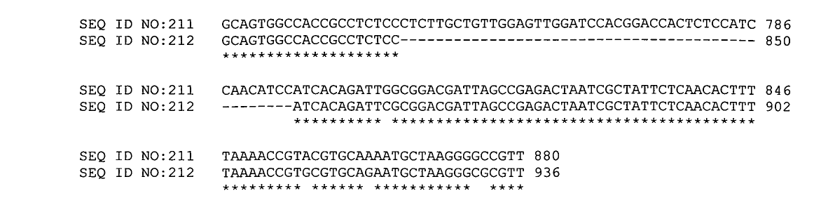

A single recombinant at MZA15490 to MZA2038 from the cross PH3DT and PH7WT was characterized and the recombination point was located inside the PCO644442. The region from intron 3 of PCO644442 to the PCO644442's promoter sequences are considered key targets for the validation of effects on variations on resistance/susceptibility responses across genotypes. FIG. 10 shows the characterization of the recombinant at MZA15490 to MZA2038; a quimeric PCO644442 was originated from PH3DT and PH7WT genotypes. The sequences at promoter region of PCO644442 of PH3DT (SEQ ID NO:212) and PH7WT (SEQ ID NO:211) are included herein, showing polymorphic sites (see FIG. 15 for sequence alignment).

### Example 9

### MRDV-Main Hybrids Characterization-Europe

A set of key European genetic materials was phenotypically and genetically characterized to confirm maize genetic marker loci associated with resistance to MRDV. By identifying such genetic markers, marker assisted selection (MAS) can be used to improve the efficiency of breeding for improved resistance of maize to MRDV.

### Maize Hybrids and Resistance Scoring

The plant varieties used in the analysis were from diverse sources, including elite germplasm, commercially released cultivars and pre-commercial hybrids representing a broad range of germplasm related to a European breeding program.

The groups of maize hybrids were planted in a field experiment in Spain. The classifications of resistance and susceptible were based solely on observations of fortuitous, naturally occurring disease incidence in field tests. The degree of plant resistance to MRDV infection varied widely, as measured using a scale of incidence of MRDV symptoms.

Data collection was typically done in one scoring time. Scoring time is placed after flowering time.

In assessing association of markers to resistance, a comparison by using the IBD information of parent lines was used. Allele origin was checked by the identity by descent approach. Using this approach, those maize lines that were considered to be representative of either the genotypic classes were used for assessing association and predict performance at hybrid level.

### Maize Genotyping

Each parent line of these hybrids has been genotyped and IBD calculations have been estimated for each line.

The underlying logic is that markers with significantly different allele distributions between the resistant and susceptible groups (i.e., non-random distributions) might be associated with the trait and can be used to separate them for purposes of marker assisted selection of maize lines with previously uncharacterized or characterized resistance or susceptibility to MRDV. The present analysis examined the IBD information at the genetic position of the region of preferred markers and determined if the allele distribution within the resistant group is significantly different from the allele distribution within the susceptible group. This analysis compares the plants' phenotypic score with the genotypes at the target loci; the genotypes were predicted by IBD.

### Results

In order to evaluate the effect of the allelic variation at this QTL at the hybrid level, a set of 212 hybrids (heterogenous genetic backgrounds) was characterized according to the presence of one (heterozygous for the QTL) or two resistant alleles (homozygous for the QTL) from the parent lines. A positive and additive effect of the resistant allele at the major QTL was observed on the hybrid combinations. Table 28 shows the field performance of hybrids with different genotypes at the major QTL. The field performance was characterized as MRDV_score, similar protocol to MRCV score.

TABLE 28

| | | Average of | |
|---|---|---|---|
| Hybrid genotype at major QTL | # hybrids | MRDV_score | STD Dev |
| AA, homozygous susceptible | 163 | 4.25 | 0.92 |
| allele | | | |
| BA, heterozygous, female | 37 | 5.45 | 1.01 |
| resistant allele | | | |
| BB, homozygous resistant allele | 3 | 6.00 | 0.88 |

FIG. 11 shows the performance of maize hybrids under MRDV infection. The field performance expressed as MRDV_score.

### Discussion/Conclusions

This example has identified chromosome intervals that correlate with MRDV resistance. Markers that lie within these intervals are useful for MAS, as well as other purposes. The prediction of MRDV increased resistance by using the preferred markers for MRCV resistance indicates that these markers may be used for MAS for different Fijivirus. A positive effect of the preferred markers for resistance to other Fijivirus, such as rice black-streaked dwarf fijivirus, is thus expected.

### Example 10

### MRCV Resistance Phenotypic Assay

What: A 1-9 score of Mal de Río Cuarto Virus with 1 meaning no resistance (stunted, internodes shortening, no ear), and 9 meaning that the genetic material is resistance to the disease (no symptoms).
   When: From flowering through harvest
   Check scores of known susceptible lines to see if their present ratings agree with historical ratings.
How:
   1. Compare ratings that you would give a few known susceptible lines today with their historical ratings to see if they agree.
   2. If the ratings are too high, then there is not enough disease pressure to score this location. However, note any plot that has more disease than the susceptible checks.
   3. Score on a plot basis. Plants within a plot may vary in symptoms due to timing of infection or some plants may escape to the disease (natural infection depends on population of vectors).
   4. Consider severity of symptoms and frequency of plants with symptoms. Scores of 1-3 are in susceptible category; 4-6 are in resistant category; 7-9 are in highly resistant category.

Description of Field Scores:

a) Scores 1-3. Susceptible category. Symptoms include severe dwarfism, severe internodes shortening, no ears or very poor ear development, premature dead of plants.
b) Scores 4-6. Resistant category. Plants with symptoms as enations and soft internodes shortening. Low frequency of plants with severe symptoms.
c) Scores 7-9. Highly resistant category. Healthy plant. Presence of enations or no symptoms.

What is claimed is:

1. A method of identifying a first maize plant or germplasm that displays newly conferred resistance or enhanced resistance to a Fijivirus, the method comprising detecting in the first maize plant or germplasm at least one allele of a first marker locus that is associated with the newly conferred resistance or enhanced resistance, wherein the first marker locus localizes within a chromosome interval flanked by and including MZA8381 and MZA18180.

2. The method of claim 1, wherein the first marker locus localizes within a chromosomal interval flanked by and including MZA4305 and MZA2803.

3. The method of claim 1, wherein the first marker locus localizes within a chromosomal interval flanked by and including MZA15490 and MZA2038.

4. The method of claim 1, wherein first marker locus is MZA625, MZA16656, MZA15451, MZA15490, MZA2038, MZA11826, or MZA9105.

5. The method of claim 1, wherein the newly conferred resistance or enhanced resistance is assayed by a phenotypic field score.

6. The method of claim 1, wherein the at least one allele comprises MZA625, MZA16656, MZA15451, MZA15490, MZA2038, MZA11826, or MZA9105.

7. The method of claim 1, wherein the first maize plant has the haplotype
G at MZA16656-19-A
G at MZA15490-801-A
C at MZA15490-137-A
A at MZA2038-71-A
T at MZA2038-76-A
A at MZA11826-801-A
C at MZA11826-803-A
A at MZA9105-8-A
and displays newly conferred resistance or enhanced resistance to a Fijivirus as compared to an isogenic maize plant lacking said haplotype.

8. The method of claim 1, wherein the at least one allele is correlated with newly conferred resistance or enhanced resistance, the method further comprising introgressing the allele in the first maize plant or germplasm into a second maize plant or germplasm to produce an introgressed maize plant or germplasm.

9. An introgressed maize plant or germplasm produced by the method of claim 8.

10. The introgressed maize plant or germplasm of claim 9, wherein the introgressed maize plant or germplasm comprises MZA625, MZA16656, MZA15451, MZA15490, MZA2038, MZA11826, and MZA9105.

11. The introgressed maize plant or germplasm of claim 9 having the haplotype:
C at MRQV_{—}08351-173
A at MRQV_{—}08351-262
G at MRQV_{—}08351-280
G at MRQV_{—}08351-323
C at MRQV_{—}08351-369
C at MRQV_{—}08351-372.

12. The introgressed maize plant or germplasm of claim 9 comprising SEQ ID NO:49.

13. The method of claim 1, wherein the Fijivirus is Mal de Río Cuarto Virus (MRCV).

14. The method of claim 1, wherein the Fijivirus is Maize Rough Dwarf Virus (MRDV).

15. The method of claim 1, wherein the first maize plant or germplasm has the haplotype:
C at MRQV_{—}08351-173
A at MRQV_{—}08351-262
G at MRQV_{—}08351-280
G at MRQV_{—}08351-323
C at MRQV_{—}08351-369
C at MRQV_{—}08351-372.

16. A method of producing a maize plant having newly conferred resistance or enhanced resistance to a Fijivirus, the method comprising introducing an exogenous nucleic acid into a target maize plant or progeny thereof, wherein the exogenous nucleic acid is derived from a nucleotide sequence that is linked to at least one favorable allele of a marker locus that is associated with newly conferred resistance or enhanced resistance to a Fijivirus, wherein the marker locus localizes within a chromosome interval flanked by and including MZA8381 and MZA18180; whereby the resulting transgenic plant displays newly conferred resistance or enhanced resistance to a Fijivirus.

17. The method of claim 16, wherein the linked marker locus is determined using the mapping population PH7WT×PH3DT or PH9TJ×PH890.

18. A method of selecting at least one maize plant by marker assisted selection of a quantitative trait locus associated with newly conferred resistance or enhanced resistance to a Fijivirus, wherein said quantitative trait locus is localized to a chromosomal interval defined by and including markers MZA8381 and MZA18180 on linkage group 2, said method comprising:
(a) testing at least one marker on said chromosomal interval for said quantitative trait locus; and
(b) selecting said maize plant comprising said quantitative trait locus.

19. A method of identifying a first maize plant or germplasm that displays susceptibility to a Fijivirus, the method comprising detecting in the first maize plant or germplasm at least one allele of a first marker locus that is associated with the susceptibility, wherein the first marker locus localizes within a chromosome interval flanked by and including MZA8381 and MZA18180.

20. The method of claim 19, wherein the first maize plant or germplasm has the haplotype:
T at MRQV_{—}08351-173
T at MRQV_{—}08351-262
A at MRQV_{—}08351-280
C at MRQV_{—}08351-323
T at MRQV_{—}08351-369
T at MRQV_{—}08351-372.
